(19) United States
(12) Patent Application Publication
(10) Pub. No.: US 2023/0348852 A1
(43) Pub. Date: Nov. 2, 2023

(54) METHODS AND COMPOSITIONS FOR OCULAR CELL THERAPY

(71) Applicants: NOVARTIS AG, Basel (CH); INTELLIA THERAPEUTICS, INC., Cambridge, MA (US)

(72) Inventors: Frada BERENSHTEYN, Cambridge, MA (US); Bo HAN, Cambridge, MA (US); Xueshi HAO, San Diego, CA (US); Jessica HEYDER, Cambridge, CA (US); Timothy Z. HOFFMAN, San Diego, CA (US); Qihui JIN, San Diego, CA (US); Arnaud LACOSTE, Cambridge, MA (US); Jun LIU, San Diego, CA (US); Yahu LIU, San Diego, CA (US); Tingting MO, Saratoga, CA (US); Bradley Andrew MURRAY, Cambridge, MA (US); Daniel Joseph O’CONNELL, Cambridge, MA (US); Jianfeng PAN, San Diego, CA (US); Yun Feng XIE, Tiburon, CA (US); Shanshan YAN, San Diego, CA (US); Yefen ZOU, San Diego, CA (US)

(21) Appl. No.: 17/997,223

(22) PCT Filed: Apr. 26, 2021
(86) PCT No.: PCT/IB2021/053413

§ 371 Date: Oct. 26, 2022

Related U.S. Application Data
(60) Provisional application No. 63/015,856, filed on Apr. 27, 2020.

(51) Int. Cl. A61P 27/02; C12N 5/079; C12N 9/22

(52) U.S. Cl. CPC C12N 5/0621; A61P 27/02; C12N 9/22; C12N 2310/20; C12N 2501/999

(57) ABSTRACT

The present invention provides ocular cells, genetically modified by a CRISPR system targeting the expression of B2M for ocular cell therapy. The invention further provides methods of generating an expanded population of genetically modified ocular cells, for example limbal stem cells (LSCs) or corneal endothelial cells (CECs), wherein the cells are expanded involving the use of a LATS inhibitor and the expression of B2M in the cells has been reduced or eliminated. The present invention also provides cell populations, preparations, uses and methods of therapy comprising said cells.

### I. SEQUENCE LISTING

The instant application contains a Sequence Listing which has been submitted electronically in ASCII format and is hereby incorporated by reference in its entirety. Said ASCII copy, created on Apr. 23, 2020, is named PAT058807_sequence_listing_2020_ST25.txt and is 224 KB in size.

### II. FIELD

The present invention relates to methods of generating an expanded population of genetically modified ocular cells, for example limbal stem cells (LSCs) or corneal endothelial cells (CECs), wherein the cells are expanded involving the use of a LATS inhibitor and the expression of B2M in the cells has been reduced or eliminated. The present invention also relates to a population of such modified cells, preparations, uses and methods of therapy comprising said cells.

### III. BACKGROUND

Organ regeneration and/or healing is an issue crucial to treat many serious health issues.

For example in the eye, it is known that corneal blindness is the third leading cause of blindness worldwide. Approximately half of all the cornea transplants worldwide are performed for treatment of corneal endothelial dysfunction.

The cornea is a transparent tissue comprising different layers: corneal epithelium, Bowman’s membrane, stroma, Descemet’s Membrane and endothelium. The corneal endothelium also comprises a monolayer of human corneal endothelial cells and helps maintain corneal transparency via its barrier and ionic pump functions. It plays a crucial role in maintaining the balance of fluid, nutrients and salts between the corneal stroma and the aqueous humor. To maintain transparency, endothelial cell density must be maintained, however endothelial cell density can be significantly decreased as a result of trauma, disease or endothelial dystrophies. The density of the cells also decreases with aging. Human corneal endothelium has a limited propensity to proliferate in vivo. If the density of cells falls too low, the barrier function may be compromised. Loss of endothelial barrier function results in corneal edema and loss of visual acuity. The clinical condition of bullous keratopathy may be one resulting complication.

Currently the only treatment for blindness caused by corneal endothelial dysfunction is corneal transplantation. Although corneal transplantation is one of the most common forms of organ transplantation, the availability of donor corneas required is extremely limited. A 2012-2013 global survey quantified the considerable shortage of corneal graft tissue, finding that only one cornea is available for every 70 needed (Gain at el., (2016) Global Survey of Corneal Transplantation and Eye Banking. JAMA Ophthalmol. 134:167-173).

New therapeutic approaches to supply corneal endothelial cells for the treatment of corneal endothelial dysfunction are thus greatly needed.

The corneal epithelium also needs to be maintained in the eye. The corneal epithelium is composed of a layer of basal cells and multiple layers of a non-keratinized, stratified, squamous epithelium. It is essential in maintaining the clarity and the regular refractive surface of the cornea. It acts as a transparent, renewable protective layer over the corneal stroma and is replenished by a stem cell population located in the limbus. In limbal stem cell deficiency, a condition in which limbal stem cells are diseased or absent, a decrease in the number of healthy limbal stem cells results in a decreased capacity for corneal epithelium renewal.

Limbal stem cell deficiency may arise as a result of injuries from chemical or thermal burns, ultraviolet and ionizing radiation, or even as a result of contact lens wear; genetic disorders like aniridia, and immune disorders such as Stevens Johnson syndrome and ocular cicatricial pemphigoid. Loss of limbal stem cells can be partial or total; and may be unilateral or bilateral. Symptoms of limbal stem cell deficiency include pain, photophobia, non healing painful corneal epithelial defects, corneal neovascularization, replacement of the corneal epithelium by conjunctival epithelium, loss of corneal transparency and decreased vision that can eventually lead to blindness.

A product for use in treating limbal stem cell deficiency was granted a conditional marketing authorisation in the European Union in 2015 (under the name Holoclar®), making it the first Advanced Therapy Medicinal Product (ATMP) containing stem cells in Europe. Holoclar is an ex vivo expanded preparation of autologous human corneal epithelial cells containing stem cells. A biopsy of healthy limbal tissue is taken from the patient, expanded ex vivo and frozen until surgery. For administration to the patient, the thawed cells are grown on a membrane comprising fibrin and then surgically implanted onto the eye of the patient. The therapy is intended for use in adults with moderate to severe limbal stem cell deficiency due to physical or chemical ocular burns (Rama P, Matuska S, Paganoni G, Spinelli A, De Luca M, Pellegrini G. (2010). Limbal stem-cell therapy and long-term corneal regeneration. N Engl J Med. 363:147-155). However the method is limited in that it is for autologous use only, and there must be enough surviving limbus in one eye to allow a minimum of one to two square millimeters of undamaged tissue to be extracted from the patient. There is also the risk that for each specific patient the culture of his/her cells may not be successful and the patient cannot receive this treatment. Furthermore feeder cells of murine origin are used to prepare the Holoclar cell preparation which introduces potential safety concerns due to the risk of disease transmission and potential immunogenicity into the preparation for use in humans. Moreover, the Holoclar cell preparation only contains approximately 5% of limbal stem cells, as identified by p63alpha staining.

New therapeutic approaches to supply limbal stem cells for the treatment of limbal stem cell deficiency are thus greatly needed.

### IV. SUMMARY

The inventions described herein relate to compositions and methods for ocular cell therapy, for example, ocular cells modified at specific target sequences in their genome, including as modified by introduction of CRISPR systems (e.g., S. pyogenes Cas9 CRISPR systems) that include gRNA molecules which target said target sequences. For example, the present disclosure relates to gRNA molecules, CRISPR systems, ocular cells, and methods using genome edited cells, e.g., modified limbal stem cells, for treating ocular diseases.

The present invention provides a modified limbal stem cell, which has reduced or eliminated expression of beta-2-microglobulin (B2M) relative to an unmodified limbal stem cell.

The present invention further provides a population of modified limbal stem cells, which have reduced or eliminated expression of B2M relative to an unmodified limbal stem cell.

In one aspect, a modified limbal stem cell includes an insertion or deletion of a base pair, e.g., more than one base pair, at or near B2M relative to an unmodified limbal stem cell. In another aspect, the invention provides a population of cells including the modified limbal stem cell, wherein in at least about 30% of the cells, at least one said insertion or deletion is a frameshift mutation, e.g., as measured by next-generation sequencing (NGS).

In certain aspects, the invention provides a modified limbal stem cell, which has reduced or eliminated expression of beta-2-microglobulin (B2M) relative to an unmodified limbal stem cell, wherein the B2M expression is reduced or eliminated by a CRISPR system (e.g., S. pyogenes Cas9 CRISPR system) comprising a gRNA molecule comprising a targeting domain complementary to a target sequence in the B2M gene.

In other aspects, the invention provides a modified limbal stem cell, which has reduced or eliminated expression of beta-2-microglobulin (B2M) relative to an unmodified limbal stem cell, wherein the B2M expression is reduced or eliminated by a CRISPR system (e.g., S. pyogenes Cas9 CRISPR system) comprising a nucleic acid molecule encoding a gRNA molecule comprising a targeting domain complementary to a target sequence in the B2M gene.

In certain aspects, the invention provides a modified limbal stem cell, which has reduced or eliminated expression of beta-2-microglobulin (B2M) relative to an unmodified limbal stem cell, wherein the B2M expression is reduced or eliminated by a CRISPR system (e.g., S. pyogenes Cas9 CRISPR system) comprising a gRNA molecule comprising a targeting domain complementary to a target sequence in the B2M gene, wherein the modified limbal stem cell was exposed to (e.g., was cultured in media comprising) a LATS inhibitor.

In other aspects, the invention provides a modified limbal stem cell, which has reduced or eliminated expression of beta-2-microglobulin (B2M) relative to an unmodified limbal stem cell, wherein the B2M expression is reduced or eliminated by a CRISPR system (e.g., S. pyogenes Cas9 CRISPR system) comprising a nucleic acid molecule encoding a gRNA molecule comprising a targeting domain complementary to a target sequence in the B2M gene, wherein the modified limbal stem cell was exposed to a LATS inhibitor.

The present invention also provides a modified corneal endothelial cell, which has reduced or eliminated expression of B2M relative to an unmodified corneal endothelial cell.

The present invention further provides a population of modified corneal endothelial cells, which have reduced or eliminated expression of B2M relative to an unmodified corneal endothelial cell.

In one aspect, a modified corneal endothelial cell includes an insertion or deletion of a base pair, e.g., more than one base pair, at or near B2M relative to an unmodified corneal endothelial cell. In another aspect, the invention provides a population of cells including the modified corneal endothelial, wherein in at least about 30% of the cells, at least one said insertion or deletion is a frameshift mutation, e.g., as measured by next-generation sequencing (NGS).

The invention further provides methods of treating a patient suffering from an ocular disease comprising: providing a population of limbal stem cells, wherein the population of limbal stem cells has been cultured in the presence of a LATS inhibitor; introducing into the population of limbal stem cells a CRISPR system (e.g., S. pyogenes Cas9 CRISPR system) comprising a gRNA molecule comprising a targeting domain complementary to a target sequence in the B2M gene; and administering the population of cells to a patient in need thereof.

The invention also provides methods of preparing a population of modified limbal stem cells for ocular cell therapy comprising: modifying a population of limbal stem cells by reducing or eliminating expression of B2M comprising introducing into the limbal stem cells a gRNA molecule with a targeting domain comprising the sequence of any one of SEQ ID NOs: 23-105 or SEQ ID NOs: 108-119 or SEQ ID NOs: 134-140, wherein the limbal stem cells have optionally been cultured in the presence of a LATS inhibitor; and further expanding the modified limbal stem cells in cell culture media comprising a LATS inhibitor.

In certain aspects, a LATS inhibitor useful in a method of the invention is a compound of Formula A1

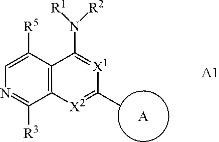

or a salt thereof.

Non-limiting embodiments of the present disclosure are described in the following embodiments:

1. A modified limbal stem cell, which has reduced or eliminated expression of beta-2-microglobulin (B2M) relative to an unmodified limbal stem cell, wherein the B2M expression is reduced or eliminated by a CRISPR system comprising a gRNA molecule comprising a targeting domain complementary to a target sequence in the B2M gene.

2. A modified limbal stem cell, which has reduced or eliminated expression of beta-2-microglobulin (B2M) relative to an unmodified limbal stem cell, wherein the B2M expression is reduced or eliminated by a CRISPR system comprising a nucleic acid molecule encoding a gRNA molecule comprising a targeting domain complementary to a target sequence in the B2M gene.

3. The modified limbal stem cell of embodiment 1 or 2, wherein the modified limbal stem cell was cultured in media comprising a large tumor suppressor kinase (“LATS”) inhibitor, optionally wherein the LATS inhibitor is a compound of Formula A1

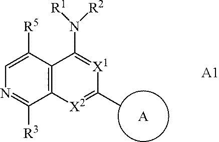

or a salt thereof, wherein
X¹ and X² are each independently CH or N;
Ring A is
   (a) a 5- or 6-membered monocyclic heteroaryl that is linked to the remainder of the molecule through a carbon ring member and comprises, as ring member, 1 to 4 heteroatoms that are independently selected from N, O and S, provided that at least one of the heteroatom ring member is an unsubstituted nitrogen (—N═) positioned at the 3- or the 4-position relative to the linking carbon ring member of the 5-membered heteroaryl or at the para ring position of the 6-membered heteroaryl; or
   (b) a 9-membered fused bicyclic heteroaryl that is selected from 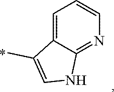 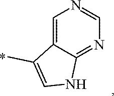 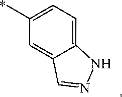 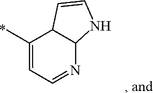 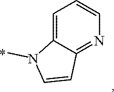
wherein “*” represents the point of attachment of ring A to the remainder of the molecule;
wherein ring A is unsubstituted or substituted by 1 to 2 substituents independently selected from halogen, cyano, C₁₋₆alkyl, C₁₋₆haloalkyl, —NH₂, C₁₋₆alkylamino, di-(C₁₋₆alkyl)amino, C₃₋₆cycloalkyl, and phenylsulfonyl;
R⁰ is hydroxyl or C₁₋₆alkoxy;
R¹ is hydrogen or C₁₋₆alkyl;
R² is selected from
   (a) C₁₋₈alkyl that is unsubstituted or substituted by 1 to 3 substituents independently selected from
      (i) halogen;
      (ii) cyano;
      (iii) oxo;
      (iv) C₂alkenyl;
      (v) C₂alkynyl;
      (vi) C₁₋₆haloalkyl;
      (vii) —OR⁶, wherein R⁶ is selected from hydrogen, C₁₋₆alkyl that is unsubstituted or substituted by R⁰ or —C(O)R⁰;
      (viii) —NR^{7a}R^{7b}, wherein R^{7a} is hydrogen or C₁₋₆alkyl, and R^{7b} is selected from hydrogen, —C(O)R⁰, C₁₋₆alkyl that is unsubstituted or substituted by —C(O)R⁰;
      (ix) —C(O)R⁸, wherein R⁸ is R⁰ or —NH—C₁₋₆alkyl-C(O)R⁰;
      (x) -S(O)₂C₁₋₆alkyl;
      (xi) monocyclic C₃₋₆cycloalkyl or polycyclic C₇₋₁₀cycloalkyl that are each unsubstituted or substituted by 1 to 2 substituents independently selected from halogen, C₁₋ ₆alkyl, hydroxyC₁₋₆alkyl, C₁₋₆haloalkyl, R⁰, —NH₂, C₁₋₆alkylamino, and di-(C₁₋ ₆alkyl)amino;
      (xii) 6-membered heterocycloalkyl comprising, as ring members, 1 to 2 heteroatoms independently selected from N, O and S and that is unsubstituted or substituted by 1 to 2 substituents independently selected from hydroxyl, halogen, C₁₋₆alkyl, C₁₋₆alkylamino, and di-(C₁₋₆alkyl)amino;
      (xiii) phenyl that is unsubstituted or substituted by halogen;
      (xiv) 5- or 6-membered monocyclic heteroaryl comprising, as ring members, 1 to 4 heteroatoms independently selected from N and O; and
      (xv) 9- or 10-membered fused bicyclic heteroaryl comprising, as ring member, 1 to 2 heteroatoms independently selected from N and O;
   (b) -S(O)₂C₁₋₆alkyl;
   (c) phenyl that is unsubstituted or substituted by 1 to 2 substituents independently selected from halogen, C₁₋₆alkyl and R⁰;
   (d) C₃₋₆cycloalkyl that is unsubstituted or substituted by 1 to 2 substituents independently selected from C₁₋₆haloalkyl, R⁰, C₁₋₆alkylamino, di-(C₁₋₆alkyl)amino, —C(O)R⁰, and C₁₋ ₆alkyl that is unsubstituted or substituted by R⁰ or —C(O)R⁰; and
   (e) 4-membered heterocycloalkyl comprising, as ring members, 1 to 2 heteroatoms selected from N, O and S and that is unsubstituted or substituted by 1 to 2 substituents independently selected from C₁₋₆haloalkyl, R⁰, C₁₋₆alkylamino, di-(C₁₋ ₆alkyl)amino, —C(O)R⁰, and C₁₋₆alkyl that is unsubstituted or substituted by R⁰ or—C(O)R⁰;
or R¹ and R² can be taken together with the nitrogen atom to which both are bound to form a 4- to 6-membered heterocycloalkyl that can include, as ring members, 1 to 2 additional heteroatoms independently selected from N, O, and S, wherein the 4- to 6-membered heterocycloalkyl formed by R¹ and R² taken together with the nitrogen atom to which both are bound is unsubstituted or substituted by 1 to 3 substituents independently selected from halogen, C₁₋₆alkyl, C₁₋₆haloalkyl, and R⁰;
R³ is selected from hydrogen, halogen and C₁₋₆alkyl; and
R⁵ is selected from hydrogen, halogen and —NH—(3- to 8-membered heteroalkyl), wherein the 3- to 8-membered heteroC₃₋₈alkyl of the —NH—(3- to 8-membered heteroalkyl) comprises 1 to 2 oxygen atoms as chain members and is unsubstituted or substituted by R⁰.

4. The modified limbal stem cell according to embodiment 3, wherein the compound is selected from: dimethyl(3-methyl-3-{[2-(pyridin-4-yl)pyrido[3,4-d]pyrimidin-4-yl]amino}butyl)amine and N¹,N¹,3-trimethyl-N³-(2-(3-methyl-1H-pyrazol-4-yl)pyrido[3,4-d]pyrimidin-4-yl)butane-1,3-diamine.

5. The modified limbal stem cell according to embodiment 3, wherein the compound is dimethyl(3-methyl-3-{[2-(pyridin-4-yl)pyrido[3,4-d]pyrimidin-4-yl]amino}butyl)amine.

6. The modified limbal stem cell according to any one of embodiments 3 to 5, wherein the compound is present in a concentration of 3 to 10 micromolar.

7. The modified limbal stem cell of embodiment any one of embodiments 1-6, wherein the targeting domain of the gRNA molecule is complementary to a sequence within a genomic region selected from: chr15:44711469-44711494, chr15:44711472-44711497, chr15:44711483-44711508, chr15:44711486-44711511, chr15:44711487-44711512, chr15:44711512-44711537, chr15:44711513-44711538, chr15:44711534-44711559, chr15:44711568-44711593, chr15:44711573-44711598, chr15:44711576-44711601, chr15:44711466-44711491, chr15:44711522-44711547, chr15:44711544-44711569, chr15:44711559-44711584, chr15:44711565-44711590, chr15:44711599-44711624, chr15:44711611-44711636, chr15:44715412-44715437, chr15:44715440-44715465, chr15:44715473-44715498, chr15:44715474-44715499, chr15:44715515-44715540, chr15:44715535-44715560, chr15:44715562-44715587, chr15:44715567-44715592, chr15:44715672-44715697, chr15:44715673-44715698, chr15:44715674-44715699, chr15:44715410-44715435, chr15:44715411-44715436, chr15:44715419-44715444, chr15:44715430-44715455, chr15:44715457-44715482, chr15:44715483-44715508, chr15:44715511-44715536, chr15:44715515-44715540, chr15:44715629-44715654, chr15:44715630-44715655, chr15:44715631-44715656, chr15:44715632-44715657, chr15:44715653-44715678, chr15:44715657-44715682, chr15:44715666-44715691, chr15:44715685-44715710, chr15:44715686-44715711, chr15:44716326-44716351, chr15:44716329-44716354, chr15:44716313-44716338, chr15:44717599-44717624, chr15:44717604-44717629, chr15:44717681-44717706, chr15:44717682-44717707, chr15:44717702-44717727, chr15:44717764-44717789, chr15:44717776-44717801, chr15:44717786-44717811, chr15:44717789-44717814, chr15:44717790-44717815, chr15:44717794-44717819, chr15:44717805-44717830, chr15:44717808-44717833, chr15:44717809-44717834, chr15:44717810-44717835, chr15:44717846-44717871, chr15:44717945-44717970, chr15:44717946-44717971, chr15:44717947-44717972, chr15:44717948-44717973, chr15:44717973-44717998, chr15:44717981-44718006, chr15:44718056-44718081, chr15:44718061-44718086, chr15:44718067-44718092, chr15:44718076-44718101, chr15:44717589-44717614, chr15:44717620-44717645, chr15:44717642-44717667, chr15:44717771-44717796, chr15:44717800-44717825, chr15:44717859-44717884, chr15:44717947-44717972, chr15:44718119-44718144, chr15:44711563-44711585, chr15:44715428-44715450, chr15:44715509-44715531, chr15:44715513-44715535, chr15:44715417-44715439, chr15:44711540-44711562, chr15:44711574-44711596, chr15:44711597-44711619, chr15:44715446-44715468, chr15:44715651-44715673, chr15:44713812-44713834, chr15:44711579-44711601, chr15:44711542-44711564, chr15:44711557-44711579, chr15:44711609-44711631, chr15:44715678-44715700, chr15:44715683-44715705, chr15:44715684-44715706, chr15:44715480-44715502.

8. The modified limbal stem cell of embodiment 7, wherein the targeting domain of the gRNA molecule is complementary to a sequence within a genomic region selected from: chr15:44715513-44715535, chr15:44711542-44711564, chr15:44711563-44711585, chr15:44715683-44715705, chr15:44711597-44711619, or chr15:44715446-44715468.

9. The modified limbal stem cell of embodiment 7, wherein the targeting domain of the gRNA molecule is complementary to a sequence within a genomic region chr15:44711563-44711585.

10. The modified limbal stem cell of any one of embodiments 1-6, wherein the targeting domain of the gRNA molecule to B2M comprises a targeting domain comprising the sequence of any one of SEQ ID NOs: 23-105 or 108-119 or 134-140.

11. The modified limbal stem cell of embodiment 10, wherein the targeting domain of the gRNA molecule to B2M comprises a targeting domain comprising the sequence of any one of SEQ ID NOs: 108, 111, 115, 116, 134 or 138.

12. The modified limbal stem cell of embodiment 10, wherein the targeting domain of the gRNA molecule to B2M comprises a targeting domain comprising the sequence of SEQ ID NO: 108.

13. The modified limbal stem cell of embodiment 10, wherein the targeting domain of the gRNA molecule to B2M comprises a targeting domain comprising the sequence of SEQ ID NO: 115.

14. The modified limbal stem cell of embodiment 10, wherein the targeting domain of the gRNA molecule to B2M comprises a targeting domain comprising the sequence of SEQ ID NO: 116.

15. The modified limbal stem cell of any one of embodiments 1-6, wherein the gRNA comprises the sequence of any one of SEQ ID NOs: 120, 160-177.

16. The modified limbal stem cell of embodiment 15, wherein the gRNA comprises the sequence of any one of SEQ ID NOs: 120, 162, 166, 167, 171, and 175.

17. The modified limbal stem cell of embodiment 15, wherein the gRNA comprises the sequence of SEQ ID NO: 120.

18. The modified limbal stem cell of embodiment 15, wherein the gRNA comprises the sequence of SEQ ID NO: 166.

19. The modified limbal stem cell of embodiment 15, wherein the gRNA comprises the sequence of SEQ ID NO: 167.

20. The modified limbal stem cell of embodiments 1-19, wherein the CRISPR system is an S. pyogenes Cas9 CRISPR system.

21. The modified limbal stem cell of embodiment 20, wherein the CRISPR system comprises a Cas9 molecule comprising SEQ ID NO: 106 or 107 or any of SEQ ID NOs: 124 to 134.

22. The modified limbal stem cell of embodiment 20, wherein the CRISPR system comprises a Cas9 molecule comprising SEQ ID NO: 106 or 107.

23. A modified limbal stem cell comprising a genome in which the b2 microglobulin (B2M) gene on chromosome 15 has been edited
(a) to delete a contiguous stretch of genomic DNA comprising the sequence of any one of SEQ ID NOs: 141 to 159, thereby eliminating surface expression of MHC Class I molecules in the cell, or
(b) to form an indel at or near the target sequence complementary to the targeting domain of the gRNA molecule comprising the sequence of any one of SEQ ID NOs: 23-105 or 108-119 or 134-140, thereby eliminating surface expression of MHC Class I molecules in the cell.

24. The modified limbal stem cell of embodiment 23 comprising a genome in which the b2 microglobulin (B2M) gene on chromosome 15 has been edited:
(a) to delete a contiguous stretch of genomic DNA comprising the sequence of any one of SEQ ID NOs: 141, 148 or 149, thereby eliminating surface expression, of MHC Class I molecules in the cell, or
(b) to form an indel at or near the target sequence complementary to the targeting domain of the gRNA molecule domain comprising the sequence of any one of SEQ ID NOs: 108, 111, 115, 116, 134 or 138, thereby eliminating surface expression of MHC Class I molecules in the cell.

25. The modified limbal stem cell of embodiment 23 comprising a genome in which the b2 microglobulin (B2M) gene on chromosome 15 has been:
(a) edited to delete a contiguous stretch of genomic DNA comprising the sequence of SEQ ID NO: 141, thereby eliminating surface expression, of MHC Class I molecules in the cell, or
(b) to form an indel at or near the target sequence complementary to the targeting domain of the gRNA molecule domain comprising the sequence of SEQ ID NO: 108, thereby eliminating surface expression of MHC Class I molecules in the cell.

26. A modified limbal stem cell comprising a genome in which the b2 microglobulin (B2M) gene on chromosome 15 has been edited:
(a) to delete a contiguous stretch of genomic DNA region selected from any one of: chr15:44711469-44711494, chr15:44711472-44711497, chr15:44711483-44711508, chr15:44711486-44711511, chr15:44711487-44711512, chr15:44711512-44711537, chr15:44711513-44711538, chr15:44711534-44711559, chr15:44711568-44711593, chr15:44711573-44711598, chr15:44711576-44711601, chr15:44711466-44711491, chr15:44711522-44711547, chr15:44711544-44711569, chr15:44711559-44711584, chr15:44711565-44711590, chr15:44711599-44711624, chr15:44711611-44711636, chr15:44715412-44715437, chr15:44715440-44715465, chr15:44715473-44715498, chr15:44715474-44715499, chr15:44715515-44715540, chr15:44715535-44715560, chr15:44715562-44715587, chr15:44715567-44715592, chr15:44715672-44715697, chr15:44715673-44715698, chr15:44715674-44715699, chr15:44715410-44715435, chr15:44715411-44715436, chr15:44715419-44715444, chr15:44715430-44715455, chr15:44715457-44715482, chr15:44715483-44715508, chr15:44715511-44715536, chr15:44715515-44715540, chr15:44715629-44715654, chr15:44715630-44715655, chr15:44715631-44715656, chr15:44715632-44715657, chr15:44715653-44715678, chr15:44715657-44715682, chr15:44715666-44715691, chr15:44715685-44715710, chr15:44715686-44715711, chr15:44716326-44716351, chr15:44716329-44716354, chr15:44716313-44716338, chr15:44717599-44717624, chr15:44717604-44717629, chr15:44717681-44717706, chr15:44717682-44717707, chr15:44717702-44717727, chr15:44717764-44717789, chr15:44717776-44717801, chr15:44717786-44717811, chr15:44717789-44717814, chr15:44717790-44717815, chr15:44717794-44717819, chr15:44717805-44717830, chr15:44717808-44717833, chr15:44717809-44717834, chr15:44717810-44717835, chr15:44717846-44717871, chr15:44717945-44717970, chr15:44717946-44717971, chr15:44717947-44717972, chr15:44717948-44717973, chr15:44717973-44717998, chr15:44717981-44718006, chr15:44718056-44718081, chr15:44718061-44718086, chr15:44718067-44718092, chr15:44718076-44718101, chr15:44717589-44717614, chr15:44717620-44717645, chr15:44717642-44717667, chr15:44717771-44717796, chr15:44717800-44717825, chr15:44717859-44717884, chr15:44717947-44717972, chr15:44718119-44718144, chr15:44711563-44711585, chr15:44715428-44715450, chr15:44715509-44715531, chr15:44715513-44715535, chr15:44715417-44715439, chr15:44711540-44711562, chr15:44711574-44711596, chr15:44711597-44711619, chr15:44715446-44715468, chr15:44715651-44715673, chr15:44713812-44713834, chr15:44711579-44711601, chr15:44711542-44711564, chr15:44711557-44711579, chr15:44711609-44711631, chr15:44715678-44715700, chr15:44715683-44715705, chr15:44715684-44715706, chr15:44715480-44715502, thereby eliminating surface expression of MHC Class I molecules in the cell, or
(b) to form an indel at or near the genomic DNA region selected from any one of: chr15:44711469-44711494, chr15:44711472-44711497, chr15:44711483-44711508, chr15:44711486-44711511, chr15:44711487-44711512, chr15:44711512-44711537, chr15:44711513-44711538, chr15:44711534-44711559, chr15:44711568-44711593, chr15:44711573-44711598, chr15:44711576-44711601, chr15:44711466-44711491, chr15:44711522-44711547, chr15:44711544-44711569, chr15:44711559-44711584, chr15:44711565-44711590, chr15:44711599-44711624, chr15:44711611-44711636, chr15:44715412-44715437, chr15:44715440-44715465, chr15:44715473-44715498, chr15:44715474-44715499, chr15:44715515-44715540, chr15:44715535-44715560, chr15:44715562-44715587, chr15:44715567-44715592, chr15:44715672-44715697, chr15:44715673-44715698, chr15:44715674-44715699, chr15:44715410-44715435, chr15:44715411-44715436, chr15:44715419-44715444, chr15:44715430-44715455, chr15:44715457-44715482, chr15:44715483-44715508, chr15:44715511-44715536, chr15:44715515-44715540, chr15:44715629-44715654, chr15:44715630-44715655, chr15:44715631-44715656, chr15:44715632-44715657, chr15:44715653-44715678, chr15:44715657-44715682, chr15:44715666-44715691, chr15:44715685-44715710, chr15:44715686-44715711, chr15:44716326-44716351, chr15:44716329-44716354, chr15:44716313-44716338, chr15:44717599-44717624, chr15:44717604-44717629, chr15:44717681-44717706, chr15:44717682-44717707, chr15:44717702-44717727, chr15:44717764-44717789, chr15:44717776-44717801, chr15:44717786-44717811, chr15:44717789-44717814, chr15:44717790-44717815, chr15:44717794-44717819, chr15:44717805-44717830, chr15:44717808-44717833, chr15:44717809-44717834, chr15:44717810-44717835, chr15:44717846-44717871, chr15:44717945-44717970, chr15:44717946-44717971, chr15:44717947-44717972, chr15:44717948-44717973, chr15:44717973-44717998, chr15:44717981-44718006, chr15:44718056-44718081, chr15:44718061-44718086, chr15:44718067-44718092, chr15:44718076-44718101, chr15:44717589-44717614, chr15:44717620-44717645, chr15:44717642-44717667, chr15:44717771-44717796, chr15:44717800-44717825, chr15:44717859-44717884, chr15:44717947-44717972, chr15:44718119-44718144, chr15:44711563-44711585, chr15:44715428-44715450, chr15:44715509-44715531, chr15:44715513-44715535, chr15:44715417-44715439, chr15:44711540-44711562, chr15:44711574-44711596, chr15:44711597-44711619, chr15:44715446-44715468, chr15:44715651-44715673, chr15:44713812-44713834, chr15:44711579-44711601, chr15:44711542-44711564, chr15:44711557-44711579, chr15:44711609-44711631, chr15:44715678-44715700, chr15:44715683-44715705, chr15:44715684-44715706, chr15:44715480-44715502, thereby eliminating surface expression, of MHC Class I molecules in the cell.

27. The modified limbal stem cell of embodiment 26 comprising a genome in which the b2 microglobulin (B2M) gene on chromosome 15 has been edited:
(a) to delete a contiguous stretch of genomic DNA region selected from: chr15:44715513-44715535, chr15:44711542-44711564, chr15:44711563-44711585, chr15:44715683-44715705, chr15:44711597-44711619, or chr15:44715446-44715468, or
(b) to form an indel at or near the genomic DNA region selected from any one of: chr15:44715513-44715535, chr15:44711542-44711564, chr15:44711563-44711585, chr15:44715683-44715705, chr15:44711597-44711619, or chr15:44715446-44715468.

28. The modified limbal stem cell of embodiment 26 comprising a genome in which the b2 microglobulin (B2M) gene on chromosome 15 has been edited
(a) to delete a contiguous stretch of genomic DNA region chr15:44711563-44711585, thereby eliminating surface expression of MHC Class I molecules in the cell, or:
(b) to form an indel at or near the genomic DNA region chr15:44711563-44711585, thereby eliminating surface expression of MHC Class I molecules in the cell.

29. The modified limbal stem cell of any one of the previous embodiments, wherein the modified limbal stem cell comprises an indel formed at or near the target sequence complementary to the targeting domain of the gRNA molecule.

30. The modified limbal stem cell of any one of embodiments 23(b), 24(b), 25(b), 26(b), 27(b) or 28(b) or 29, wherein wherein the indel comprises a deletion of 10 or greater than 10 nucleotides, optionally 11, 12, 13, 14, 15, 16, 17, 18, 19, 20, 21, 22, 23, 24, 25, 26, 27, 28, 29, 30, 31,32, 33, 34, or 35 nucleotides.

31. The modified limbal stem cell any one of embodiments 23 to 30, wherein the modified limbal stem cell was cultured in media comprising a large tumor suppressor kinase (“LATS”) inhibitor, optionally wherein the LATS inhibitor is a compound of Formula A1

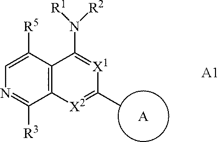

or a salt thereof, wherein
X¹ and X² are each independently CH or N;
Ring A is
   (a) a 5- or 6-membered monocyclic heteroaryl that is linked to the remainder of the molecule through a carbon ring member and comprises, as ring member, 1 to 4 heteroatoms that are independently selected from N, O and S, provided that at least one of the heteroatom ring member is an unsubstituted nitrogen (—N═) positioned at the 3- or the 4-position relative to the linking carbon ring member of the 5-membered heteroaryl or at the para ring position of the 6-membered heteroaryl; or
   (b) a 9-membered fused bicyclic heteroaryl that is selected from 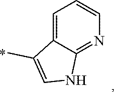 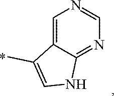 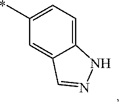 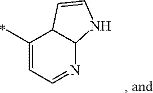 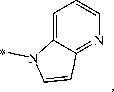
wherein “*” represents the point of attachment of ring A to the remainder of the molecule;
wherein ring A is unsubstituted or substituted by 1 to 2 substituents independently selected from halogen, cyano, C₁₋₆alkyl, C₁₋₆haloalkyl, —NH₂, C₁₋₆alkylamino, di-(C₁₋₆alkyl)amino, C₃₋₆cycloalkyl, and phenylsulfonyl;
R⁰ is hydroxyl or C₁₋₆alkoxy;
R¹ is hydrogen or C₁₋₆alkyl;
R² is selected from
   (a) C₁₋₈alkyl that is unsubstituted or substituted by 1 to 3 substituents independently selected from
      (i) halogen;
      (ii) cyano;
      (iii) oxo;
      (iv) C₂alkenyl;
      (v) C₂alkynyl;
      (vi) C₁₋₆haloalkyl;
      (vii) —OR⁶, wherein R⁶ is selected from hydrogen, C₁₋₆alkyl that is unsubstituted or substituted by R⁰ or —C(O)R⁰;
      (viii) —NR^{7a}R^{7b}, wherein R^{7a} is hydrogen or C₁₋₆alkyl, and R^{7b} is selected from hydrogen, —C(O)R⁰, C₁₋₆alkyl that is unsubstituted or substituted by —C(O)R⁰;
      (ix) —C(O)R⁸, wherein R⁸ is R⁰ or —NH—C₁₋₆alkyl-C(O)R⁰;
      (x) -S(O)₂C₁₋₆alkyl;
      (xi) monocyclic C₃₋₆cycloalkyl or polycyclic C₇₋₁₀cycloalkyl that are each unsubstituted or substituted by 1 to 2 substituents independently selected from halogen, C₁₋ ₆alkyl, hydroxyC₁₋₆alkyl, C₁₋₆haloalkyl, R⁰, —NH₂, C₁₋₆alkylamino, and di-(C₁₋ ₆alkyl)amino;
      (xii) 6-membered heterocycloalkyl comprising, as ring members, 1 to 2 heteroatoms independently selected from N, O and S and that is unsubstituted or substituted by 1 to 2 substituents independently selected from hydroxyl, halogen, C₁₋₆alkyl, C₁₋₆alkylamino, and di-(C₁₋₆alkyl)amino;
      (xiii) phenyl that is unsubstituted or substituted by halogen;
      (xiv) 5- or 6-membered monocyclic heteroaryl comprising, as ring members, 1 to 4 heteroatoms independently selected from N and O; and
      (xv) 9- or 10-membered fused bicyclic heteroaryl comprising, as ring member, 1 to 2 heteroatoms independently selected from N and O;
   (b) -S(O)₂C₁₋₆alkyl;
   (c) phenyl that is unsubstituted or substituted by 1 to 2 substituents independently selected from halogen, C₁₋₆alkyl and R⁰;
   (d) C₃₋₆cycloalkyl that is unsubstituted or substituted by 1 to 2 substituents independently selected from C₁₋₆haloalkyl, R⁰, C₁₋₆alkylamino, di-(C₁₋₆alkyl)amino, —C(O)R⁰, and C₁₋ ₆alkyl that is unsubstituted or substituted by R⁰ or —C(O)R⁰; and
   (e) 4-membered heterocycloalkyl comprising, as ring members, 1 to 2 heteroatoms selected from N, O and S and that is unsubstituted or substituted by 1 to 2 substituents independently selected from C₁₋₆haloalkyl, R⁰, C₁₋₆alkylamino, di-(C₁₋ ₆alkyl)amino, —C(O)R⁰, and C₁₋₆alkyl that is unsubstituted or substituted by R⁰ or-C(O)R⁰;
or R¹ and R² can be taken together with the nitrogen atom to which both are bound to form a 4- to 6-membered heterocycloalkyl that can include, as ring members, 1 to 2 additional heteroatoms independently selected from N, O, and S, wherein the 4- to 6-membered heterocycloalkyl formed by R¹ and R² taken together with the nitrogen atom to which both are bound is unsubstituted or substituted by 1 to 3 substituents independently selected from halogen, C₁₋₆alkyl, C₁₋₆haloalkyl, and R⁰;
R³ is selected from hydrogen, halogen and C₁₋₆alkyl; and
R⁵ is selected from hydrogen, halogen and —NH—(3- to 8-membered heteroalkyl), wherein the 3- to 8-membered heteroC₃₋₈alkyl of the —NH—(3- to 8-membered heteroalkyl) comprises 1 to 2 oxygen atoms as chain members and is unsubstituted or substituted by R⁰.

32. The modified limbal stem cell according to embodiment 31, wherein the compound is selected from: dimethyl(3-methyl-3-{[2-(pyridin-4-yl)pyrido[3,4-d]pyrimidin-4-yl]amino}butyl)amine and N¹,N¹,3-trimethyl-N³-(2-(3-methyl-1H-pyrazol-4-yl)pyrido[3,4-d]pyrimidin-4-yl)butane-1,3-diamine.

33. The modified limbal stem cell according to embodiment 31, wherein the compound is dimethyl(3-methyl-3-{[2-(pyridin-4-yl)pyrido[3,4-d]pyrimidin-4-yl]amino}butyl)amine.

34. The modified limbal stem cell according to any one of embodiments 31 to 33, wherein the compound is present in a concentration of 3 to 10 micromolar.

35. The modified limbal stem cell of any of embodiments 1-34, wherein the cell is autologous with respect to a patient to be administered said cell.

36. The modified limbal stem cell of any of embodiments 1-34, wherein the cell is allogeneic with respect to a patient to be administered said cell.

37. A method of preparing a modified limbal stem cell or a population of modified limbal stem cells for ocular cell therapy comprising,
a) modifying a limbal stem cell or a population of limbal stem cells by reducing or eliminating expression of B2M comprising introducing into the limbal stem cell or the population of limbal stem cells a CRISPR system comprising a gRNA molecule with a targeting domain
   (i) comprising the sequence of any one of SEQ ID NOs: 23-105 or 108-119, or 134 to 140, or
   (ii) complementary to a sequence within a genomic region selected from: chr15:44711469-44711494, chr15:44711472-44711497, chr15:44711483-44711508, chr15:44711486-44711511, chr15:44711487-44711512, chr15:44711512-44711537, chr15:44711513-44711538, chr15:44711534-44711559, chr15:44711568-44711593, chr15:44711573-44711598, chr15:44711576-44711601, chr15:44711466-44711491, chr15:44711522-44711547, chr15:44711544-44711569, chr15:44711559-44711584, chr15:44711565-44711590, chr15:44711599-44711624, chr15:44711611-44711636, chr15:44715412-44715437, chr15:44715440-44715465, chr15:44715473-44715498, chr15:44715474-44715499, chr15:44715515-44715540, chr15:44715535-44715560, chr15:44715562-44715587, chr15:44715567-44715592, chr15:44715672-44715697, chr15:44715673-44715698, chr15:44715674-44715699, chr15:44715410-44715435, chr15:44715411-44715436, chr15:44715419-44715444, chr15:44715430-44715455, chr15:44715457-44715482, chr15:44715483-44715508, chr15:44715511-44715536, chr15:44715515-44715540, chr15:44715629-44715654, chr15:44715630-44715655, chr15:44715631-44715656, chr15:44715632-44715657, chr15:44715653-44715678, chr15:44715657-44715682, chr15:44715666-44715691, chr15:44715685-44715710, chr15:44715686-44715711, chr15:44716326-44716351, chr15:44716329-44716354, chr15:44716313-44716338, chr15:44717599-44717624, chr15:44717604-44717629, chr15:44717681-44717706, chr15:44717682-44717707, chr15:44717702-44717727, chr15:44717764-44717789, chr15:44717776-44717801, chr15:44717786-44717811, chr15:44717789-44717814, chr15:44717790-44717815, chr15:44717794-44717819, chr15:44717805-44717830, chr15:44717808-44717833, chr15:44717809-44717834, chr15:44717810-44717835, chr15:44717846-44717871, chr15:44717945-44717970, chr15:44717946-44717971, chr15:44717947-44717972, chr15:44717948-44717973, chr15:44717973-44717998, chr15:44717981-44718006, chr15:44718056-44718081, chr15:44718061-44718086, chr15:44718067-44718092, chr15:44718076-44718101, chr15:44717589-44717614, chr15:44717620-44717645, chr15:44717642-44717667, chr15:44717771-44717796, chr15:44717800-44717825, chr15:44717859-44717884, chr15:44717947-44717972, chr15:44718119-44718144, chr15:44711563-44711585, chr15:44715428-44715450, chr15:44715509-44715531, chr15:44715513-44715535, chr15:44715417-44715439, chr15:44711540-44711562, chr15:44711574-44711596, chr15:44711597-44711619, chr15:44715446-44715468, chr15:44715651-44715673, chr15:44713812-44713834, chr15:44711579-44711601, chr15:44711542-44711564, chr15:44711557-44711579, chr15:44711609-44711631, chr15:44715678-44715700, chr15:44715683-44715705, chr15:44715684-44715706, chr15:44715480-44715502, wherein the limbal stem cell or the population of limbal stem cells have optionally been cultured in the presence of a LATS inhibitor; and
b) further expanding the modified limbal stem cell or thepopulation of modified limbal stem cells in cell culture media comprising a LATS inhibitor; and
c) optionally, enriching the population of limbal stem cells with the limbal stem cells having reduced or eliminated expression of B2M by fluorescene activated cell sorting (FACS) or magnetic activated cell sorting (MACS).

38. The method of embodiment 37, wherein the LATS inhibitor is a compound of Formula A1

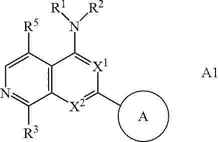

or a salt thereof, wherein
X¹ and X² are each independently CH or N;
Ring A is
   (a) a 5- or 6-membered monocyclic heteroaryl that is linked to the remainder of the molecule through a carbon ring member and comprises, as ring member, 1 to 4 heteroatoms that are independently selected from N, O and S, provided that at least one of the heteroatom ring member is an unsubstituted nitrogen (—N═) positioned at the 3- or the 4-position relative to the linking carbon ring member of the 5-membered heteroaryl or at the para ring position of the 6-membered heteroaryl; or
   (b) a 9-membered fused bicyclic heteroaryl that is selected from 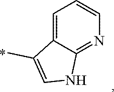 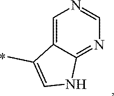 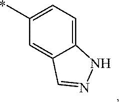 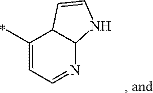 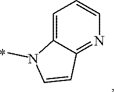
wherein “*” represents the point of attachment of ring A to the remainder of the molecule;
wherein ring A is unsubstituted or substituted by 1 to 2 substituents independently selected from halogen, cyano, C₁₋₆alkyl, C₁₋₆haloalkyl, —NH₂, C₁₋₆alkylamino, di-(C₁₋₆alkyl)amino, C₃₋₆cycloalkyl, and phenylsulfonyl;
R⁰ is hydroxyl or C₁₋₆alkoxy;
R¹ is hydrogen or C₁₋₆alkyl;
R² is selected from
   (a) C₁₋₈alkyl that is unsubstituted or substituted by 1 to 3 substituents independently selected from
      (i) halogen;
      (ii) cyano;
      (iii) oxo;
      (iv) C₂alkenyl;
      (v) C₂alkynyl;
      (vi) C₁₋₆haloalkyl;
      (vii) —OR⁶, wherein R⁶ is selected from hydrogen, C₁₋₆alkyl that is unsubstituted or substituted by R⁰ or —C(O)R⁰;
      (viii) —NR^{7a}R^{7b}, wherein R^{7a} is hydrogen or C₁₋₆alkyl, and R^{7b} is selected from hydrogen, —C(O)R⁰, C₁₋₆alkyl that is unsubstituted or substituted by —C(O)R⁰;
      (ix) —C(O)R⁸, wherein R⁸ is R⁰ or —NH—C₁₋₆alkyl-C(O)R⁰;
      (x) -S(O)₂C₁₋₆alkyl;
      (xi) monocyclic C₃₋₆cycloalkyl or polycyclic C₇₋₁₀cycloalkyl that are each unsubstituted or substituted by 1 to 2 substituents independently selected from halogen, C₁₋ ₆alkyl, hydroxyC₁₋₆alkyl, C₁₋₆haloalkyl, R⁰, —NH₂, C₁₋₆alkylamino, and di-(C₁₋ ₆alkyl)amino;
      (xii) 6-membered heterocycloalkyl comprising, as ring members, 1 to 2 heteroatoms independently selected from N, O and S and that is unsubstituted or substituted by 1 to 2 substituents independently selected from hydroxyl, halogen, C₁₋₆alkyl, C₁₋₆alkylamino, and di-(C₁₋₆alkyl)amino;
      (xiii) phenyl that is unsubstituted or substituted by halogen;
      (xiv) 5- or 6-membered monocyclic heteroaryl comprising, as ring members, 1 to 4 heteroatoms independently selected from N and O; and
      (xv) 9- or 10-membered fused bicyclic heteroaryl comprising, as ring member, 1 to 2 heteroatoms independently selected from N and O;
   (b) -S(O)₂C₁₋₆alkyl;
   (c) phenyl that is unsubstituted or substituted by 1 to 2 substituents independently selected from halogen, C₁₋₆alkyl and R⁰;
   (d) C₃₋₆cycloalkyl that is unsubstituted or substituted by 1 to 2 substituents independently selected from C₁₋₆haloalkyl, R⁰, C₁₋₆alkylamino, di-(C₁₋₆alkyl)amino, —C(O)R⁰, and C₁₋ ₆alkyl that is unsubstituted or substituted by R⁰ or —C(O)R⁰; and
   (e) 4-membered heterocycloalkyl comprising, as ring members, 1 to 2 heteroatoms selected from N, O and S and that is unsubstituted or substituted by 1 to 2 substituents independently selected from C₁₋₆haloalkyl, R⁰, C₁₋₆alkylamino, di-(C₁₋ ₆alkyl)amino, —C(O)R⁰, and C₁₋₆alkyl that is unsubstituted or substituted by R⁰ or —C(O)R⁰;
or R¹ and R² can be taken together with the nitrogen atom to which both are bound to form a 4- to 6-membered heterocycloalkyl that can include, as ring members, 1 to 2 additional heteroatoms independently selected from N, O, and S, wherein the 4- to 6-membered heterocycloalkyl formed by R¹ and R² taken together with the nitrogen atom to which both are bound is unsubstituted or substituted by 1 to 3 substituents independently selected from halogen, C₁₋₆alkyl, C₁₋₆haloalkyl, and R⁰;
R³ is selected from hydrogen, halogen and C₁₋₆alkyl; and
R⁵ is selected from hydrogen, halogen and —NH—(3- to 8-membered heteroalkyl), wherein the 3- to 8-membered heteroC₃₋₈alkyl of the —NH—(3- to 8-membered heteroalkyl) comprises 1 to 2 oxygen atoms as chain members and is unsubstituted or substituted by R⁰.

39. The method according to embodiment 38, wherein the compound is selected from: dimethyl(3-methyl-3-{[2-(pyridin-4-yl)pyrido[3,4-d]pyrimidin-4-yl]amino}butyl)amine and N¹,N¹,3-trimethyl-N³-(2-(3-methyl-1H-pyrazol-4-yl)pyrido[3,4-d]pyrimidin-4-yl)butane-1,3-diamine.

40. The method according to embodiment 38, wherein the compound is dimethyl(3-methyl-3-{[2-(pyridin-4-yl)pyrido[3,4-d]pyrimidin-4-yl]amino}butyl)amine.

41. The method according to any one of embodiments 38 to 40, wherein the compound is present in a concentration of 3 to 10 micromolar.

42. The method of any one of embodiments 37-41, wherein the CRISPR system is an S. pyogenes Cas9 CRISPR system.

43. The method of embodiment 42, wherein the CRISPR system comprises a Cas 9 molecule comprising SEQ ID NO: 106 or 107or 107 or any of SEQ ID NOs: 124 to 134.

44. The method of embodiment 42, wherein the CRISPR system comprises a Cas 9 molecule comprising SEQ ID NO: 106 or 107or 107.

45. A cell population comprising the modified limbal stem cell of any one of embodiments 1 to 36 or the modified limbal stem cell obtained by the method of any one of embodiments 37-44.

46. The cell population of embodiment 45, wherein the modified limbal stem cell comprises an indel formed at or near the target sequence complementary to the targeting domain of the gRNA molecule domain.

47. The cell population of embodiment 46, wherein the indel comprises a deletion of 10 or greater than 10 nucleotides, optionally 11, 12, 13, 14, 15, 16, 17, 18, 19, 20, 21, 22, 23, 24, 25, 26, 27, 28, 29, 30, 31, 32, 33, 34, or 35 nucleotides.

48. The cell population of embodiment 45 or 46, wherein the indel is formed in at least about 40%, e.g., at least about 50%, e.g., at least about 60%, e.g., at least about 70%, e.g., at least about 80%, e.g., at least about 90%, e.g., at least about 95%, e.g., at least about 96%, e.g., at least about 97%, e.g., at least about 98%, e.g., at least about 99%, of the cells of the cell population, e.g., as detectible by next generation sequencing and/or a nucleotide insertional assay.

49. The cell population of any one of embodiments 45 to 48, wherein an off-target indel is detected in no more than about 5%, e.g., no more than about 1%, e.g., no more than about 0.1%, e.g., no more than about 0.01%, of the cells of the cell population, e.g., as detectible by next generation sequencing and/or a nucleotide insertional assay.

50. A composition comprising the modified limbal stem cell of any one of embodiments 1 to 36 or the modified limbal stem cell obtained by the method of any one of embodiments 37-44 or the cell population of any one of embodiments 45-49 or the population of modified limbal stem cells obtained by the method of any one of embodiments 37-44.

51. The composition of embodiment 50, wherein the modified limbal stem cell comprises an indel formed at or near the target sequence complementary to the targeting domain of the gRNA molecule domain.

52. The composition of embodiment 51, wherein the indel comprises a deletion of 10 or greater than 10 nucleotides, optionally 11, 12, 13, 14, 15, 16, 17, 18, 19, 20, 21, 22, 23, 24, 25, 26, 27, 28, 29, 30, 31, 32, 33, 34, or 35 nucleotides.

53. The composition of embodiment 51 or 52, wherein the indel is formed in at least about 40%, e.g., at least about 50%, e.g., at least about 60%, e.g., at least about 70%, e.g., at least about 80%, e.g., at least about 90%, e.g., at least about 95%, e.g., at least about 96%, e.g., at least about 97%, e.g., at least about 98%, e.g., at least about 99%, of the cells of the population.

54. The composition of any one of embodiments 51 to 53, wherein an off-target indel is detected in no more than about 5%, e.g., no more than about 1%, e.g., no more than about 0.1%, e.g., no more than about 0.01%, of the cells of the population of cells e.g., as detectible by next generation sequencing and/or a nucleotide insertional assay.

55. The modified limbal stem cell of any one of embodiments 1 to 36 or the cell population of any one of embodiments 45 to 49 or the composition of any one of embodiments 50 to 54 for use in treatment of an an ocular disease.

56. The modified limbal stem cell or the cell population or the composition for use according to embodiment 55, wherein the ocular disease is limbal stem cell deficiency.

57. The modified limbal stem cell or the cell population or the composition for use according to embodiment 56, wherein the ocular disease is unilateral limbal stem cell deficiency.

58. The modified limbal stem cell or the cell population or the composition for use according to embodiment 56, wherein the ocular disease is bilateral limbal stem cell deficiency.

59. The modified limbal stem cell or the cell population or the composition for use according to any one of embodiments 50 to 53, wherein the cell is autologous with respect to a patient to be administered said cell.

60. The modified limbal stem cell or the cell population or the composition for use according to any one of embodiments 50 to 53, wherein the cell is allogeneic with respect to a patient to be administered said cell.

61. A method of treating a patient suffering from an ocular disease comprising the step of administering to the patient in need thereof the modified limbal stem cell of any one of embodiments 1-36 or the cell population of any one of embodiments 45 to 49 or the composition of any one of embodiments 50 to 54.

62. The method of embodiment 61, wherein the ocular disease is limbal stem cell deficiency.

63. The method of embodiment 62, wherein the ocular disease is unilateral limbal stem cell deficiency.

64. The method of embodiment 62, wherein the ocular disease is bilateral limbal stem cell deficiency.

65. The method of any one of embodiments 62 to 64, wherein the cell is autologous with respect to a patient to be administered said cell.

66. The method of any one of embodiments 62 to 64, wherein the cell is allogeneic with respect to a patient to be administered said cell.

67. Use of the modified limbal stem cell of any one of embodiments 1 to 36 or the cell population of any one of embodiments 45 to 49 or the composition of any one of embodiments 50 to54 for the treatment of an ocular disease.

68. Use of embodiment 67, wherein the ocular disease is limbal stem cell deficiency.

Other features and advantages of the present invention will be apparent from the following detailed description and claims.

### V. BRIEF DESCRIPTION OF THE FIGURES

FIG. 1: LATS inhibitors (compound ex. 3 and ex. 4) induce YAP dephosphorylation in LSCs within one hour of treatment as shown by Western blot.

FIG. 2: Immunolabelling of p63-alpha in limbal stem cell cultures indicates that the LSC population can be expanded when it is maintained in medium comprising the LATS inhibitors (compound ex. 3 and ex. 4). FIG. 2A: In the presence of growth medium and DMSO, only a few isolated cells attach to the culture dish and survive up to 6 days. Most cells expressed the human nuclear marker, but few expressed p63alpha. FIGS. 2B and 2C: In contrast, in the presence of LATS inhibitors: compound example no. 3 and example no. 4, the cells formed colonies and expressed p63alpha. This result indicated that the LATS inhibitors promote the expansion of the population of cells with the p63alpha-positive phenotype. FIG. 2D: Passaging cells and culturing them in the presence of LATS inhibitor compound for two weeks enabled cell population expansion and the formation of confluent cultures expressing p63alpha.

FIG. 3: FACS analyses show CRISPR-mediated deletion of B2M with sgRNA SEQ ID NO: 120 and subsequent elimination of HLA A, B and C occurred in about 70 percent of the LSCs.

FIG. 4: Graphs showing the results of gene edited LSCs (CRISPR-mediated deletion of B2M with sgRNA SEQ ID NO: 120) co-cultured with CD8+ T-cells from 4 different donors.

FIG. 5: Efficiency of B2M deletion. FIG. 5 shows FACS data detecting B2M surface protein on gene edited limbal stem cells, which were CRISPR-edited with sgRNA CR00442, CR000446, CR000455, 1-CR004366, 4-CR004366, 6-HEYJA000001, 8-HEYJA000004, and 9-HEYJA000005 represented in Table 6. All sgRNAs show a B2M surface protein knockout of between 27-62%.

FIG. 6: Efficiency of HLA A,B,C elimination. FIG. 6 shows FACS data detecting HLA-ABC surface protein on gene edited limbal stem cells, which were CRISPR-edited with sgRNA CR00442, CR000446, CR000455, 1-CR004366, 4-CR004366, 6-HEYJA000001, 8-HEYJA000004, and 9-HEYJA000005 represented in Table 6. All sgRNAs show an HLA-ABC surface protein elimination of between 28-60%.

FIG. 7: MACS-mediated selection of B2M-negative LSCs. FIG. 7 shows FACS data detecting B2M surface protein on gene edited limbal stem cells, which were MACS treated after nucleofection to obtain a B2M negative LSC culture. All sgRNAs tested (CR00442, CR000446, CR000455, 1-CR004366, 4-CR004366, 6-HEYJA000001, 8-HEYJA000004, and 9-HEYJA000005, represented in Table 6) show a pure (~99 to100%) B2M negative LSC culture.

FIG. 8: MACS-mediated selection of HLA A,B,C-negative LSCs. FIG. 8 shows FACS data detecting HLA-ABC surface protein on gene edited limbal stem cells, which were MACS treated after nucleofection to obtain a B2M/HLA-ABC negative LSC culture. All sgRNAs tested (CR00442, CR000446, CR000455, 1-CR004366, 4-CR004366, 6-HEYJA000001, 8-HEYJA000004, and 9-HEYJA000005, represented in Table 6) show a pure (~99 to 100%) HLA-ABC negative LSC culture.

### VI. DETAILED DESCRIPTION

### LATS

LATS is the abbreviated name of the large tumor suppressor kinase. LATS as used herein refers to LATS1 and/or LATS2. LATS1 as used herein refers to the large tumor suppressor kinase 1 and LATS2 refers to the large tumor suppressor kinase 2. LATS1 and LATS2 both have serine/threonine protein kinase activity. LATS1 and LATS2 have been given the Human Genome Organisation (HUGO) Gene Nomenclature Committee identifiers: HGNC ID 6514 and HGNC ID 6515 respectively. LATS1 is sometimes also referred to in the art as WARTS or wts, and LATS2 is sometimes referred to in the art as KPM. Representative LATS sequences, include, but are not limited to, the protein sequences available from the National Center for Biotechnology Information protein database with the accession numbers NP_004681.1 (LATS1) and NP_001257448.1 (LATS1) and NP_055387.2 (LATS 2), as shown below.

LATS1: NP_004681.1 (Serine/threonine-protein kinase LATS1 isoform 1, homo sapiens)(SEQ ID NO: 1:)

1 mkrsekpegy rqmrpktfpa snytvssrqm lqeireslrn Iskpsdaaka ehnmskmste
61 dprqvrnppk fgthhkalqe irnsllpfan etnssrstse vnpqmlqdlq aagfdedmvi
121 qalqktnnrs ieaaiefisk msyqdprreq maaaaarpin asmkpgnvqq svnrkqswkg
181 skeslvpqrh gpplgesvay hsespnsqtd vgrplsgsgi safvqahpsn gqrvnppppp
241 qvrsvtpppp prgqtppprg ttppppswep nsqtkrysgn meyvisrisp vppgawqegy
301 pppplntspm nppnqgqrgi ssvpvgrqpi imqssskfnf psgrpgmqng tgqtdfmihq
361 nvvpagtvnr qppppyplta angqspsalq tggsaapssy tngsipqsmm vpnrnshnme
421 lynisvpglq tnwpqsssap aqsspssghe iptwqpnipv rsnsfnnplg nrashsansq
481 psattvtait papiqqpvks mrvlkpelqt alapthpswi pqpiqtvqps pfpegtasnv
541 tvmppvaeap nyqgppppyp khllhqnpsv ppyesiskps kedqpslpke deseksyenv
601 dsgdkekkqi ttspitvrkn kkdeerresr iqsyspqafk ffmeqhvenv Ikshqqrlhr
661 kkqlenemmr vglsqdaqdq mrkmlcqkes nyirlkrakm dksmfvkikt Igigafgevc
721 larkvdtkal yatktlrkkd vllrnqvahv kaerdilaea dnewvvrlyy sfqdkdnlyf
781 vmdyipggdm msllirmgif peslarfyia eltcavesvh kmgfihrdik pdnilidrdg
841 hikltdfglc tgfrwthdsk yyqsgdhprq dsmdfsnewg dpsscrcgdr Ikplerraar
901 qhqrclahsl vgtpnyiape vllrtgytql cdwwsvgvil femlvgqppf laqtpletqm
961 kvinwqtslh ippqaklspe asdliiklcr gpedrlgkng adeikahpff ktidfssdlr
1021 qqsasyipki thptdtsnfd pvdpdklwsd dneeenvndt Ingwykngkh pehafyeftf
1081 rrffddngyp ynypkpieye yinsqgseqq sdeddqntgs eiknrdlvyv

LATS1: serine/threonine-protein kinase LATS1 isoform 2 [Homo sapiens] NCBI Reference Sequence: NP_001257448.1 (SEQ ID NO: 2:)

1 mkrsekpegy rqmrpktfpa snytvssrqm lqeireslrn Iskpsdaaka ehnmskmste
61 dprqvrnppk fgthhkalqe irnsllpfan etnssrstse vnpqmlqdlq aagfdedmvi
121 qalqktnnrs ieaaiefisk msyqdprreq maaaaarpin asmkpgnvqq svnrkqswkg
181 skeslvpqrh gpplgesvay hsespnsqtd vgrplsgsgi safvqahpsn gqrvnppppp
241 qvrsvtpppp prgqtppprg ttppppswep nsqtkrysgn meyvisrisp vppgawqegy
301 pppplntspm nppnqgqrgi ssvpvgrqpi imqssskfnf psgrpgmqng tgqtdfmihq
361 nvvpagtvnr qppppyplta angqspsalq tggsaapssy tngsipqsmm vpnrnshnme
421 lynisvpglq tnwpqsssap aqsspssghe iptwqpnipv rsnsfnnplg nrashsansq
481 psattvtait papiqqpvks mrvlkpelqt alapthpswi pqpiqtvqps pfpegtasnv
541 tvmppvaeap nyqgppppyp khllhqnpsv ppyesiskps kedqpslpke deseksyenv
601 dsgdkekkqi ttspitvrkn kkdeerresr iqsyspqafk ffmeqhvenv Ikshqqrlhr
661 kkqlenemmr vkpfkmsifi Inhlfawclf

LATS 2: NP_055387.2 serine/threonine-protein kinase LATS2 [Homo sapiens]. ((SEQ ID NO: 3:)

1 mrpktfpatt ysgnsrqrlq eireglkqps kssvqglpag pnsdtsldak vlgskdatrq
61 qqqmratpkf gpyqkalrei rysllpfane sgtsaaaevn rqmlqelvna gcdqemagra
121 lkqtgsrsie aaleyiskmg yldprneqiv rvikqtspgk glmptpvtrr psfegtgdsf
181 asyhqlsgtp yegpsfgadg ptaleemprp yvdylfpgvg phgpghqhqh ppkgygasve
241 aagahfplqg ahygrphllv pgeplgygvq rspsfqsktp petggyaslp tkgqggppga
301 glafpppaag lyvphphhkq agpaahqlhv lgsrsqvfas dsppqslltp srnslnvdly
361 elgstsvqqw paatlarrds Iqkpgleapp rahvafrpdc pvpsrtnsfn shqprpgppg
421 kaepslpapn tvtavtaahi lhpvksvrvl rpepqtavgp shpawvpapa papapapapa
481 aegldakeeh alalggagaf pldveyggpd rrcppppypk hlllrskseq ydldslcagm
541 eqslragpne peggdksrks akgdkggkdk kqiqtspvpv rknsrdeekr esriksyspy
601 afkffmeqhv enviktyqqk vnrrlqleqe makaglceae qeqmrkilyq kesnynrlkr
661 akmdksmfvk iktlgigafg evclackvdt halyamktlr kkdvlnrnqv ahvkaerdil
721 aeadnewvvk lyysfqdkds lyfvmdyipg gdmmsllirm evfpehlarf yiaeltlaie
781 svhkmgfihr dikpdnilid Idghikltdf glctgfrwth nskyyqkgsh vrqdsmepsd
841 lwddvsncrc gdrlktleqr arkqhqrcla hslvgtpnyi apevllrkgy tqlcdwwsvg
901 vilfemlvgq ppflaptpte tqlkvinwen tlhipaqvkl speardlitk Iccsadhrlg
961 rngaddlkah pffsaidfss dirkqpapyv ptishpmdts nfdpvdeesp wndasegstk
1021 awdtltspnn khpehafyef tfrrffddng ypfrcpkpsg aeasqaessd lessdlvdqt
1081 egcqpvyv

LATS is thought to negatively regulate YAP1 activity. “YAP1” refers to the yes-associated protein 1, also known as YAP or YAP65, which is a protein that acts as a transcriptional regulator of genes involved in cell proliferation. LATS kinases are serine/threonine protein kinases that have been shown to directly phosphorylate YAP which results in its cytoplasmic retention and inactivation. Without phosphorylation by LATS, YAP translocates into the nucleus, forming a complex with a DNA binding protein, TEAD, and results in downstream gene expression. (Barry ER & Camargo FD (2013) The Hippo superhighway: signaling crossroads converging on the Hippo/Yap pathway in stem cells and development. Current opinion in cell biology 25(2):247-253.; Mo JS, Park HW, & Guan KL (2014) The Hippo signaling pathway in stem cell biology and cancer. EMBO reports 15(6):642-656; Pan D (2010) The hippo signaling pathway in development and cancer. Developmental cell 19(4):491-505.)

The Hippo/YAP pathway is involved in numerous cell types and tissues in mammalian systems, including various cancers. In particular, the Hippo pathway is evidently involved in the intestine, stomach and esophagus, pancreas, salivary gland, skin, mammary gland, ovary, prostate, brain and nervous system, bone, chrondrocytes, adipose cells, myocytes, T lymphocytes, B lymphocytes, myeloid cells, kidney, and lung. See Nishio et al., 2017, Genes to Cells 22:6-31.

### LATS1 and LATS2 Inhibition

Compounds of Formula A1 or subformulae thereof (e.g., Formula A2), in free form or in salt form are potent inhibitors of LATS1 and/or LATS2.

In a preferred embodiment, the compounds of Formula A2 or subformulae thereof, in free form or in salt form are potent inhibitors of LATS1 and LATS2.

### LATS Inhibitors

The invention therefore relates to a compound of Formula A2:

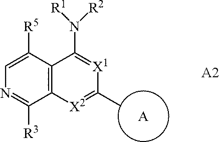

or a salt, or stereoisomer thereof, wherein
X¹ is CH or N;
Ring A is
   (a) a 5- or 6-membered monocyclic heteroaryl that is linked to the remainder of the molecule through a carbon ring member and comprises, as ring member, 1 to 4 heteroatoms that are independently selected from N, O and S, provided that at least one of the heteroatom ring member is an unsubstituted nitrogen (—N═) positioned at the 3- or the 4-position relative to the linking carbon ring member of the 5-membered heteroaryl or at the para ring position of the 6-membered heteroaryl; or a 9-membered fused bicyclic heteroaryl that is selected from 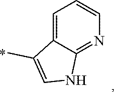 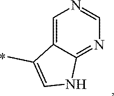 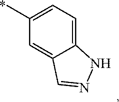 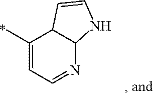 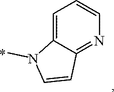
wherein “*” represents the point of attachment of ring A to the remainder of the molecule; and
wherein ring A is unsubstituted or substituted by 1 to 2 substituents independently selected from halogen, cyano, C₁₋₆alkyl, C₁₋₆haloalkyl, —NH₂, C₁₋₆alkylamino, di-(C₁₋₆alkyl)amino, C₃₋₆cycloalkyl, and phenylsulfonyl;
R⁰ is hydroxyl or C₁₋₆alkoxy;
R¹ is hydrogen or C₁₋₆alkyl;
R² is selected from
   (a) C₁₋₈alkyl that is unsubstituted or substituted by 1 to 3 substituents independently selected from
      (i) halogen;
      (ii) cyano;
      (iii) oxo;
      (iv) C₂alkenyl;
      (v) C₂alkynyl;
      (vi) C₁₋₆haloalkyl;
      (vii) —OR⁶, wherein R⁶ is selected from hydrogen, C₁₋₆alkyl that is unsubstituted or substituted by R⁰ or —C(O)R⁰;
      (viii) —NR^{7a}R^{7b}, wherein R^{7a} is hydrogen or C₁₋₆alkyl, and R^{7b} is selected from hydrogen, —C(O)R⁰, C₁₋₆alkyl that is unsubstituted or substituted by —C(O)R⁰;
      (ix) —C(O)R⁸, wherein R⁸ is R⁰ or —NH—C₁₋₆alkyl-C(O)R⁰;
      (x) -S(O)₂C₁₋₆alkyl;
      (xi) monocyclic C₃₋₆cycloalkyl or polycyclic C₇₋₁₀cycloalkyl that are each unsubstituted or substituted by 1 to 2 substituents independently selected from halogen, C₁₋ ₆alkyl, hydroxyC₁₋₆alkyl, C₁₋₆haloalkyl, R⁰, —NH₂, C₁₋₆alkylamino, and di-(C₁₋ ₆alkyl)amino;
      (xii) 6-membered heterocycloalkyl comprising, as ring members, 1 to 2 heteroatoms independently selected from N, O and S and that is unsubstituted or substituted by 1 to 2 substituents independently selected from hydroxyl, halogen, C₁₋₆alkyl, C₁₋₆alkylamino, and di-(C₁₋₆alkyl)amino;
      (xiii) phenyl that is unsubstituted or substituted by halogen;
      (xiv) 5- or 6-membered monocyclic heteroaryl comprising, as ring members, 1 to 4 heteroatoms independently selected from N and O; and
      (xv) 9- or 10-membered fused bicyclic heteroaryl comprising, as ring member, 1 to 2 heteroatoms independently selected from N and O;
   (b) -S(O)₂C₁₋₆alkyl;
   (c) phenyl that is unsubstituted or substituted by 1 to 2 substituents independently selected from halogen, C₁₋₆alkyl and R⁰;
   (d) C₃₋₆cycloalkyl that is unsubstituted or substituted by 1 to 2 substituents independently selected from C₁₋₆haloalkyl, R⁰, C₁₋₆alkylamino, di-(C₁₋₆alkyl)amino, —C(O)R⁰, and C₁₋ ₆alkyl that is unsubstituted or substituted by R⁰ or —C(O)R⁰; and
   (e) 4-membered heterocycloalkyl comprising, as ring members, 1 to 2 heteroatoms selected from N, O and S and that is unsubstituted or substituted by 1 to 2 substituents independently selected from C₁₋₆haloalkyl, R⁰, C₁₋₆alkylamino, di-(C₁₋ ₆alkyl)amino, —C(O)R⁰, and C₁₋₆alkyl that is unsubstituted or substituted by R⁰ or-C(O)R⁰;
or, provided that when X¹ is CH, R¹ and R² can be taken together with the nitrogen atom to which both are bound to form a 4- to 6-membered heterocycloalkyl that can include, as ring members, 1 to 2 additional heteroatoms independently selected from N, O, and S, wherein the 4- to 6-membered heterocycloalkyl formed by R¹ and R² taken together with the nitrogen atom to which both are bound is unsubstituted or substituted by 1 to 3 substituents independently selected from halogen, C₁₋₆alkyl, C₁₋₆haloalkyl, and R⁰;
R³ is selected from hydrogen, halogen and C₁₋₆alkyl; and
R⁵ is selected from hydrogen, halogen and —NH—(3- to 8-membered heteroalkyl), wherein the 3- to 8-membered heteroC₃₋₈alkyl of the —NH—(3- to 8-membered heteroalkyl) comprises 1 to 2 oxygen atoms as chain members and is unsubstituted or substituted by R⁰;
with the proviso that:
(1) when X¹ is N, ring A is 4-primidinyl or 3-fluoro-4-primidinyl, R¹ is H or methyl, R³ is H or Cl and R⁵ is H; then R² is not C₂₋₄alkyl that is substituted with a substituent selected from —NH₂, C₁₋₆alkylamino or t-butyl-carbamoyl-amino and that is optionally further substituted with unsubstituted phenyl; and
(2) when X¹ is N, ring A is indazol-5-yl, R¹, R³ and R⁵ are H; then R² is not C₄alkyl that is substituted with —NH₂.

Unless specified otherwise, the term “compounds of the present invention” refers to compounds of Formula A1 or subformulae thereof (e.g., Formula A2), or salts thereof, as well as all stereoisomers (including diastereoisomers and enantiomers), rotamers, tautomers and isotopically labeled compounds (including deuterium substitutions), as well as inherently formed moieties.

Various (enumerated) embodiments of the invention are described herein. It will be recognized that features specified in each embodiment may be combined with other specified features to provide further embodiments of the present invention. When an embodiment is described as being “according to” a previous embodiment, the previous embodiment includes sub-embodiments thereof, for example such that when Embodiment 20 is described as being “according to” embodiments 1 to 19, embodiments 1 to 19 includes embodiments 19 and 19A.

Embodiment 1. A method of cell population expansion, comprising the step of a) culturing a population of cells comprising limbal stem cells in the presence of a LATS inhibitor to generate an expanded population of cells comprising limbal stem cells, wherein the limbal stem cells have reduced or eliminated expression of B2M by a CRISPR system (e.g., S. pyogenes Cas9 CRISPR system), for example, a CRISPR system comprising a gRNA selected from those described in Table 1 or Table 4 or Table 6.

Embodiment 2. A method of cell population expansion, comprising the step of a) culturing a population of cells comprising corneal endothelial cells in the presence of a LATS inhibitor to generate an expanded population of cells comprising corneal endothelial cells, wherein the corneal endothelial cells have reduced or eliminated expression of B2M by a CRISPR system, for example, a CRISPR system (e.g., S. pyogenes Cas9 CRISPR system) comprising a gRNA selected from those described in Table 1 or Table 4 or Table 6.

Embodiment 3. The method of cell population expansion according to Embodiment 1 or Embodiment 2, wherein the LATS inhibitor is a compound of Formula A1

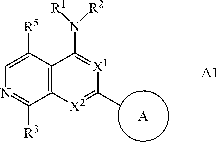

or a salt thereof, wherein
X¹ and X² are each independently CH or N;
Ring A is
   (a) a 5- or 6-membered monocyclic heteroaryl that is linked to the remainder of the molecule through a carbon ring member and comprises, as ring member, 1 to 4 heteroatoms that are independently selected from N, O and S, provided that at least one of the heteroatom ring member is an unsubstituted nitrogen (—N═) positioned at the 3- or the 4-position relative to the linking carbon ring member of the 5-membered heteroaryl or at the para ring position of the 6-membered heteroaryl; or
   (b) a 9-membered fused bicyclic heteroaryl that is selected from 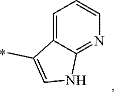 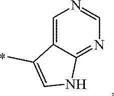 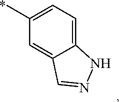 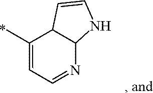 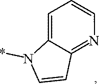
wherein “*” represents the point of attachment of ring A to the remainder of the molecule;
wherein ring A is unsubstituted or substituted by 1 to 2 substituents independently selected from halogen, cyano, C₁₋₆alkyl, C₁₋₆haloalkyl, —NH₂, C₁₋₆alkylamino, di-(C₁₋₆alkyl)amino, C₃₋₆cycloalkyl, and phenylsulfonyl;
R⁰ is hydroxyl or C₁₋₆alkoxy;
R¹ is hydrogen or C₁₋₆alkyl;
R² is selected from
   (a) C₁₋₈alkyl that is unsubstituted or substituted by 1 to 3 substituents independently selected from
      (i) halogen;
      (ii) cyano;
      (iii) oxo;
      (iv) C₂alkenyl;
      (v) C₂alkynyl;
      (vi) C₁₋₆haloalkyl;
      (vii) —OR⁶, wherein R⁶ is selected from hydrogen, C₁₋₆alkyl that is unsubstituted or substituted by R⁰ or —C(O)R⁰;
      (viii) —NR^{7a}R^{7b}, wherein R^{7a} is hydrogen or C₁₋₆alkyl, and R^{7b} is selected from hydrogen, —C(O)R⁰, C₁₋₆alkyl that is unsubstituted or substituted by —C(O)R⁰;
      (ix) —C(O)R⁸, wherein R⁸ is R⁰ or —NH—C₁₋₆alkyl-C(O)R⁰;
      (x) -S(O)₂C₁₋₆alkyl;
      (xi) monocyclic C₃₋₆cycloalkyl or polycyclic C₇₋₁₀cycloalkyl that are each unsubstituted or substituted by 1 to 2 substituents independently selected from halogen, C₁₋ ₆alkyl, hydroxyC₁₋₆alkyl, C₁₋₆haloalkyl, R⁰, —NH₂, C₁₋₆alkylamino, and di-(C₁₋ ₆alkyl)amino;
      (xii) 6-membered heterocycloalkyl comprising, as ring members, 1 to 2 heteroatoms independently selected from N, O and S and that is unsubstituted or substituted by 1 to 2 substituents independently selected from hydroxyl, halogen, C₁₋₆alkyl, C₁₋₆alkylamino, and di-(C₁₋₆alkyl)amino;
      (xiii) phenyl that is unsubstituted or substituted by halogen;
      (xiv) 5- or 6-membered monocyclic heteroaryl comprising, as ring members, 1 to 4 heteroatoms independently selected from N and O; and
      (xv) 9- or 10-membered fused bicyclic heteroaryl comprising, as ring member, 1 to 2 heteroatoms independently selected from N and O;
   (b) -S(O)₂C₁₋₆alkyl;
   (c) phenyl that is unsubstituted or substituted by 1 to 2 substituents independently selected from halogen, C₁₋₆alkyl and R⁰;
   (d) C₃₋₆cycloalkyl that is unsubstituted or substituted by 1 to 2 substituents independently selected from C₁₋₆haloalkyl, R⁰, C₁₋₆alkylamino, di-(C₁₋₆alkyl)amino, —C(O)R⁰, and C₁₋ ₆alkyl that is unsubstituted or substituted by R⁰ or —C(O)R⁰; and
   (e) 4-membered heterocycloalkyl comprising, as ring members, 1 to 2 heteroatoms selected from N, O and S and that is unsubstituted or substituted by 1 to 2 substituents independently selected from C₁₋₆haloalkyl, R⁰, C₁₋₆alkylamino, di-(C₁₋ ₆alkyl)amino, —C(O)R⁰, and C₁₋₆alkyl that is unsubstituted or substituted by R⁰ or-C(O)R⁰;
or R¹ and R² can be taken together with the nitrogen atom to which both are bound to form a 4- to 6-membered heterocycloalkyl that can include, as ring members, 1 to 2 additional heteroatoms independently selected from N, O, and S, wherein the 4- to 6-membered heterocycloalkyl formed by R¹ and R² taken together with the nitrogen atom to which both are bound is unsubstituted or substituted by 1 to 3 substituents independently selected from halogen, C₁₋₆alkyl, C₁₋₆haloalkyl, and R⁰;
R³ is selected from hydrogen, halogen and C₁₋₆alkyl; and
R⁵ is selected from hydrogen, halogen and —NH—(3- to 8-membered heteroalkyl), wherein the 3- to 8-membered heteroC₃₋₈alkyl of the —NH—(3- to 8-membered heteroalkyl) comprises 1 to 2 oxygen atoms as chain members and is unsubstituted or substituted by R⁰..

Embodiment 4. A method of cell population expansion, comprising the step of a) culturing a seeding population of cells comprising limbal stem cells in the presence of a compound of Formula A1,

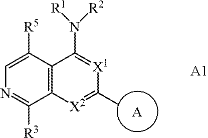

or a salt thereof to generate an expanded population of cells comprising limbal stem cells, wherein
X¹ and X² are each independently CH or N;
Ring A is
   (a) a 5- or 6-membered monocyclic heteroaryl that is linked to the remainder of the molecule through a carbon ring member and comprises, as ring member, 1 to 4 heteroatoms that are independently selected from N, O and S, provided that at least one of the heteroatom ring member is an unsubstituted nitrogen (—N═) positioned at the 3- or the 4-position relative to the linking carbon ring member of the 5-membered heteroaryl or at the para ring position of the 6-membered heteroaryl; or
   (b) a 9-membered fused bicyclic heteroaryl that is selected from 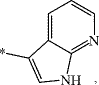 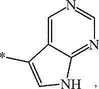 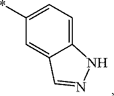 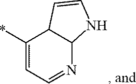 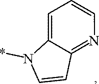
wherein “*” represents the point of attachment of ring A to the remainder of the molecule;
wherein ring A is unsubstituted or substituted by 1 to 2 substituents independently selected from halogen, cyano, C₁₋₆alkyl, C₁₋₆haloalkyl, —NH₂, C₁₋₆alkylamino, di-(C₁₋₆alkyl)amino, C₃₋₆cycloalkyl, and phenylsulfonyl;
R⁰ is hydroxyl or C₁₋₆alkoxy;
R¹ is hydrogen or C₁₋₆alkyl;
R² is selected from
   (a) C₁₋₈alkyl that is unsubstituted or substituted by 1 to 3 substituents independently selected from
      (i) halogen;
      (ii) cyano;
      (iii) oxo;
      (iv) C₂alkenyl;
      (v) C₂alkynyl;
      (vi) C₁₋₆haloalkyl;
      (vii) —OR⁶, wherein R⁶ is selected from hydrogen, C₁₋₆alkyl that is unsubstituted or substituted by R⁰ or —C(O)R⁰;
      (viii) —NR^{7a}R^{7b}, wherein R^{7a} is hydrogen or C₁₋₆alkyl, and R^{7b} is selected from hydrogen, —C(O)R⁰, C₁₋₆alkyl that is unsubstituted or substituted by —C(O)R⁰;
      (ix) —C(O)R⁸, wherein R⁸ is R⁰ or —NH—C₁₋₆alkyl-C(O)R⁰;
      (x) -S(O)₂C₁₋₆alkyl;
      (xi) monocyclic C₃₋₆cycloalkyl or polycyclic C₇₋₁₀cycloalkyl that are each unsubstituted or substituted by 1 to 2 substituents independently selected from halogen, C₁₋ ₆alkyl, hydroxyC₁₋₆alkyl, C₁₋₆haloalkyl, R⁰, —NH₂, C₁₋₆alkylamino, and di-(C₁₋ ₆alkyl)amino;
      (xii) 6-membered heterocycloalkyl comprising, as ring members, 1 to 2 heteroatoms independently selected from N, O and S and that is unsubstituted or substituted by 1 to 2 substituents independently selected from hydroxyl, halogen, C₁₋₆alkyl, C₁₋₆alkylamino, and di-(C₁₋₆alkyl)amino;
      (xiii) phenyl that is unsubstituted or substituted by halogen;
      (xiv) 5- or 6-membered monocyclic heteroaryl comprising, as ring members, 1 to 4 heteroatoms independently selected from N and O; and
      (xv) 9- or 10-membered fused bicyclic heteroaryl comprising, as ring member, 1 to 2 heteroatoms independently selected from N and O;
   (b) -S(O)₂C₁₋₆alkyl;
   (c) phenyl that is unsubstituted or substituted by 1 to 2 substituents independently selected from halogen, C₁₋₆alkyl and R⁰;
   (d) C₃₋₆cycloalkyl that is unsubstituted or substituted by 1 to 2 substituents independently selected from C₁₋₆haloalkyl, R⁰, C₁₋₆alkylamino, di-(C₁₋₆alkyl)amino, —C(O)R⁰, and C₁₋ ₆alkyl that is unsubstituted or substituted by R⁰ or —C(O)R⁰; and
   (e) 4-membered heterocycloalkyl comprising, as ring members, 1 to 2 heteroatoms selected from N, O and S and that is unsubstituted or substituted by 1 to 2 substituents independently selected from C₁₋₆haloalkyl, R⁰, C₁₋₆alkylamino, di-(C₁₋ ₆alkyl)amino, —C(O)R⁰, and C₁₋₆alkyl that is unsubstituted or substituted by R⁰ or —C(O)R⁰;
or R¹ and R² can be taken together with the nitrogen atom to which both are bound to form a 4- to 6-membered heterocycloalkyl that can include, as ring members, 1 to 2 additional heteroatoms independently selected from N, O, and S, wherein the 4- to 6-membered heterocycloalkyl formed by R¹ and R² taken together with the nitrogen atom to which both are bound is unsubstituted or substituted by 1 to 3 substituents independently selected from halogen, C₁₋₆alkyl, C₁₋₆haloalkyl, and R⁰;
R³ is selected from hydrogen, halogen and C₁₋₆alkyl; and
R⁵ is selected from hydrogen, halogen and —NH—(3- to 8-membered heteroalkyl), wherein the 3- to 8-membered heteroC₃₋₈alkyl of the —NH—(3- to 8-membered heteroalkyl) comprises 1 to 2 oxygen atoms as chain members and is unsubstituted or substituted by R⁰.

Embodiment 5. A method of cell population expansion, comprising the step of a) culturing a seeding population of cells comprising corneal endothelial cells in the presence of a compound of Formula A1,

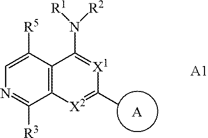

or a salt thereof to generate an expanded population of cells comprising corneal endothelial cells, wherein
X¹ and X² are each independently CH or N;
Ring A is
   (a) a 5- or 6-membered monocyclic heteroaryl that is linked to the remainder of the molecule through a carbon ring member and comprises, as ring member, 1 to 4 heteroatoms that are independently selected from N, O and S, provided that at least one of the heteroatom ring member is an unsubstituted nitrogen (—N═) positioned at the 3- or the 4-position relative to the linking carbon ring member of the 5-membered heteroaryl or at the para ring position of the 6-membered heteroaryl; or
   (b) a 9-membered fused bicyclic heteroaryl that is selected from 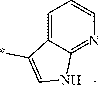 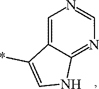 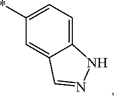 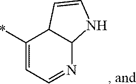 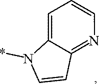
wherein “*” represents the point of attachment of ring A to the remainder of the molecule;
wherein ring A is unsubstituted or substituted by 1 to 2 substituents independently selected from halogen, cyano, C₁₋₆alkyl, C₁₋₆haloalkyl, —NH₂, C₁₋₆alkylamino, di-(C₁₋₆alkyl)amino, C₃₋₆cycloalkyl, and phenylsulfonyl;
R⁰ is hydroxyl or C₁₋₆alkoxy;
R¹ is hydrogen or C₁₋₆alkyl;
R² is selected from
   (a) C₁₋₈alkyl that is unsubstituted or substituted by 1 to 3 substituents independently selected from
      (i) halogen;
      (ii) cyano;
      (iii) oxo;
      (iv) C₂alkenyl;
      (v) C₂alkynyl;
      (vi) C₁₋₆haloalkyl;
      (vii) —OR⁶, wherein R⁶ is selected from hydrogen, C₁₋₆alkyl that is unsubstituted or substituted by R⁰ or —C(O)R⁰;
      (viii) —NR^{7a}R^{7b}, wherein R^{7a} is hydrogen or C₁₋₆alkyl, and R^{7b} is selected from hydrogen, —C(O)R⁰, C₁₋₆alkyl that is unsubstituted or substituted by —C(O)R⁰;
      (ix) —C(O)R⁸, wherein R⁸ is R⁰ or —NH—C₁₋₆alkyl-C(O)R⁰;
      (x) -S(O)₂C₁₋₆alkyl;
      (xi) monocyclic C₃₋₆cycloalkyl or polycyclic C₇₋₁₀cycloalkyl that are each unsubstituted or substituted by 1 to 2 substituents independently selected from halogen, C₁₋ ₆alkyl, hydroxyC₁₋₆alkyl, C₁₋₆haloalkyl, R⁰, —NH₂, C₁₋₆alkylamino, and di-(C₁₋ ₆alkyl)amino;
      (xii) 6-membered heterocycloalkyl comprising, as ring members, 1 to 2 heteroatoms independently selected from N, O and S and that is unsubstituted or substituted by 1 to 2 substituents independently selected from hydroxyl, halogen, C₁₋₆alkyl, C₁₋₆alkylamino, and di-(C₁₋₆alkyl)amino;
      (xiii) phenyl that is unsubstituted or substituted by halogen;
      (xiv) 5- or 6-membered monocyclic heteroaryl comprising, as ring members, 1 to 4 heteroatoms independently selected from N and O; and
      (xv) 9- or 10-membered fused bicyclic heteroaryl comprising, as ring member, 1 to 2 heteroatoms independently selected from N and O;
   (b) -S(O)₂C₁₋₆alkyl;
   (c) phenyl that is unsubstituted or substituted by 1 to 2 substituents independently selected from halogen, C₁₋₆alkyl and R⁰;
   (d) C₃₋₆cycloalkyl that is unsubstituted or substituted by 1 to 2 substituents independently selected from C₁₋₆haloalkyl, R⁰, C₁₋₆alkylamino, di-(C₁₋₆alkyl)amino, —C(O)R⁰, and C₁₋ ₆alkyl that is unsubstituted or substituted by R⁰ or —C(O)R⁰; and
   (e) 4-membered heterocycloalkyl comprising, as ring members, 1 to 2 heteroatoms selected from N, O and S and that is unsubstituted or substituted by 1 to 2 substituents independently selected from C₁₋₆haloalkyl, R⁰, C₁₋₆alkylamino, di-(C₁₋ ₆alkyl)amino, —C(O)R⁰, and C₁₋₆alkyl that is unsubstituted or substituted by R⁰ or —C(O)R⁰;
or R¹ and R² can be taken together with the nitrogen atom to which both are bound to form a 4- to 6-membered heterocycloalkyl that can include, as ring members, 1 to 2 additional heteroatoms independently selected from N, O, and S, wherein the 4- to 6-membered heterocycloalkyl formed by R¹ and R² taken together with the nitrogen atom to which both are bound is unsubstituted or substituted by 1 to 3 substituents independently selected from halogen, C₁₋₆alkyl, C₁₋₆haloalkyl, and R⁰;
R³ is selected from hydrogen, halogen and C₁₋₆alkyl; and
R⁵ is selected from hydrogen, halogen and —NH—(3- to 8-membered heteroalkyl), wherein the 3- to 8-membered heteroC₃₋₈alkyl of the —NH—(3- to 8-membered heteroalkyl) comprises 1 to 2 oxygen atoms as chain members and is unsubstituted or substituted by R⁰.

Embodiment 6. The method of cell population expansion according to Embodiment 3 to Embodiment 5, wherein the compound is selected from: N-methyl-2-(pyridin-4-yl)-N-(1,1,1-trifluoropropan-2-yl)pyrido[3,4-d]pyrimidin-4-amine; 2-methyl-1-(2-methyl-2-{[2-(pyridin-4-yl)pyrido[3,4-d]pyrimidin-4-yl]amino}propoxy)propan-2-ol; 2,4-dimethyl-4-{[2-(pyridin-4-yl)pyrido[3,4-d]pyrimidin-4-yl]amino}pentan-2-ol; N-tert-butyl-2-(pyrimidin-4-yl)-1,7-naphthyridin-4-amine; 2-(pyridin-4-yl)-N-[1-(trifluoromethyl)cyclobutyl]pyrido[3,4-d]pyrimidin-4-amine; N-propyl-2-(pyridin-4-yl)pyrido[3,4-d]pyrimidin-4-amine; N-(propan-2-yl)-2-(pyridin-4-yl)pyrido[3,4-d]pyrimidin-4-amine; 3-(pyridin-4-yl)-N-(1-(trifluoromethyl)cyclopropyl)-2,6-naphthyridin-1-amine; 2-(3-methyl-1H-pyrazol-4-yl)-N-(1-methylcyclopropyl)pyrido[3,4-d]pyrimidin-4-amine; 2-methyl-2-{[2-(pyridin-4-yl)pyrido[3,4-d]pyrimidin-4-yl]amino}propan-1-ol; 2-(pyridin-4-yl)-4-(3-(trifluoromethyl)piperazin-1-yl)pyrido[3,4-d]pyrimidine; N-cyclopentyl-2-(pyridin-4-yl)pyrido[3,4-d]pyrimidin-4-amine; N-propyl-2-(3-(trifluoromethyl)-1H-pyrazol-4-yl)pyrido[3,4-d]pyrimidin-4-amine; N-(2-methylcyclopentyl)-2-(pyridin-4-yl)pyrido[3,4-d]pyrimidin-4-amine; 2-(3-chloropyridin-4-yl)-N-(1,1,1-trifluoro-2-methylpropan-2-yl)pyrido[3,4-d]pyrimidin-4-amine; 2-(2-methyl-2-{[2-(pyridin-4-yl)pyrido[3,4-d]pyrimidin-4-yl]amino}propoxy)ethan-1-ol; N-(1-methylcyclopropyl)-7-(pyridin-4-yl)isoquinolin-5-amine; (1S,2S)-2-{[2-(pyridin-4-yl)pyrido[3,4-d]pyrimidin-4-yl]amino}cyclopentan-1-ol; N-methyl-2-(pyridin-4-yl)-N-[(2S)-1,1,1-trifluoropropan-2-yl]pyrido[3,4-d]pyrimidin-4-amine; N-methyl-N-(propan-2-yl)-2-(pyridin-4-yl)pyrido[3,4-d]pyrimidin-4-amine; N-(propan-2-yl)-2-(pyridin-4-yl)pyrido[3,4-d]pyrimidin-4-amine; 3-(pyridin-4-yl)-N-(1-(trifluoromethyl)cyclopropyl)-2,6-naphthyridin-1-amine and N-methyl-2-(pyridin-4-yl)-N-[(2R)-1,1,1-trifluoropropan-2-yl]pyrido[3,4-d]pyrimidin-4-amine.

Embodiment 7. The method of cell population expansion according to Embodiment 3 to Embodiment 5, wherein the compound is selected from 3-(pyridin-4-yl)-N-(1-(trifluoromethyl)cyclopropyl)-2,6-naphthyridin-1-amine; N-(1-methylcyclopropyl)-7-(pyridin-4-yl)isoquinolin-5-amine; 2-(pyridin-4-yl)-4-(3-(trifluoromethyl)piperazin-1-yl)pyrido[3,4-d]pyrimidine; N-(tert-butyl)-2-(pyridin-4-yl)-1,7-naphthyridin-4-amine; and N-methyl-2-(pyridin-4-yl)-N-[(2S)-1,1,1-trifluoropropan-2-yl]pyrido[3,4-d]pyrimidin-4-amine.

Embodiment 8. The method of cell population expansion according to Embodiment 3 to Embodiment 5, wherein the compound is selected from 3-(pyridin-4-yl)-N-(1-(trifluoromethyl)cyclopropyl)-2,6-naphthyridin-1-amine; N-(1-methylcyclopropyl)-7-(pyridin-4-yl)isoquinolin-5-amine; and 2-(pyridin-4-yl)-4-(3-(trifluoromethyl)piperazin-1-yl)pyrido[3,4-d]pyrimidine.

Embodiment 9. The method of cell population expansion according to Embodiment 3 to Embodiment 5, wherein the compound is selected from: dimethyl(3-methyl-3-{[2-(pyridin-4-yl)pyrido[3,4-d]pyrimidin-4-yl]amino}butyl)amine and N¹,N¹,3-trimethyl-N³-(2-(3-methyl-1H-pyrazol-4-yl)pyrido[3,4-d]pyrimidin-4-yl)butane-1,3-diamine.

Embodiment 10. The method of cell population expansion according to Embodiment 3 to Embodiment 5, wherein the compound is selected is dimethyl(3-methyl-3-{[2-(pyridin-4-yl)pyrido[3,4-d]pyrimidin-4-yl]amino}butyl)amine.

Embodiment 11. The method of cell population expansion according to Embodiment 3 to Embodiment 10, wherein said compound is present in a concentration of 0.5 to 100 micromolar, preferably 0.5 to 25 micromolar, more preferably 1 to 20 micromolar, particularly preferably of about 3 to 10 micromolar.

Embodiment 12. The method of cell population expansion according to Embodiment 3 to Embodiment 10, wherein in step a) the compound is present for one to two weeks and subsequently step b) is performed wherein the cells are cultured for a period in growth medium without supplementation with said compound, preferably wherein the period is one to two weeks.

Embodiment 13. The method of cell population expansion according to Embodiment 1 to Embodiment 10, wherein the method produces greater than 10 fold expansion of the seeded amount of cells.

Embodiment 14. The method of cell population expansion according to Embodiment 1 to Embodiment 10, wherein the method produces 15 fold to 600 fold, preferably 20 fold to 550 fold expansion of the seeded amount of cells.

Embodiment 15. The method of cell population expansion according to Embodiment 1 or Embodiment 2, wherein the LATS inhibitor inhibits LATS1 and LATS2.

Embodiment 16. The method of cell population expansion according to any one of Embodiment 2 to Embodiment 3 or Embodiment 5 to Embodiment 15, wherein said method further comprises genetically modifying said corneal endothelial cells.

Embodiment 17. The method of cell population expansion according to any one of Embodiment 1 or Embodiment 4 or Embodiment 6 to Embodiment 15, wherein said method further comprises genetically modifying said limbal stem cells.

Embodiment 18. The method of cell population expansion according to Embodiment 16 or Embodiment 17, wherein said genetically modifying comprises reducing or eliminating the expression and/or function of a gene associated with facilitating a host versus graft immune response.

Embodiment 19. The method of cell population expansion according to any one of Embodiment 16 to Embodiment 18, wherein said genetically modifying comprises introducing into said cell a gene editing system which specifically targets a gene associated with facilitating a host versus graft immune response.

Embodiment 20. The method of cell population expansion according to Embodiment 19, wherein said gene editing system is a CRISPR gene editing system.

Embodiment 21. The method of cell population expansion according to any one of Embodiment 16 to Embodiment 20, wherein said gene is B2M.

Embodiment 22. The method of cell population expansion according to any one of Embodiment 1 to Embodiment 21, which comprises the further step after generation of an expanded population of cells of rinsing those cells to substantially remove the compound.

Embodiment 23. A cell population obtainable by the method of any one of Embodiment 1 to Embodiment 22.

Embodiment 24. A cell population obtained by the method of any one of Embodiment 1 to Embodiment 22.

Embodiment 25. A cell population comprising corneal endothelial cells or the cell population of Embodiment 23 or Embodiment 24, wherein one or more of said cells comprises a non-naturally occurring insertion or deletion of one or more nucleic acid residues of a gene associated with facilitating a host vs graft immune response, wherein insertion and/or deletion results in reduced or eliminated expression or function of said gene.

Embodiment 26. The cell population according to Embodiment 25, wherein said gene is B2M.

Embodiment 27. A composition comprising the cell population according to Embodiment 25 or Embodiment 26.

Embodiment 28. A method of culturing cells comprising culturing a population of cells comprising corneal endothelial cells in the presence of a LATS inhibitor.

Embodiment 29. The method of culturing cells according to Embodiment 28, wherein the LATS inhibitor is a compound of Formula A1,

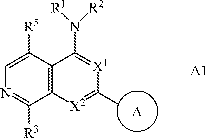

or a salt thereof, wherein
X¹ and X² are each independently CH or N;
Ring A is
   (a) a 5- or 6-membered monocyclic heteroaryl that is linked to the remainder of the molecule through a carbon ring member and comprises, as ring member, 1 to 4 heteroatoms that are independently selected from N, O and S, provided that at least one of the heteroatom ring member is an unsubstituted nitrogen (—N═) positioned at the 3- or the 4-position relative to the linking carbon ring member of the 5-membered heteroaryl or at the para ring position of the 6-membered heteroaryl; or
   (b) a 9-membered fused bicyclic heteroaryl that is selected from 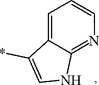 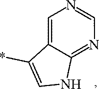 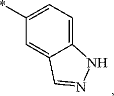 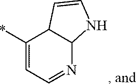 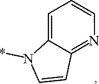
wherein “*” represents the point of attachment of ring A to the remainder of the molecule;
wherein ring A is unsubstituted or substituted by 1 to 2 substituents independently selected from halogen, cyano, C₁₋₆alkyl, C₁₋₆haloalkyl, —NH₂, C₁₋₆alkylamino, di-(C₁₋₆alkyl)amino, C₃₋₆cycloalkyl, and phenylsulfonyl;
R⁰ is hydroxyl or C₁₋₆alkoxy;
R¹ is hydrogen or C₁₋₆alkyl;
R² is selected from
   (a) C₁₋₈alkyl that is unsubstituted or substituted by 1 to 3 substituents independently selected from
      (i) halogen;
      (ii) cyano;
      (iii) oxo;
      (iv) C₂alkenyl;
      (v) C₂alkynyl;
      (vi) C₁₋₆haloalkyl;
      (vii) —OR⁶, wherein R⁶ is selected from hydrogen, C₁₋₆alkyl that is unsubstituted or substituted by R⁰ or —C(O)R⁰;
      (viii) —NR^{7a}R^{7b}, wherein R^{7a} is hydrogen or C₁₋₆alkyl, and R^{7b} is selected from hydrogen, —C(O)R⁰, C₁₋₆alkyl that is unsubstituted or substituted by —C(O)R⁰;
      (ix) —C(O)R⁸, wherein R⁸ is R⁰ or —NH—C₁₋₆alkyl-C(O)R⁰;
      (x) -S(O)₂C₁₋₆alkyl;
      (xi) monocyclic C₃₋₆cycloalkyl or polycyclic C₇₋₁₀cycloalkyl that are each unsubstituted or substituted by 1 to 2 substituents independently selected from halogen, C₁₋ ₆alkyl, hydroxyC₁₋₆alkyl, C₁₋₆haloalkyl, R⁰, —NH₂, C₁₋₆alkylamino, and di-(C₁₋ ₆alkyl)amino;
      (xii) 6-membered heterocycloalkyl comprising, as ring members, 1 to 2 heteroatoms independently selected from N, O and S and that is unsubstituted or substituted by 1 to 2 substituents independently selected from hydroxyl, halogen, C₁₋₆alkyl, C₁₋₆alkylamino, and di-(C₁₋₆alkyl)amino;
      (xiii) phenyl that is unsubstituted or substituted by halogen;
      (xiv) 5- or 6-membered monocyclic heteroaryl comprising, as ring members, 1 to 4 heteroatoms independently selected from N and O; and
      (xv) 9- or 10-membered fused bicyclic heteroaryl comprising, as ring member, 1 to 2 heteroatoms independently selected from N and O;
   (b) -S(O)₂C₁₋₆alkyl;
   (c) phenyl that is unsubstituted or substituted by 1 to 2 substituents independently selected from halogen, C₁₋₆alkyl and R⁰;
   (d) C₃₋₆cycloalkyl that is unsubstituted or substituted by 1 to 2 substituents independently selected from C₁₋₆haloalkyl, R⁰, C₁₋₆alkylamino, di-(C₁₋₆alkyl)amino, —C(O)R⁰, and C₁₋ ₆alkyl that is unsubstituted or substituted by R⁰ or —C(O)R⁰; and
   (e) 4-membered heterocycloalkyl comprising, as ring members, 1 to 2 heteroatoms selected from N, O and S and that is unsubstituted or substituted by 1 to 2 substituents independently selected from C₁₋₆haloalkyl, R⁰, C₁₋₆alkylamino, di-(C₁₋ ₆alkyl)amino, —C(O)R⁰, and C₁₋₆alkyl that is unsubstituted or substituted by R⁰ or —C(O)R⁰;
or R¹ and R² can be taken together with the nitrogen atom to which both are bound to form a 4- to 6-membered heterocycloalkyl that can include, as ring members, 1 to 2 additional heteroatoms independently selected from N, O, and S, wherein the 4- to 6-membered heterocycloalkyl formed by R¹ and R² taken together with the nitrogen atom to which both are bound is unsubstituted or substituted by 1 to 3 substituents independently selected from halogen, C₁₋₆alkyl, C₁₋₆haloalkyl, and R⁰;
R³ is selected from hydrogen, halogen and C₁₋₆alkyl; and
R⁵ is selected from hydrogen, halogen and —NH—(3- to 8-membered heteroalkyl), wherein the 3- to 8-membered heteroC₃₋₈alkyl of the —NH—(3- to 8-membered heteroalkyl) comprises 1 to 2 oxygen atoms as chain members and is unsubstituted or substituted by R⁰.

Embodiment 30. A method of culturing cells comprising culturing a population of cells comprising corneal endothelial cells in the presence of a compound of Formula A1,

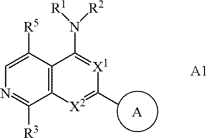

or a salt thereof, wherein
X¹ and X² are each independently CH or N;
Ring A is
   (a) a 5- or 6-membered monocyclic heteroaryl that is linked to the remainder of the molecule through a carbon ring member and comprises, as ring member, 1 to 4 heteroatoms that are independently selected from N, O and S, provided that at least one of the heteroatom ring member is an unsubstituted nitrogen (—N═) positioned at the 3- or the 4-position relative to the linking carbon ring member of the 5-membered heteroaryl or at the para ring position of the 6-membered heteroaryl; or
   (b) a 9-membered fused bicyclic heteroaryl that is selected from 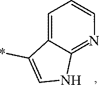 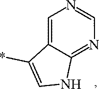 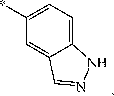 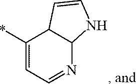 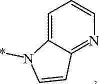
wherein “*” represents the point of attachment of ring A to the remainder of the molecule;
wherein ring A is unsubstituted or substituted by 1 to 2 substituents independently selected from halogen, cyano, C₁₋₆alkyl, C₁₋₆haloalkyl, —NH₂, C₁₋₆alkylamino, di-(C₁₋₆alkyl)amino, C₃₋₆cycloalkyl, and phenylsulfonyl;
R⁰ is hydroxyl or C₁₋₆alkoxy;
R¹ is hydrogen or C₁₋₆alkyl;
R² is selected from
   (a) C₁₋₈alkyl that is unsubstituted or substituted by 1 to 3 substituents independently selected from
      (i) halogen;
      (ii) cyano;
      (iii) oxo;
      (iv) C₂alkenyl;
      (v) C₂alkynyl;
      (vi) C₁₋₆haloalkyl;
      (vii) —OR⁶, wherein R⁶ is selected from hydrogen, C₁₋₆alkyl that is unsubstituted or substituted by R⁰ or —C(O)R⁰;
      (viii) —NR^{7a}R^{7b}, wherein R^{7a} is hydrogen or C₁₋₆alkyl, and R^{7b} is selected from hydrogen, —C(O)R⁰, C₁₋₆alkyl that is unsubstituted or substituted by —C(O)R⁰;
      (ix) —C(O)R⁸, wherein R⁸ is R⁰ or —NH—C₁₋₆alkyl-C(O)R⁰;
      (x) -S(O)₂C₁₋₆alkyl;
      (xi) monocyclic C₃₋₆cycloalkyl or polycyclic C₇₋₁₀cycloalkyl that are each unsubstituted or substituted by 1 to 2 substituents independently selected from halogen, C₁₋ ₆alkyl, hydroxyC₁₋₆alkyl, C₁₋₆haloalkyl, R⁰, —NH₂, C₁₋₆alkylamino, and di-(C₁₋ ₆alkyl)amino;
      (xii) 6-membered heterocycloalkyl comprising, as ring members, 1 to 2 heteroatoms independently selected from N, O and S and that is unsubstituted or substituted by 1 to 2 substituents independently selected from hydroxyl, halogen, C₁₋₆alkyl, C₁₋₆alkylamino, and di-(C₁₋₆alkyl)amino;
      (xiii) phenyl that is unsubstituted or substituted by halogen;
      (xiv) 5- or 6-membered monocyclic heteroaryl comprising, as ring members, 1 to 4 heteroatoms independently selected from N and O; and
      (xv) 9- or 10-membered fused bicyclic heteroaryl comprising, as ring member, 1 to 2 heteroatoms independently selected from N and O;
   (b) -S(O)₂C₁₋₆alkyl;
   (c) phenyl that is unsubstituted or substituted by 1 to 2 substituents independently selected from halogen, C₁₋₆alkyl and R⁰;
   (d) C₃₋₆cycloalkyl that is unsubstituted or substituted by 1 to 2 substituents independently selected from C₁₋₆haloalkyl, R⁰, C₁₋₆alkylamino, di-(C₁₋₆alkyl)amino, —C(O)R⁰, and C₁₋ ₆alkyl that is unsubstituted or substituted by R⁰ or —C(O)R⁰; and
   (e) 4-membered heterocycloalkyl comprising, as ring members, 1 to 2 heteroatoms selected from N, O and S and that is unsubstituted or substituted by 1 to 2 substituents independently selected from C₁₋₆haloalkyl, R⁰, C₁₋₆alkylamino, di-(C₁₋ ₆alkyl)amino, —C(O)R⁰, and C₁₋₆alkyl that is unsubstituted or substituted by R⁰ or —C(O)R⁰;
or R¹ and R² can be taken together with the nitrogen atom to which both are bound to form a 4- to 6-membered heterocycloalkyl that can include, as ring members, 1 to 2 additional heteroatoms independently selected from N, O, and S, wherein the 4- to 6-membered heterocycloalkyl formed by R¹ and R² taken together with the nitrogen atom to which both are bound is unsubstituted or substituted by 1 to 3 substituents independently selected from halogen, C₁₋₆alkyl, C₁₋₆haloalkyl, and R⁰;
R³ is selected from hydrogen, halogen and C₁₋₆alkyl; and
R⁵ is selected from hydrogen, halogen and —NH—(3- to 8-membered heteroalkyl), wherein the 3- to 8-membered heteroC₃₋₈alkyl of the —NH—(3- to 8-membered heteroalkyl) comprises 1 to 2 oxygen atoms as chain members and is unsubstituted or substituted by R⁰.

Embodiment 31. A method of culturing cells comprising culturing a population of cells comprising limbal stem cells in the presence of a LATS inhibitor.

Embodiment 32. The method of culturing cells according to Embodiment 31, wherein the LATS inhibitor is a compound of Formula A1,

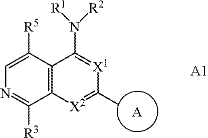

or a salt thereof, wherein
X¹ and X² are each independently CH or N;
Ring A is
   (a) a 5- or 6-membered monocyclic heteroaryl that is linked to the remainder of the molecule through a carbon ring member and comprises, as ring member, 1 to 4 heteroatoms that are independently selected from N, O and S, provided that at least one of the heteroatom ring member is an unsubstituted nitrogen (—N═) positioned at the 3- or the 4-position relative to the linking carbon ring member of the 5-membered heteroaryl or at the para ring position of the 6-membered heteroaryl; or
   (b) a 9-membered fused bicyclic heteroaryl that is selected from 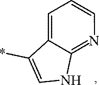 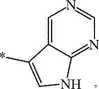 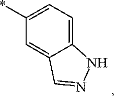 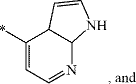 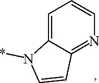
wherein “*” represents the point of attachment of ring A to the remainder of the molecule;
wherein ring A is unsubstituted or substituted by 1 to 2 substituents independently selected from halogen, cyano, C₁₋₆alkyl, C₁₋₆haloalkyl, —NH₂, C₁₋₆alkylamino, di-(C₁₋₆alkyl)amino, C₃₋₆cycloalkyl, and phenylsulfonyl;
R⁰ is hydroxyl or C₁₋₆alkoxy;
R¹ is hydrogen or C₁₋₆alkyl;
R² is selected from
   (a) C₁₋₈alkyl that is unsubstituted or substituted by 1 to 3 substituents independently selected from
      (i) halogen;
      (ii) cyano;
      (iii) oxo;
      (iv) C₂alkenyl;
      (v) C₂alkynyl;
      (vi) C₁₋₆haloalkyl;
      (vii) —OR⁶, wherein R⁶ is selected from hydrogen, C₁₋₆alkyl that is unsubstituted or substituted by R⁰ or —C(O)R⁰;
      (viii) —NR^{7a}R^{7b}, wherein R^{7a} is hydrogen or C₁₋₆alkyl, and R^{7b} is selected from hydrogen, —C(O)R⁰, C₁₋₆alkyl that is unsubstituted or substituted by —C(O)R⁰;
      (ix) —C(O)R⁸, wherein R⁸ is R⁰ or —NH—C₁₋₆alkyl-C(O)R⁰;
      (x) -S(O)₂C₁₋₆alkyl;
      (xi) monocyclic C₃₋₆cycloalkyl or polycyclic C₇₋₁₀cycloalkyl that are each unsubstituted or substituted by 1 to 2 substituents independently selected from halogen, C₁₋ ₆alkyl, hydroxyC₁₋₆alkyl, C₁₋₆haloalkyl, R⁰, —NH₂, C₁₋₆alkylamino, and di-(C₁₋ ₆alkyl)amino;
      (xii) 6-membered heterocycloalkyl comprising, as ring members, 1 to 2 heteroatoms independently selected from N, O and S and that is unsubstituted or substituted by 1 to 2 substituents independently selected from hydroxyl, halogen, C₁₋₆alkyl, C₁₋₆alkylamino, and di-(C₁₋₆alkyl)amino;
      (xiii) phenyl that is unsubstituted or substituted by halogen;
      (xiv) 5- or 6-membered monocyclic heteroaryl comprising, as ring members, 1 to 4 heteroatoms independently selected from N and O; and
      (xv) 9- or 10-membered fused bicyclic heteroaryl comprising, as ring member, 1 to 2 heteroatoms independently selected from N and O;
   (b) -S(O)₂C₁₋₆alkyl;
   (c) phenyl that is unsubstituted or substituted by 1 to 2 substituents independently selected from halogen, C₁₋₆alkyl and R⁰;
   (d) C₃₋₆cycloalkyl that is unsubstituted or substituted by 1 to 2 substituents independently selected from C₁₋₆haloalkyl, R⁰, C₁₋₆alkylamino, di-(C₁₋₆alkyl)amino, —C(O)R⁰, and C₁₋ ₆alkyl that is unsubstituted or substituted by R⁰ or —C(O)R⁰; and
   (e) 4-membered heterocycloalkyl comprising, as ring members, 1 to 2 heteroatoms selected from N, O and S and that is unsubstituted or substituted by 1 to 2 substituents independently selected from C₁₋₆haloalkyl, R⁰, C₁₋₆alkylamino, di-(C₁₋ ₆alkyl)amino, —C(O)R⁰, and C₁₋₆alkyl that is unsubstituted or substituted by R⁰ or —C(O)R⁰;
or R¹ and R² can be taken together with the nitrogen atom to which both are bound to form a 4- to 6-membered heterocycloalkyl that can include, as ring members, 1 to 2 additional heteroatoms independently selected from N, O, and S, wherein the 4- to 6-membered heterocycloalkyl formed by R¹ and R² taken together with the nitrogen atom to which both are bound is unsubstituted or substituted by 1 to 3 substituents independently selected from halogen, C₁₋₆alkyl, C₁₋₆haloalkyl, and R⁰;
R³ is selected from hydrogen, halogen and C₁₋₆alkyl; and
R⁵ is selected from hydrogen, halogen and —NH—(3- to 8-membered heteroalkyl), wherein the 3- to 8-membered heteroC₃₋₈alkyl of the —NH—(3- to 8-membered heteroalkyl) comprises 1 to 2 oxygen atoms as chain members and is unsubstituted or substituted by R⁰.

Embodiment 33. A method of culturing cells comprising culturing a population of cells comprising limbal stem cells in the presence of a compound of Formula A1,

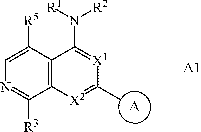

or a salt thereof, wherein
X¹ and X² are each independently CH or N;
Ring A is
   (a) a 5- or 6-membered monocyclic heteroaryl that is linked to the remainder of the molecule through a carbon ring member and comprises, as ring member, 1 to 4 heteroatoms that are independently selected from N, O and S, provided that at least one of the heteroatom ring member is an unsubstituted nitrogen (—N═) positioned at the 3- or the 4-position relative to the linking carbon ring member of the 5-membered heteroaryl or at the para ring position of the 6-membered heteroaryl; or
   (b) a 9-membered fused bicyclic heteroaryl that is selected from 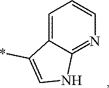 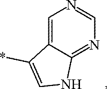 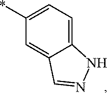 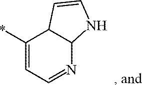 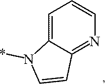
wherein “*” represents the point of attachment of ring A to the remainder of the molecule;
wherein ring A is unsubstituted or substituted by 1 to 2 substituents independently selected from halogen, cyano, C₁₋₆alkyl, C₁₋₆haloalkyl, —NH₂, C₁₋₆alkylamino, di-(C₁₋₆alkyl)amino, C₃₋₆cycloalkyl, and phenylsulfonyl;
R⁰ is hydroxyl or C₁₋₆alkoxy;
R¹ is hydrogen or C₁₋₆alkyl;
R² is selected from
   (a) C₁₋₈alkyl that is unsubstituted or substituted by 1 to 3 substituents independently selected from
      (i) halogen;
      (ii) cyano;
      (iii) oxo;
      (iv) C₂alkenyl;
      (v) C₂alkynyl;
      (vi) C₁₋₆haloalkyl;
      (vii) —OR⁶, wherein R⁶ is selected from hydrogen, C₁₋₆alkyl that is unsubstituted or substituted by R⁰ or —C(O)R⁰;
      (viii) —NR^{7a}R^{7b}, wherein R^{7a} is hydrogen or C₁₋₆alkyl, and R^{7b} is selected from hydrogen, —C(O)R⁰, C₁₋₆alkyl that is unsubstituted or substituted by —C(O)R⁰;
      (ix) —C(O)R⁸, wherein R⁸ is R⁰ or —NH—C₁₋₆alkyl-C(O)R⁰;
      (x) -S(O)₂C₁₋₆alkyl;
      (xi) monocyclic C₃₋₆cycloalkyl or polycyclic C₇₋₁₀cycloalkyl that are each unsubstituted or substituted by 1 to 2 substituents independently selected from halogen, C₁₋ ₆alkyl, hydroxyC₁₋₆alkyl, C₁₋₆haloalkyl, R⁰, —NH₂, C₁₋₆alkylamino, and di-(C₁₋ ₆alkyl)amino;
      (xii) 6-membered heterocycloalkyl comprising, as ring members, 1 to 2 heteroatoms independently selected from N, O and S and that is unsubstituted or substituted by 1 to 2 substituents independently selected from hydroxyl, halogen, C₁₋₆alkyl, C₁₋₆alkylamino, and di-(C₁₋₆alkyl)amino;
      (xiii) phenyl that is unsubstituted or substituted by halogen;
      (xiv) 5- or 6-membered monocyclic heteroaryl comprising, as ring members, 1 to 4 heteroatoms independently selected from N and O; and
      (xv) 9- or 10-membered fused bicyclic heteroaryl comprising, as ring member, 1 to 2 heteroatoms independently selected from N and O;
   (b) -S(O)₂C₁₋₆alkyl;
   (c) phenyl that is unsubstituted or substituted by 1 to 2 substituents independently selected from halogen, C₁₋₆alkyl and R⁰;
   (d) C₃₋₆cycloalkyl that is unsubstituted or substituted by 1 to 2 substituents independently selected from C₁₋₆haloalkyl, R⁰, C₁₋₆alkylamino, di-(C₁₋₆alkyl)amino, —C(O)R⁰, and C₁₋ ₆alkyl that is unsubstituted or substituted by R⁰ or —C(O)R⁰; and
   (e) 4-membered heterocycloalkyl comprising, as ring members, 1 to 2 heteroatoms selected from N, O and S and that is unsubstituted or substituted by 1 to 2 substituents independently selected from C₁₋₆haloalkyl, R⁰, C₁₋₆alkylamino, di-(C₁₋ ₆alkyl)amino, —C(O)R⁰, and C₁₋₆alkyl that is unsubstituted or substituted by R⁰ or —C(O)R⁰;
or R¹ and R² can be taken together with the nitrogen atom to which both are bound to form a 4- to 6-membered heterocycloalkyl that can include, as ring members, 1 to 2 additional heteroatoms independently selected from N, O, and S, wherein the 4- to 6-membered heterocycloalkyl formed by R¹ and R² taken together with the nitrogen atom to which both are bound is unsubstituted or substituted by 1 to 3 substituents independently selected from halogen, C₁₋₆alkyl, C₁₋₆haloalkyl, and R⁰;
R³ is selected from hydrogen, halogen and C₁₋₆alkyl; and
R⁵ is selected from hydrogen, halogen and —NH—(3- to 8-membered heteroalkyl), wherein the 3- to 8-membered heteroC₃₋₈alkyl of the —NH—(3- to 8-membered heteroalkyl) comprises 1 to 2 oxygen atoms as chain members and is unsubstituted or substituted by R⁰.

Embodiment 34. Use of a compound of the Formula A1, or a salt thereof,

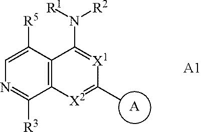

in a method of generating an expanded population of limbal stem cells, preferably ex vivo, wherein
X¹ and X² are each independently CH or N;
Ring A is
   (a) a 5- or 6-membered monocyclic heteroaryl that is linked to the remainder of the molecule through a carbon ring member and comprises, as ring member, 1 to 4 heteroatoms that are independently selected from N, O and S, provided that at least one of the heteroatom ring member is an unsubstituted nitrogen (—N═) positioned at the 3- or the 4-position relative to the linking carbon ring member of the 5-membered heteroaryl or at the para ring position of the 6-membered heteroaryl; or
   (b) a 9-membered fused bicyclic heteroaryl that is selected from 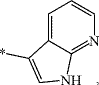 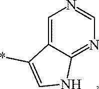 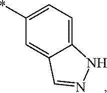 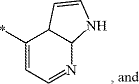 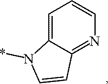
wherein “*” represents the point of attachment of ring A to the remainder of the molecule;
wherein ring A is unsubstituted or substituted by 1 to 2 substituents independently selected from halogen, cyano, C₁₋₆alkyl, C₁₋₆haloalkyl, —NH₂, C₁₋₆alkylamino, di-(C₁₋₆alkyl)amino, C₃₋₆cycloalkyl, and phenylsulfonyl;
R° is hydroxyl or C₁₋₆alkoxy;
R¹ is hydrogen or C₁₋₆alkyl;
R² is selected from
   (a) C₁₋₈alkyl that is unsubstituted or substituted by 1 to 3 substituents independently selected from
      (i) halogen;
      (ii) cyano;
      (iii) oxo;
      (iv) C₂alkenyl;
      (v) C₂alkynyl;
      (vi) C₁₋₆haloalkyl;
      (vii) —OR⁶, wherein R⁶ is selected from hydrogen, C₁₋₆alkyl that is unsubstituted or substituted by R° or —C(O)R⁰;
      (viii) —NR^{7a}R^{7b}, wherein R^{7a} is hydrogen or C₁₋₆alkyl, and R^{7b} is selected from hydrogen, —C(O)R⁰, C₁₋₆alkyl that is unsubstituted or substituted by —C(O)R⁰;
      (ix) —C(O)R⁸, wherein R⁸ is R° or —NH—C₁₋₆alkyl-C(0)R⁰;
      (x) -S(O)₂C₁₋₆alkyl;
      (xi) monocyclic C₃₋₆cycloalkyl or polycyclic C₇₋₁₀cycloalkyl that are each unsubstituted or substituted by 1 to 2 substituents independently selected from halogen, C₁₋ ₆alkyl, hydroxyC₁₋₆alkyl, C₁₋₆haloalkyl, R⁰, —NH₂, C₁₋₆alkylamino, and di-(C₁₋ ₆alkyl)amino;
      (xii) 6-membered heterocycloalkyl comprising, as ring members, 1 to 2 heteroatoms independently selected from N, O and S and that is unsubstituted or substituted by 1 to 2 substituents independently selected from hydroxyl, halogen, C₁₋₆alkyl, C₁₋₆alkylamino, and di-(C₁₋₆alkyl)amino;
      (xiii) phenyl that is unsubstituted or substituted by halogen;
      (xiv) 5- or 6-membered monocyclic heteroaryl comprising, as ring members, 1 to 4 heteroatoms independently selected from N and O; and
      (xv) 9- or 10-membered fused bicyclic heteroaryl comprising, as ring member, 1 to 2 heteroatoms independently selected from N and O;
   (b) -S(O)₂C₁₋₆alkyl;
   (c) phenyl that is unsubstituted or substituted by 1 to 2 substituents independently selected from halogen, C₁₋₆alkyl and R⁰;
   (d) C₃₋₆cycloalkyl that is unsubstituted or substituted by 1 to 2 substituents independently selected from C₁₋₆haloalkyl, R⁰, C₁₋₆alkylamino, di-(C₁₋₆alkyl)amino, —C(O)R⁰, and C₁₋ ₆alkyl that is unsubstituted or substituted by R⁰ or —C(O)R⁰; and
   (e) 4-membered heterocycloalkyl comprising, as ring members, 1 to 2 heteroatoms selected from N, O and S and that is unsubstituted or substituted by 1 to 2 substituents independently selected from C₁₋₆haloalkyl, R⁰, C₁₋₆alkylamino, di-(C₁₋ ₆alkyl)amino, —C(O)R⁰, and C₁₋₆alkyl that is unsubstituted or substituted by R⁰ or —C(O)R⁰;
or R¹ and R² can be taken together with the nitrogen atom to which both are bound to form a 4- to 6-membered heterocycloalkyl that can include, as ring members, 1 to 2 additional heteroatoms independently selected from N, O, and S, wherein the 4- to 6-membered heterocycloalkyl formed by R¹ and R² taken together with the nitrogen atom to which both are bound is unsubstituted or substituted by 1 to 3 substituents independently selected from halogen, C₁₋₆alkyl, C₁₋₆haloalkyl, and R⁰;
R³ is selected from hydrogen, halogen and C₁₋₆alkyl; and
R⁵ is selected from hydrogen, halogen and —NH—(3- to 8-membered heteroalkyl), wherein the 3- to 8-membered heteroC₃₋₈alkyl of the —NH—(3- to 8-membered heteroalkyl) comprises 1 to 2 oxygen atoms as chain members and is unsubstituted or substituted by _{RO}.

Embodiment 35. Use of a compound of the Formula A1, or a salt thereof,

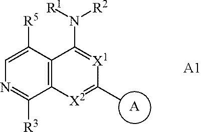

in a method of generating an expanded corneal endothelial cell population, preferably ex vivo, wherein
X¹ and X² are each independently CH or N;
Ring A is
   (a) a 5- or 6-membered monocyclic heteroaryl that is linked to the remainder of the molecule through a carbon ring member and comprises, as ring member, 1 to 4 heteroatoms that are independently selected from N, O and S, provided that at least one of the heteroatom ring member is an unsubstituted nitrogen (—N═) positioned at the 3- or the 4-position relative to the linking carbon ring member of the 5-membered heteroaryl or at the para ring position of the 6-membered heteroaryl; or
   (b) a 9-membered fused bicyclic heteroaryl that is selected from 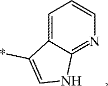 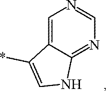 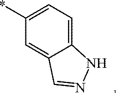 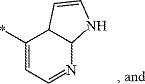 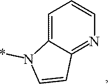
wherein “*” represents the point of attachment of ring A to the remainder of the molecule;
wherein ring A is unsubstituted or substituted by 1 to 2 substituents independently selected from halogen, cyano, C₁₋₆alkyl, C₁₋₆haloalkyl, —NH₂, C₁₋₆alkylamino, di-(C₁₋₆alkyl)amino, C₃₋₆cycloalkyl, and phenylsulfonyl;
R° is hydroxyl or C₁₋₆alkoxy;
R¹ is hydrogen or C₁₋₆alkyl;
R² is selected from
   (a) C₁₋₈alkyl that is unsubstituted or substituted by 1 to 3 substituents independently selected from
      (i) halogen;
      (ii) cyano;
      (iii) oxo;
      (iv) C₂alkenyl;
      (v) C₂alkynyl;
      (vi) C₁₋₆haloalkyl;
      (vii) —OR⁶, wherein R⁶ is selected from hydrogen, C₁₋₆alkyl that is unsubstituted or substituted by R⁰ or —C(O)R⁰;
      (viii) —NR^{7a}R^{7b}, wherein R^{7a} is hydrogen or C₁₋₆alkyl, and R^{7b} is selected from hydrogen, —C(O)R⁰, C₁₋₆alkyl that is unsubstituted or substituted by —C(O)R⁰;
      (ix) —C(O)R⁸, wherein R⁸ is R⁰ or —NH—C₁₋₆alkyl-C(0)R⁰;
      (x) -S(O)₂C₁₋₆alkyl;
      (xi) monocyclic C₃₋₆cycloalkyl or polycyclic C₇₋₁₀cycloalkyl that are each unsubstituted or substituted by 1 to 2 substituents independently selected from halogen, C₁₋ ₆alkyl, hydroxyC₁₋₆alkyl, C₁₋₆haloalkyl, R⁰, —NH₂, C₁₋₆alkylamino, and di-(C₁₋ ₆alkyl)amino;
      (xii) 6-membered heterocycloalkyl comprising, as ring members, 1 to 2 heteroatoms independently selected from N, O and S and that is unsubstituted or substituted by 1 to 2 substituents independently selected from hydroxyl, halogen, C₁₋₆alkyl, C₁₋₆alkylamino, and di-(C₁₋₆alkyl)amino;
      (xiii) phenyl that is unsubstituted or substituted by halogen;
      (xiv) 5- or 6-membered monocyclic heteroaryl comprising, as ring members, 1 to 4 heteroatoms independently selected from N and O; and
      (xv) 9- or 10-membered fused bicyclic heteroaryl comprising, as ring member, 1 to 2 heteroatoms independently selected from N and O;
   (b) -S(O)₂C₁₋₆alkyl;
   (c) phenyl that is unsubstituted or substituted by 1 to 2 substituents independently selected from halogen, C₁₋₆alkyl and R⁰;
   (d) C₃₋₆cycloalkyl that is unsubstituted or substituted by 1 to 2 substituents independently selected from C₁₋₆haloalkyl, R⁰, C₁₋₆alkylamino, di-(C₁₋₆alkyl)amino, —C(O)R⁰, and C₁₋ ₆alkyl that is unsubstituted or substituted by R⁰ or —C(O)R⁰; and
   (e) 4-membered heterocycloalkyl comprising, as ring members, 1 to 2 heteroatoms selected from N, O and S and that is unsubstituted or substituted by 1 to 2 substituents independently selected from C₁₋₆haloalkyl, R⁰, C₁₋₆alkylamino, di-(C₁₋ ₆alkyl)amino, —C(O)R⁰, and C₁₋₆alkyl that is unsubstituted or substituted by R⁰ or —C(O)R⁰;
or R¹ and R² can be taken together with the nitrogen atom to which both are bound to form a 4- to 6-membered heterocycloalkyl that can include, as ring members, 1 to 2 additional heteroatoms independently selected from N, O, and S, wherein the 4- to 6-membered heterocycloalkyl formed by R¹ and R² taken together with the nitrogen atom to which both are bound is unsubstituted or substituted by 1 to 3 substituents independently selected from halogen, C₁₋₆alkyl, C₁₋₆haloalkyl, and R⁰;
R³ is selected from hydrogen, halogen and C₁₋₆alkyl; and
R⁵ is selected from hydrogen, halogen and —NH—(3- to 8-membered heteroalkyl), wherein the 3- to 8-membered heteroC₃₋₈alkyl of the —NH—(3- to 8-membered heteroalkyl) comprises 1 to 2 oxygen atoms as chain members and is unsubstituted or substituted by R⁰.

Embodiment 36. Use of a compound of the Formula A1 or a salt thereof, according to Embodiment 34 or Embodiment 35, wherein the compound is of the formula selected from Formulae I to IV:

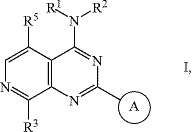

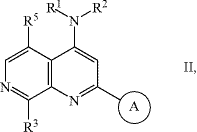

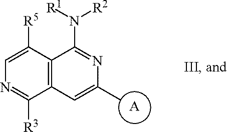

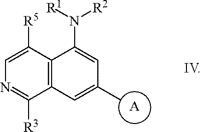

Embodiment 37. Use of a compound of Formula A1 or a salt thereof, according to Embodiment 34 or Embodiment 35, wherein the compound is selected from 3-(pyridin-4-yl)-N-(1-(trifluoromethyl)cyclopropyl)-2,6-naphthyridin-1-amine; N-(1-methylcyclopropyl)-7-(pyridin-4-yl)isoquinolin-5-amine; and 2-(pyridin-4-yl)-4-(3-(trifluoromethyl)piperazin-1-yl)pyrido[3,4-d]pyrimidine.

Embodiment 38. Use of a compound of the Formula Al, or a salt thereof, according to Embodiment 34 or Embodiment 35, wherein the compound is selected from: N-methyl-2-(pyridin-4-yl)-N-(1,1,1-trifluoropropan-2-yl)pyrido[3,4-d]pyrimidin-4-amine; 2-methyl-1-(2-methyl-2-{[2-(pyridin-4-yl)pyrido[3,4-d]pyrimidin-4-yl]amino}propoxy)propan-2-ol; 2,4-dimethyl-4-{[2-(pyridin-4-yl)pyrido[3,4-d]pyrimidin-4-yl]amino}pentan-2-ol; N-tert-butyl-2-(pyrimidin-4-yl)-1,7-naphthyridin-4-amine; 2-(pyridin-4-yl)-N-[1-(trifluoromethyl)cyclobutyl]pyrido[3,4-d]pyrimidin-4-amine; N-propyl-2-(pyridin-4-yl)pyrido[3,4-d]pyrimidin-4-amine; N-(propan-2-yl)-2-(pyridin-4-yl)pyrido[3,4-d]pyrimidin-4-amine; 3-(pyridin-4-yl)-N-(1-(trifluoromethyl)cyclopropyl)-2,6-naphthyridin-1-amine; 2-(3-methyl-1H-pyrazol-4-yl)-N-(1-methylcyclopropyl)pyrido[3,4-d]pyrimidin-4-amine; 2-methyl-2-{[2-(pyridin-4-yl)pyrido[3,4-d]pyrimidin-4-yl]amino}propan-1-ol; 2-(pyridin-4-yl)-4-(3-(trifluoromethyl)piperazin-1-yl)pyrido[3,4-d]pyrimidine; N-cyclopentyl-2-(pyridin-4-yl)pyrido[3,4-d]pyrimidin-4-amine; N-propyl-2-(3-(trifluoromethyl)-1H-pyrazol-4-yl)pyrido[3,4-d]pyrimidin-4-amine; N-(2-methylcyclopentyl)-2-(pyridin-4-yl)pyrido[3,4-d]pyrimidin-4-amine; 2-(3-chloropyridin-4-yl)-N-(1,1,1-trifluoro-2-methylpropan-2-yl)pyrido[3,4-d]pyrimidin-4-amine; 2-(2-methyl-2-{[2-(pyridin-4-yl)pyrido[3,4-d]pyrimidin-4-yl]amino}propoxy)ethan-1-ol; N-(1-methylcyclopropyl)-7-(pyridin-4-yl)isoquinolin-5-amine; (1S,2S)-2-{[2-(pyridin-4-yl)pyrido[3,4-d]pyrimidin-4-yl]amino}cyclopentan-1-ol; N-methyl-2-(pyridin-4-yl)-N-[(2S)-1,1,1-trifluoropropan-2-yl]pyrido[3,4-d]pyrimidin-4-amine; N-methyl-N-(propan-2-yl)-2-(pyridin-4-yl)pyrido[3,4-d]pyrimidin-4-amine; N-(propan-2-yl)-2-(pyridin-4-yl)pyrido[3,4-d]pyrimidin-4-amine; 3-(pyridin-4-yl)-N-(1-(trifluoromethyl)cyclopropyl)-2,6-naphthyridin-1-amine and N-methyl-2-(pyridin-4-yl)-N-[(2R)-1,1,1-trifluoropropan-2-yl]pyrido[3,4-d]pyrimidin-4-amine.

Embodiment 39. Use of a compound of the Formula Al,or a salt thereof, according to Embodiment 34 or Embodiment 35, wherein the compound is selected from: N-(tert-butyl)-2-(pyridin-4-yl)-1,7-naphthyridin-4-amine; and N-methyl-2-(pyridin-4-yl)-N-[(2S)-1,1,1-trifluoropropan-2-yl]pyrido[3,4-d]pyrimidin-4-amine, in particular N-(tert-butyl)-2-(pyridin-4-yl)-1,7-naphthyridin-4-amine.

Embodiment 40. Use of a compound of the Formula Al, or a salt thereof, according to Embodiment 34 or Embodiment 35, wherein the compound is selected from: dimethyl(3-methyl-3-{[2-(pyridin-4-yl)pyrido[3,4-d]pyrimidin-4-yl]amino}butyl)amine and N′,N′,3-trimethyl-N³-(2-(3-methyl-1H-pyrazol-4-yl)pyrido[3,4-d]pyrimidin-4-yl)butane-1,3-diamine, in particular dimethyl(3-methyl-3-{[2-(pyridin-4-yl)pyrido[3,4-d]pyrimidin-4-yl]amino}butyl)amine.

Embodiment 41. A method of treatment of an ocular disease or disorder comprising administering to a subject in need thereof a modified cell population, wherein the cell population has been grown in the presence of a compound of Formula A1, or a salt thereof,

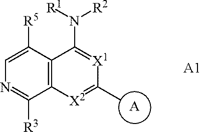

wherein
X¹ and X² are each independently CH or N;
Ring A is
   (a) a 5- or 6-membered monocyclic heteroaryl that is linked to the remainder of the molecule through a carbon ring member and comprises, as ring member, 1 to 4 heteroatoms that are independently selected from N, O and S, provided that at least one of the heteroatom ring member is an unsubstituted nitrogen (—N═) positioned at the 3- or the 4-position relative to the linking carbon ring member of the 5-membered heteroaryl or at the para ring position of the 6-membered heteroaryl; or
   (b) a 9-membered fused bicyclic heteroaryl that is selected from 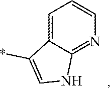 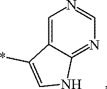 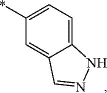 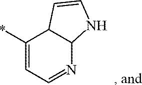 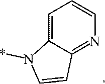
wherein “*” represents the point of attachment of ring A to the remainder of the molecule;
wherein ring A is unsubstituted or substituted by 1 to 2 substituents independently selected from halogen, cyano, C₁₋₆alkyl, C₁₋₆haloalkyl, —NH₂, C₁₋₆alkylamino, di-(C₁₋₆alkyl)amino, C₃₋₆cycloalkyl, and phenylsulfonyl;
R⁰ is hydroxyl or C₁₋₆alkoxy;
R¹ is hydrogen or C₁₋₆alkyl;
R² is selected from
   (a) C₁₋₈alkyl that is unsubstituted or substituted by 1 to 3 substituents independently selected from
      (i) halogen;
      (ii) cyano;
      (iii) oxo;
      (iv) C₂alkenyl;
      (v) C₂alkynyl;
      (vi) C₁₋₆haloalkyl;
      (vii) —OR⁶, wherein R⁶ is selected from hydrogen, C₁₋₆alkyl that is unsubstituted or substituted by R⁰ or —C(O)R⁰;
      (viii) —NR^{7a}R^{7b}, wherein R^{7a} is hydrogen or C₁₋₆alkyl, and R^{7b} is selected from hydrogen, —C(O)R⁰, C₁₋₆alkyl that is unsubstituted or substituted by —C(O)R⁰;
      (ix) —C(O)R⁸, wherein R⁸ is R° or —NH—C₁₋₆alkyl-C(0)R⁰;
      (x) -S(O)₂C₁₋₆alkyl;
      (xi) monocyclic C₃₋₆cycloalkyl or polycyclic C₇₋₁₀cycloalkyl that are each unsubstituted or substituted by 1 to 2 substituents independently selected from halogen, C₁₋₆alkyl, hydroxyC₁₋₆alkyl, C₁₋₆haloalkyl, R⁰, —NH₂, C₁₋₆alkylamino, and di-(C₁₋₆alkyl)amino;
      (xii) 6-membered heterocycloalkyl comprising, as ring members, 1 to 2 heteroatoms independently selected from N, O and S and that is unsubstituted or substituted by 1 to 2 substituents independently selected from hydroxyl, halogen, C₁₋₆alkyl, C₁₋₆alkylamino, and di-(C₁₋₆alkyl)amino;
      (xiii) phenyl that is unsubstituted or substituted by halogen;
      (xiv) 5- or 6-membered monocyclic heteroaryl comprising, as ring members, 1 to 4 heteroatoms independently selected from N and O; and
      (xv) 9- or 10-membered fused bicyclic heteroaryl comprising, as ring member, 1 to 2 heteroatoms independently selected from N and O;
   (b) -S(O)₂C₁₋₆alkyl;
   (c) phenyl that is unsubstituted or substituted by 1 to 2 substituents independently selected from halogen, C₁₋₆alkyl and R⁰;
   (d) C₃₋₆cycloalkyl that is unsubstituted or substituted by 1 to 2 substituents independently selected from C₁₋₆haloalkyl, R⁰, C₁₋₆alkylamino, di-(C₁₋₆alkyl)amino, —C(O)R⁰, and C₁₋ ₆alkyl that is unsubstituted or substituted by R⁰ or —C(O)R⁰; and
   (e) 4-membered heterocycloalkyl comprising, as ring members, 1 to 2 heteroatoms selected from N, O and S and that is unsubstituted or substituted by 1 to 2 substituents independently selected from C₁₋₆haloalkyl, R⁰, C₁₋₆alkylamino, di-(C₁₋ ₆alkyl)amino, —C(O)R⁰, and C₁₋₆alkyl that is unsubstituted or substituted by R⁰ or —C(O)R⁰;
or, R¹ and R² can be taken together with the nitrogen atom to which both are bound to form a 4- to 6-membered heterocycloalkyl that can include, as ring members, 1 to 2 additional heteroatoms independently selected from N, O, and S, wherein the 4- to 6-membered heterocycloalkyl formed by R¹ and R² taken together with the nitrogen atom to which both are bound is unsubstituted or substituted by 1 to 3 substituents independently selected from halogen, C₁₋₆alkyl, C₁₋₆haloalkyl, and R⁰; R³ is selected from hydrogen, halogen and C₁₋₆alkyl; and
R⁵ is selected from hydrogen, halogen and —NH—(3- to 8-membered heteroalkyl), wherein the 3- to 8-membered heteroC₃₋₈alkyl of the —NH—(3- to 8-membered heteroalkyl) comprises 1 to 2 oxygen atoms as chain members and is unsubstituted or substituted by R⁰.

Embodiment 42. A method of treatment of an ocular disease or disorder comprising administering to a subject in need thereof a modified limbal stem cell population, wherein said population has been grown in the presence of a compound of Formula A1, or a salt thereof,

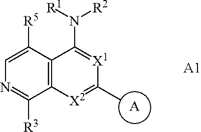

wherein
X¹ and X² are each independently CH or N;
Ring A is
   (a) a 5- or 6-membered monocyclic heteroaryl that is linked to the remainder of the molecule through a carbon ring member and comprises, as ring member, 1 to 4 heteroatoms that are independently selected from N, O and S, provided that at least one of the heteroatom ring member is an unsubstituted nitrogen (—N═) positioned at the 3- or the 4-position relative to the linking carbon ring member of the 5-membered heteroaryl or at the para ring position of the 6-membered heteroaryl; or
   (b) a 9-membered fused bicyclic heteroaryl that is selected from 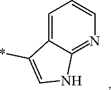 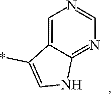 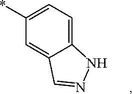 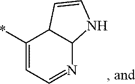 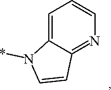
wherein “*” represents the point of attachment of ring A to the remainder of the molecule;
wherein ring A is unsubstituted or substituted by 1 to 2 substituents independently selected from halogen, cyano, C₁₋₆alkyl, C₁₋₆haloalkyl, —NH₂, C₁₋₆alkylamino, di-(C₁₋₆alkyl)amino, C₃₋₆cycloalkyl, and phenylsulfonyl;
R⁰ is hydroxyl or C₁₋₆alkoxy;
R¹ is hydrogen or C₁₋₆alkyl;
R² is selected from
   (a) C₁₋₈alkyl that is unsubstituted or substituted by 1 to 3 substituents independently selected from
      (i) halogen;
      (ii) cyano;
      (iii) oxo;
      (iv) C₂alkenyl;
      (v) C₂alkynyl;
      (vi) C₁₋₆haloalkyl;
      (vii) —OR⁶, wherein R⁶ is selected from hydrogen, C₁₋₆alkyl that is unsubstituted or substituted by R⁰ or —C(O)R⁰;
      (viii) —NR^{7a}R^{7b}, wherein R^{7a} is hydrogen or C₁₋₆alkyl, and R^{7b} is selected from hydrogen, —C(O)R⁰, C₁₋₆alkyl that is unsubstituted or substituted by —C(O)R⁰;
      (ix) —C(O)R⁸, wherein R⁸ is R⁰ or —NH—C₁₋₆alkyl-C(0)R⁰;
      (x) -S(O)₂C₁₋₆alkyl;
      (xi) monocyclic C₃₋₆cycloalkyl or polycyclic C₇₋₁₀cycloalkyl that are each unsubstituted or substituted by 1 to 2 substituents independently selected from halogen, C₁₋₆alkyl, hydroxyC₁₋₆alkyl, C₁₋₆haloalkyl, R⁰, —NH₂, C₁₋₆alkylamino, and di-(C₁₋₆alkyl)amino;
      (xii) 6-membered heterocycloalkyl comprising, as ring members, 1 to 2 heteroatoms independently selected from N, O and S and that is unsubstituted or substituted by 1 to 2 substituents independently selected from hydroxyl, halogen, C₁₋₆alkyl, C₁₋ ₆alkylamino, and di-(C₁₋₆alkyl)amino;
      (xiii) phenyl that is unsubstituted or substituted by halogen;
      (xiv) 5- or 6-membered monocyclic heteroaryl comprising, as ring members, 1 to 4 heteroatoms independently selected from N and O; and
      (xv) 9- or 10-membered fused bicyclic heteroaryl comprising, as ring member, 1 to 2 heteroatoms independently selected from N and O;
   (b) -S(O)₂C₁₋₆alkyl;
   (c) phenyl that is unsubstituted or substituted by 1 to 2 substituents independently selected from halogen, C₁₋₆alkyl and R⁰;
   (d) C₃₋₆cycloalkyl that is unsubstituted or substituted by 1 to 2 substituents independently selected from C₁₋₆haloalkyl, R⁰, C₁₋₆alkylamino, di-(C₁₋₆alkyl)amino, —C(O)R⁰, and C₁₋ ₆alkyl that is unsubstituted or substituted by R⁰ or —C(O)R⁰; and
   (e) 4-membered heterocycloalkyl comprising, as ring members, 1 to 2 heteroatoms selected from N, O and S and that is unsubstituted or substituted by 1 to 2 substituents independently selected from C₁₋₆haloalkyl, R⁰, C₁₋₆alkylamino, di-(C₁₋ ₆alkyl)amino, —C(O)R⁰, and C₁₋₆alkyl that is unsubstituted or substituted by R⁰ or —C(O)R⁰;
or, R¹ and R² can be taken together with the nitrogen atom to which both are bound to form a 4- to 6-membered heterocycloalkyl that can include, as ring members, 1 to 2 additional heteroatoms independently selected from N, O, and S, wherein the 4- to 6-membered heterocycloalkyl formed by R¹ and R² taken together with the nitrogen atom to which both are bound is unsubstituted or substituted by 1 to 3 substituents independently selected from halogen, C₁₋₆alkyl, C₁₋₆haloalkyl, and R⁰; R³ is selected from hydrogen, halogen and C₁₋₆alkyl; and
R⁵ is selected from hydrogen, halogen and —NH—(3- to 8-membered heteroalkyl), wherein the 3- to 8-membered heteroC₃₋₈alkyl of the —NH—(3- to 8-membered heteroalkyl) comprises 1 to 2 oxygen atoms as chain members and is unsubstituted or substituted by R⁰.

Embodiment 43. A method of treatment of an ocular disease or disorder comprising administering to a subject in need thereof a modified corneal endothelial cell population, wherein the population has been grown in the presence of a compound of Formula A1, or a salt thereof,

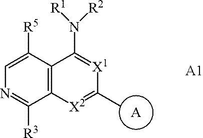

wherein
X¹ and X² are each independently CH or N;
Ring A is
   (a) a 5- or 6-membered monocyclic heteroaryl that is linked to the remainder of the molecule through a carbon ring member and comprises, as ring member, 1 to 4 heteroatoms that are independently selected from N, O and S, provided that at least one of the heteroatom ring member is an unsubstituted nitrogen (—N═) positioned at the 3- or the 4-position relative to the linking carbon ring member of the 5-membered heteroaryl or at the para ring position of the 6-membered heteroaryl; or
   (b) a 9-membered fused bicyclic heteroaryl that is selected from 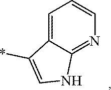 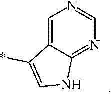 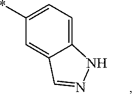 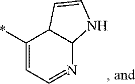 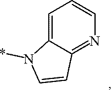
wherein “*” represents the point of attachment of ring A to the remainder of the molecule;
wherein ring A is unsubstituted or substituted by 1 to 2 substituents independently selected from halogen, cyano, C₁₋₆alkyl, C₁₋₆haloalkyl, —NH₂, C₁₋₆alkylamino, di-(C₁₋₆alkyl)amino, C₃₋₆cycloalkyl, and phenylsulfonyl;
R° is hydroxyl or C₁₋₆alkoxy;
R¹ is hydrogen or C₁₋₆alkyl;
R² is selected from
   (a) C₁₋₈alkyl that is unsubstituted or substituted by 1 to 3 substituents independently selected from
      (i) halogen;
      (ii) cyano;
      (iii) oxo;
      (iv) C₂alkenyl;
      (v) C₂alkynyl;
      (vi) C₁₋₆haloalkyl;
      (vii) —OR⁶, wherein R⁶ is selected from hydrogen, C₁₋₆alkyl that is unsubstituted or substituted by R° or —C(O)R⁰;
      (viii) —NR^{7a}R^{7b}, wherein R^{7a} is hydrogen or C₁₋₆alkyl, and R^{7b} is selected from hydrogen, —C(O)R⁰, C₁₋₆alkyl that is unsubstituted or substituted by —C(O)R⁰;
      (ix) —C(O)R⁸, wherein R⁸ is R° or —NH—C₁₋₆alkyl-C(0)R⁰;
      (x) -S(O)₂C₁₋₆alkyl;
      (xi) monocyclic C₃₋₆cycloalkyl or polycyclic C₇₋₁₀cycloalkyl that are each unsubstituted or substituted by 1 to 2 substituents independently selected from halogen, C₁₋₆alkyl, hydroxyC₁₋₆alkyl, C₁₋₆haloalkyl, R⁰, —NH₂, C₁₋₆alkylamino, and di-(C₁₋₆alkyl)amino;
      (xii) 6-membered heterocycloalkyl comprising, as ring members, 1 to 2 heteroatoms independently selected from N, O and S and that is unsubstituted or substituted by 1 to 2 substituents independently selected from hydroxyl, halogen, C₁₋₆alkyl, C₁₋₆alkylamino, and di-(C₁₋₆alkyl)amino;
      (xiii) phenyl that is unsubstituted or substituted by halogen;
      (xiv) 5- or 6-membered monocyclic heteroaryl comprising, as ring members, 1 to 4 heteroatoms independently selected from N and O; and
      (xv) 9- or 10-membered fused bicyclic heteroaryl comprising, as ring member, 1 to 2 heteroatoms independently selected from N and O;
   (b) -S(O)₂C₁₋₆alkyl;
   (c) phenyl that is unsubstituted or substituted by 1 to 2 substituents independently selected from halogen, C₁₋₆alkyl and R⁰;
   (d) C₃₋₆cycloalkyl that is unsubstituted or substituted by 1 to 2 substituents independently selected from C₁₋₆haloalkyl, R⁰, C₁₋₆alkylamino, di-(C₁₋₆alkyl)amino, —C(O)R⁰, and C₁₋ ₆alkyl that is unsubstituted or substituted by R⁰ or —C(O)R⁰; and
   (e) 4-membered heterocycloalkyl comprising, as ring members, 1 to 2 heteroatoms selected from N, O and S and that is unsubstituted or substituted by 1 to 2 substituents independently selected from C₁₋₆haloalkyl, R⁰, C₁₋₆alkylamino, di-(C₁₋ ₆alkyl)amino, —C(O)R⁰, and C₁₋₆alkyl that is unsubstituted or substituted by R⁰ or —C(O)R⁰;
or, R¹ and R² can be taken together with the nitrogen atom to which both are bound to form a 4- to 6-membered heterocycloalkyl that can include, as ring members, 1 to 2 additional heteroatoms independently selected from N, O, and S, wherein the 4- to 6-membered heterocycloalkyl formed by R¹ and R² taken together with the nitrogen atom to which both are bound is unsubstituted or substituted by 1 to 3 substituents independently selected from halogen, C₁₋₆alkyl, C₁₋₆haloalkyl, and R⁰; R³ is selected from hydrogen, halogen and C₁₋₆alkyl; and
R⁵ is selected from hydrogen, halogen and —NH—(3- to 8-membered heteroalkyl), wherein the 3- to 8-membered heteroC₃₋₈alkyl of the —NH—(3- to 8-membered heteroalkyl) comprises 1 to 2 oxygen atoms as chain members and is unsubstituted or substituted by R⁰.

Embodiment 44. A method of treatment of an ocular disease or disorder according to Embodiment 41 to Embodiment 43, wherein the compound is of the formula selected from Formulae I to IV:

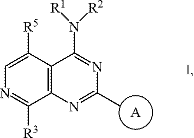

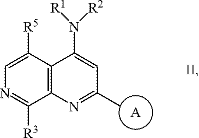

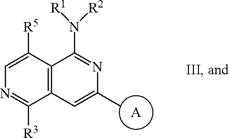

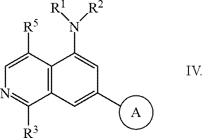

Embodiment 45. A method of treatment of an ocular disease or disorder according to Embodiment 41 to Embodiment 43, wherein the compound is selected from 3-(pyridin-4-yl)-N-(1-(trifluoromethyl)cyclopropyl)-2,6-naphthyridin-1-amine; N-(1-methylcyclopropyl)-7-(pyridin-4-yl)isoquinolin-5-amine; 2-(pyridin-4-yl)-4-(3-(trifluoromethyl)piperazin-1-yl)pyrido[3,4-d]pyrimidine; N-(tert-butyl)-2-(pyridin-4-yl)-1,7-naphthyridin-4-amine; and N-methyl-2-(pyridin-4-yl)-N-[(2S)-1,1,1-trifluoropropan-2-yl]pyrido[3,4-d]pyrimidin-4-amine.

Embodiment 46. A method of treatment of an ocular disease or disorder according to Embodiment 41 to Embodiment 43, wherein the compound is selected from: N-methyl-2-(pyridin-4-yl)-N-(1,1,1-trifluoropropan-2-yl)pyrido[3,4-d]pyrimidin-4-amine; 2-methyl-1-(2-methyl-2-{[2-(pyridin-4-yl)pyrido[3,4-d]pyrimidin-4-yl]amino}propoxy)propan-2-ol; 2,4-dimethyl-4-{[2-(pyridin-4-yl)pyrido[3,4-d]pyrimidin-4-yl]amino}pentan-2-ol; N-tert-butyl-2-(pyrimidin-4-yl)-1,7-naphthyridin-4-amine; 2-(pyridin-4-yl)-N-[1-(trifluoromethyl)cyclobutyl]pyrido[3,4-d]pyrimidin-4-amine; N-propyl-2-(pyridin-4-yl)pyrido[3,4-d]pyrimidin-4-amine; N-(propan-2-yl)-2-(pyridin-4-yl)pyrido[3,4-d]pyrimidin-4-amine; 3-(pyridin-4-yl)-N-(1-(trifluoromethyl)cyclopropyl)-2,6-naphthyridin-1-amine; 2-(3-methyl-1H-pyrazol-4-yl)-N-(1-methylcyclopropyl)pyrido[3,4-d]pyrimidin-4-amine; 2-methyl-2-{[2-(pyridin-4-yl)pyrido[3,4-d]pyrimidin-4-yl]amino}propan-1-ol; 2-(pyridin-4-yl)-4-(3-(trifluoromethyl)piperazin-1-yl)pyrido[3,4-d]pyrimidine; N-cyclopentyl-2-(pyridin-4-yl)pyrido[3,4-d]pyrimidin-4-amine; N-propyl-2-(3-(trifluoromethyl)-1H-pyrazol-4-yl)pyrido[3,4-d]pyrimidin-4-amine; N-(2-methylcyclopentyl)-2-(pyridin-4-yl)pyrido[3,4-d]pyrimidin-4-amine; 2-(3-chloropyridin-4-yl)-N-(1,1,1-trifluoro-2-methylpropan-2-yl)pyrido[3,4-d]pyrimidin-4-amine; 2-(2-methyl-2-{[2-(pyridin-4-yl)pyrido[3,4-d]pyrimidin-4-yl]amino}propoxy)ethan-1-ol; N-(1-methylcyclopropyl)-7-(pyridin-4-yl)isoquinolin-5-amine; (1S,2S)-2-{[2-(pyridin-4-yl)pyrido[3,4-d]pyrimidin-4-yl]amino}cyclopentan-1-ol; N-methyl-2-(pyridin-4-yl)-N-[(2S)-1,1,1-trifluoropropan-2-yl]pyrido[3,4-d]pyrimidin-4-amine; N-methyl-N-(propan-2-yl)-2-(pyridin-4-yl)pyrido[3,4-d]pyrimidin-4-amine; N-(propan-2-yl)-2-(pyridin-4-yl)pyrido[3,4-d]pyrimidin-4-amine; 3-(pyridin-4-yl)-N-(1-(trifluoromethyl)cyclopropyl)-2,6-naphthyridin-1-amine and N-methyl-2-(pyridin-4-yl)-N-[(2R)-1,1,1-trifluoropropan-2-yl]pyrido[3,4-d]pyrimidin-4-amine.

Embodiment 47. A method of treatment of an ocular disease or disorder according to Embodiment 41 to Embodiment 43, wherein the compound is selected from: N-(tert-butyl)-2-(pyridin-4-yl)-1,7-naphthyridin-4-amine; and N-methyl-2-(pyridin-4-yl)-N-[(2S)-1,1,1-trifluoropropan-2-yl]pyrido[3,4-d]pyrimidin-4-amine, in particular N-(tert-butyl)-2-(pyridin-4-yl)-1,7-naphthyridin-4-amine.

Embodiment 48. A method of treatment of an ocular disease or disorder according to Embodiment 41 to Embodiment 43, wherein the compound is selected from dimethyl(3-methyl-3-{[2-(pyridin-4-yl)pyrido[3,4-d]pyrimidin-4-yl]amino}butyl)amine and N¹,N¹,3-trimethyl-N³-(2-(3-methyl-1H-pyrazol-4-yl)pyrido[3,4-d]pyrimidin-4-yl)butane-1,3-diamine, in particular dimethyl(3-methyl-3-{[2-(pyridin-4-yl)pyrido[3,4-d]pyrimidin-4-yl]amino}butyl)amine.

Embodiment 49. A method of promoting cell proliferation of modified limbal stem cells or modified corneal endothelial cells, the method comprising culturing the modified limbal stem cells or modified corneal endothelial cells in a cell proliferation medium comprising a compound of Formula A1, or a salt thereof,

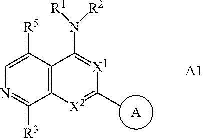

wherein
X¹ and X² are each independently CH or N;
Ring A is
   (a) a 5- or 6-membered monocyclic heteroaryl that is linked to the remainder of the molecule through a carbon ring member and comprises, as ring member, 1 to 4 heteroatoms that are independently selected from N, O and S, provided that at least one of the heteroatom ring member is an unsubstituted nitrogen (—N═) positioned at the 3- or the 4-position relative to the linking carbon ring member of the 5-membered heteroaryl or at the para ring position of the 6-membered heteroaryl; or
   (b) a 9-membered fused bicyclic heteroaryl that is selected from 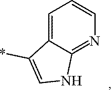 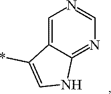 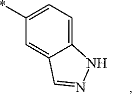 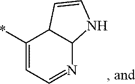 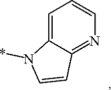
wherein “*” represents the point of attachment of ring A to the remainder of the molecule;
wherein ring A is unsubstituted or substituted by 1 to 2 substituents independently selected from halogen, cyano, C₁₋₆alkyl, C₁₋₆haloalkyl, —NH₂, C₁₋₆alkylamino, di-(C₁₋₆alkyl)amino, C₃₋₆cycloalkyl, and phenylsulfonyl;
R° is hydroxyl or C₁₋₆alkoxy;
R¹ is hydrogen or C₁₋₆alkyl;
R² is selected from
   (a) C₁₋₈alkyl that is unsubstituted or substituted by 1 to 3 substituents independently selected from
      (i) halogen;
      (ii) cyano;
      (iii) oxo;
      (iv) C₂alkenyl;
      (v) C₂alkynyl;
      (vi) C₁₋₆haloalkyl;
      (vii) —OR⁶, wherein R⁶ is selected from hydrogen, C₁₋₆alkyl that is unsubstituted or substituted by R⁰ or —C(O)R⁰;
      (viii) —NR^{7a}R^{7b}, wherein R^{7a} is hydrogen or C₁₋₆alkyl, and R^{7b} is selected from hydrogen, —C(O)R⁰, C₁₋₆alkyl that is unsubstituted or substituted by —C(O)R⁰;
      (ix) —C(O)R⁸, wherein R⁸ is R⁰ or —NH—C₁₋₆alkyl-C(O)R⁰;
      (x) -S(O)₂C₁₋₆alkyl;
      (xi) monocyclic C₃₋₆cycloalkyl or polycyclic C₇₋₁₀cycloalkyl that are each unsubstituted or substituted by 1 to 2 substituents independently selected from halogen, C₁₋₆alkyl, hydroxyC₁₋₆alkyl, C₁₋₆haloalkyl, R⁰, —NH₂, C₁₋₆alkylamino, and di-(C₁₋₆alkyl)amino;
      (xii) 6-membered heterocycloalkyl comprising, as ring members, 1 to 2 heteroatoms independently selected from N, O and S and that is unsubstituted or substituted by 1 to 2 substituents independently selected from hydroxyl, halogen, C₁₋₆alkyl, C₁₋₆alkylamino, and di-(C₁₋₆alkyl)amino;
      (xiii) phenyl that is unsubstituted or substituted by halogen;
      (xiv) 5- or 6-membered monocyclic heteroaryl comprising, as ring members, 1 to 4 heteroatoms independently selected from N and O; and
      (xv) 9- or 10-membered fused bicyclic heteroaryl comprising, as ring member, 1 to 2 heteroatoms independently selected from N and O;
   (b) -S(O)₂C₁₋₆alkyl;
   (c) phenyl that is unsubstituted or substituted by 1 to 2 substituents independently selected from halogen, C₁₋₆alkyl and R⁰;
   (d) C₃₋₆cycloalkyl that is unsubstituted or substituted by 1 to 2 substituents independently selected from C₁₋₆haloalkyl, R⁰, C₁₋₆alkylamino, di-(C₁₋₆alkyl)amino, —C(O)R⁰, and C₁₋ ₆alkyl that is unsubstituted or substituted by R⁰ or —C(O)R⁰; and
   (e) 4-membered heterocycloalkyl comprising, as ring members, 1 to 2 heteroatoms selected from N, O and S and that is unsubstituted or substituted by 1 to 2 substituents independently selected from C₁₋₆haloalkyl, R⁰, C₁₋₆alkylamino, di-(C₁₋ ₆alkyl)amino, —C(O)R⁰, and C₁₋₆alkyl that is unsubstituted or substituted by R⁰ or —C(O)R⁰;
or, R¹ and R² can be taken together with the nitrogen atom to which both are bound to form a 4- to 6-membered heterocycloalkyl that can include, as ring members, 1 to 2 additional heteroatoms independently selected from N, O, and S, wherein the 4- to 6-membered heterocycloalkyl formed by R¹ and R² taken together with the nitrogen atom to which both are bound is unsubstituted or substituted by 1 to 3 substituents independently selected from halogen, C₁₋₆alkyl, C₁₋₆haloalkyl, and R⁰; R³ is selected from hydrogen, halogen and C₁₋₆alkyl; and
R⁵ is selected from hydrogen, halogen and —NH—(3- to 8-membered heteroalkyl), wherein the 3- to 8-membered heteroC₃₋₈alkyl of the —NH—(3- to 8-membered heteroalkyl) comprises 1 to 2 oxygen atoms as chain members and is unsubstituted or substituted by R⁰.

Embodiment 50. A cell preparation comprising a LATS inhibitor and modified corneal endothelial cells.

Embodiment 51. The cell preparation according to Embodiment 50, wherein the LATS inhibitor is a compound of Formula A1,

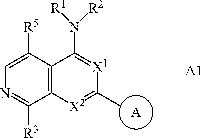

wherein
X¹ and X² are each independently CH or N;
Ring A is
   (a) a 5- or 6-membered monocyclic heteroaryl that is linked to the remainder of the molecule through a carbon ring member and comprises, as ring member, 1 to 4 heteroatoms that are independently selected from N, O and S, provided that at least one of the heteroatom ring member is an unsubstituted nitrogen (—N═) positioned at the 3- or the 4-position relative to the linking carbon ring member of the 5-membered heteroaryl or at the para ring position of the 6-membered heteroaryl; or
   (b) a 9-membered fused bicyclic heteroaryl that is selected from 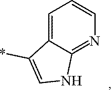 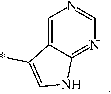 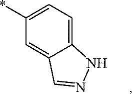 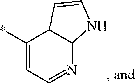 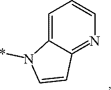
wherein “*” represents the point of attachment of ring A to the remainder of the molecule;
wherein ring A is unsubstituted or substituted by 1 to 2 substituents independently selected from halogen, cyano, C₁₋₆alkyl, C₁₋₆haloalkyl, —NH₂, C₁₋₆alkylamino, di-(C₁₋₆alkyl)amino, C₃₋₆cycloalkyl, and phenylsulfonyl;
R⁰ is hydroxyl or C₁₋₆alkoxy;
R¹ is hydrogen or C₁₋₆alkyl;
R² is selected from
   (a) C₁₋₈alkyl that is unsubstituted or substituted by 1 to 3 substituents independently selected from
      (i) halogen;
      (ii) cyano;
      (iii) oxo;
      (iv) C₂alkenyl;
      (v) C₂alkynyl;
      (vi) C₁₋₆haloalkyl;
      (vii) —OR⁶, wherein R⁶ is selected from hydrogen, C₁₋₆alkyl that is unsubstituted or substituted by R⁰ or —C(O)R⁰;
      (viii) —NR^{7a}R^{7b}, wherein R^{7a} is hydrogen or C₁₋₆alkyl, and R^{7b} is selected from hydrogen, —C(O)R⁰, C₁₋₆alkyl that is unsubstituted or substituted by —C(O)R⁰;
      (ix) —C(O)R⁸, wherein R⁸ is R⁰ or —NH—C₁₋₆alkyl-C(O)R⁰;
      (x) -S(O)₂C₁₋₆alkyl;
      (xi) monocyclic C₃₋₆cycloalkyl or polycyclic C₇₋₁₀cycloalkyl that are each unsubstituted or substituted by 1 to 2 substituents independently selected from halogen, C₁₋₆alkyl, hydroxyC₁₋₆alkyl, C₁₋₆haloalkyl, R⁰, —NH₂, C₁₋₆alkylamino, and di-(C₁₋₆alkyl)amino;
      (xii) 6-membered heterocycloalkyl comprising, as ring members, 1 to 2 heteroatoms independently selected from N, O and S and that is unsubstituted or substituted by 1 to 2 substituents independently selected from hydroxyl, halogen, C₁₋₆alkyl, C₁₋₆alkylamino, and di-(C₁₋₆alkyl)amino;
      (xiii) phenyl that is unsubstituted or substituted by halogen;
      (xiv) 5- or 6-membered monocyclic heteroaryl comprising, as ring members, 1 to 4 heteroatoms independently selected from N and O; and
      (xv) 9- or 10-membered fused bicyclic heteroaryl comprising, as ring member, 1 to 2 heteroatoms independently selected from N and O;
   (b) -S(O)₂C₁₋₆alkyl;
   (c) phenyl that is unsubstituted or substituted by 1 to 2 substituents independently selected from halogen, C₁₋₆alkyl and R⁰;
   (d) C₃₋₆cycloalkyl that is unsubstituted or substituted by 1 to 2 substituents independently selected from C₁₋₆haloalkyl, R⁰, C₁₋₆alkylamino, di-(C₁₋₆alkyl)amino, —C(O)R⁰, and C₁₋ ₆alkyl that is unsubstituted or substituted by R⁰ or —C(O)R⁰; and
   (e) 4-membered heterocycloalkyl comprising, as ring members, 1 to 2 heteroatoms selected from N, O and S and that is unsubstituted or substituted by 1 to 2 substituents independently selected from C₁₋₆haloalkyl, R⁰, C₁₋₆alkylamino, di-(C₁₋ ₆alkyl)amino, —C(O)R⁰, and C₁₋₆alkyl that is unsubstituted or substituted by R⁰ or —C(O)R⁰;
or, R¹ and R² can be taken together with the nitrogen atom to which both are bound to form a 4- to 6-membered heterocycloalkyl that can include, as ring members, 1 to 2 additional heteroatoms independently selected from N, O, and S, wherein the 4- to 6-membered heterocycloalkyl formed by R¹ and R² taken together with the nitrogen atom to which both are bound is unsubstituted or substituted by 1 to 3 substituents independently selected from halogen, C₁₋₆alkyl, C₁₋₆haloalkyl, and R⁰; R³ is selected from hydrogen, halogen and C₁₋₆alkyl; and
R⁵ is selected from hydrogen, halogen and —NH—(3- to 8-membered heteroalkyl), wherein the 3- to 8-membered heteroC₃₋₈alkyl of the —NH—(3- to 8-membered heteroalkyl) comprises 1 to 2 oxygen atoms as chain members and is unsubstituted or substituted by R⁰.

Embodiment 52. A cell preparation comprising a compound of Formula A1,

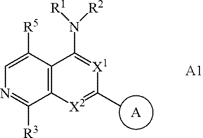

and modified corneal endothelial cells, wherein
X¹ and X² are each independently CH or N;
Ring A is
   (a) a 5- or 6-membered monocyclic heteroaryl that is linked to the remainder of the molecule through a carbon ring member and comprises, as ring member, 1 to 4 heteroatoms that are independently selected from N, O and S, provided that at least one of the heteroatom ring member is an unsubstituted nitrogen (—N═) positioned at the 3- or the 4-position relative to the linking carbon ring member of the 5-membered heteroaryl or at the para ring position of the 6-membered heteroaryl; or
   (b) a 9-membered fused bicyclic heteroaryl that is selected from 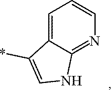 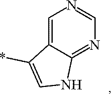 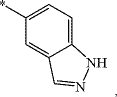 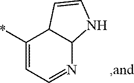 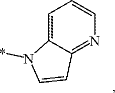
wherein “*” represents the point of attachment of ring A to the remainder of the molecule;
wherein ring A is unsubstituted or substituted by 1 to 2 substituents independently selected from halogen, cyano, C₁₋₆alkyl, C₁₋₆haloalkyl, —NH₂, C₁₋₆alkylamino, di-(C₁₋₆alkyl)amino, C₃₋₆cycloalkyl, and phenylsulfonyl;
R⁰ is hydroxyl or C₁₋₆alkoxy;
R¹ is hydrogen or C₁₋₆alkyl;
R² is selected from
   (a) C₁₋₈alkyl that is unsubstituted or substituted by 1 to 3 substituents independently selected from
      (i) halogen;
      (ii) cyano;
      (iii) oxo;
      (iv) C₂alkenyl;
      (v) C₂alkynyl;
      (vi) C₁₋₆haloalkyl;
      (vii) —OR⁶, wherein R⁶ is selected from hydrogen, C₁₋₆alkyl that is unsubstituted or substituted by R⁰ or —C(O)R⁰;
      (viii) —NR^{7a}R^{7b}, wherein R^{7a} is hydrogen or C₁₋₆alkyl, and R^{7b} is selected from hydrogen, —C(O)R⁰, C₁₋₆alkyl that is unsubstituted or substituted by —C(O)R⁰;
      (ix) —C(O)R⁸, wherein R⁸ is R⁰ or —NH—C₁₋₆alkyl-C(O)R⁰;
      (x) -S(O)₂C₁₋₆alkyl;
      (xi) monocyclic C₃₋₆cycloalkyl or polycyclic C₇₋₁₀cycloalkyl that are each unsubstituted or substituted by 1 to 2 substituents independently selected from halogen, C₁₋₆alkyl, hydroxyC₁₋₆alkyl, C₁₋₆haloalkyl, R⁰, —NH₂, C₁₋₆alkylamino, and di-(C₁₋₆alkyl)amino;
      (xii) 6-membered heterocycloalkyl comprising, as ring members, 1 to 2 heteroatoms independently selected from N, O and S and that is unsubstituted or substituted by 1 to 2 substituents independently selected from hydroxyl, halogen, C₁₋₆alkyl, C₁₋₆alkylamino, and di-(C₁₋₆alkyl)amino;
      (xiii) phenyl that is unsubstituted or substituted by halogen;
      (xiv) 5- or 6-membered monocyclic heteroaryl comprising, as ring members, 1 to 4 heteroatoms independently selected from N and O; and
      (xv) 9- or 10-membered fused bicyclic heteroaryl comprising, as ring member, 1 to 2 heteroatoms independently selected from N and O;
   (b) -S(O)₂C₁₋₆alkyl;
   (c) phenyl that is unsubstituted or substituted by 1 to 2 substituents independently selected from halogen, C₁₋₆alkyl and R⁰;
   (d) C₃₋₆cycloalkyl that is unsubstituted or substituted by 1 to 2 substituents independently selected from C₁₋₆haloalkyl, R⁰, C₁₋₆alkylamino, di-(C₁₋₆alkyl)amino, —C(O)R⁰, and C₁₋ ₆alkyl that is unsubstituted or substituted by R⁰ or —C(O)R⁰; and
   (e) 4-membered heterocycloalkyl comprising, as ring members, 1 to 2 heteroatoms selected from N, O and S and that is unsubstituted or substituted by 1 to 2 substituents independently selected from C₁₋₆haloalkyl, R⁰, C₁₋₆alkylamino, di-(C₁₋ ₆alkyl)amino, —C(O)R⁰, and C₁₋₆alkyl that is unsubstituted or substituted by R⁰ or —C(O)R⁰;
or, R¹ and R² can be taken together with the nitrogen atom to which both are bound to form a 4- to 6-membered heterocycloalkyl that can include, as ring members, 1 to 2 additional heteroatoms independently selected from N, O, and S, wherein the 4- to 6-membered heterocycloalkyl formed by R¹ and R² taken together with the nitrogen atom to which both are bound is unsubstituted or substituted by 1 to 3 substituents independently selected from halogen, C₁₋₆alkyl, C₁₋₆haloalkyl, and R⁰; R³ is selected from hydrogen, halogen and C₁₋₆alkyl; and
R⁵ is selected from hydrogen, halogen and —NH—(3- to 8-membered heteroalkyl), wherein the 3- to 8-membered heteroC₃₋₈alkyl of the —NH—(3- to 8-membered heteroalkyl) comprises 1 to 2 oxygen atoms as chain members and is unsubstituted or substituted by R⁰.

Embodiment 53. A cell preparation comprising a LATS inhibitor and modified limbal stem cells.

Embodiment 54. The cell preparation according to Embodiment 53, wherein the LATS inhibitor is a compound of Formula A1,

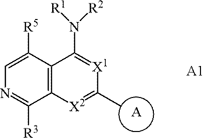

wherein
X¹ and X² are each independently CH or N;
Ring A is
   (a) a 5- or 6-membered monocyclic heteroaryl that is linked to the remainder of the molecule through a carbon ring member and comprises, as ring member, 1 to 4 heteroatoms that are independently selected from N, O and S, provided that at least one of the heteroatom ring member is an unsubstituted nitrogen (—N═) positioned at the 3- or the 4-position relative to the linking carbon ring member of the 5-membered heteroaryl or at the para ring position of the 6-membered heteroaryl; or
   (b) a 9-membered fused bicyclic heteroaryl that is selected from 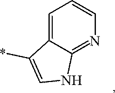 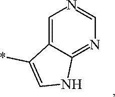 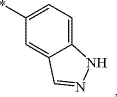 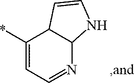 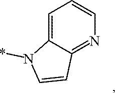
wherein “*” represents the point of attachment of ring A to the remainder of the molecule;
wherein ring A is unsubstituted or substituted by 1 to 2 substituents independently selected from halogen, cyano, C₁₋₆alkyl, C₁₋₆haloalkyl, —NH₂, C₁₋₆alkylamino, di-(C₁₋₆alkyl)amino, C₃₋₆cycloalkyl, and phenylsulfonyl;
R⁰ is hydroxyl or C₁₋₆alkoxy;
R¹ is hydrogen or C₁₋₆alkyl;
R² is selected from
   (a) C₁₋₈alkyl that is unsubstituted or substituted by 1 to 3 substituents independently selected from
      (i) halogen;
      (ii) cyano;
      (iii) oxo;
      (iv) C₂alkenyl;
      (v) C₂alkynyl;
      (vi) C₁₋₆haloalkyl;
      (vii) —OR⁶, wherein R⁶ is selected from hydrogen, C₁₋₆alkyl that is unsubstituted or substituted by R⁰ or —C(O)R⁰;
      (viii) —NR^{7a}R^{7b}, wherein R^{7a} is hydrogen or C₁₋₆alkyl, and R^{7b} is selected from hydrogen, —C(O)R⁰, C₁₋₆alkyl that is unsubstituted or substituted by —C(O)R⁰;
      (ix) —C(O)R⁸, wherein R⁸ is R⁰ or —NH—C₁₋₆alkyl-C(O)R⁰;
      (x) -S(O)₂C₁₋₆alkyl;
      (xi) monocyclic C₃₋₆cycloalkyl or polycyclic C₇₋₁₀cycloalkyl that are each unsubstituted or substituted by 1 to 2 substituents independently selected from halogen, C₁₋₆alkyl, hydroxyC₁₋₆alkyl, C₁₋₆haloalkyl, R⁰, —NH₂, C₁₋₆alkylamino, and di-(C₁₋₆alkyl)amino;
      (xii) 6-membered heterocycloalkyl comprising, as ring members, 1 to 2 heteroatoms independently selected from N, O and S and that is unsubstituted or substituted by 1 to 2 substituents independently selected from hydroxyl, halogen, C₁₋₆alkyl, C₁₋₆alkylamino, and di-(C₁₋₆alkyl)amino;
      (xiii) phenyl that is unsubstituted or substituted by halogen;
      (xiv) 5- or 6-membered monocyclic heteroaryl comprising, as ring members, 1 to 4 heteroatoms independently selected from N and O; and
      (xv) 9- or 10-membered fused bicyclic heteroaryl comprising, as ring member, 1 to 2 heteroatoms independently selected from N and O;
   (b) -S(O)₂C₁₋₆alkyl;
   (c) phenyl that is unsubstituted or substituted by 1 to 2 substituents independently selected from halogen, C₁₋₆alkyl and R⁰;
   (d) C₃₋₆cycloalkyl that is unsubstituted or substituted by 1 to 2 substituents independently selected from C₁₋₆haloalkyl, R⁰, C₁₋₆alkylamino, di-(C₁₋₆alkyl)amino, —C(O)R⁰, and C₁₋ ₆alkyl that is unsubstituted or substituted by R⁰ or —C(O)R⁰; and
   (e) 4-membered heterocycloalkyl comprising, as ring members, 1 to 2 heteroatoms selected from N, O and S and that is unsubstituted or substituted by 1 to 2 substituents independently selected from C₁₋₆haloalkyl, R⁰, C₁₋₆alkylamino, di-(C₁₋ ₆alkyl)amino, —C(O)R⁰, and C₁₋₆alkyl that is unsubstituted or substituted by R⁰ or —C(O)R⁰;
or, R¹ and R² can be taken together with the nitrogen atom to which both are bound to form a 4- to 6-membered heterocycloalkyl that can include, as ring members, 1 to 2 additional heteroatoms independently selected from N, O, and S, wherein the 4- to 6-membered heterocycloalkyl formed by R¹ and R² taken together with the nitrogen atom to which both are bound is unsubstituted or substituted by 1 to 3 substituents independently selected from halogen, C₁₋₆alkyl, C₁₋₆haloalkyl, and R⁰; R³ is selected from hydrogen, halogen and C₁₋₆alkyl; and
R⁵ is selected from hydrogen, halogen and —NH—(3- to 8-membered heteroalkyl), wherein the 3- to 8-membered heteroC₃₋₈alkyl of the —NH—(3- to 8-membered heteroalkyl) comprises 1 to 2 oxygen atoms as chain members and is unsubstituted or substituted by R⁰.

Embodiment 55. A cell preparation comprising a compound of Formula A1,

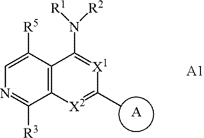

and modified limbal stem cells, wherein
X¹ and X² are each independently CH or N;
Ring A is
   (a) a 5- or 6-membered monocyclic heteroaryl that is linked to the remainder of the molecule through a carbon ring member and comprises, as ring member, 1 to 4 heteroatoms that are independently selected from N, O and S, provided that at least one of the heteroatom ring member is an unsubstituted nitrogen (—N═) positioned at the 3- or the 4-position relative to the linking carbon ring member of the 5-membered heteroaryl or at the para ring position of the 6-membered heteroaryl; or
   (b) a 9-membered fused bicyclic heteroaryl that is selected from 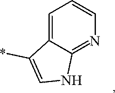 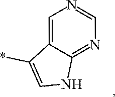 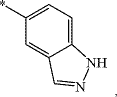 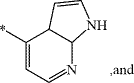 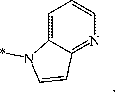
wherein “*” represents the point of attachment of ring A to the remainder of the molecule;
wherein ring A is unsubstituted or substituted by 1 to 2 substituents independently selected from halogen, cyano, C₁₋₆alkyl, C₁₋₆haloalkyl, —NH₂, C₁₋₆alkylamino, di-(C₁₋₆alkyl)amino, C₃₋₆cycloalkyl, and phenylsulfonyl;
R⁰ is hydroxyl or C₁₋₆alkoxy;
R¹ is hydrogen or C₁₋₆alkyl;
R² is selected from
   (a) C₁₋₈alkyl that is unsubstituted or substituted by 1 to 3 substituents independently selected from
      (i) halogen;
      (ii) cyano;
      (iii) oxo;
      (iv) C₂alkenyl;
      (v) C₂alkynyl;
      (vi) C₁₋₆haloalkyl;
      (vii) —OR⁶, wherein R⁶ is selected from hydrogen, C₁₋₆alkyl that is unsubstituted or substituted by R⁰ or —C(O)R⁰;
      (viii) —NR^{7a}R^{7b}, wherein R^{7a} is hydrogen or C₁₋₆alkyl, and R^{7b} is selected from hydrogen, —C(O)R⁰, C₁₋₆alkyl that is unsubstituted or substituted by —C(O)R⁰;
      (ix) —C(O)R⁸, wherein R⁸ is R⁰ or —NH—C₁₋₆alkyl-C(O)R⁰;
      (x) -S(O)₂C₁₋₆alkyl;
      (xi) monocyclic C₃₋₆cycloalkyl or polycyclic C₇₋₁₀cycloalkyl that are each unsubstituted or substituted by 1 to 2 substituents independently selected from halogen, C₁₋₆alkyl, hydroxyC₁₋₆alkyl, C₁₋₆haloalkyl, R⁰, —NH₂, C₁₋₆alkylamino, and di-(C₁₋₆alkyl)amino;
      (xii) 6-membered heterocycloalkyl comprising, as ring members, 1 to 2 heteroatoms independently selected from N, O and S and that is unsubstituted or substituted by 1 to 2 substituents independently selected from hydroxyl, halogen, C₁₋₆alkyl, C₁₋₆alkylamino, and di-(C₁₋₆alkyl)amino;
      (xiii) phenyl that is unsubstituted or substituted by halogen;
      (xiv) 5- or 6-membered monocyclic heteroaryl comprising, as ring members, 1 to 4 heteroatoms independently selected from N and O; and
      (xv) 9- or 10-membered fused bicyclic heteroaryl comprising, as ring member, 1 to 2 heteroatoms independently selected from N and O;
   (b) -S(O)₂C₁₋₆alkyl;
   (c) phenyl that is unsubstituted or substituted by 1 to 2 substituents independently selected from halogen, C₁₋₆alkyl and R⁰;
   (d) C₃₋₆cycloalkyl that is unsubstituted or substituted by 1 to 2 substituents independently selected from C₁₋₆haloalkyl, R⁰, C₁₋₆alkylamino, di-(C₁₋₆alkyl)amino, —C(O)R⁰, and C₁₋ ₆alkyl that is unsubstituted or substituted by R⁰ or —C(O)R⁰; and
   (e) 4-membered heterocycloalkyl comprising, as ring members, 1 to 2 heteroatoms selected from N, O and S and that is unsubstituted or substituted by 1 to 2 substituents independently selected from C₁₋₆haloalkyl, R⁰, C₁₋₆alkylamino, di-(C₁₋ ₆alkyl)amino, —C(O)R⁰, and C₁₋₆alkyl that is unsubstituted or substituted by R⁰ or —C(O)R⁰;
or, R¹ and R² can be taken together with the nitrogen atom to which both are bound to form a 4- to 6-membered heterocycloalkyl that can include, as ring members, 1 to 2 additional heteroatoms independently selected from N, O, and S, wherein the 4- to 6-membered heterocycloalkyl formed by R¹ and R² taken together with the nitrogen atom to which both are bound is unsubstituted or substituted by 1 to 3 substituents independently selected from halogen, C₁₋₆alkyl, C₁₋₆haloalkyl, and R⁰; R³ is selected from hydrogen, halogen and C₁₋₆alkyl; and
R⁵ is selected from hydrogen, halogen and —NH—(3- to 8-membered heteroalkyl), wherein the 3- to 8-membered heteroC₃₋₈alkyl of the —NH—(3- to 8-membered heteroalkyl) comprises 1 to 2 oxygen atoms as chain members and is unsubstituted or substituted by R⁰.

Embodiment 56. The cell preparation according to any one of Embodiment 50 to Embodiment 55, which further comprises a growth medium, wherein the growth medium is selected from the group consisting of Dulbecco’s Modified Eagle’s Medium supplemented with Fetal Bovine Serum, human endothelial serum free medium with human serum, X-VIVO15 medium and mesenchymal stem cell-conditioned medium; preferably X-VIVO15 medium.

Embodiment 57. A method for expanding a population of modified cells ex vivo which comprises contacting the cells with a compound of Formula A1,

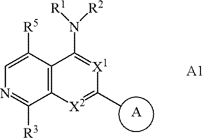

wherein
X¹ and X² are each independently CH or N;
Ring A is
   (a) a 5- or 6-membered monocyclic heteroaryl that is linked to the remainder of the molecule through a carbon ring member and comprises, as ring member, 1 to 4 heteroatoms that are independently selected from N, O and S, provided that at least one of the heteroatom ring member is an unsubstituted nitrogen (—N═) positioned at the 3- or the 4-position relative to the linking carbon ring member of the 5-membered heteroaryl or at the para ring position of the 6-membered heteroaryl; or
   (b) a 9-membered fused bicyclic heteroaryl that is selected from 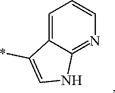 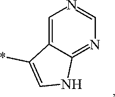 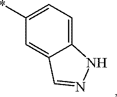 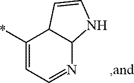 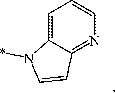
wherein “*” represents the point of attachment of ring A to the remainder of the molecule;
wherein ring A is unsubstituted or substituted by 1 to 2 substituents independently selected from halogen, cyano, C₁₋₆alkyl, C₁₋₆haloalkyl, —NH₂, C₁₋₆alkylamino, di-(C₁₋₆alkyl)amino, C₃₋₆cycloalkyl, and phenylsulfonyl;
R⁰ is hydroxyl or C₁₋₆alkoxy;
R¹ is hydrogen or C₁₋₆alkyl;
R² is selected from
   (a) C₁₋₈alkyl that is unsubstituted or substituted by 1 to 3 substituents independently selected from
      (i) halogen;
      (ii) cyano;
      (iii) oxo;
      (iv) C₂alkenyl;
      (v) C₂alkynyl;
      (vi) C₁₋₆haloalkyl;
      (vii) —OR⁶, wherein R⁶ is selected from hydrogen, C₁₋₆alkyl that is unsubstituted or substituted by R⁰ or —C(O)R⁰;
      (viii) —NR^{7a}R^{7b}, wherein R^{7a} is hydrogen or C₁₋₆alkyl, and R^{7b} is selected from hydrogen, —C(O)R⁰, C₁₋₆alkyl that is unsubstituted or substituted by —C(O)R⁰;
      (ix) —C(O)R⁸, wherein R⁸ is R⁰ or —NH—C₁₋₆alkyl-C(O)R⁰;
      (x) -S(O)₂C₁₋₆alkyl;
      (xi) monocyclic C₃₋₆cycloalkyl or polycyclic C₇₋₁₀cycloalkyl that are each unsubstituted or substituted by 1 to 2 substituents independently selected from halogen, C₁₋₆alkyl, hydroxyC₁₋₆alkyl, C₁₋₆haloalkyl, R⁰, —NH₂, C₁₋₆alkylamino, and di-(C₁₋₆alkyl)amino;
      (xii) 6-membered heterocycloalkyl comprising, as ring members, 1 to 2 heteroatoms independently selected from N, O and S and that is unsubstituted or substituted by 1 to 2 substituents independently selected from hydroxyl, halogen, C₁₋₆alkyl, C₁₋₆alkylamino, and di-(C₁₋₆alkyl)amino;
      (xiii) phenyl that is unsubstituted or substituted by halogen;
      (xiv) 5- or 6-membered monocyclic heteroaryl comprising, as ring members, 1 to 4 heteroatoms independently selected from N and O; and
      (xv) 9- or 10-membered fused bicyclic heteroaryl comprising, as ring member, 1 to 2 heteroatoms independently selected from N and O;
   (b) -S(O)₂C₁₋₆alkyl;
   (c) phenyl that is unsubstituted or substituted by 1 to 2 substituents independently selected from halogen, C₁₋₆alkyl and R⁰;
   (d) C₃₋₆cycloalkyl that is unsubstituted or substituted by 1 to 2 substituents independently selected from C₁₋₆haloalkyl, R⁰, C₁₋₆alkylamino, di-(C₁₋₆alkyl)amino, —C(O)R⁰, and C₁₋ ₆alkyl that is unsubstituted or substituted by R⁰ or —C(O)R⁰; and
   (e) 4-membered heterocycloalkyl comprising, as ring members, 1 to 2 heteroatoms selected from N, O and S and that is unsubstituted or substituted by 1 to 2 substituents independently selected from C₁₋₆haloalkyl, R⁰, C₁₋₆alkylamino, di-(C₁₋ ₆alkyl)amino, —C(O)R⁰, and C₁₋₆alkyl that is unsubstituted or substituted by R⁰ or —C(O)R⁰;
or, R¹ and R² can be taken together with the nitrogen atom to which both are bound to form a 4- to 6-membered heterocycloalkyl that can include, as ring members, 1 to 2 additional heteroatoms independently selected from N, O, and S, wherein the 4- to 6-membered heterocycloalkyl formed by R¹ and R² taken together with the nitrogen atom to which both are bound is unsubstituted or substituted by 1 to 3 substituents independently selected from halogen, C₁₋₆alkyl, C₁₋₆haloalkyl, and R⁰; R³ is selected from hydrogen, halogen and C₁₋₆alkyl; and
R⁵ is selected from hydrogen, halogen and —NH—(3- to 8-membered heteroalkyl), wherein the 3- to 8-membered heteroC₃₋₈alkyl of the —NH—(3- to 8-membered heteroalkyl) comprises 1 to 2 oxygen atoms as chain members and is unsubstituted or substituted by R⁰.

Embodiment 58. The method according to Embodiment 57, wherein said modified cells are gene edited cells.

Embodiment 59. Cells obtained by the method according to any one of Embodiment 57 to 58.

In one embodiment, said compound of the present invention is present in a concentration of about 0.5 to about 100 micromolar, preferably of about 0.5 to about 25 micromolar, more preferably of about 1 to about 20 micromolar, particularly preferably of about 3 to about 10 micromolar. In one embodiment, said compound of the present invention is present in a concentration of 0.5 to 100 micromolar, preferably 0.5 to 25 micromolar, more preferably 1 to 20 micromolar, particularly preferably of 3 to 10 micromolar. In a specific embodiment, said compound of the present invention is present in a concentration of 3 to 10 micromolar.

In another embodiment, the present invention relates to a method of treatment of an ocular disease or disorder comprising administering to a subject in need thereof a cell population (e.g., cell population comprising modified limbal stem cell with reduced or eliminated expression of B2M by a CRISPR system), wherein the population has been grown in the presence of an agent capable of inhibiting the activity of LATS1 and LATS2 kinases; thereby inducing YAP translocation and driving downstream gene expression for cell proliferation. In a further embodiment, the agent is a compound of Formula A1 or subformulae thereof (e.g., Formula A2), or pharmaceutically acceptable salt thereof.

In another embodiment, the present invention relates to a method of treatment of an ocular disease or disorder comprising administering to a subject in need thereof a limbal stem cell population (e.g., cell population comprising modified limbal stem cell with reduced or eliminated expression of B2M by a CRISPR system), wherein the population has been grown in the presence of an agent capable of inhibiting the activity of LATS1 and LATS2 kinases; thereby inducing YAP translocation and driving downstream gene expression for cell proliferation. In a further embodiment, the agent is a compound of Formula A1 or subformulae thereof (e.g., Formula A2), or pharmaceutically acceptable salt thereof.

In another embodiment, the present invention relates to a method of treatment of an ocular disease or disorder comprising administering to a subject in need thereof a corneal endothelial cell population (e.g., cell population comprising modified corneal endothelial cells with reduced or eliminated expression of B2M by a CRISPR system), wherein the population has been grown in the presence of an agent capable of inhibiting the activity of LATS1 and LATS2 kinases; thereby inducing YAP translocation and driving downstream gene expression for cell proliferation. In a further embodiment, the agent is a compound of Formula A1 or subformulae thereof (e.g., Formula A2), or a pharmaceutically acceptable salt thereof.

In another embodiment, the present invention relates to a method of promoting ocular wound healing comprising administering to an eye of a subject a therapeutically effective amount of a cell population (e.g., cell population comprising modified cells with reduced or eliminated expression of B2M by a CRISPR system) obtainable or obtained by the method of cell population expansion according to the invention. In one embodiment, the ocular wound is a corneal wound. In other embodiments, the ocular wound is an injury or surgical wound.

### Definitions

The general terms used hereinbefore and hereinafter preferably have within the context of this invention the following meanings, unless otherwise indicated, where more general terms wherever used may, independently of each other, be replaced by more specific definitions or remain, thus defining more detailed embodiments of the invention.

All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed.

As used herein, the terms “a,” “an,” “the” and similar terms used in the context of the present invention (especially in the context of the claims) are to be construed to cover both the singular and plural unless otherwise indicated herein or clearly contradicted by the context.

As used herein, the term “C₁₋₈alkyl” refers to a straight or branched hydrocarbon chain radical consisting solely of carbon and hydrogen atoms, containing no unsaturation, having from one to eight carbon atoms, and which is attached to the rest of the molecule by a single bond. The term “C₁₋₄alkyl” is to be construed accordingly. As used herein, the term “n-alkyl” refers to straight chain (un-branced) alkyl radical as defined herein. Examples of C₁₋ ₈alkyl include, but are not limited to, methyl, ethyl, n-propyl, 1-methylethyl (iso-propyl), n-butyl, n-pentyl, 1,1-dimethylethyl (t-butyl), —C(CH₃)₂CH₂CH(CH₃)₂ and —C(CH₃)₂CH₃.

As used herein, the term “C₂₋₆alkenyl” refers to a straight or branched hydrocarbon chain radical group consisting solely of carbon and hydrogen atoms, containing at least one double bond, having from two to six carbon atoms, which is attached to the rest of the molecule by a single bond. As used herein, the term “C₂₋₄alkenyl” is to be construed accordingly. Examples of C₂₋₆alkenyl include, but are not limited to, ethenyl, prop-1-enyl, but-1-enyl, pent-1-enyl, pent-4-enyl and penta-1,4-dienyl.

As used herein, the term “alkylene” refers to a divalent alkyl group. For example, as used herein, the term “C₁₋₆alkylene” or “C₁ to C₆ alkylene” refers to a divalent, straight, or branched aliphatic group containing 1 to 6 carbon atoms. Examples of alkylene include, but are not limited to methylene (—CH₂—), ethylene (—CH₂CH₂—), n-propylene (—CH₂CH₂CH₂—), isopropylene (—CH(CH₃)CH₂—), n-butylene, sec-butylene, iso-butylene, tert-butylene, n-pentylene, isopentylene, neopentylene and n-hexylene.

As used herein, the term “C₂₋₆alkynyl” refers to a straight or branched hydrocarbon chain radical group consisting solely of carbon and hydrogen atoms, containing at least one triple bond, having from two to six carbon atoms, and which is attached to the rest of the molecule by a single bond. As used herein, the term “C₂₋₄alkynyl” is to be construed accordingly.

Examples of C₂₋₆alkynyl include, but are not limited to, ethynyl, prop-1-ynyl, but-1-ynyl, pent-1-ynyl, pent-4-ynyl and penta-1,4-diynyl.

As used herein, the term “C₁₋₆alkoxy” refers to a radical of the formula —ORₐ, where Rₐ is a C₁₋₆alkyl radical as generally defined above. As used herein, the term “C₁-₆ alkoxy” or “C₁ to C₆ alkoxy” is intended to include C₁, C₂, C₃, C₄, C₅, and C₆ alkoxy groups (that is 1 to 6 carbons in the alkyl chain). Examples of C₁₋₆alkoxy include, but are not limited to, methoxy, ethoxy, propoxy, isopropoxy, butoxy, isobutoxy, pentoxy, and hexoxy.

As used herein, the term “C₁₋₆alkylamino” refers to a radical of the formula —NH—Rₐ, where Rₐ is a C₁₋₄alkyl radical as defined above.

As used herein, the term “di-(C₁₋₆alkyl)amino” refers to a radical of the formula —N(Rₐ)—Rₐ, where each Rₐ is a C₁₋₄alkyl radical, which may be the same or different, as defined above.

As used herein, the term “cyano” means the radical ∗—C≡N.

As used herein, the term “cycloalkyl” refers to nonaromatic carbocyclic ring that is a fully hydrogenated ring, including mono-, bi- or poly-cyclic ring systems. “C₃₋₁₀cycloalkyl” or “C₃ to C₁₀ cycloalkyl” is intended to include C₃, C₄, C₅, C₆, C₇, C₈, C₉ and C₁₀ cycloalkyl groups that is 3 to 10 carbon ring members). Example cycloalkyl groups include, but are not limited to, cyclopropyl, cyclobutyl, cyclopentyl, cyclohexyl and norbornyl.

As used herein, the term “fused ring”refers to a multi-ring assembly wherein the rings comprising the ring assembly are so linked that the ring atoms that are common to two rings are directly bound to each other. The fused ring assemblies may be saturated, partially saturated, aromatics, carbocyclics, heterocyclics, and the like. Non-exclusive examples of common fused rings include decalin, naphthalene, anthracene, phenanthrene, indole, benzofuran, purine, quinoline, and the like.

As used herein, the term “halogen” refers to bromo, chloro, fluoro or iodo; preferably fluoro, chloro or bromo.

As used herein, the term “haloalkyl” is intended to include both branched and straight-chain saturated alkyl groups as defined above having the specified number of carbon atoms, substituted with one or more halogens. For example, “C₁-₆haloalkyl” or “C₁ to C₆ haloalkyl” is intended to include C₁, C₂, C₃, C₄, C₅, and C₆ alkyl chain. Examples of haloalkyl include, but are not limited to, fluoromethyl, difluoromethyl, trifluoromethyl, trichloromethyl, pentafluoroethyl, pentachloroethyl, 2,2,2-trifluoroethyl, 1,3-dibromopropan-2-yl, 3-bromo-2-fluoropropyl and 1,4,4-trifluorobutan-2-yl, heptafluoropropyl, and heptachloropropyl.

As used herein, the term “heteroalkyl”refers to an alkyl, as defined herein, where one or more of the carbon atoms within the alkyl chain are replaced by heteroatoms independently selected from N, O and S. In C_{X-Y}hetereoalkyl or x- to y-membered heteroalkyl, as used herein, x-y describe the number of chain atoms (carbon and heteroatoms) on the heteroalkyl. For example C₃₋₈heteroalkyl refers to an alkyl chain with 3 to 8 chain atoms. Unless otherwise indicated, the atom linking the radical to the remainder of the molecule must be a carbon. Representative example of 3- to 8-membered heteroalkyl include, but are not limited to —(CH₂)OCH₃, —(CH₂)₂OCH(CH₃)₂, —(CH₂)₂—O—(CH₂)₂—OH and —(CH₂)₂—(O—(CH₂)₂)₂—OH.

As used herein, the term “heteroaryl” refers to aromatic moieties containing at least one heteroatom (e.g., oxygen, sulfur, nitrogen or combinations thereof) within a 5- to 10-membered aromatic ring system. Examples of heteroaryl include, but are not limited to pyrrolyl, pyridyl, pyrazolyl, indolyl, indazolyl, thienyl, furanyl, benzofuranyl, oxazolyl, isoxazolyl, imidazolyl, triazolyl, tetrazolyl, triazinyl, pyrimidinyl, pyrazinyl, thiazolyl, purinyl, benzimidazolyl, quinolinyl, isoquinolinyl, quinoxalinyl, benzopyranyl, benzothiophenyl, benzoimidazolyl, benzoxazolyl and 1H-benzo[d][1,2,3]triazolyl. The heteroaromatic moiety may consist of a single or fused ring system. A typical single heteroaryl ring is a 5- to 6-membered ring containing one to four heteroatoms independently selected from N, O and S and a typical fused heteroaryl ring system is a 9- to 10-membered ring system containing one to four heteroatoms independently selected from N, O and S. The fused heteroaryl ring system may consist of two heteroaryl rings fused together or a heteroaryl fused to an aryl (e.g., phenyl).

As used herein, the term “heteroatoms” refers to nitrogen (N), oxygen (O) or sulfur (S) atoms. Unless otherwise indicated, any heteroatom with unsatisfied valences is assumed to have hydrogen atoms sufficient to satisfy the valences, and when the heteroatom is sulfur, it can be unoxidized (S) or oxidized to S(O) or S(O)₂.

As used herein, the term “hydroxyl” or “hydroxy”refers to the radical —OH.

As used herein, the term “heterocycloalkyl” means cycloalkyl, as defined in this application, provided that one or more of the ring carbons indicated, are replaced by a moiety selected from —O—, —N═, —NH—, —S—, —S(O)— and —S(O)₂—. Examples of 3 to 8 membered heterocycloalkyl include, but are not limited to, oxiranyl, aziridinyl, azetidinyl, imidazolidinyl, pyrazolidinyl, tetrahydrofuranyl, tetrahydrothienyl, tetrahydrothienyl 1,1-dioxide, oxazolidinyl, thiazolidinyl, pyrrolidinyl, pyrrolidinyl-2-one, morpholinyl, piperazinyl, piperidinyl, pyrazolidinyl, hexahydropyrimidinyl, 1,4-dioxa-8-aza-spiro[4.5]dec-8-yl, thiomorpholinyl, sulfanomorpholinyl, sulfonomorpholinyl and octahydropyrrolo[3,2-b]pyrrolyl.

As used herein, the term “oxo”refers to the divalent radical =O.

As used herein, the term “substituted” means that at least one hydrogen atom is replaced with a non-hydrogen group, provided that normal valencies are maintained and that the substitution results in a stable compound. When a substituent is oxo (i.e., =O), then two hydrogens on the atom are replaced. In cases wherein there are nitrogen atoms (e.g., amines) present in compounds of the present invention, these may be converted to N-oxides by treatment with an oxidizing agent (e.g., mCPBA and/or hydrogen peroxides) to afford other compounds of the present invention.

As used herein, the term “unsubstituted nitrogen” refers to a nitrogen ring atom that has no capacity for substitution due to its linkage to its adjacent ring atoms by a double bond and a single bond (—N═). For example, the nitrogen at the para position of the 4-pyridyl

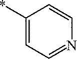 [SMILES: *c1ccncc1]

is an “unsubstituted” nitrogen, andthe nitrogen at the 4-position, in reference to the linking C-ring atom, of 1H-pyrazol-4-yl,

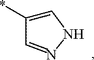

is an “unsubstituted” nitrogen.

As a person of ordinary skill in the art would be able to understand, for example, a ketone (—CH—C(═O)—) group in a molecule may tautomerize to its enol form(—C═C(OH)—). Thus, the invention is intended to cover all possible tautomers even when a structure depicts only one of them.

As used herein,

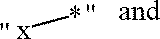

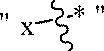

are symbols denoting the point of attachment of X, to other part of the molecule.

When any variable occurs more than one time in any constituent or formula for a compound of the present invention, its definition at each occurrence is independent of its definition at every other occurrence. Thus, for example, if a group is shown to be substituted with 0-3 R groups, then said group may be unsubstituted or substituted with up to three R groups, and at each occurrence R is selected independently from the definition of R.

Unless specified otherwise, the term “compound of the present invention” or “compounds of the present invention” refers to compounds of Formula A1 and subformulae thereof (e.g., Formula A2), as well as isomers, such as stereoisomers (including diastereoisomers, enantiomers and racemates), geometrical isomers, conformational isomers (including rotamers and astropisomers), tautomers, isotopically labeled compounds (including deuterium substitutions), and inherently formed moieties (e.g., polymorphs, solvates and/or hydrates). When a moiety is present that is capable of forming a salt, then salts are included as well, in particular pharmaceutically acceptable salts.

It will be recognized by those skilled in the art that the compounds of the present invention may contain chiral centers and as such may exist in different isomeric forms. As used herein, the term “isomers” refers to different compounds of the present invention that have the same molecular formula but differ in arrangement and configuration of the atoms.

As used herein, the term “enantiomers” are a pair of stereoisomers that are non-superimposable mirror images of each other. A 1:1 mixture of a pair of enantiomers is a “racemic” mixture. As used herein, the term is used to designate a racemic mixture where appropriate. When designating the stereochemistry for the compounds of the present invention, a single stereoisomer with known relative and absolute configuration of the two chiral centers is designated using the conventional RS system (e.g., (1S,2S)); a single stereoisomer with known relative configuration but unknown absolute configuration is designated with stars (e.g., (1R*,2R*)); and a racemate with two letters (e.g, (1RS,2RS) as a racemic mixture of (1R,2R) and (1S,2S); (1RS,2SR) as a racemic mixture of (1R,2S) and (1S,2R)). As used herein, the term “diastereoisomers” are stereoisomers that have at least two asymmetric atoms, but which are not mirror-images of each other. The absolute stereochemistry is specified according to the Cahn-Ingold-Prelog R-S system. When a compound of the present invention is a pure enantiomer, the stereochemistry at each chiral carbon may be specified by either R or S. Resolved compounds of the present invention whose absolute configuration is unknown can be designated (+) or (-) depending on the direction (dextro- or levorotatory) which they rotate plane polarized light at the wavelength of the sodium D line. Alternatively, the resolved compounds of the present invention can be defined by the respective retention times for the corresponding enantiomers/diastereomers via chiral HPLC.

Certain of the compounds of the present invention described herein contain one or more asymmetric centers or axes and may thus give rise to enantiomers, diastereomers, and other stereoisomeric forms that may be defined, in terms of absolute stereochemistry, as (R)- or (S)-.

Geometric isomers may occur when a compound of the present invention contains a double bond or some other feature that gives the molecule a certain amount of structural rigidity. If the compound contains a double bond, the substituent may be E or Z configuration. If the compound contains a disubstituted cycloalkyl, the cycloalkyl substituent may have a cis- or trans-configuration.

As used herein, the term “conformational isomers” or “conformers” are isomers that can differ by rotations about one or more bonds. Rotamers are conformers that differ by rotation about only a single bond.

As used herein, the term “atropisomer” refers to a structural isomer based on axial or planar chirality resulting from restricted rotation in the molecule.

Unless specified otherwise, the compounds of the present invention are meant to include all such possible isomers, including racemic mixtures, optically pure forms and intermediate mixtures. Optically active (R)- and (S)- isomers may be prepared using chiral synthons or chiral reagents, or resolved using conventional techniques (e.g., separated on chiral SFC or HPLC chromatography columns, such as CHIRALPAK® and CHIRALCEL® available from DAICEL Corp. using the appropriate solvent or mixture of solvents to achieve good separation).

The compounds of the present invention can be isolated in optically active or racemic forms. Optically active forms may be prepared by resolution of racemic forms or by synthesis from optically active starting materials. All processes used to prepare compounds of the present invention and intermediates made therein are considered to be part of the present invention. When enantiomeric or diastereomeric products are prepared, they may be separated by conventional methods, for example, by chromatography or fractional crystallization.

As used herein, the term “LATS” is the abbreviated name of the large tumor suppressor protein kinase. As used herein, the term “LATS” refers to LATS1 and/or LATS2. As used herein, the term “LATS1” refers to the large tumor suppressor kinase 1 and the term “LATS2” refers to the large tumor suppressor kinase 2. LATS1 and LATS2 both have serine/threonine protein kinase activity.

As used herein, the term “YAP1” refers to the yes-associated protein 1, also known as YAP or YAP65, which is a protein that acts as a transcriptional regulator of genes involved in cell proliferation.

As used herein, the term “MST1/2” refers to mammalian sterile 20-like kinase -1 and -2.

The term “effective amount” or “therapeutically effective amount” are used interchangeably herein, and refer to an amount of a compound, formulation, material, or composition, as described herein effective to achieve a particular biological result.

As used herein, the term “a therapeutically effective amount” of a compound of the present invention refers to an amount of the compound of the present invention that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” as used herein refers to the amount of the LATS compound of the present invention that, when administered to a subject, is effective to (1) at least partially alleviate, inhibit, prevent and/or ameliorate a condition, or a disorder or a disease (i) mediated by LATS activity, or (ii) characterized by activity (normal or abnormal) of LATS; or (2) reduce or inhibit the activity of LATS; or (3) reduce or inhibit the expression of LATS. In another non-limiting embodiment, the term “a therapeutically effective amount” as used herein refers to the amount of the compound of the present invention that, when administered to a cell, or a tissue, or a non-cellular biological material, or a medium, is effective to at least partially reducing or inhibiting the activity of LATS; or at least partially reducing or inhibiting the expression of LATS.

Further, as used herein, the term “a therapeutically effective amount” of a modified limbal stem cell of the present invention refers to an amount of the cells of the present invention that will elicit the biological or medical response of a subject, for example, ameliorate symptoms, alleviate conditions, slow or delay disease progression, inhibit or prevent a disease, in particular ocular disease, in particular limbal stem cell deficiency.

As used herein, the term “subject” includes human and non-human animals. Non-human animals include vertebrates, e.g., mammals and non-mammals, such as non-human primates, sheep, cats, horses, cows, chickens, dog, mouse, rat, goat, rabbit, and pig. Preferably, the subject is human. Except when noted, the terms “patient” or “subject” are used herein interchangeably.

As used herein, the term “IC₅₀” refers to the molar concentration of an inhibitor that produces 50% of the inhibition effect.

As used herein, the term “treat”, “treating” or “treatment” of any disease or disorder refers to alleviating or ameliorating the disease or disorder (i.e., slowing or arresting the development of the disease or at least one of the clinical symptoms thereof); or alleviating or ameliorating at least one physical parameter or biomarker associated with the disease or disorder, including those which may not be discernible to the patient.

As used herein, the term “prevent”, “preventing” or “prevention” of any disease or disorder refers to the prophylactic treatment of the disease or disorder; or delaying the onset or progression of the disease or disorder.

As used herein, a subject is “in need of” a treatment if such subject would benefit biologically, medically or in quality of life from such treatment.

Depending on the process conditions, the compounds of the present invention are obtained either in free (neutral) or salt form. Both the free form and salt form, and particularly “pharmaceutically acceptable salts” of these compounds, are within the scope of the invention.

As used herein, the terms “salt” or “salts” refers to an acid addition or base addition salt of a compound of the present invention. “Salts” include, in particular, “pharmaceutically acceptable salts”. As used herein, the term “pharmaceutically acceptable salts” refers to salts that retain the biological effectiveness and properties of the compounds of the present invention and, which typically are not biologically or otherwise undesirable. In many cases, the compounds of the present invention are capable of forming acid and/or base salts by virtue of the presence of amino and/or carboxyl groups or groups similar thereto.Pharmaceutically acceptable acid addition salts can be formed with inorganic acids and organic acids.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

In another aspect, the present invention provides compounds of Formula A1 or subformulae thereof (e.g., Formula A2) in acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethandisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate, mandelate, mesylate, methylsulphate, mucate, naphthoate, napsylate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate trifenatate, trifluoroacetate or xinafoate salt form.

Any formula given herein is also intended to represent unlabeled forms as well as isotopically labeled forms of the compounds. Isotopically labeled compounds of the present invention have structures depicted by the formulas given herein except that one or more atoms are replaced by an atom having a selected atomic mass or mass number. Isotopes that can be incorporated into compounds of the present invention include, for example, isotopes of hydrogen.

Further, incorporation of certain isotopes, particularly deuterium (i.e., ²H or D), may afford certain therapeutic advantages resulting from greater metabolic stability, for example increased in vivo half-life or reduced dosage requirements or an improvement in therapeutic index or tolerability. It is understood that deuterium in this context is regarded as a substituent of a compound of Formula A1 or subformulae thereof (e.g., Formula A2). The concentration of deuterium may be defined by the isotopic enrichment factor. As used herein, the term “isotopic enrichment factor” means the ratio between the isotopic abundance and the natural abundance of a specified isotope. If a substituent in a compound of the present invention is denoted as being deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). It should be understood that the term “isotopic enrichment factor” as used herein can be applied to any isotope in the same manner as described for deuterium.

Other examples of isotopes that can be incorporated into compounds of the present invention include isotopes of hydrogen, carbon, nitrogen, oxygen, phosphorous, fluorine, and chlorine, such as ³H, ¹¹C, ¹³C, ¹⁴C, ¹⁵N, ¹⁸F ³¹P, ³²P, ³⁵S, ³⁶CI, ¹²³I, ¹²⁴I, and ¹²⁵I, respectively. Accordingly it should be understood that the invention includes compounds that incorporate one or more of any of the aforementioned isotopes, including for example, radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically-labeled compounds of the present invention can generally be prepared by conventional techniques known to those skilled in the art or by processes analogous to those described in the accompanying Examples and Preparations using an appropriate isotopically-labeled reagents in place of the non-labeled reagent previously employed.

Any asymmetric atom (e.g., carbon or the like) of the compound(s) of the present invention can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated double bonds may, if possible, be present in cis- (Z)- or trans- (E)- form.

Accordingly, as used herein a compound of the present invention can be in the form of one of the possible stereoisomers, rotamers, atropisomers, tautomers or mixtures thereof, for example, as substantially pure geometric (cis or trans) stereoisomers, diastereomers, optical isomers (antipodes), racemates or mixtures thereof.

Any resulting mixtures of stereoisomers of the compounds of the present invention can be separated on the basis of the physicochemical differences of the constituents, into the pure or substantially pure geometric or optical isomers, diastereomers, racemates, for example, by chromatography and/or fractional crystallization.

Any resulting racemates of final compounds of the present invention or intermediates thereof can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

As used herein, the term “percentage of sequence identity” is determined by comparing two optimally aligned sequences over a comparison window, wherein the portion of the polynucleotide sequence in the comparison window may comprise additions or deletions (i.e., gaps) as compared to the reference sequence (e.g., a polypeptide of the invention), which does not comprise additions or deletions, for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

As used herein, the terms “identical” or percent “identity,” in the context of two or more nucleic acids or polypeptide sequences, refer to two or more sequences or subsequences that are the same sequences. Two sequences are “substantially identical” if two sequences have a specified percentage of amino acid residues or nucleotides that are the same (i.e., at least 75%, 80%, 85%, 90%, 95%, 96%, 97%, 98% or 99% sequence identity over a specified region, or, when not specified, over the entire sequence of a reference sequence), when compared and aligned for maximum correspondence over a comparison window, or designated region as measured using one of the following sequence comparison algorithms or by manual alignment and visual inspection. The invention provides polypeptides or polynucleotides that are substantially identical to the polypeptides or polynucleotides, respectively, exemplified herein.

As used herein, the term “isolated” means altered or removed from the natural state. For example, a nucleic acid or a peptide or cell naturally present in a living animal is not “isolated,” but the same nucleic acid or peptide or cell partially or completely separated from the coexisting materials of its natural state is “isolated.”

As used herein, the term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

As used herein, the term “cell population” or “population of cells” comprises cells that proliferate in the presence of a LATS1 and/or LATS2 inhibitor in vivo or ex vivo. In such cells, Hippo signaling typically suppresses cell growth, but will proliferate when the pathway is disrupted by LATS inhibition. In certain embodiments, a cell population useful in a method, preparation, medium, agent, or kit of the invention comprises cells from tissues described above or cells described or provided herein. Such cells include, but are not limited to ocular cells (e.g., limbal stem cells, corneal endothelial cells), epithelial cells (e.g., from skin), neural stem cells, mesenchymal stem cells, basal stem cells of the lungs, embryonic stem cells, adult stem cells, induced pluripotent stem cells and liver progenitor cells.

### Pharmacology and Utility

In one embodiment, the present invention relates to ex vivo cell therapies involvining expansion of cells using small molecule LATS kinase inhibitors, said cells being modified as described herein.

Ex vivo cell therapies generally involve expansion of a cell population isolated from a patient or healthy donor to be transplanted to a patient to establish a transient or stable graft of the expanded cells. Ex vivo cell therapies can be used to deliver a gene or biotherapeutic molecule to a patient, wherein gene transfer or expression of the biotherapeutic molecule is achieved in the isolated cells. Non-limiting examples of ex vivo cell therapies include, but are not limited to, stem cell transplantation (e.g., hematopoietic stem cell transplantation, autologous stem cell transplantation, or cord blood stem cell transplantation), tissue regeneration, cellular immunotherapy, and gene therapy. See, e.g., Naldini, 2011, Nature Reviews Genetics volume 12, pages 301-315.

Ex vivo procedures are well known in the art and are discussed more fully below. Briefly, cells are isolated from a mammal (e.g., a human) and genetically modified (i.e., transduced or transfected in vitro) with a gRNA molecule of the invention. The modified cell can be administered to a mammalian recipient to provide a therapeutic benefit. The mammalian recipient may be a human and the cell can be autologous with respect to the recipient. Alternatively, the cells can be allogeneic with respect to the recipient.

The term “autologous” refers to any material derived from the same individual into whom it is introduced.

The term “allogeneic” refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

### Pharmaceutical Composition and Administration

Pharmaceutical compositions of the present invention may comprise a cell (e.g., a modified cell, such as LSC or CEC, with reduced or eliminated expression of B2M by a CRISPR system), e.g., a plurality of cells, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

In one embodiment, the pharmaceutical compositions of the present invention are cryopreserved compositions. The cryopreserved compositions comprise a cell (e.g., a modified cell, such as LSC or CEC, with reduced or eliminated expression of B2M by a CRISPR system), e.g., a plurality of cells) and a cryoprotectant. The term “cryoprotectant”, as used herein, refers to chemical compounds which are added to biological samples in order to minimize the deleterious effects of cryopreservation procedures. In one embodiment, the cryopreserved compositions comprise a cell (e.g., a modified cell, such as LSC or CEC, with reduced or eliminated expression of B2M by a CRISPR system), e.g., a plurality of cells) and a cryoprotectant selected from the list of glycerol, DMSO (dimethylsulfoxide) polyvinylpyrrolidone, hydroxyethyl starch, propylene glycol, acetamide, monosaccharides, algae-derived polysaccharides, and sugar alcohols, or a combination thereof. In a more specific embodiment, the cryopreserved compositions comprise a cell (e.g., a modified cell, such as LSC or CEC, with reduced or eliminated expression of B2M by a CRISPR system), e.g., a plurality of cells) and DMSO concentration of 0.5% to 10%, e.g., 1%- 10%, 2%-7%, 3%-6%, 4% - 5%, preferably 5%. DMSO acts as a cryoprotecting agent against formation of water crystals within and outside the cells, which could lead to cell damage during cryopreservation steps. In a further embodiment, the cryopreserved compositions further comprise a suitable buffer, for example CryoStor CS5 buffer (BioLife Solutions).

Compositions of the present invention are in one aspect formulated for intravenous administration. Composition of the present invention are in one aspect formulated for topical administration, in particular for topical eye administartion.

Pharmaceutical compositions of the present invention may be administered in a manner appropriate to the disease to be treated (or prevented). The quantity and frequency of administration will be determined by such factors as the condition of the patient, and the type and severity of the patient’s disease, although appropriate dosages may be determined by clinical trials.

In one embodiment, the pharmaceutical composition is substantially free of, e.g., there are no detectable levels of a contaminant, e.g., selected from the group consisting of endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium and a fungus. In one embodiment, the bacterium is at least one selected from the group consisting of Alcaligenes faecalis, Candida albicans, Escherichia coli, Haemophilus influenza, Neisseria meningitides, Pseudomonas aeruginosa, Staphylococcus aureus, Streptococcus pneumonia, and Streptococcus pyogenes group A.

In another aspect, in embodiments of the invention relating to in vivo use, the present invention provides a pharmaceutical composition comprising a modified limbal stem cell of the present invention, or a cell population obtainable or obtained by the method of cell population expansion according to the invention, and a pharmaceutically acceptable carrier. In a further embodiment, the composition comprises at least two pharmaceutically acceptable carriers, such as those described herein.

In certain instances, it may be advantageous to administer the cell population (e.g., a cell population comprising modified cells, such as LSCs or CECs, with reduced or eliminated expression of B2M by a CRISPR system, e.g., S. pyogenes Cas9 CRISPR system) obtainable or obtained by the method of cell population expansion according to the invention in combination with at least one additional pharmaceutical (or therapeutic) agent, such as an immunosuppressant for example corticosteroids, cyclosporine, tacrolimus, and combinations of immunosuppressants. In particular, compositions will either be formulated together as a combination therapeutic or administered separately.

### Preparation of Lats Inhibitor Compounds

The LATS inhibitor compounds useful in methods of the present invention can be prepared in a number of ways known to one skilled in the art of organic synthesis in view of the methods, reaction schemes and examples provided herein. Such compounds of the present invention can be synthesized using the methods described in U.S. Pat. Application No. 15/963,816, filed Apr. 26, 2018, and International Application No. PCT/IB2018/052919 (WO 2018/198077), filed Apr. 26, 2018, which are incorporated herein in itheir entirety.

For example, LATS inhibitor compounds can be synthesized using the methods described below, together with synthetic methods known in the art of synthetic organic chemistry, or by variations thereon as appreciated by those skilled in the art. Preferred methods include, but are not limited to, those described below. The reactions are performed in a solvent or solvent mixture appropriate to the reagents and materials employed and suitable for the transformations being effected. It will be understood by those skilled in the art of organic synthesis that the functionality present on the molecule should be consistent with the transformations proposed. This will sometimes require a judgment to modify the order of the synthetic steps or to select one particular process scheme over another in order to obtain a desired compound of the present invention.

The starting materials are generally available from commercial sources such as Aldrich Chemicals (Milwaukee, Wis.) or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), Larock, R.C., Comprehensive Organic Transformations, 2^{nd}-ed., Wiley-VCH Weinheim, Germany (1999), or Beilsteins Handbuch der organischen Chemie, 4, Aufl. ed. Springer-Verlag, Berlin, including supplements (also available via the Beilstein online database).

For illustrative purposes, the reaction schemes depicted below provide potential routes for synthesizing the compounds of the present invention as well as key intermediates. For a more detailed description of the individual reaction steps, see the Examples section below. Those skilled in the art will appreciate that other synthetic routes may be used to synthesize the inventive compounds. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

In the preparation of compounds of the present invention, protection of remote functionality of intermediates may be necessary. The need for such protection will vary depending on the nature of the remote functionality and the conditions of the preparation methods. The need for such protection is readily determined by one skilled in the art. For a general description of protecting groups and their use, see Greene, T.W. et al., Protecting Groups in Organic Synthesis, 4th Ed., Wiley (2007). Protecting groups incorporated in making of the compounds of the present invention, such as the trityl protecting group, may be shown as one regioisomer but may also exist as a mixture of regioisomers.

### Abbreviations

Abbreviations as used herein, are defined as follows: “1x” for once, “2x” for twice, “3x” for thrice, “°C” for degrees Celsius, “aq” for aqueous, “Col” for column, “eq” for equivalent or equivalents, “g” for gram or grams, “mg” for milligram or milligrams, “nm” for nanometer or nanometers, “L” for liter or liters, “mL” or “ml” for milliliter or milliliters, “ul”, “uL”, “µl”, or “µL” for microliter or microliters, “nL” or “nl” for nanoliter or nanoliters, ” “N” for normal, “uM” or “µM” micromolar, “nM” for nanomolar, “mol” for mole or moles, “mmol” for millimole or millimoles, “min” for minute or minutes, “h” or “hrs” for hour or hours, “RT” for room temperature, “ON” for overnight, “atm” for atmosphere, “psi” for pounds per square inch, “conc.” for concentrate, “aq” for aqueous, “sat” or “sat’d” for saturated, “MW” for molecular weight, “mw” or “µwave” for microwave, “mp” for melting point, “Wt” for weight, “MS” or “Mass Spec” for mass spectrometry, “ESI” for electrospray ionization mass spectroscopy, “HR” for high resolution, “HRMS” for high resolution mass spectrometry, “LCMS” for liquid chromatography mass spectrometry, “HPLC” for high pressure liquid chromatography, “RP HPLC” for reverse phase HPLC, “TLC” or “tlc” for thin layer chromatography, “NMR” for nuclear magnetic resonance spectroscopy, “nOe” for nuclear Overhauser effect spectroscopy, “1H” for proton, “δ ” for delta, “s” for singlet, “d” for doublet, “t” for triplet, “q” for quartet, “m” for multiplet, “br” for broad, “Hz” for hertz, “ee” for “enantiomeric excess” and “α”, “β”, “R”,“r”, “S”, “s”, “E”, and “Z” are stereochemical designations familiar to one skilled in the art.

The following abbreviations used herein below have the corresponding meanings:

| | |
|---|---|
| AC | Active Control |
| AIBN | azobisisobutyronitrile |
| ATP | adenosine triphosphate |
| Bn | benzyl |
| Boc | tert-butoxy carbonyl |
| Boc₂O | di-tert-butyl dicarbonate |
| BSA | bovine serum albumin |
| Bu | butyl |
| Cs₂CO₃ | cesium carbonate anhydrous |
| CHCl₃ | chloroform |
| DAST | diethylaminosulfurtrifluoride |
| DBU | 2,3,4,6,7,8,9,10-octahydropyrimido[1,2-a]azepine |
| DCM | dichloromethane |
| DMAP | 4-dimethylaminopyridine |
| DMEM | Dulbecco’s modified Eagle’s medium |
| DMF | dimethylformamide |
| DMSO | dimethylsulfoxide |
| DPPA | diphenylphosphoryl azide |
| DTT | dithiolthreitol |
| EA | ethyl acetate |
| EDTA | ethylenediaminetetraacetic acid |
| Equiv. | equivalence |
| Et | ethyl |
| Et₂O | diethyl ether |
| EtOH | ethanol |
| EtOAc | ethyl acetate |
| FBS | fetal bovine serum |
| HATU | 2-(7-Aza-1H-benzotriazole-1-yl)-1,1,3,3-tetramethyluronium hexafluorophosphate |
| HCl | hydrochloric acid |
| HEPES | (4-(2-hydroxyethyl)-1-piperazineethanesulfonic acid |
| HPMC | (hydroxypropyl)methyl cellulose |
| HTRF | homogeneous time resolved fluorescence |
| i-Bu | isobutyl |
| i-Pr | isopropyl |
| KOAc | potassium acetate |
| LiAlH₄ | lithium aluminium hydride |
| LATS | large tumor suppressor |
| LSC | limbal stem cell |
| LSCD | limbal stem cell deficiency |
| Me | methyl |
| mCPBA | 3-chloroperoxybenzoic acid |
| MeCN | acetonitrile |
| MnO₂ | manganese dioxide |
| N₂ | nitrogen |
| NaBH₄ | sodium borohydride |
| NaHCO₃ | sodium bicarbonate |
| Na₂SO₄ | sodium sulfate |
| NBS | N-Bromosuccinimide |
| NC | Neutral Control |
| PBS | phosphate buffered saline |
| PFA | paraformaldehyde |
| Ph | phenyl |
| PPh₃ | triphenylphosphine |
| Ph₃P=O | triphenylphosphine oxide |
| pYAP | phospho-YAP |
| R_{f} | retention factor |
| RT | room temperature (°C) |
| Ser | serine |
| t-Bu or Bu^{t} | tert-butyl |
| T3P® | Propane phosphonic acid anhydride |
| TEA | triethylamine |
| TFA | trifluoroacetic acid |
| THF | tetrahydrofuran |
| UVA | Ultraviolet A |
| YAP | Yes associated protein (NCBI Gene ID: 10413; official symbol: (YAP1) |

### I. General Synthetic Routes

Compounds of Formulae I to VI can be prepared as illustrated in the General Schemes I to III and in greater details in Schemes 1 to 6 below.

### General Scheme I for the Preparation of Compounds of Formula I or II

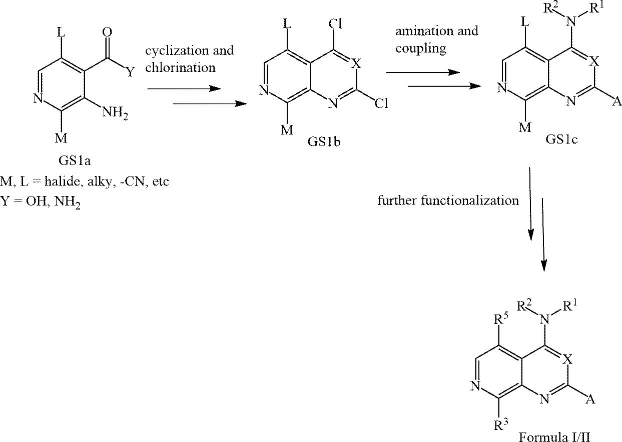

The bicyclic dichloride GS1b could be commercially available when X = C or could be prepared from aminoisonicotinic acid/amide GS1a through cyclization and chlorination. The dichloride of GS1b could be aminated and coupled with the appropriate agents to form GS1c, which further functionalized to yield Formula I or Formula II through any necessary functionalization, such as but not limited to protection and de-protection steps, reduction, hydrolysis, alkylation, amination, coupling, etc

### General Scheme II for the Preparation of Compounds of Formula III

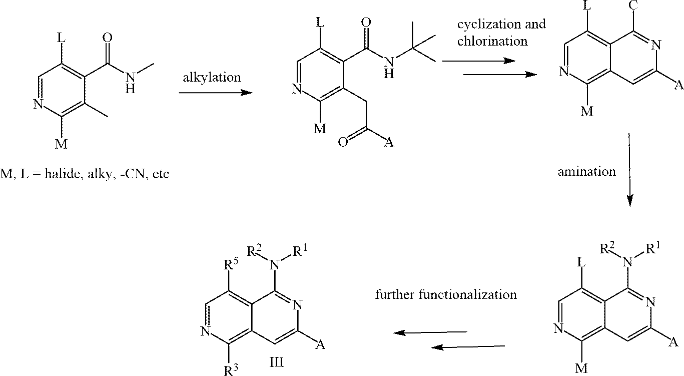

### General Scheme III for the Preparation of Compounds of Formula IV

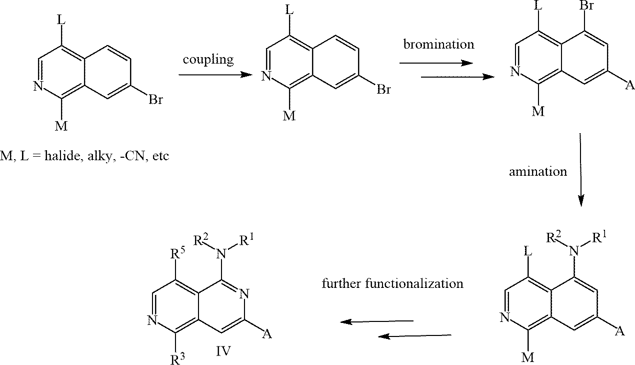

### Scheme 1.

Compounds of Formula V can be prepared as illustrated in Scheme 1 below. Step C could include amination and any necessary functionalization, such as but not limited to protection and de-protection steps, reduction, hydrolysis, alkylation, etc.

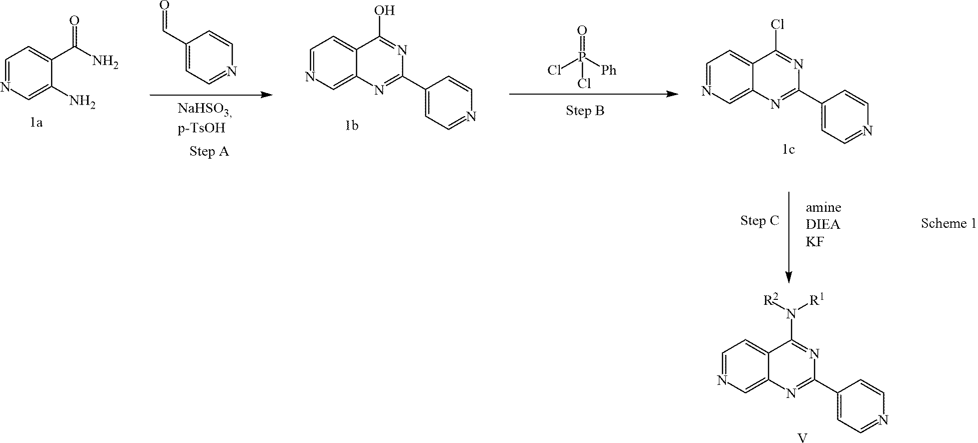

### Scheme 2.

Alternatively, compounds of Formula V can be prepared as illustrated in Scheme 2. Step C could include amination and any necessary functionalization, such as but not limited to protection and de-protection steps, reduction, hydrolysis, alkylation, etc. Further functionalization of mono-chloride intermediate 2d by but not limited to metal mediated coupling, amination, alkylation etc. and necessary protection and de-protection steps, leads to compounds of Formula V.

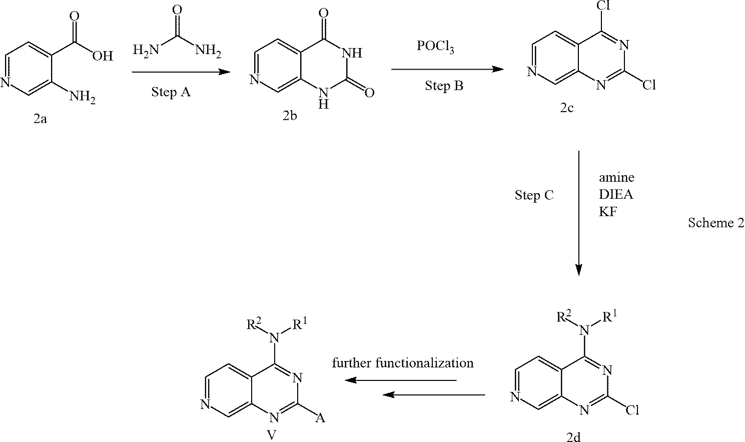

### Scheme 3.

Compounds of Formula I where R⁵ is hydrogen can be prepared as illustrated in Scheme 3. Step C could include amination and any necessary functionalization, such as but not limited to protection and de-protection steps, reduction, hydrolysis, alkylation, etc. Further functionalization of mono-chloride intermediate 3d by but not limited to metal mediated coupling, amination, alkylation etc. and necessary protection and de-protection steps, leads to compounds of Formula (I) where R⁵ is hydrogen.

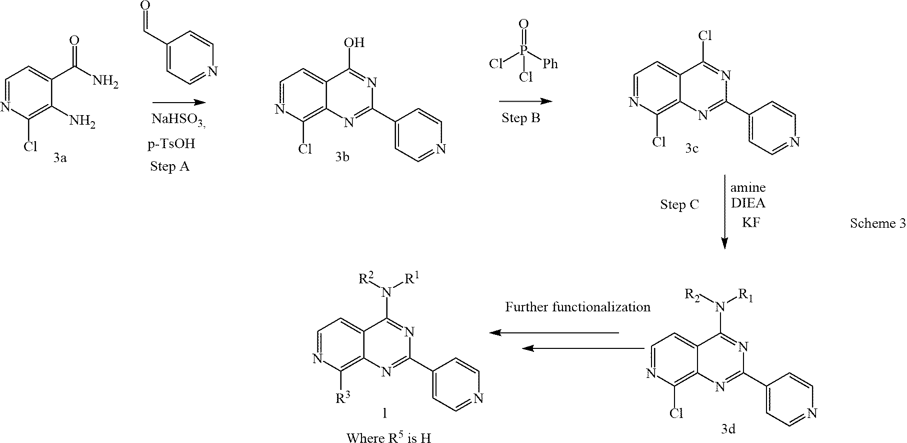

### Scheme 4.

Compounds of Formula I, where R³ and R⁵ are both hydrogen, can be prepared as illustrated in Scheme 4. Step C could include amination and any necessary functionalization, such as but not limited to protection and de-protection steps, reduction, hydrolysis, alkylation, etc. leads to compounds of Formula I where R³ and R⁵ are both hydrogen.

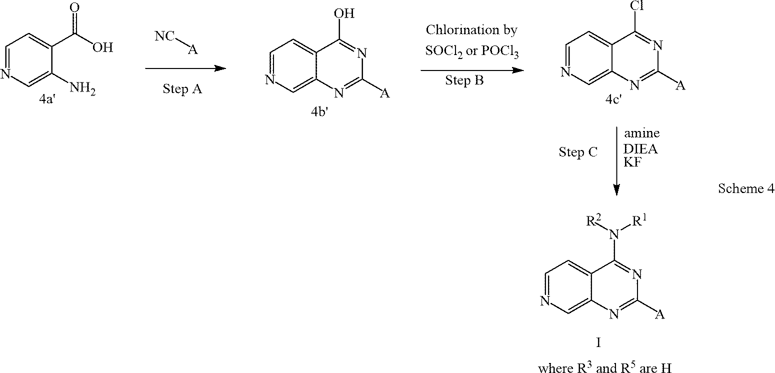

### Scheme 5.

Compounds of Formula I, where R³ is hydrogen, can be prepared as illustrated in Scheme 5. Step D could include amination and any necessary functionalization, such as but not limited to protection and de-protection steps, reduction, hydrolysis, alkylation, etc. Further functionalization of mono- chloride intermediate 5d by, but not limited to, metal mediated coupling, amination, alkylation etc. and necessary protection and de-protection steps, leads to compounds of Formula I where R³ is hydrogen,

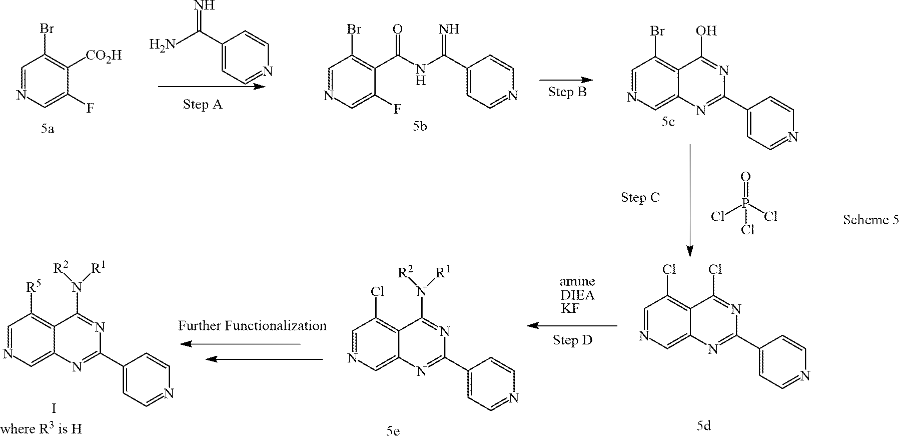

### Scheme 6.

Compounds of Formula VI can be prepared from commercially available dichloride 6a′ (2,4-dichloro-1,7-naphthyridine, Aquila Pharmatech) as illustrated in Scheme 6. Step A could include metal mediated coupling and any necessary functionalization, such as but not limited to protection and de-protection steps, cyclization, reduction, hydrolysis, alkylation, etc. Step B could include amination and any necessary functionalization, such as but not limited to protection and de-protection steps, reduction, hydrolysis, alkylation, etc.

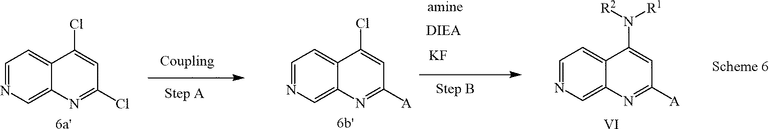

### Preparation of Exemplified Examples

The following Examples have been prepared, isolated and characterized using the methods disclosed herein. The following examples demonstrate a partial scope of the invention and are not meant to be limiting of the scope of the invention.

Unless specified otherwise, starting materials are generally available from a non-excluding commercial sources such as TCI Fine Chemicals (Japan), Shanghai Chemhere Co., Ltd.(Shanghai, China), Aurora Fine Chemicals LLC (San Diego, CA), FCH Group (Ukraine), Aldrich Chemicals Co. (Milwaukee, Wis.), Lancaster Synthesis, Inc. (Windham, N.H.), Acros Organics (Fairlawn, N.J.), Maybridge Chemical Company, Ltd. (Cornwall, England), Tyger Scientific (Princeton, N.J.), AstraZeneca Pharmaceuticals (London, England), Chembridge Corporation (USA), Matrix Scientific (USA), Conier Chem & Pharm Co., Ltd (China), Enamine Ltd (Ukraine), Combi-Blocks, Inc. (San Diego, USA), Oakwood Products, Inc. (USA), Apollo Scientific Ltd. (UK), Allichem LLC. (USA) and Ukrorgsyntez Ltd (Latvia).

### LCMS Methods Employed in Characterization of Examples

Analytical LC/MS is carried out on Agilent systems using ChemStation software. The systems consist of:
Agilent G1312 Binary Pump
Agilent G1367 Well Plate Autosampler
Agilent G1316 Thermostated Column Compartment
Agilent G1315 Diode Array Detector
Agilent 6140/6150 Mass Spectrometer
SOFTA Evaporative Light Scattering Detector

Typical method conditions are as follows:
Flow Rate: 0.9 mL/min
Column: 1.8 micrometres 2.1×50 mm Waters Acquity HSS T3 C18 column
Mobile Phase A: Water+0.05% TFA
Mobile Phase B: Acetonitrile+0.035% TFA
Run Time: 2.25 minutes
The system runs a gradient from 10% B to 90% B in 1.35 minutes. A 0.6 minute wash at 100% B follows the gradient. The remaining duration of the method returns the system to initial conditions.
Typical mass spectrometer Scan range is 100 to 1000 amu.

### NMR Employed in Characterization of Examples

Proton spectra are recorded on a Bruker AVANCE II 400 MHz with 5 mm QNP Cryoprobe or a Bruker AVANCE III 500 MHz with 5 mm QNP probe unless otherwise noted. Chemical shifts are reported in ppm relative to dimethyl sulfoxide (δ 2.50), chloroform (δ 7.26), methanol (δ 3.34), or dichloromethane (δ 5.32). A small amount of the dry sample (2-5 mg) is dissolved in an appropriate deuterated solvent (1 mL).

### Reagents and Materials

Solvents and reagents were purchased from suppliers and used without any further purification. Basic ion exchange resin cartridges PoraPakTM Rxn CX 20 cc (2 g) were purchased from Waters. Phase separator cartridges (Isolute Phase Separator) were purchased from Biotage. Isolute absorbant (Isolute HM-N) was purchased from Biotage.

### ISCO Methods Employed in Purification of Examples

ISCO flash chromatography is carried on Teledyne COMBIFLASH® system with prepacked silica RediSep® column.

### Preparative HPLC Methods Employed in Purification of Examples

Preparative HPLC is carried out on Waters Autoprep systems using MassLynx and FractionLynx software. The systems consist of:
Waters 2767 Autosampler/Fraction Collector
Waters 2525 Binary Pump
Waters 515 Makeup pump
Waters 2487 Dual Wavelength UV Detector
Waters ZQ Mass Spectrometer

Typical method conditions are as follows:
Flow Rate: 100 mL/min
Column: 10 micrometres 19×50 mm Waters Atlantis T3 C18 column
Injection Volume: 0-1000 microlitres
Mobile Phase A: Water+0.05% TFA
Mobile Phase B: Acetonitrile+0.035% TFA
Run Time: 4.25 minutes

The system runs a gradient from x% B to y% B as appropriate for the examples in 3 minutes following a 0.25 minute hold at initial conditions. A 0.5 minute wash at 100%B follows the gradient. The remaining duration of the method returns the system to initial conditions.

Fraction collection is triggered by mass detection through FractionLynx software.

### Chiral Preparative HPLC Methods Employed in Purification of Examples

SFC chiral screening is carried out on a Thar Instruments Prep Investigator system coupled to a Waters ZQ mass spectrometer. The Thar Prep Investigator system consists of:
Leap HTC PAL autosampler
Thar Fluid Delivery Module (0 to 10 mL/min)
Thar SFC 10 position column oven
Waters 2996 PDA
Jasco CD-2095 Chiral Detector
Thar Automated Back Pressure Regulator.

All of the Thar components are part of the SuperPure Discovery Series line.

The system flows at 2 mL/min (4 mL/min for the WhelkO-1 column) and is kept at 30° C. The system back pressure is set to 125 bar. Each sample is screened through a battery of six 3 micrometre columns:
3 micrometre 4.6×50 mm ChiralPak AD
3 micrometre 4.6×50 mm ChiralCel OD
3 micrometre 4.6×50 mm ChiralCel OJ
3 micrometre 4.6×250 mm Whelk O-1
3 micrometre 4.6×50 mm ChiralPak AS
3 micrometre 4.6×50 mm Lux-Cellulose-2

The system runs a gradient from 5% co-solvent to 50% co-solvent in 5 minutes followed by a 0.5 minute hold at 50% co-solvent, a switch back to 5% co-solvent and a 0.25 minute hold at initial conditions. In between each gradient there is a 4 minute equilibration method the flows at 5% co-solvent through the next column to be screened. The typical solvents screened are MeOH, MeOH+20mM NH₃, MeOH+0.5%DEA, IPA, and IPA+20mM NH₃.

Once separation is detected using one of the gradient methods an isocratic method will be developed and, if necessary, scaled up for purification on the Thar Prep80 system.

### Example 1: N-methyl-2-(pyridin-4-yl)-N-(1,1,1-trifluoropropan-2-yl)pyrido[3,4-d]pyrimidin-4-amine

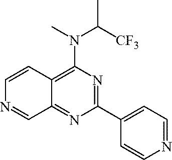 [SMILES: CC(N(C)c1nc(-c2ccncc2)nc2cnccc12)C(F)(F)F]

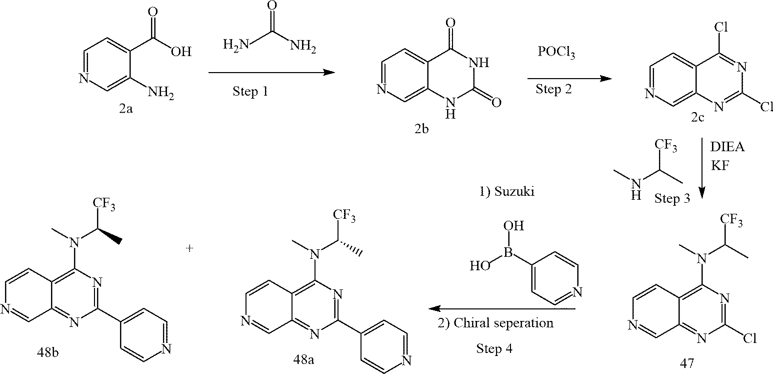

Step 1: A mixture of urea (40.00 g, 666.00 mmol) and 3-aminoisonicotinic acid (2a, 18.40 g, 133.20 mmol) was heated at 210° C. for 1 hr (NOTE: no solvent was used). NaOH (2N, 320 mL) was added, and the mixture was stirred at 90° C. for 1h. The solid was collected by filtration, and washed with water. The crude product thus obtained was suspended in HOAc (400 mL), and stirred at 100° C. for 1h. The mixture was cooled to RT, filtered, and the solid was washed with a large amount of water, and then dried under the vacuum to give pyrido[3,4-d]pyrimidine-2,4(1H,3H)-dione (2b, 17.00 g, 78% yield) without further purification. LCMS (m/z [M+H]⁺): 164.0.

Step 2: To a mixture of pyrido[3,4-d]pyrimidine-2,4(1H,3H)-dione (2b, 20.00 g, 122.60 mmol) and POCl₃ (328.03 g, 2.14 mol) in toluene (200 mL) was added DIEA (31.69 g, 245.20 mmol) dropwise and this reaction mixture stirred at 25° C. overnight (18 hr) to give suspension.

The solvent and POCl₃ was removed under vacuum, diluted with DCM (50 mL), neutralized with DIEA to pH=7 at -20° C. and concentrated again, the residue was purified by column (20-50% EA/PE) to give 2,4-dichloropyrido[3,4-d]pyrimidine (2c, 20.00 g, 99.99 mmol, 82% yield) as a yellow solid. 1H NMR (400 MHz, CHLOROFORM-d) δ 9.52 (s, 1 H), 8.92 (d, J=5.6 Hz, 1 H), 8.04 (d, J=5.6 Hz, 1 H). LCMS (m/z [M+H]⁺): 200.0.

Step 3: In a 20 mL vial 2,4-dichloropyrido[3,4-d]pyrimidine (600 mg, 3.0 mmol) was stirred in DMSO (0.7 mL) at room temperature and degassed with N₂. DIEA (1 mL, 6 mmol) was added and stirred for 5 minutes then KF (174 mg, 3 mmol). This mixture was stirred at room temperature for 15 minutes then racemic 1,1,1-trifluoro-N-methylpropan-2-amine (419 mg, 3.3 mmol) was added and degassed then stirred at 60° C. for 4 hours. The reaction was then concentrated and purified by flash chromatography on a COMBIFLASH® system (ISCO) using 0-10% MeOH/DCM to afford 2-chloro-N-methyl-N-(1,1,1-trifluoropropan-2-yl)pyrido[3,4-d]pyrimidin-4-amine (680 mg, 74%). 1H NMR (500 MHz, Acetone-d6) δ 9.09 (d, J = 0.9 Hz, 1H), 8.59 (d, J = 5.9 Hz, 1H), 8.22 (dd, J = 5.9, 0.9 Hz, 1H), 5.93 (dddd, J = 15.3, 8.3, 7.0, 1.2 Hz, 1H), 3.61 (q, J = 1.0 Hz, 3H), 1.63 (d, J = 7.0 Hz, 3H). LCMS (m/z [M+H]⁺): 291.7.

Step 4: In a 20 mL microwave reactor was added PalladiumTetrakis (99 mg, 0.086 mmol), potassium carbonate (2.15 mL, 4.3 mmol), and 2 chloro-N-methyl-N-(1,1,1-trifluoropropan-2-yl)pyrido[3,4-d]pyrimidin-4-amine (500 mg, 1.72 mmol) and pyridin-4-ylboronic acid (233 mg, 1.89 mmol) in acetonitrile (8 mL) to give an yellow suspension. The reaction mixture was stirred at 130° C. for 30 min under microwave. The crude mixture was diluted with DCM, H₂O, separated and extracted with DCM x3. Combined the organic layers and dried Na₂SO₄, filtered and concentrated. The residue was purified by flash chromatography on a COMBIFLASH® system (ISCO) using 0-10% MeOH/DCM to give Example 1, the racemic product, then followed by chiral HPLC (21×250 mm OJ-H column with 85% CO₂ as phase A and 15% MeOH as phase B, flow rate 2 mL/min, 30° C., 3.5 min elution time) to separate the enantiomers to afford Examples 1a and 1b.

### Example 1a: N-methyl-2-(pyridin-4-yl)-N-[(2S)-1,1,1-trifluoropropan-2-yl]pyrido[3,4-d]pyrimidin-4-amine

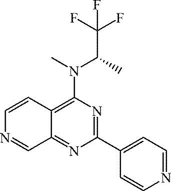 [SMILES: C[C@H](N(C)c1nc(-c2ccncc2)nc2cnccc12)C(F)(F)F]

1H NMR (500 MHz, DMSO-d6) δ 9.33 (d, J = 0.8 Hz, 1H), 8.86 - 8.75 (m, 2H), 8.63 (d, J = 5.9 Hz, 1H), 8.38 - 8.30 (m, 2H), 8.20 (dd, J = 6.0, 0.9 Hz, 1H), 6.11 (qt, J = 8.5, 7.4 Hz, 1H), 3.50 (d, J = 1.1 Hz, 3H), 1.61 (d, J = 7.0 Hz, 3H). LCMS (m/z [M+H]⁺): 334.1. Chiral HPLC T_{R} = 1.73 min. Absolute stereochemistry was confirmed by X-ray crystal structure.

### Example 1b: N-methyl-2-(pyridin-4-yl)-N-[(2R)-1,1,1-trifluoropropan-2-yl]pyrido[3,4-d]pyrimidin-4-amine

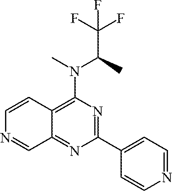 [SMILES: C[C@@H](N(C)c1nc(-c2ccncc2)nc2cnccc12)C(F)(F)F]

1H NMR (500 MHz, DMSO-d6) δ 9.33 (d, J = 0.8 Hz, 1H), 8.86 - 8.75 (m, 2H), 8.63 (d, J = 5.9 Hz, 1H), 8.38 - 8.30 (m, 2H), 8.20 (dd, J = 6.0, 0.9 Hz, 1H), 6.11 (qt, J = 8.5, 7.4 Hz, 1H), 3.50 (d, J = 1.1 Hz, 3H), 1.61 (d, J = 7.0 Hz, 3H). LCMS (m/z [M+H]⁺): 334.1. Chiral HPLC T_{R} = 1.25 min. Absolute stereochemistry was confirmed by X-ray crystal structure.

### Example 2: N-(tert-butyl)-2-(pyridin-4-yl)-1,7-naphthyridin-4-amine:

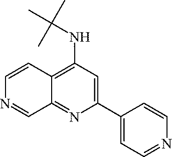 [SMILES: CC(C)(C)Nc1cc(-c2ccncc2)nc2cnccc12]

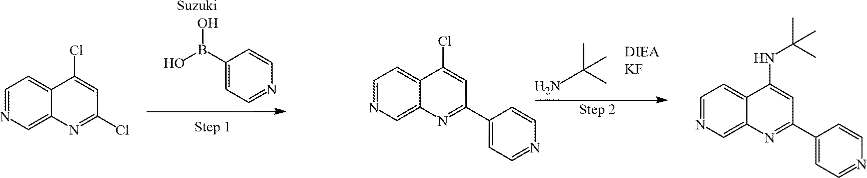

Step 1: In a 20 mL microwave reactor was added PalladiumTetrakis (58.1 mg, 0.050 mmol), potassium carbonate (1.256 mL, 2.51 mmol), and 2,4-dichloro-1,7-naphthyridine (200 mg, 1.005 mmol) and pyridin-4-ylboronic acid (130 mg, 1.055 mmol) in Acetonitrile (Volume: 2 mL) to give an orange suspension. The reaction mixture was stirred at 120° C. for 60 min under microwave. The crude mixture was diluted with DCM, H₂O, separated and extracted with DCM x3. Combined the organic layers and dried Na₂SO₄, filtered and concentrated. The residue was purified by flash chromatography on a COMBIFLASH® system (ISCO) using 0-10% MeOH/DCM to give the product (62%). 1H NMR (400 MHz, DMSO-d6) δ 9.58 (d, J = 0.9 Hz, 1H), 8.85 - 8.78 (m, 4H), 8.32 - 8.29 (m, 2H), 8.11 (dd, J = 5.8, 0.9 Hz, 1H). LCMS [M+H] = 242.

Step 2: In a 40ml vial was added potassium fluoride (11.54 mg, 0.199 mmol), 4-chloro-2-(pyridin-4-yl)-1,7-naphthyridine (40 mg, 0.166 mmol), and 2-methylpropan-2-amine (0.035 mL, 0.331 mmol) in DMSO (Volume: 2 mL) to give a yellow suspension. The reaction mixture was stirred at 130° C. for 24 hrs. Solvent was evaporated under air flow. The residue was purified by flash chromatography on a COMBIFLASH® system (ISCO) using 0-10% MeOH/DCM to give the product (82%). 1H NMR (400 MHz, DMSO-d6) δ 9.22 (d, J = 0.7 Hz, 1H), 8.78 - 8.72 (m, 2H), 8.48 (d, J = 5.8 Hz, 1H), 8.30 (dd, J = 6.0, 0.9 Hz, 1H), 8.15 -8.06 (m, 2H), 7.28 (s, 1H), 6.73 (s, 1H), 1.56 (s, 9H). LCMS [M+H] = 279.2.

### Example 3: 2,4-dimethyl-4-{[2-(pyridin-4-yl)pyrido[3,4-d]pyrimidin-4-yl]amino}pentan-2-ol

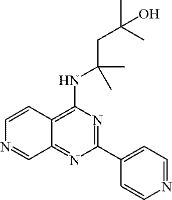 [SMILES: CC(C)(O)CC(C)(C)Nc1nc(-c2ccncc2)nc2cnccc12]

1H NMR (400 MHz, Acetone-d6) δ 9.57 (s, 1H), 9.15 (d, J = 0.9 Hz, 1H), 8.82 - 8.72 (m, 2H), 8.56 (d, J = 5.6 Hz, 1H), 8.44 - 8.37 (m, 2H), 7.69 (dd, J = 5.6, 0.9 Hz, 1H), 2.08 (s, 2H), 1.87 (s, 6H), 1.48 (d, J = 0.8 Hz, 6H). LCMS (m/z [M+H]⁺): 338.2.

### Example 4: 2-(3-methyl-1H-pyrazol-4-yl)-N-(1-methylcyclopropyl)pyrido[3,4-d]pyrimidin-4-amine

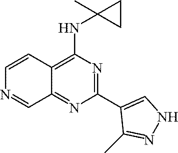 [SMILES: Cc1n[nH]cc1-c1nc(NC2(C)CC2)c2ccncc2n1]

1H NMR (500 MHz, Methanol-d4) δ 9.01 (s, 1H), 8.41 (d, J = 5.7 Hz, 1H), 8.26 (s, 1H), 7.91 (dd, J = 5.7, 0.9 Hz, 1H), 2.83 (s, 3H), 1.60 (s, 3H), 1.05 - 0.94 (m, 2H), 0.91 -0.82 (m, 2H). LCMS (m/z [M+H]+): 281.1.

### Example 5: Dimethyl(3-methyl-3-{[2-(pyridin-4-yl)pyrido[3,4-d]pyrimidin-4-yl]amino}butyl)amine

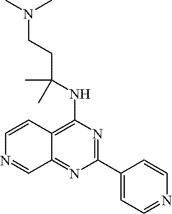 [SMILES: CN(C)CCC(C)(C)Nc1nc(-c2ccncc2)nc2cnccc12]

1H NMR (400 MHz, DMSO-d6) δ 9.23 (s, 1H), 8.96 (t, J = 5.7 Hz, 1H), 8.88 - 8.82 (m, 2H), 8.70 (d, J = 5.5 Hz, 1H), 8.51 - 8.44 (m, 2H), 8.20 (dd, J = 5.6, 1.0 Hz, 1H), 7.65 (s, 2H), 3.80 - 3.72 (m, 2H), 2.86 (td, J = 7.5, 5.5 Hz, 2H), 1.85 - 1.73 (m, 2H), 1.67 (ddt, J = 12.8, 9.9, 5.8 Hz, 2H). LCMS (M/Z [M+H]+): 295.2.

### Example 6: N¹,N¹,3-trimethyl-N³-(2-(3-methyl-1H-pyrazol-4-yl)pyrido[3,4-d]pyrimidin-4-yl)butane-1,3-diamine

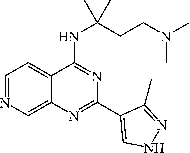 [SMILES: Cc1n[nH]cc1-c1nc(NC(C)(C)CCN(C)C)c2ccncc2n1]

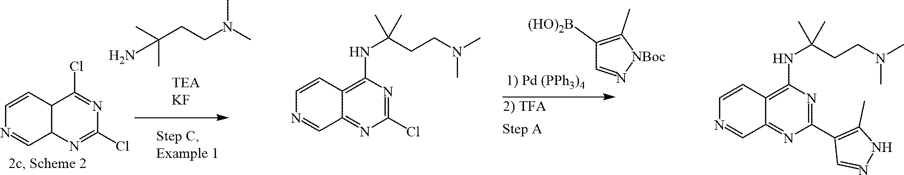

The title compound was synthesized in 2 steps from 2,4-dichloropyrido[3,4-d]pyrimidine (2c) and N′,N′,3-trimethylbutane-1,3-diamine (step C) and from N³-(2-chloropyrido[3,4-d]pyrimidin-4-yl)-N¹,N¹,3-trimethylbutane-1,3-diamine and tert-butyl 3-methyl-4-(4,4,5,5-tetramethyl-1,3,2-dioxaborolan-2-yl)-1H-pyrazole-1-carboxylate (CAS no.1009071-34-4, step A).

1H NMR (400 MHz, DMSO-d₆) δ ppm 12.57 - 12.95 (m, 1 H), 8.89 - 9.00 (m, 2 H), 8.48 (d, J=5.50 Hz, 1 H), 7.93 - 8.29 (m, 1 H), 7.75 (br d, J=5.38 Hz, 1 H), 2.55 - 2.84 (m, 5 H), 2.25 (s, 6 H), 1.99 (br s, 2 H), 1.61 (s, 6 H). LCMS (m/z [M+H]⁺): 340.3, Rt₁=0.48 min.

### Starting Material to Prepare an Expanded Population of Cells

### Autologous Method

The seeding population of cells for use in the method of cell population expansion to obtain an expanded population of cells may be obtained from a recipient himself/herself. In patients where tissue, organ, or cell deficiency is partial, for example healthy cells are present, the seeding population of cells may be obtained from non-affected tissue or organ or cell source. For example, in the case of unilateral ocular cell deficiency, the seeding population may be obtained from a biopsy on the non-affected eye. It may also be obtained from healthy tissue remaining in an organ that is partially damaged.

### Allogenic Method

In a preferred embodiment, the seeding population of cells for use in the method of cell population expansion to obtain an expanded population of cells may be obtained from cells originally derived from donor tissue (e.g., human, rabbit, monkey etc., preferably human). For example a source of human tissue is from cadaveric donors or tissues from living donors, including living relatives.

From autologous or allogenic tissue derived as described above under autologous and allogenic methods which has been removed from the body, the cells may, for example, be extracted and prepared as follows: The desired area may be dissected, for example, using scalpels and the cells then dissociated (e.g. using collagenase, dispase, trypsin, accutase or TripLE; for example 1 mg/ml collagenase at 37° C.), until cell detachment becomes apparent by microscopic observation (e.g., using a Zeiss Axiovert inverted microscope) from 45 minutes to 3 hours.

Suitably, the cells, e.g., LSCs or CECs, isolated from several corneas or from different donors may be pooled for further processing, such as cell population expansion and B2M-gene-editting.

For use in the cell population expansion method according to the invention the isolated cells are then added to medium, for example by pipetting, as described below in the section “Cell population expansion”.

In a preferred embodiment according to the invention, an assessment of the quality of cellular material harvested from the donor is performed. For example, approximately 24 hours after harvesting the cells and beginning culturing in medium (growth or cell proliferation medium, as described below), a visual assessment under brightfield microscope to look for floating cells present (as an indicator of dead cells) may be performed. Ideally this assessment is to show that there is approximately less than 10% as floating cells for the material to be suitable for use to generate an expanded population of cells according to the invention.

The number of cells suitable for use in the method of cell population expansion according to the invention is not limited, but as an example for illustrative purposes, the seeding cell population suitable for use in the method of cell population expansion according to the invention may comprise approximately 1000 cells.

If it is desired to measure the cell numbers in the seeding cell population, this may be done for example by manual or automated cell counting using a light microscope, immunohistochemistry or FACS according to standard protocols well known in the art.

### Ex-Vivo Ocular Cell Population Expansion and Use in Therapy

Described below in more detail is a description of the methodology relating to expansion of ocular cell populations (preparation of starting material, followed by cell population expansion phase, storage of cells) as applied to ocular cells with the specific examples of limbal stem cells and corneal endothelial cells.

### Starting Material to Prepare an Expanded Population of Limbal Stem Cells Corneal Epithelial and Limbal Cells

### Autologous Method

The seeding population of cells for use in the method of cell population expansion to obtain an expanded population of limbal stem cells may be obtained from the recipient himself/herself. In patients where limbal stem cell deficiency is partial, the seeding population of cells may be obtained from non-affected parts of the limbus. For example, in the case of unilateral limbal stem cell deficiency, the seeding population may be obtained from a biopsy on the non-affected eye. It may also be obtained from healthy tissue remaining in a limbus that is partially damaged.

### Allogenic Method

In a preferred embodiment, the seeding population of cells for use in the method of cell population expansion to obtain an expanded population of limbal stem cells may be obtained from cells originally derived from donor mammalian corneal tissue (e.g., human, rabbit, monkey etc., preferably human).

For example a source of human corneal tissue is from cadaveric donors (for example sourced through eye banks) or tissues from living donors, including living relatives. A range of donor limbal tissue is suitable for use according to the invention. In a preferred embodiment limbal tissue is obtained from living relatives or donors with a compatible HLA profile.

The tissue that is used to obtain the LSCs may, for example, be a ring of limbal tissue of approximately 4 mm in width and 1 mm in height.

From the corneal tissue as described above under autologous and allogenic methods which has been removed from the body, the LSCs may, for example, be extracted and prepared as follows: The limbal epithelial area may be dissected, for example, using scalpels and the cells then dissociated (e.g., using collagenase, dispase, trypsin, accutase or TripLE; for example 1 mg/ml collagenase at 37° C.), until cell detachment becomes apparent by microscopic observation (e.g., using a Zeiss Axiovert inverted microscope) from 45 minutes to 3 hours.

Suitably, the cells, e.g., LSCs or CECs, isolated from several corneas or from different donors may be pooled for further processing, such as cell population expansion and B2M-gene-editting.

For use in the cell population expansion method according to the invention the isolated cells are then added to medium, for example by pipetting, as described below in the section “Cell population expansion”.

In a preferred embodiment according to the invention, an assessment of the quality of cellular material harvested from the donor cornea is performed. For example, approximately 24 hours after harvesting the cells and beginning culturing in medium (growth or cell proliferation medium, as described below), a visual assessment under brightfield microscope to look for floating cells present (as an indicator of dead cells) may be performed. Ideally this assessment is to show that there is approximately less than 10% as floating cells for the material to be suitable for use to generate an expanded population of cells according to the invention.

The number of cells suitable for use in the method of cell population expansion according to the invention is not limited, but as an example for illustrative purposes, the seeding cell population suitable for use in the method of cell population expansion according to the invention may comprise approximately 1,000 limbal stem cells.

If it is desired to measure the cell numbers in the seeding cell population, this may be done for example by manual or automated cell counting using a light microscope, immunohistochemistry or FACS according to standard protocols well known in the art.

### Starting Material to Prepare an Expanded Population of Corneal Endothelial Cells

The seeding population of corneal endothelial cells (CECs) for use in the method of cell population expansion may be obtained from cells originally derived from mammalian corneal tissue (e.g., human, rabbit, monkey etc., preferably human). For example, a source of human corneal tissue is from cadaveric human donors (which may be sourced through eye banks).

The age of the donors can range, for example, from infancy to 70 years of age. Preferably also suitable donors are those who have no history of corneal disease or trauma. In one embodiment according to the invention, preferred donor corneas are those where the corneal endothelial cell count is above 2 000 cells/mm² (area). In a more preferred embodiment according to the invention the corneal endothelial cell count is 2 000 to 3 500 cells/mm² (area). This is measured for example by examining the cornea of the donor material under a direct light microscope or a specular microscope as per standard Eye Bank techniques known in the art for evaluation of donor tissue before transplantation to patients (see Tran et al (2016) Comparison of Endothelial Cell Measurements by Two Eye Bank Specular Micorscopes; International Journal of Eye Banking; vol 4., no 2; 1-8, which is herein incorporated by reference).

The surface of cornea that is used to obtain the CECs is not limited, but may, for example, be an area of approx. 8-10 mm in diameter.

The CECs may, for example, be extracted and prepared as follows from the donor corneal tissue: The corneal endothelial cell layer and Descemet’s membrane (DM) are scored, for example with a surgical-grade reverse Sinsky endothelial stripper. The DM-endothelial cell layer is peeled off the corneal stroma and cells are dissociated from the DM, for example using 1 mg/ml collagenase at 37° C. until cell detachment becomes apparent by microscopic observation (e.g. using a Zeiss Axiovert inverted microscope) (from 45 minutes to 3 hours). As the DM only carries corneal endothelial cells in the cornea, the cell population isolated in this manner is a population of CECs, which is suitable for use as a seeding population of cells according to the invention.

For use in the method of cell population expansion according to the invention the isolated corneal endothelial cells may be added to medium as described below in the section “Cell population expansion”.

In a preferred embodiment according to the invention, an assessment of the quality of cellular material harvested from the donor cornea is performed. For example, approximately 24 hours after harvesting the cells and beginning culturing in medium (growth or cell proliferation medium, as described below), a visual assessment under brightfield microscope to look for floating cells present (as an indicator of dead cells) may be performed. Ideally this assessment is to show that there is approximately less than 10% as floating cells for the material to be suitable for use to generate an expanded population of cells according to the invention.

The starting number of cells suitable for use in the method of cell population expansion according to the invention is not limited, but as an example for illustrative purposes, the seeding population of corneal endothelial cells suitable for use in the method of cell population expansion according to the invention may be 100 000 to 275 000 cells.

If it is desired to measure the cell numbers in the seeding cell population, this may be done for example by taking an aliquot and performing immunocytochemistry (e.g., to count nuclei stained with Sytox Orange) or by live cell imaging under brightfield microscope to count the number of cells.

The Sytox Orange assay may be performed according to standard protocols known in the art. In brief, after cells have attached to the cell culture dish (typically 24h after cell plating), the cells are fixed in paraformaldehyde. The cells are then permeabilized (e.g., using a solution of 0.3% Triton X-100) and they are then labeled in a solution of Sytox Orange (e.g., using 0.5 micromolar of Sytox Orange in PBS). The number of nuclei stained with Sytox Orange per surface area are then counted under a Zeiss epifluorescence microscope.

### Cell Population Expansion

In one embodiment of the invention, a population of cells comprising cells from a patient or a donor, can be grown in medium in a culture container known in the art, such as plates, multi-well plates, and cell culture flasks. For example, a culture dish may be used which is non-coated or coated with collagen, synthemax, gelatin or fibronectin. A preferred example of a suitable culture container is a non-coated plate. Standard culturing containers and equipment such as bioreactors known in the art for industrial use may also be used.

The term “culture medium”, “cell culture medium”, “cell medium” or “medium” is used to describe (i) a cellular growth medium in which cells are grown, for example, stem cells, progenitor cells, or differentiated cells or (ii) a cell proliferation medium in which cells are prolifirated, for example, stem cells, progenitor cells, or differentiated cells.

The medium used may be a growth medium or a cell proliferation medium. In general, a growth medium is a culture medium supporting the growth and maintenance of a population of cells. Those of skill in art can readily determine an appropriate growth medium for a particular type of cell population. Suitable growth mediums are known in the art for stem cell culture or epithelial cell culture are for example: DMEM (Dulbecco’s Modified Eagle’s Medium) supplemented with FBS (Fetal Bovine Serum) (Invitrogen), human endothelial SF (serum free) medium (Invitrogen) supplemented with human serum, X-VIVO15 medium (Lonza), or DMEM/F12 (Thermo Fischer Scientific) (optionally supplemented with calcium chloride). These may be additionally supplemented with growth factors (e.g. bFGF), and/or antibiotics such as penicillin and streptomycin.

Alternatively, isolated cells may be added first to a cell proliferation medium according to the invention. The cell proliferation medium as defined herein comprises a growth medium and a LATS inhibitor according to the invention.

In certain embodiments, a cell proliferation medium of the invention comprises a growth medium and a LATS inhibitor according to the invention. The LATS inhibitor is preferably selected from the group comprising compounds according to Formula A1 or subformulae thereof (e.g., Formula A2) and as further described under the section “LATS inhibitors”.

In a preferred embodiment the LATS inhibitors according to Formula A1 or subformulae thereof (e.g., Formula A2) are added at a concentration of about 0.5 to 100 micromolar, preferably about 0.5 to 25 micromolar, more preferably about 1 to 20 micromolar. In a further embodiment the LATS inhibitors according to Formula A1 or subformulae thereof (e.g., Formula A2) are added at a concentration of 0.5 to 100 micromolar, preferably 0.5 to 25 micromolar, more preferably 1 to 20 micromolar. In a specific embodiment the LATS inhibitors according to Formula A1 or subformulae thereof (e.g., Formula A2) are added at a concentration of about 3 to 10 micromolar. In a more specific embodiment the LATS inhibitors according to Formula A1 or subformulae thereof (e.g., Formula A2) are added at a concentration of 3 to 10 micromolar.

In one embodiment, the stock solution of the compound according to Formula A1 or subformulae thereof (e.g., Formula A2) may be prepared by dissolving the compound powder to a stock concentration of 1 mM to 100 mM in DMSO, e.g., 1 mM to 50 mM, 5 mM to 20 mM, 10 mM to 20 mM, in particularly 10 mM. In one embodiment, the stock solution of the compound according to Formula A1 or subformulae thereof (e.g., Formula A2) may be prepared by dissolving the compound powder to a stock concentration of 10 mM in DMSO.

In one aspect of the invention the LATS inhibitor according to the invention inhibits LATS1 and/or LATS2 activity in the cell population. In a preferred embodiment the LATS inhibitor inhibits LATS1 and LATS2.

In one embodiment, a cell proliferation medium of the invention optionally further comprises a rho-associated protein kinase (ROCK) inhibitor. The addition of a ROCK inhibitor was found to prevent cell death and promote attachment of cells in suspensions, especially when culturing stem cells. The ROCK inhibitor are known in the art and in one example, selected from (R)-(+)-trans-4-(1-aminoethyl)-N-(4-Pyridyl)cyclohexanecarboxamide dihydrochloride monohydrate ((1R,4r)-4-((R)-1-aminoethyl)-N-(pyridin-4-yl)cyclohexanecarboxamide; Y-27632; Sigma-Aldrich), 5-(1,4-diazepan-1-ylsulfonyl) isoquinoline (fasudil or HA 1077; Cayman Chemical), H-1152, H-1152P, (S)-(+)-2-Methyl-1-[(4-methyl-5-isoquinolinyl)sulfonyl]homopiperazine, 2HCl, ROCK Inhibitor, Dimethylfasudil (diMF, H-1152P), N-(4-Pyridyl)-N′-(2,4,6-trichlorophenyl)urea, Y-39983, Wf-536, SNJ-1656, and (S)-+)-2-methyl-1-[(4-methyl-5-isoquinolinyl)sulfonyl]-hexahydro-1H-1,4-diazepine dihydrochloride (H-1152; Tocris Bioscience), (S)-4-(3-amino-1-(isoquinolin-6-yl-amino)-1 oxopropan-2-yl) benzyl 2,4-dimethylbenzoate dimesylate (Netarsudil, AR-11324), ripasudil (K-115), verosudil (AR-12286), and its derivatives and analogs. Additional ROCK inhibitors include imidazole-containing benzodiazepines and analogs (see, e.g., WO 97/30992). Others include those described in International Application Publication Nos.: WO 01/56988; WO 02/100833; WO 03/059913; WO 02/076976; WO 04/029045; WO 03/064397; WO 04/039796; WO 05/003101; WO 02/085909; WO 03/082808; WO 03/080610; WO 04/112719; WO 03/062225; and WO 03/062227, for example. In some of these cases, motifs in the inhibitors include an indazole core; a 2-aminopyridine/pyrimidine core; a 9-deazaguanine derivative; benzamide-comprising; aminofurazan-comprising; and/or a combination thereof. Rock inhibitors also include negative regulators of ROCK activation such as small GTP-binding proteins (e.g., Gem, RhoE, and Rad), which can attenuate ROCK activity. In specific embodiments of the disclosure, ROCK1 is targeted instead of ROCK2, for example, WO 03/080610 relates to imidazopyridine derivatives as kinase inhibitors, such as ROCK inhibitors, and methods for inhibiting the effects of ROCK1 and/or ROCK2. The disclosures of the applications cited above are incorporated herein by reference. The Rho inhibitor can also act downstream by interaction with ROCK (Rho-activated kinase) leading to an inhibition of Rho. Such inhibitors are described in U.S. Pat. No. 6,642,263 (the disclosures of which are incorporated by reference herein in their entirety). Other Rho inhibitors that may be used are described in U.S. Pat. Nos. 6,642,263, and 6,451,825. Such inhibitors can be identified using conventional cell screening assays, e.g., described in U.S. Pat. No. 6,620,591 (all of which are herein incorporated by reference in their entirety).

In a preferred embodiment, the ROCK inhibitor used in the cell proliferation medium of the present invention is (R)-(+)-trans-4-(1-aminoethyl)-N-(4-Pyridyl)cyclohexanecarboxamidedihydrochloride monohydrate ((1R,4r)-4-((R)-1-aminoethyl)-N-(pyridin-4-yl)cyclohexanecarboxamide; Y-27632; Sigma-Aldrich; described in Nature 1997, vol. 389, pp. 990-994; JP4851003, JP11130751; JP2770497; US5478838; US6218410, all of which are herein incorporated by reference in their entirety).

In one embodiment, said ROCK inhibitor, in particular Y-27632, is present in a concentration of about 0.5 to about 100 micromolar, preferably of about 0.5 to about 25 micromolar, more preferably of about 1 to about 20 micromolar, particularly preferably of about 10 micromolar. In one embodiment, said compound of the present invention is present in a concentration of 0.5 to 100 micromolar, preferably 0.5 to 25 micromolar, more preferably 1 to 20 micromolar, particularly preferably 10 micromolar. In a specific embodiment, said ROCK inhibitor, in particular Y-27632, is present in a concentration of 10 micromolar.

In a specific embodiment, a cell proliferation medium of the invention comprises DMEM/F12 (1:1), 5-20% human serum or fetal bovine serum or a serum substitute, 1-2 mM calcium chloride, 1 micromolar to 0 micromolar LATS inhibitor, and optionally, 1 micromolar to 20 micromolar ROCK inhibitor. In a more specific embodiment, a cell proliferation medium of the invention comprises DMEM/F12 (1:1), 10-20% human serum or fetal bovine serum or a serum substitute, e.g., 10% human serum or fetal bovine serum or a serum substitute, 1-2 mM calcium chloride, 3 micromolar to 10 micromolar LATS inhibitor, and optionally, 10 micromolar ROCK inhibitor.

The cells may go through a round or rounds of addition of fresh growth medium and/or cell proliferation medium. The cells do not need to be passaged in order for fresh medium to be added, but passaging cells is also a way to add fresh medium.

A series of mediums may be also used, in various combinations of orders: for example a cell proliferation medium, followed by addition of a growth medium (which is not supplemented with LATS inhibitors according to the invention, and may be different to the growth medium used as the base for the cell proliferation medium).

The cell population expansion phase according to the invention occurs during the period the cells are exposed to the cell proliferation medium.

Standard temperature conditions known in the art for culturing cells may be used, for example preferably about 30° C. to 40° C. Particularly preferably cell growth, as well as the cell population expansion phase is carried out at about 37° C. A conventional cell incubator with 5-10% CO₂ levels may be used. Preferably the cells are exposed to 5% CO₂.

The cells may be passaged during the culturing in the growth or cell proliferation medium as necessary. Cells may be passaged when they are sub-confluent or confluent. Preferably the cells are passaged when they reach approximately 90%-100% confluency, although lower percentage confluency levels may also be performed. The passaging of cells is done according to standard protocols known in the art. For example, in brief cells are passaged by treating cultures with Accutase (e.g., for 10 minutes), rinsing the cell suspension by centrifugation and plating cells in fresh growth medium or cell proliferation medium as desired. Cell splitting ratios range, for example, from 1:2 to 1:5.

For the cell population expansion phase of the method of cell population expansion according to the invention, the expansion of the seeding cell population in the cell proliferation medium may be performed until the required amount of cellular material is obtained.

The cells may be exposed to the cell proliferation medium for a range of time periods in order to expand the cell population.

In a preferred embodiment the seeding cell population is exposed to the LATS inhibitors according to the invention (such as those compounds according to Formula A1 or subformulae thereof (e.g., Formula A2)) directly after cell isolation from the patient or donor tissue and maintained for the entire time that cell proliferation is required, for example 12 to 16 days.

In one embodiment according to the invention, a gene editing technique may optionally be performed to genetically modify cells and/or to express a biotherapeutic compound. For example, the cells may be modified to reduce or eliminate the expression and/or function of an immune response mediating gene, which may otherwise contribute to immune rejection when the cell population is delivered to the patient. The application of gene editing techniques in the method of cell population expansion according to the invention is optional, and the administration to the patient of topical immunosuppressants and/or antiinflammatory agents (as described further under the section Immunosuppressant and Antiinflammatory agent) may instead be used if desired to mitigate issues with immunorejection of the transplanted material in the patient.

According to one aspect of the invention, genetically modifying comprises reducing or eliminating the expression and/or function of a gene associated with facilitating a host versus graft immune response. In a preferred embodiment, genetically modifying comprises introducing into an isolated stem cell or stem cell population a gene editing system which specifically targets a gene associated with facilitating a host versus graft immune response. In a specific embodiment, said gene editing system is CRISPR (CRISPR: clustered regularly interspaced short palindromic repeats, also known as CRISPR/Cas systems).

The gene editing technique may be performed at different points, such as for example (1) on tissue, before cell isolation or (2) at the time of cell isolation or (3) during the cell population expansion phase in vitro (when the cells are exposed to a LATS inhibitor according to the invention in vitro) or (4) in vitro at the end of the cell population expansion phase (after the cells are exposed to a LATS inhibitor according to the invention in vitro). In one embodiment, CRISPR is used after two weeks of in vitro expansion of the cell population in the presence of the LATS inhibitor according to the invention.

The gene editing techniques suitable for use in the method of cell population expansion are further described under the section “reduction of immunorejection”.

In the method of cell population expansion according to the invention the LATS inhibitors, which are preferably compounds, produce greater than 2 fold expansion of the seeded population of cells.

In one aspect of the method of cell population expansion according to the invention the compounds according to Formula A1 or subformulae thereof (e.g., Formula A2) produce greater than 30 fold expansion of the seeded population of isolated cells (i.e., cells obtained from a patient or a donor). In a specific embodiment of the method of cell population expansion according to the invention, the LATS inhibitors according to Formula A1 or subformulae thereof produce 100 fold to 2200 fold expansion of the seeded population of isolated cells. In a more specific embodiment of the method of cell population expansion according to the invention, the LATS inhibitors according to Formula A1 or subformulae thereof (e.g., Formula A2) produce 600 fold to 2200 fold expansion of the seeded population of isolated cells. The fold expansion factor achieved by the method of cell population expansion according to the invention may be achieved in one or more passages of the cells. In another aspect of the invention the fold expansion factor achieved by the method of cell population expansion according to the invention may be achieved after exposure to the compound according to Formula A1 or subformulae thereof (e.g., Formula A2) for about 12 to 16 days, preferably about 14 days. In one mebodiment, the expanded seeded population of isolated LSCs according to the invention comprises at least 40% of undifferentiated LSCs, e.g., at least 45%, at least 50%, at least 55%, at least 60%, at least 65%, at least 70%, at least 75%, at least 80%, at least 85%, at least 90%, at least 95% of undifferentiated LSCs. In a specific embodiment, the expanded seeded population of isolated LSCs according to the invention comprises at least 60% of undifferentiated LSCs. In a more specific embodiment, the expanded seeded population of isolated LSCs according to the invention comprises at least 80% of undifferentiated LSCs. In a preferred embodiment, the expanded seeded population of isolated LSCs according to the invention comprises at least 90% of undifferentiated LSCs.

If it is desired to measure the cell number or expansion of the cell population, this may be done for example by taking an aliquot and performing immunocytochemistry (e.g., to count nuclei stained with Sytox Orange) or by live cell imaging under brightfield microscope to count the number of cells or by performing real-time quantitative live-cell analysis of cell confluence at various time points during the cell population expansion phase of the method according to the invention.

The Sytox Orange assay may be performed according to standard protocols known in the art. In brief, after cells have attached to the cell culture dish (typically 24h after cell plating), the cells are fixed in paraformaldehyde. The cells are then permeabilized (e.g. using a solution of 0.3% Triton X-100) and they are then labeled in a solution of Sytox Orange (e.g., using 0.5 micromolar of Sytox Orange in PBS). The number of nuclei stained with Sytox Orange per surface area are then counted under a Zeiss epifluorescence microscope. The cell population expanded by the method of cell population expansion according to the invention may be added to a solution and then stored, for example in a preservation or cryopreservation solution (such as those described below), or added directly to a composition suitable for delivery to a patient. The preservation, cryopreservation solution or composition suitable for ocular delivery may optionally comprise a LATS inhibitor according to the invention.

In a more preferred embodiment according to the invention, the cell population preparation which is delivered to a patient comprises very low to negligible levels of a LATS inhibitor compound. Thus in a specific embodiment, the method of cell population expansion according to the invention comprises the further step of rinsing to substantially remove the compound of the present invention (such as the compound according to Formula A1 or subformulae thereof (e.g., Formula A2)). This may involve rinsing the cells after the cell population expansion phase according to the invention. To rinse the cells, the cells are detached from the culture dish (e.g., by treating with Accutase), the detached cells are then centrifuged, and a cell suspension is made in PBS or growth medium according to the invention. This step may be performed multiple times, e.g., one to ten times, to rinse out the cells. Finally the cells may be resuspended in a preservation solution, cryopreservation solution, a composition suitable for ocular delivery, growth medium or combinations thereof as desired.

The expanded population of cells prepared by the method of cell population expansion and rinsed of cell proliferation medium comprising a LATS inhibitor according the invention may be transferred to a composition suitable for delivery to a patient, such as for example a localising agent. Optionally the cell population is stored for a period before addition to a localising agent suitable for delivery to a patient. In a preferred embodiment, the expanded cell population may first be added to a solution suitable for preservation or cryopreservation, which preferably does not comprise a LATS inhibitor, and the cell population stored (optionally with freezing) before addition to a localising agent suitable for delivery to a patient, which also preferably does not comprise a LATS inhibitor.

Typical solutions suitable for cryopreservation, glycerol, dimethyl sulfoxide, propylene glycol or acetamide may be used in the cryopreservation solution of the present invention. The cryopreserved preparation of cells is typically kept at -20° C. or -80° C. In one embodiment, the cryopreserved compositions comprise a cell (e.g., a modified cell, such as LSC or CEC, with reduced or eliminated expression of B2M by a CRISPR system), e.g., a plurality of cells) and a cryoprotectant selected from the list of glycerol, DMSO (dimethylsulfoxide) polyvinylpyrrolidone, hydroxyethyl starch, propylene glycol, acetamide, monosaccharides, algae-derived polysaccharides, and sugar alcohols, or a combination thereof. In a more specific embodiment, the cryopreserved compositions comprise a cell (e.g., a modified cell, such as LSC or CEC, with reduced or eliminated expression of B2M by a CRISPR system), e.g., a plurality of cells) and DMSO concentration of 0.5% to 10%, e.g., 1%- 10%, 2%-7%, 3%-6%, 4% - 5%, preferably 5%. DMSO acts as a cryoprotecting agent against formation of water crystals within and outside the cells, which could lead to cell damage during cryopreservation steps. In a further embodiment, the cryopreserved compositions further comprise a suitable buffer, for example CryoStor CS5 buffer (BioLife Solutions).

### Cell Population Expansion: To Prepare an Expanded Population of Limbal Stem Cells

In one embodiment of the invention, a population of cells comprising corneal epithelial and limbal cells, including limbal stem cells, for example obtained as described in the section “Starting material to prepare an expanded population of limbal stem cells: Corneal epithelial and limbal cells”, can be grown in medium in a culture container known in the art, such as plates, multi-well plates, and cell culture flasks . For example, a culture dish may be used which is non-coated or coated with collagen, synthemax, gelatin or fibronectin. A preferred example of a suitable culture container is a non-coated plate. Standard culturing containers and equipment such as bioreactors known in the art for industrial use may also be used.

The medium used may be a growth medium or a cell proliferation medium. A growth medium is defined herein as a culture medium supporting the growth and maintenance of a population of cells. Suitable growth mediums are known in the art for stem cell culture or epithelial cell culture are for example: DMEM (Dulbecco’s Modified Eagle’s Medium) supplemented with FBS (Fetal Bovine Serum) (Invitrogen), human endothelial SF (serum free) medium (Invitrogen) supplemented with human serum, X-VIVO15 medium (Lonza), or DMEM/F12 (Thermo Fischer Scientific) (optionally supplemented with calcium chloride). These may be additionally supplemented with growth factors (e.g., bFGF), and/or antibiotics such as penicillin and streptomycin. A preferred growth medium according to the invention is X-VIVO15 medium (which is not additionally supplemented with growth factors).

Alternatively, the isolated cells may be added first to a cell proliferation medium according to the invention. The cell proliferation medium as defined herein comprises a growth medium and a LATS inhibitor according to the invention. In the cell proliferation medium according to the invention the growth medium component is selected from the group consisting of DMEM (Dulbecco’s Modified Eagle’s Medium) supplemented with FBS (Fetal Bovine Serum) (Invitrogen), human endothelial SF (serum free) medium (Invitrogen) supplemented with human serum, X-VIVO15 medium (Lonza or DMEM/F12 (Thermo Fischer Scientific) (optionally supplemented with calcium chloride). These may be additionally supplemented with growth factors (e.g., bFGF), and/or antibiotics such as penicillin and streptomycin.

A preferred cell proliferation medium according to the invention is X-VIVO15 medium (Lonza) with a LATS inhibitor according to the invention. This cell proliferation medium has the advantage that it does not need additional growth factors or feeder cells to facilitate the proliferation of the LSCs. X-VIVO medium comprises inter alia pharmaceutical grade human albumin, recombinant human insulin, and pasteurized human transferrin. Optionally antibiotics may be added to X-VIVO15 medium. In a preferred embodiment, X-VIVO15 medium is used without the addition of antibiotics.

Suitably, in a specific embodiment, a cell proliferation medium according to the invention is DMEM/F12 medium supplemented with serum albumin, e.g., human serum or fetal bovone serum or a serum substitute, and further comprising a LATS inhibitor according to the invention. Optionally antibiotics may be added to DMEM/F12 medium. In a preferred embodiment, DMEM/F12 medium is used without the addition of antibiotics.

The cell proliferation medium comprises a growth medium and a LATS inhibitor according to the invention. The LATS inhibitor is preferably selected from the group comprising compounds according to Formula A1 or subformulae thereof (e.g., Formula A2) and as further described under the section “LATS inhibitors”.

In a preferred embodiment the LATS inhibitors according to Formula A1 or subformulae thereof (e.g., Formula A2) are added at a concentration of about 0.5 to 100 micromolar, preferably about 0.5 to 25 micromolar, more preferably about 1 to 20 micromolar. In a preferred embodiment the LATS inhibitors according to Formula A1 or subformulae thereof (e.g., Formula A2) are added at a concentration of 0.5 to 100 micromolar, preferably 0.5 to 25 micromolar, more preferably 1 to 20 micromolar. In a specific embodiment the LATS inhibitors according to Formula A1 or subformulae thereof (e.g., Formula A2) are added at a concentration of about 3 to 10 micromolar. In a more specific embodiment the LATS inhibitors according to Formula A1 or subformulae thereof (e.g., Formula A2) are added at a concentration of 3 to 10 micromolar.

In one embodiment, the stock solution of the compound according to Formula A1 or subformulae thereof (e.g., Formula A2) may be prepared by dissolving the compound powder to a stock concentration of 10 mM in DMSO. In one embodiment, the stock solution of the compound according to Formula A1 or subformulae thereof (e.g., Formula A2) may be prepared by dissolving the compound powder to a stock concentration of 1 mM to 100 mM in DMSO, e.g., 1 mM to 50 mM, 5 mM to 20 mM, 10 mM to 20 mM, in particularly 10 mM.

In one aspect of the invention the LATS inhibitor according to the invention inhibits LATS1 and/or LATS2 activity in the limbal cells. In a preferred embodiment the LATS inhibitor inhibits LATS1 and LATS2.

In one embodiment, a cell proliferation medium of the invention optionally further comprises a rho-associated protein kinase (ROCK) inhibitor. The addition of a ROCK inhibitor was found to prevent cell death and promote attachment of cells in suspensions, especially when culturing stem cells. The ROCK inhibitor are known in the art and in one example, selected from (R)-(+)-trans-4-(1-aminoethyl)-N-(4-Pyridyl)cyclohexanecarboxamide dihydrochloride monohydrate ((1R,4r)-4-((R)-1-aminoethyl)-N-(pyridin-4-yl)cyclohexanecarboxamide; Y-27632; Sigma-Aldrich), 5-(1,4-diazepan-1-ylsulfonyl) isoquinoline (fasudil or HA 1077; Cayman Chemical), H-1152, H-1152P, (S)-(+)-2-Methyl-1-[(4-methyl-5-isoquinolinyl)sulfonyl]homopiperazine, 2HCl, ROCK Inhibitor, Dimethylfasudil (diMF, H-1152P), N-(4-Pyridyl)-N′-(2,4,6-trichlorophenyl)urea, Y-39983, Wf-536, SNJ-1656, and (S)-+)-2-methyl-1-[(4-methyl-5-isoquinolinyl)sulfonyl]-hexahydro-1H-1,4-diazepine dihydrochloride (H-1152; Tocris Bioscience), (S)-4-(3-amino-1-(isoquinolin-6-yl-amino)-1 oxopropan-2-yl) benzyl 2,4-dimethylbenzoate dimesylate (Netarsudil, AR-11324), ripasudil (K-115), verosudil (AR-12286), and its derivatives and analogs. Additional ROCK inhibitors include imidazole-containing benzodiazepines and analogs (see, e.g., WO 97/30992). Others include those described in International Application Publication Nos.: WO 01/56988; WO 02/100833; WO 03/059913; WO 02/076976; WO 04/029045; WO 03/064397; WO 04/039796; WO 05/003101; WO 02/085909; WO 03/082808; WO 03/080610; WO 04/112719; WO 03/062225; and WO 03/062227, for example. In some of these cases, motifs in the inhibitors include an indazole core; a 2-aminopyridine/pyrimidine core; a 9-deazaguanine derivative; benzamide-comprising; aminofurazan-comprising; and/or a combination thereof. Rock inhibitors also include negative regulators of ROCK activation such as small GTP-binding proteins (e.g., Gem, RhoE, and Rad), which can attenuate ROCK activity. In specific embodiments of the disclosure, ROCK1 is targeted instead of ROCK2, for example, WO 03/080610 relates to imidazopyridine derivatives as kinase inhibitors, such as ROCK inhibitors, and methods for inhibiting the effects of ROCK1 and/or ROCK2. The disclosures of the applications cited above are incorporated herein by reference. The Rho inhibitor can also act downstream by interaction with ROCK (Rho-activated kinase) leading to an inhibition of Rho. Such inhibitors are described in U.S. Pat. No. 6,642,263 (the disclosures of which are incorporated by reference herein in their entirety). Other Rho inhibitors that may be used are described in U.S. Pat. Nos. 6,642,263, and 6,451,825. Such inhibitors can be identified using conventional cell screening assays, e.g., described in U.S. Pat. No. 6,620,591 (all of which are herein incorporated by reference in their entirety).

In a preferred embodiment, the ROCK inhibitor used in the cell proliferation medium of the present invention is (R)-(+)-trans-4-(1-aminoethyl)-N-(4-Pyridyl)cyclohexanecarboxamide dihydrochloride monohydrate ((1R,4r)-4-((R)-1-aminoethyl)-N-(pyridin-4-yl)cyclohexanecarboxamide; Y-27632; Sigma-Aldrich; described in Nature 1997, vol. 389, pp. 990-994; JP4851003, JP11130751; JP2770497; US5478838; US6218410, all of which are herein incorporated by reference in their entirety).

In one embodiment, said ROCK inhibitor, in particular Y-27632, is present in a concentration of about 0.5 to about 100 micromolar, preferably of about 0.5 to about 25 micromolar, more preferably of about 1 to about 20 micromolar, particularly preferably of about 10 micromolar. In one embodiment, said compound of the present invention is present in a concentration of 0.5 to 100 micromolar, preferably 0.5 to 25 micromolar, more preferably 1 to 20 micromolar, particularly preferably 10 micromolar. In a specific embodiment, said ROCK inhibitor, in particularY-27632, is present in a concentration of 10 micromolar.

In a specific embodiment, a cell proliferation medium of the invention comprises DMEM/F12 (1:1), 5-20% human serum or fetal bovine serum or a serum substitute, 1-2 mM calcium chloride, 1 micromolar to 20 micromolar LATS inhibitor, and optionally, 1 micromolar to 20 micromolar ROCK inhibitor. In a more specific embodiment, a cell proliferation medium of the invention comprises DMEM/F12 (1:1), 10-20% human serum or fetal bovine serum or a serum substitute, e.g., 10% human serum or fetal bovine serum or a serum substitute, 1-2 mM calcium chloride, 3 micromolar to 10 micromolar LATS inhibitor, and optionally, 10 micromolar ROCK inhibitor.

The cells may go through a round or rounds of addition of fresh growth medium and/or cell proliferation medium. The cells do not need to be passaged in order for fresh medium to be added, but passaging cells is also a way to add fresh medium.

A series of mediums may be also used, in various combinations of orders: for example a cell proliferation medium, followed by addition of a growth medium (which is not supplemented with LATS inhibitors according to the invention, and may be different to the growth medium used as the base for the cell proliferation medium).

The cell population expansion phase according to the invention occurs during the period the cells are exposed to the cell proliferation medium.

Standard temperature conditions known in the art for culturing cells may be used, for example preferably about 30° C. to 40° C. Particularly preferably cell growth, as well as the cell population expansion phase is carried out at about 37° C. A conventional cell incubator with 5-10% CO₂ levels may be used. Preferably the cells are exposed to 5% CO₂.

The cells may be passaged during the culturing in the growth or cell proliferation medium as necessary. Cells may be passaged when they are sub-confluent or confluent. Preferably the cells are passaged when they reach approximately 90%-100% confluency, although lower percentage confluency levels may also be performed. The passaging of cells is done according to standard protocols known in the art. For example, in brief cells are passaged by treating cultures with Accutase (e.g., for 10 minutes), rinsing the cell suspension by centrifugation and plating cells in fresh growth medium or cell proliferation medium as desired. Cell splitting ratios range, for example, from 1:2 to 1:5.

For the cell population expansion phase of the method of cell population expansion according to the invention, the expansion of the seeding cell population in the cell proliferation medium may be performed until the required amount of cellular material is obtained.

The cells may be exposed to the cell proliferation medium for a range of time periods in order to expand the cell population. For example this may include the entire time that the LSCs are kept in culture, or for the first week after LSC isolation or for 24 hours after dissection of the limbus from the cornea.

In a preferred embodiment the seeding cell population is exposed to the LATS inhibitors according to the invention (such as those compounds according to Formula A1 or subformulae thereof (e.g., Formula A2)) directly after cell isolation from the cornea and maintained for the entire time that LSC proliferation is required, for example 12 to 16 days.

In one embodiment according to the invention, a gene editing technique may optionally be performed to genetically modify cells, to reduce or eliminate the expression and/or function of an immune response mediating gene which may otherwise contribute to immune rejection when the cell population is delivered to the patient. The application of gene editing techniques in the method of cell population expansion according to the invention is optional, and the administration to the patient of topical immunosuppressants and/or antiinflammatory agents (as described further under the section Immunosuppressant and Antiinflammatory agent) may instead be used if desired to mitigate issues with immunorejection of the transplanted material in the patient.

According to one aspect of the invention, genetically modifying comprises reducing or eliminating the expression and/or function of a gene associated with facilitating a host versus graft immune response. In a preferred embodiment, genetically modifying comprises introducing into a limbal stem cell a gene editing system which specifically targets a gene associated with facilitating a host versus graft immune response. In a specific embodiment, said gene editing system is CRISPR (CRISPR: clustered regularly interspaced short palindromic repeats, also known as CRISPR/Cas systems).

The gene editing technique may be performed at different points, such as for example (1) on limbal epithelial tissue, before LSC isolation or (2) at the time of cell isolation or (3) during the cell population expansion phase in vitro (when the cells are exposed to a LATS inhibitor according to the invention in vitro) or (4) in vitro at the end of the cell population expansion phase (after the cells are exposed to a LATS inhibitor according to the invention in vitro). In a one embodiment CRISPR is used after two weeks of in vitro expansion of the cell population in the presence of the LATS inhibitor according to the invention.

The gene editing techniques suitable for use in the method of cell population expansion are further described under the section “reduction of immunorejection”.

In the method of cell population expansion according to the invention the LATS inhibitors, which are preferably compounds, produce greater than 2 fold expansion of the seeded population of cells.

In one aspect of the method of cell population expansion according to the invention the compounds according to Formula A1 or subformulae thereof (e.g., Formula A2) produce greater than 30 fold expansion of the seeded population of limbal cells. In a specific embodiment of the method of cell population expansion according to the invention, the LATS inhibitors according to Formula A1 or subformulae thereof (e.g., Formula A2) produce 100 fold to 2200 fold expansion of the seeded population of limbal cells. In a more specific embodiment of the method of cell population expansion according to the invention, the LATS inhibitors according to Formula A1 or subformulae thereof (e.g., Formula A2) produce 600 fold to 2200 fold expansion of the seeded population of limbal cells. The fold expansion factor achieved by the method of cell population expansion according to the invention may be achieved in one or more passages of the cells. In another aspect of the invention the fold expansion factor achieved by the method of cell population expansion according to the invention may be achieved after exposure to the compound according to Formula A1 or subformulae thereof (e.g., Formula A2) for about 12 to 16 days, preferably about 14 days.

In one aspect of the method of cell population expansion according to the invention, the LATS inhibitors according to Formula A1 or subformulae thereof (e.g., Formula A2) produce a cell population with more than 6% of p63alpha positive cells compared to the total amount of cells. In a specific embodiment of the method of cell population expansion according to the invention, the LATS inhibitors according to Formula A1 or subformulae thereof (e.g., Formula A2) produce a cell population with more than 20% of p63alpha positive cells compared to the total amount of cells. In another specific embodiment of the method of cell population expansion according to the invention, the LATS inhibitors according to Formula A1 or subformulae thereof (e.g., Formula A2) produce a cell population with more than 70% of p63alpha positive cells compared to the total amount of cells. In yet another specific embodiment of the method of cell population expansion according to the invention the LATS inhibitors according to Formula A1 or subformulae thereof (e.g., Formula A2) produce a cell population with more than 95% of p63alpha positive cells compared to the total amount of cells. The increase in the percentage of p63alpha positive cells achieved by the method of cell population expansion according to the invention may be achieved in one or more passages of the cells. In another aspect of the invention the increase in the percentage of p63alpha positive cells achieved by the method of cell population expansion according to the invention may be achieved after exposure to the compound according to Formula A1 or subformulae thereof (e.g., Formula A2) for about 12 to 16 days, preferably about 14 days.

If it is desired to measure the cell number or expansion of the cell population, this may be done for example by taking an aliquot and performing immunocytochemistry (e.g. to count nuclei stained with Sytox Orange) or by live cell imaging under brightfield microscope to count the number of cells or by performing real-time quantitative live-cell analysis of cell confluence at various time points during the cell population expansion phase of the method according to the invention.

The Sytox Orange assay may be performed according to standard protocols known in the art. In brief, after cells have attached to the cell culture dish (typically 24 h after cell plating), the cells are fixed in paraformaldehyde. The cells are then permeabilized (e.g., using a solution of 0.3% Triton X-100) and they are then labeled in a solution of Sytox Orange (e.g., using 0.5 micromolar of Sytox Orange in PBS). The number of nuclei stained with Sytox Orange per surface area are then counted under a Zeiss epifluorescence microscope.

Suitably, according to the invention the LSCs obtainable or obtained by the method of cell population expansion can be isolated from the other cells in the culture using a variety of methods known to those of skill in the art such as immunolabeling and fluorescence sorting, for example solid phase adsorption, fluorescence-activated cell sorting (FACS), magnetic-affinity cell sorting (MACS), and the like. In certain embodiments, the LSCs are isolated through sorting, for example immunofluorescence sorting of certain cell-surface markers. Two preferred methods of sorting well known to those of skill in the art are MACS and FACS. The LSCs markers suitable for said cell-sorting are p63alpha, ABCB5, ABCG2, and C/EBPδ.

Thus, in one aspect, the present invention relates to a method of preparing a modified limbal stem cell or a population of modified limbal stem cells for ocular cell therapy comprising,
a) modifying a limbal stem cell or a population of limbal stem cells by reducing or eliminating expression of B2M comprising introducing into the limbal stem cell or the population of limbal stem cells a CRISPR system comprising a gRNA molecule with a targeting domain
   (i) comprising the sequence of any one of SEQ ID NOs: 23-105 or 108-119, or 134 to 140, or
   (ii) complementary to a sequence within a genomic region selected from: chr15:44711469-44711494, chr15:44711472-44711497, chr15:44711483-44711508, chr15:44711486-44711511, chr15:44711487-44711512, chr15:44711512-44711537, chr15:44711513-44711538, chr15:44711534-44711559, chr15:44711568-44711593, chr15:44711573-44711598, chr15:44711576-44711601, chr15:44711466-44711491, chr15:44711522-44711547, chr15:44711544-44711569, chr15:44711559-44711584, chr15:44711565-44711590, chr15:44711599-44711624, chr15:44711611-44711636, chr15:44715412-44715437, chr15:44715440-44715465, chr15:44715473-44715498, chr15:44715474-44715499, chr15:44715515-44715540, chr15:44715535-44715560, chr15:44715562-44715587, chr15:44715567-44715592, chr15:44715672-44715697, chr15:44715673-44715698, chr15:44715674-44715699, chr15:44715410-44715435, chr15:44715411-44715436, chr15:44715419-44715444, chr15:44715430-44715455, chr15:44715457-44715482, chr15:44715483-44715508, chr15:44715511-44715536, chr15:44715515-44715540, chr15:44715629-44715654, chr15:44715630-44715655, chr15:44715631-44715656, chr15:44715632-44715657, chr15:44715653-44715678, chr15:44715657-44715682, chr15:44715666-44715691, chr15:44715685-44715710, chr15:44715686-44715711, chr15:44716326-44716351, chr15:44716329-44716354, chr15:44716313-44716338, chr15:44717599-44717624, chr15:44717604-44717629, chr15:44717681-44717706, chr15:44717682-44717707, chr15:44717702-44717727, chr15:44717764-44717789, chr15:44717776-44717801, chr15:44717786-44717811, chr15:44717789-44717814, chr15:44717790-44717815, chr15:44717794-44717819, chr15:44717805-44717830, chr15:44717808-44717833, chr15:44717809-44717834, chr15:44717810-44717835, chr15:44717846-44717871, chr15:44717945-44717970, chr15:44717946-44717971, chr15:44717947-44717972, chr15:44717948-44717973, chr15:44717973-44717998, chr15:44717981-44718006, chr15:44718056-44718081, chr15:44718061-44718086, chr15:44718067-44718092, chr15:44718076-44718101, chr15:44717589-44717614, chr15:44717620-44717645, chr15:44717642-44717667, chr15:44717771-44717796, chr15:44717800-44717825, chr15:44717859-44717884, chr15:44717947-44717972, chr15:44718119-44718144, chr15:44711563-44711585, chr15:44715428-44715450, chr15:44715509-44715531, chr15:44715513-44715535, chr15:44715417-44715439, chr15:44711540-44711562, chr15:44711574-44711596, chr15:44711597-44711619, chr15:44715446-44715468, chr15:44715651-44715673, chr15:44713812-44713834, chr15:44711579-44711601, chr15:44711542-44711564, chr15:44711557-44711579, chr15:44711609-44711631, chr15:44715678-44715700, chr15:44715683-44715705, chr15:44715684-44715706, chr15:44715480-44715502,
   wherein the limbal stem cell or the population of limbal stem cells have optionally been cultured in the presence of a LATS inhibitor; and
b) further expanding the modified limbal stem cell or the population of limbal stem cells in cell culture media comprising a LATS inhibitor, and, optionally, ROCK inhibitor; and
c) optionally, enriching the population of limbal stem cells with the undifferentiated limbal stem cells having expression of LSCs bionarkers, such as p63alpha, ABCB5, ABCG2, and C/EBPδ, by fluorescene activated cell sorting (FACS) or magnetic activated cell sorting (MACS), and
d) optionally, enriching the population of limbal stem cells with the limbal stem cells having reduced or eliminated expression of B2M by fluorescene activated cell sorting (FACS) or magnetic activated cell sorting (MACS).

In one aspect, the present invention relates to a cell population comprising the modified LSC of the present invention or the modified LSC obtained by the method of the present invention.

In one embodiment, the cell population of the present invention comprises the modified limbal stem cell of the present invention or the modified limbal stem cell obtained by the method of the present invention, wherein the modified limbal stem cell comprises an indel formed at or near the target sequence complementary to the targeting domain of the gRNA molecule domain. In one embodiment, the indel comprises a deletion of 10 or greater than 10 nucleotides, optionally 11, 12, 13, 14, 15, 16, 17, 18, 19, 20, 21, 22, 23, 24, 25, 26, 27, 28, 29, 30, 31,32, 33, 34, or 35 nucleotides. In a further embodiment, the indel is formed in at least about 40%, e.g., at least about 50%, e.g., at least about 60%, e.g., at least about 70%, e.g., at least about 80%, e.g., at least about 90%, e.g., at least about 95%, e.g., at least about 96%, e.g., at least about 97%, e.g., at least about 98%, e.g., at least about 99%, of the cells of the cell population, e.g., as detectible by next generation sequencing and/or a nucleotide insertional assay.

In one embodiment, the cell population of the present invention comprises the modified limbal stem cell of the present invention or the modified limbal stem cell obtained by the method of the present invention, wherein the modified limbal stem cell comprises an indel formed at or near the target sequence complementary to the targeting domain of the gRNA molecule domain, and wherein an off-target indel is detected in no more than about 5%, e.g., no more than about 1%, e.g., no more than about 0.1%, e.g., no more than about 0.01%, of the cells of the cell population, e.g., as detectible by next generation sequencing and/or a nucleotide insertional assay.

In one aspect according to the invention the LSC population obtainable or obtained by the method of cell population expansion according to the invention preferably shows at least one of the following characteristics. More preferably, it shows two or more, more preferably all, of the following characteristics.

The cell preparation is positive for p63alpha cells. The expression of p63alpha may be estimated by standard techniques known in the art, such as for example immunohistochemistry and quantitative RT-PCR.

The cell preparation comprises more than 6% p63alpha positive cells. Preferably the cell preparation comprises more than 10%, 20%, 30%, 40%, 50%, 60%, 70%, 80% or 90% p63alpha positive cells. In a preferred embodiment the cell preparation comprises more than 95% p63alpha positive cells. The percentage of p63alpha cells may be measured by immunohistochemistry or FACS.

The cells express one or more of ABCB5, ABCG2, and C/EBPδ. The expression of ABCB5, ABCG2, and C/EBPδ may be estimated by standard techniques known in the art, such as for example immunohistochemistry and quantitative RT-PCR.

The cells can differentiate into corneal epithelium cells as observed by keratin-12 expression. These characteristics can be observed by immunohistochemistry or FACS.

The cell preparation comprises more than 50% B2M and /or HLA-ABC negative cells. Preferably the cell preparation comprises more than 50%, 55%, 60%, 65%, 70%, 75%, 80%, 85%, 90%, 95% or 99% B2M and /or HLA-ABC negative cells. In a preferred embodiment the cell preparation comprises more than 95% B2M and /or HLA-ABC negative cells. The percentage of B2M and /or HLA-ABC negative cells may be measured by immunohistochemistry or FACS or MACS.

In a preferred embodiment, the cell preparation comprises more than 95% p63alpha positive cells and more than 95% B2M and /or HLA-ABC negative cells.

The cell population expanded by the method of cell population expansion according to the invention may be added to a solution and then stored, for example in a preservation or cryopreservation solution (such as those described below), or added directly to a composition suitable for ocular delivery. The preservation, cryopreservation solution or composition suitable for ocular delivery may optionally comprise a LATS inhibitor according to the invention.

In a more preferred embodiment according to the invention, the cell population preparation which is delivered to the eye comprises very low (e.g., low trace level) to neglible levels of a LATS inhibitor compound. Thus in a specific embodiment, the method of cell population expansion according to the invention comprises the further step of rinsing to substantially remove the compound of the present invention (such as the compound according to Formula A1 or subformulae thereof). This may involve rinsing the cells after the cell population expansion phase according to the invention. To rinse the cells, the cells are detached from the culture dish (e.g. by treating with Accutase), the detached cells are then centrifuged, and a cell suspension is made in PBS or growth medium according to the invention. This step may be performed multiple times, e.g., one to ten times, to rinse out the cells. Finally the cells may be resuspended in a preservation solution, cryopreservation solution, a composition suitable for ocular delivery, growth medium or combinations thereof as desired.

The expanded population of cells prepared by the method of cell population expansion and rinsed of cell proliferation medium comprising a LATS inhibitor according the invention may be transferred to a composition suitable for ocular delivery, such as for example a localising agent. Optionally the cell population is stored for a period before addition to a localising agent suitable for ocular delivery. In a preferred embodiment, the expanded cell population may first be added to a solution suitable for preservation or cryopreservation, which preferably does not comprise a LATS inhibitor, and the cell population stored (optionally with freezing) before addition to a localising agent suitable for ocular delivery, which also preferably does not comprise a LATS inhibitor.

Typical solutions suitable for preservation of LSCs are Optisol or PBS or CryoStor CS5 buffer (BioLife Solutions), preferably Optisol. Optisol is a corneal storage medium comprising chondroitin sulfate and dextran to enhance corneal dehydration during storage (see for example Kaufman et al., (1991) Optisol corneal storage medium; Arch Ophthalmol Jun; 109(6): 864-8). For cryopreservation, glycerol, dimethyl sulfoxide, propylene glycol or acetamide may be used in the cryopreservation solution of the present invention. The cryopreserved preparation of cells is typically kept at -20° C. or -80° C. In one embodiment, the cryopreserved compositions comprise a cell (e.g., a modified cell, such as LSC or CEC, with reduced or eliminated expression of B2M by a CRISPR system), e.g., a plurality of cells) and a cryoprotectant selected from the list of glycerol, DMSO (dimethylsulfoxide) polyvinylpyrrolidone, hydroxyethyl starch, propylene glycol, acetamide, monosaccharides, algae-derived polysaccharides, and sugar alcohols, or a combination thereof. In a more specific embodiment, the cryopreserved compositions comprise a cell (e.g., a modified cell, such as LSC or CEC, with reduced or eliminated expression of B2M by a CRISPR system), e.g., a plurality of cells) and DMSO concentration of 0.5% to 10%, e.g., 1%- 10%, 2%-7%, 3%-6%, 4% - 5%, preferably 5%. DMSO acts as a cryoprotecting agent against formation of water crystals within and outside the cells, which could lead to cell damage during cryopreservation steps. In a further embodiment, the cryopreserved compositions further comprise a suitable buffer, for example CryoStor CS5 buffer (BioLife Solutions).

In one aspect the invention relates to a preserved or cryopreserved preparation of limbal stem cells obtainable by the method of cell population expansion according to the invention. In an alternative aspect the invention relates to a fresh cell preparation where limbal stem cells obtainable by the method of cell population expansion according to the invention are in suspension in PBS and/or growth medium or combined with a localising agent. The fresh cell preparation is typically kept at about 15 to 37° C. Standard cell cultures containers known in the art may be used to store the cells, such as a vial or a flask.

In a preferred embodiment according to the invention, before use in the eye, a cryopreserved preparation of cells is thawed (for example by incubating at a temperature of about 37° C. in an incubator or waterbath). Preferably 10 volumes of PBS or growth medium may be added to rinse off the cells from the cryopreservant solution. Cells may then be rinsed by centrifugation, and a cell suspension may be made in PBS and/or growth medium, before combination with a localising agent for ocular delivery, which also preferably does not comprise a LATS inhibitor.

In one aspect of the invention the expanded population of cells prepared by the method of cell population expansion, are prepared as a suspension (for example in PBS and/or growth medium, such as for example X-VIVO medium or DMEM/F12) and combined with a localising agent suitable for ocular delivery, (such as a biomatrix like GelMA or fibrin glue). In a specific embodiment of the method of treatment according to the invention, this combination of cells, PBS and/or growth medium, and biomatrix is delivered to the eye via a carrier (such as a contact lens). In yet another specific embodiment this combination of cells, PBS and/or growth medium, and biomatrix comprises at most only trace levels of a LATS inhibitor.

The term “trace levels” as used herein means less than 5% w/v (e.g., no more than 5% w/v, 4% w/v, 3% w/v, 2% w/v, or 1% w/v), and preferably less than 0.01% w/v (e.g., no more than 0.01% w/v, 0.009% w/v, 0.008% w/v, 0.007% w/v, 0.006% w/v, 0.005% w/v, 0.004% w/v, 0.003% w/v, 0.002% w/v, or 0.001 % w/v), which can be measured, for example using high-resolution chromatography as described in the Examples herein. In certain embodiments, trace levels of a LATS inhibitor compound of the invention are the levels of residual compounds present after one or more wash steps, which collectively are below the cellular potency of such compounds, and accordingly they do not induce biological effect in vivo. Accordingly, residual levels of compounds are below the amount expected to have a biological effect on cell population expansion in cell culture or in a subject (e.g., after transplantation of an expanded cell population to the subject). Trace levels can be measured, for example, as the wash-off efficiency, which can be calculated as follows: Wash-off efficiency = 100 - (average concentration in post-wash pellet x pellet volume x molecule weight) / (compound concentration x culture media volume x molecule weight). As used herein, “rinsing to substantially remove” a LATS inhibitor compound of the invention from cells refers to steps for establishing trace levels of the LATS inhibitor compound.

Alternatively, the cells may be cultured and the cell population proliferation phase may occur in cell proliferation medium on a localising agent suitable for cell delivery to the ocular surface (for example fibrin, collagen).

In one aspect, the present invention relates to a composition comprising the modified limbal stem cell of the present invention or the modified limbal stem cell obtained by the method of the present invention or the cell population of the present invention or the population of modified limbal stem cells obtained by the method of the present invention. Suitably, the modified limbal stem cell of the composition comprises an indel formed at or near the target sequence complementary to the targeting domain of the gRNA molecule domain. Suitably, the indel comprises a deletion of 10 or greater than 10 nucleotides, optionally 11, 12, 13, 14, 15, 16, 17, 18, 19, 20, 21, 22, 23, 24, 25, 26, 27, 28, 29, 30, 31,32, 33, 34, or 35 nucleotides. Suitably, the indel is formed in at least about 40%, e.g., at least about 50%, e.g., at least about 60%, e.g., at least about 70%, e.g., at least about 80%, e.g., at least about 90%, e.g., at least about 95%, e.g., at least about 96%, e.g., at least about 97%, e.g., at least about 98%, e.g., at least about 99%, of the cells of the population. In one embodiment, an off-target indel is detected in no more than about 5%, e.g., no more than about 1%, e.g., no more than about 0.1%, e.g., no more than about 0.01%, of the cells of the population of cells e.g., as detectible by next generation sequencing and/or a nucleotide insertional assay.

### Cell Population Expansion: To Prepare an Expanded Population of Corneal Endothelial Cells

In a preferred embodiment of the invention, corneal endothelial cells, for example isolated and obtainable as described in the section “Starting material to prepare an expanded population of corneal endothelial cells”, can be grown in medium in a culture container known in the art, such as plates, multi-well plates, and cell culture flasks. For example, a culture dish may be used which is non-coated or coated with collagen, synthemax, gelatin or fibronectin. A preferred example of a suitable culture container is a non-coated plate. Standard culturing containers and equipment such as bioreactors known in the art for industrial use may also be used.

The medium used may be a growth medium or a cell proliferation medium. A growth medium is defined herein as a culture medium supporting the growth and maintenance of a population of cells. Suitable growth mediums are known in the art for corneal endothelial cell culture are for example: DMEM (Dulbecco’s Modified Eagle’s Medium) supplemented with FBS (Fetal Bovine Serum) (Invitrogen), human endothelial SF (serum free) medium (Invitrogen) supplemented with human serum, X-VIVO15 medium (Lonza) or mesenchymal stem cell-conditioned medium. These may be additionally supplemented with growth factors (e.g., bFGF), and/or antibiotics such as penicillin and streptomycin. A preferred growth medium according to the invention is X-VIVO15 medium (which is not additionally supplemented with growth factors).

Alternatively, the isolated cells may be added first to a cell proliferation medium according to the invention. The cell proliferation medium as defined herein comprises a growth medium and a LATS inhibitor according to the invention. In the cell proliferation medium according to the invention the growth medium component is selected from the group consisting of DMEM (Dulbecco’s Modified Eagle’s Medium) supplemented with FBS (Fetal Bovine Serum) (Invitrogen), human endothelial SF (serum free) medium (Invitrogen) supplemented with human serum, X-VIVO15 medium (Lonza) or mesenchymal stem cell-conditioned medium. These may be additionally supplemented with growth factors (e.g., bFGF), and/or antibiotics such as penicillin and streptomycin.

A preferred cell proliferation medium according to the invention is X-VIVO15 medium (Lonza) with a LATS inhibitor according to the invention. This cell proliferation medium has the advantage that it does not need additional growth factors or feeder cells to facilitate the proliferation of the CECs. X-VIVO medium comprises inter alia pharmaceutical grade human albumin, recombinant human insulin, and pasteurised human transferrin. Optionally antibiotics may be added to X-VIVO15 medium. In a preferred embodiment, X-VIVO15 medium is used without the addition of antibiotics.

The cell proliferation medium comprises a growth medium and a LATS inhibitor according to the invention. The LATS inhibitor is preferably selected from the group comprising compounds according to Formula A1 or subformulae thereof (e.g., Formula A2) and as further described under the section “LATS Inhibitors”.

In a preferred embodiment the LATS inhibitors according to Formula A1 or subformulae thereof (e.g., Formula A2) are added at a concentration of about 0.5 to 100 micromolar, preferably about 0.5 to 25 micromolar, more preferably about 1 to 20 micromolar. In a preferred embodiment the LATS inhibitors according to Formula A1 or subformulae thereof (e.g., Formula A2) are added at a concentration of 0.5 to 100 micromolar, preferably 0.5 to 25 micromolar, more preferably 1 to 20 micromolar. In a specific embodiment the LATS inhibitors according to Formula A1 or subformulae thereof (e.g., Formula A2) are added at a concentration of about 3 to 10 micromolar. In a more specific embodiment the LATS inhibitors according to Formula A1 or subformulae thereof (e.g., Formula A2) are added at a concentration of 3 to 10 micromolar.

In one embodiment, the stock solution of the compound according to Formula A1 or subformulae thereof (e.g., Formula A2) may be prepared by dissolving the compound powder to a stock concentration of 10 mM in DMSO. In one embodiment, the stock solution of the compound according to Formula A1 or subformulae thereof (e.g., Formula A2) may be prepared by dissolving the compound powder to a stock concentration of 1 mM to 100 mM in DMSO, e.g., 1 mM to 50 mM, 5 mM to 20 mM, 10 mM to 20 mM, in particularly 10 mM.

In one aspect of the invention the LATS inhibitor according to the invention inhibits LATS1 and/or LATS2 activity in the corneal endothelial cells. In a preferred embodiment the LATS inhibitor inhibits LATS1 and LATS2.

In one embodiment, a cell proliferation medium of the invention optionally further comprises a rho-associated protein kinase (ROCK) inhibitor. The addition of a ROCK inhibitor was found to prevent cell death and promote attachment of cells in suspensions, especially when culturing stem cells. In a preferred embodiment, the ROCK inhibitor used in the cell proliferation medium of the present invention is (R)-(+)-trans-4-(1-aminoethyl)-N-(4-Pyridyl)cyclohexanecarboxamide dihydrochloride monohydrate ((1R,4r)-4-((R)-1-aminoethyl)-N-(pyridin-4-yl)cyclohexanecarboxamide; Y-27632; Sigma-Aldrich; described in Nature 1997, vol. 389, pp. 990-994; JP4851003, JP11130751; JP2770497; US5478838; US6218410, all of which are herein incorporated by reference in their entirety).

In one embodiment, said ROCK inhibitor, in particular Y-27632, is present in a concentration of about 0.5 to about 100 micromolar, preferably of about 0.5 to about 25 micromolar, more preferably of about 1 to about 20 micromolar, particularly preferably of about 10 micromolar. In one embodiment, said compound of the present invention is present in a concentration of 0.5 to 100 micromolar, preferably 0.5 to 25 micromolar, more preferably 1 to 20 micromolar, particularly preferably 10 micromolar. In a specific embodiment, said ROCK inhibitor, in particular Y-27632, is present in a concentration of 10 micromolar.

In a specific embodiment, a cell proliferation medium of the invention comprises DMEM/F12 (1:1), 5-20% human serum or fetal bovine serum or a serum substitute, 1-2 mM calcium chloride, 1 micromolar to 20 micromolar LATS inhibitor, and optionally, 1 micromolar to 20 micromolar ROCK inhibitor. In a more specific embodiment, a cell proliferation medium of the invention comprises DMEM/F12 (1:1), 10-20% human serum or fetal bovine serum or a serum substitute, e.g., 10% human serum or fetal bovine serum or a serum substitute, 1-2 mM calcium chloride, 3 micromolar to 10 micromolar LATS inhibitor, and optionally, 10 micromolar ROCK inhibitor.

The cells may go through a round or rounds of addition of fresh growth medium and/or cell proliferation medium. The cells do not need to be passaged in order for fresh medium to be added, but passaging cells is also a way to add fresh medium.

A series of mediums may be also used, in various combinations of orders: for example a cell proliferation medium, followed by addition of a growth medium (which is not supplemented with LATS inhibitors according to the invention, and may be different to the growth medium used as the base for the cell proliferation medium).

The cell population expansion phase according to the invention occurs during the period the cells are exposed to the cell proliferation medium.

Standard temperature conditions known in the art for culturing cells may be used, for example preferably about 30° C. to 40° C. Particularly preferably cell growth, as well as the cell population expansion phase is carried out at about 37° C. A conventional cell incubator with 5-10% CO₂ levels may be used. Preferably the cells are exposed to 5% CO₂.

The cells may be passaged during the culturing in the growth or cell proliferation medium as necessary. Cells may be passaged when they are sub-confluent or confluent. Preferably the cells are passaged when they reach approximately 90%-100% confluency, although lower percentage confluency levels may also be performed. The passaging of cells is done according to standard protocols known in the art. For example, in brief the cells are detached from the culture container, for example using collagenase. The cells are then centrifuged and rinsed in PBS or the cell growth medium according to the invention and plated in fresh growth or cell proliferation medium as desired at a dilution of, for example, 1:2 to 1:4.

For the cell population expansion phase of the method of cell population expansion according to the invention, the expansion of the seeding cell population in the cell proliferation medium may be performed until the required amount of cellular material is obtained.

The cells may be exposed to the cell proliferation medium for a range of time periods in order to expand the cell population. For example, this may include the entire time that the CECs are kept in culture, or only for the first one to two weeks after CEC isolation or only for 24 hours after dissection of the cornea.

In a preferred embodiment, the corneal endothelial cells are exposed to the LATS inhibitors according to the invention (such as those compounds according to Formula A1 or subformulae thereof (e.g., Formula A2)) directly after cell isolation from the cornea, and maintained for the entire time that CEC proliferation is required, for example one to two weeks.

In a more preferred embodiment of the invention, after the cell population expansion phase in vitro (i.e., after the cells are exposed to a LATS inhibitor according to the invention for a period of time to expand the population of cells), the method of cell population expansion according to the invention comprises a further step wherein the cells may be grown for a period of time (e.g., two weeks) in growth medium without supplementation of a LATS inhibitor, to enable a mature corneal endothelium to form. A mature corneal endothelium is defined herein as a monolayer of CECs with hexagonal morphology, ZO-1-positive tight junctions and expression of Na/K ATPase. In a preferred embodiment the cells are not passaged while the mature corneal endothelium is formed.

In one embodiment according to the invention, a gene editing technique may optionally be performed to genetically modify cells, to reduce or eliminate the expression and/or function of an immune response mediating gene which may otherwise contribute to immune rejection when the cell population is delivered to the patient. The application of gene editing techniques in the method of cell population expansion according to the invention is optional, and the administration to the patient of topical immunosuppressants and/or antiinflammatory agents (as described further under the section Immunosuppressant and Antiinflammatory agent) may instead be used if desired to mitigate issues with immunorejection of the transplanted material in the patient.

According to one aspect of the invention, for the scenario that a gene editing technique is used, genetically modifying comprises reducing or eliminating the expression and/or function of a gene associated with facilitating a host versus graft immune response. In a preferred embodiment, genetically modifying comprises introducing into a corneal endothelial cell a gene editing system which specifically targets a gene associated with facilitating a host versus graft immune response. In a specific embodiment, said gene editing system is CRISPR (CRISPR: clustered regularly interspaced short palindromic repeats, also known as CRISPR/Cas systems).

A gene editing technique, if it is to be used, may be performed at different points, such as for example (1) on corneal tissue, before CEC isolation or (2) at the time of cell isolation or (3) during the cell population expansion phase in vitro (when the cells are exposed to a LATS inhibitor according to the invention in vitro) or (4) in vitro at the end of the cell population expansion phase (after the cells are exposed to a LATS inhibitor according to the invention in vitro).

The gene editing techniques suitable for use in the method of cell population expansion are further described under the section “reduction of immunorejection”.

In the method of cell population expansion according to the invention the LATS inhibitors, which are preferably compounds, produce greater than 2 fold expansion of the seeded population of cells.

In one aspect of the method of cell population expansion according to the invention the compounds according to Formula A1 or subformulae thereof (e.g., Formula A2) produce greater than 10 fold expansion of the seeded population of corneal endothelial cells. In a specific embodiment of the method of cell population expansion according to the invention, the LATS inhibitors according to Formula A1 or subformulae thereof (e.g., Formula A2) produce 15 fold to 600 fold expansion of the seeded population of corneal endothelial cells. In a more specific embodiment of the method of cell population expansion according to the invention, the LATS inhibitors according to Formula A1 or subformulae thereof (e.g., Formula A2) produce 20 fold to 550 fold expansion of the seeded population of corneal endothelial cells. The fold expansion factor achieved by the method of cell population expansion according to the invention may be achieved in one or more passages of the cells. In another aspect of the invention the fold expansion factor achieved by the method of cell population expansion according to the invention may be achieved after exposure to the compound according to Formula A1 or subformulae thereof (e.g., Formula A2) for one to two weeks, preferably after about 10 days.

If it is desired to measure the cell number or expansion of the cell population, this may be done for example by taking an aliquot and performing immunocytochemistry (e.g., to count nuclei stained with Sytox Orange) or by live cell imaging under brightfield microscope to count the number of cells or by performing real-time quantitative live-cell analysis of cell confluence at various time points during the cell population expansion phase of the method according to the invention.

Suitably, according to the invention the CECs obtainable or obtained by the method of cell population expansion can be isolated from the other cells in the culture using a variety of methods known to those of skill in the art such as immunolabeling and fluorescence sorting, for example solid phase adsorption, fluorescence-activated cell sorting (FACS), magnetic-affinity cell sorting (MACS), and the like. In certain embodiments, the CECs are isolated through sorting, for example immunofluorescence sorting of certain cell-surface markers. Two preferred methods of sorting well known to those of skill in the art are MACS and FACS. The CECs markers suitable for said cell-sorting are Na/K ATPase, 8a2, AQP1 and SLC4A11.

Thus, in one aspect, the present invention relates to a method of preparing a modified CEC or a population of modified CECs for ocular cell therapy comprising,
a) modifying a CEC or a population of CECs by reducing or eliminating expression of B2M comprising introducing into the CE or the population of CECs a CRISPR system comprising a gRNA molecule with a targeting domain
   (i) comprising the sequence of any one of SEQ ID NOs: 23-105 or 108-119, or 134 to 140, or
   (ii) complementary to a sequence within a genomic region selected from: chr15:44711469-44711494, chr15:44711472-44711497, chr15:44711483-44711508, chr15:44711486-44711511, chr15:44711487-44711512, chr15:44711512-44711537, chr15:44711513-44711538, chr15:44711534-44711559, chr15:44711568-44711593, chr15:44711573-44711598, chr15:44711576-44711601, chr15:44711466-44711491, chr15:44711522-44711547, chr15:44711544-44711569, chr15:44711559-44711584, chr15:44711565-44711590, chr15:44711599-44711624, chr15:44711611-44711636, chr15:44715412-44715437, chr15:44715440-44715465, chr15:44715473-44715498, chr15:44715474-44715499, chr15:44715515-44715540, chr15:44715535-44715560, chr15:44715562-44715587, chr15:44715567-44715592, chr15:44715672-44715697, chr15:44715673-44715698, chr15:44715674-44715699, chr15:44715410-44715435, chr15:44715411-44715436, chr15:44715419-44715444, chr15:44715430-44715455, chr15:44715457-44715482, chr15:44715483-44715508, chr15:44715511-44715536, chr15:44715515-44715540, chr15:44715629-44715654, chr15:44715630-44715655, chr15:44715631-44715656, chr15:44715632-44715657, chr15:44715653-44715678, chr15:44715657-44715682, chr15:44715666-44715691, chr15:44715685-44715710, chr15:44715686-44715711, chr15:44716326-44716351, chr15:44716329-44716354, chr15:44716313-44716338, chr15:44717599-44717624, chr15:44717604-44717629, chr15:44717681-44717706, chr15:44717682-44717707, chr15:44717702-44717727, chr15:44717764-44717789, chr15:44717776-44717801, chr15:44717786-44717811, chr15:44717789-44717814, chr15:44717790-44717815, chr15:44717794-44717819, chr15:44717805-44717830, chr15:44717808-44717833, chr15:44717809-44717834, chr15:44717810-44717835, chr15:44717846-44717871, chr15:44717945-44717970, chr15:44717946-44717971, chr15:44717947-44717972, chr15:44717948-44717973, chr15:44717973-44717998, chr15:44717981-44718006, chr15:44718056-44718081, chr15:44718061-44718086, chr15:44718067-44718092, chr15:44718076-44718101, chr15:44717589-44717614, chr15:44717620-44717645, chr15:44717642-44717667, chr15:44717771-44717796, chr15:44717800-44717825, chr15:44717859-44717884, chr15:44717947-44717972, chr15:44718119-44718144, chr15:44711563-44711585, chr15:44715428-44715450, chr15:44715509-44715531, chr15:44715513-44715535, chr15:44715417-44715439, chr15:44711540-44711562, chr15:44711574-44711596, chr15:44711597-44711619, chr15:44715446-44715468, chr15:44715651-44715673, chr15:44713812-44713834, chr15:44711579-44711601, chr15:44711542-44711564, chr15:44711557-44711579, chr15:44711609-44711631, chr15:44715678-44715700, chr15:44715683-44715705, chr15:44715684-44715706, chr15:44715480-44715502,
   wherein the CEC or the population of CECs have optionally been cultured in the presence of a LATS inhibitor; and
b) further expanding the modified CEC or the population of CECs in cell culture media comprising a LATS inhibitor, and, optionally, ROCK inhibitor; and
c) optionally, enriching the population of CECs with the undiffirentiated CECs having expression of CECs biomarkers, such as Na/K ATPase, 8a2, AQP1 and SLC4A11, by fluorescene activated cell sorting (FACS) or magnetic activated cell sorting (MACS), and
d) optionally, enriching the population of CECs with the CECs having reduced or eliminated expression of B2M by fluorescene activated cell sorting (FACS) or magnetic activated cell sorting (MACS).

In one aspect, the present invention relates to a cell population comprising the modified CEC of the present invention or the modified CEC obtained by the method of the present invention.

In one embodiment, the cell population of the present invention comprises the modified CEC of the present invention or the modified CEC obtained by the method of the present invention, wherein the modified CEC comprises an indel formed at or near the target sequence complementary to the targeting domain of the gRNA molecule domain. In one embodiment, the indel comprises a deletion of 10 or greater than 10 nucleotides, optionally 11, 12, 13, 14, 15, 16, 17, 18, 19, 20, 21, 22, 23, 24, 25, 26, 27, 28, 29, 30, 31,32, 33, 34, or 35 nucleotides. In a further embodiment, the indel is formed in at least about 40%, e.g., at least about 50%, e.g., at least about 60%, e.g., at least about 70%, e.g., at least about 80%, e.g., at least about 90%, e.g., at least about 95%, e.g., at least about 96%, e.g., at least about 97%, e.g., at least about 98%, e.g., at least about 99%, of the cells of the cell population, e.g., as detectible by next generation sequencing and/or a nucleotide insertional assay.

In one embodiment, the cell population of the present invention comprises the modified CEC of the present invention or the modified CEC obtained by the method of the present invention, wherein the modified CEC comprises an indel formed at or near the target sequence complementary to the targeting domain of the gRNA molecule domain, and wherein an off-target indel is detected in no more than about 5%, e.g., no more than about 1%, e.g., no more than about 0.1%, e.g., no more than about 0.01%, of the cells of the cell population, e.g., as detectible by next generation sequencing and/or a nucleotide insertional assay.

In one aspect according to the invention the CEC population obtainable or obtained by the method of cell population expansion according to the invention preferably shows at least one of the following characteristics. More preferably, it shows two or more, particularly preferably all, of the following characteristics.

The cells express Na/K ATPase. The expression of Na/K ATPase may be estimated by standard techniques known in the art, such as for example immunohistochemistry, quantitative RT-PCR or by FACS analysis.

The cells express one or more of Collagen 8a2, AQP1 (aquaporin 1) and SLC4A11 (Solute Carrier Family 4 Member 11). Preferably the relative expression levels are higher than cells which do not typically express collagen 8a2, AQP1 and SLC4A11, such as, for example, in dermal fibroblasts. The expression of Collagen 8a2, AQP1 or SLC4A11 may be estimated by standard techniques known in the art, such as for example immunohistochemistry, quantitative RT-PCR or by FACS analysis.

The cells do not express (or at most express relatively low levels of) RPE65 (a marker of retinal pigmented epithelium) and/or CD31 (a marker of vascular endothelium). The relative expression levels are similar to cells which do not typically express RPE65, CD31, such as in dermal fibroblasts. The expression of RPE65 and CD31 may be estimated by standard techniques known in the art, such as for example quantitative RT-PCR, immunohistochemistry or FACS analysis.

The cells express relatively low levels of CD73. The relative expression levels are lower than cells which have undergone endothelial to mesenchymal transition. The expression of CD73 may be estimated by standard techniques known in the art, such as for example FACS analysis or immunohistochemistry.

The cell preparation comprises more than 50% B2M and /or HLA-ABC negative cells. Preferably the cell preparation comprises more than 50%, 55%, 60%, 65%, 70%, 75%, 80%, 85%, 90%, 95% or 99% B2M and /or HLA-ABC negative cells. In a preferred embodiment the cell preparation comprises more than 95% B2M and /or HLA-ABC negative cells. The percentage of B2M and /or HLA-ABC negative cells may be measured by immunohistochemistry or FACS or MACS.

In a preferred embodiment, the cell preparation comprises more than 95% Na/K ATPase, 8a2, AQP1 or SLC4A11 positive cells and more than 95% B2M and /or HLA-ABC negative cells.

In another aspect according to the invention, when in a layer, for example when cultured on a plate, the CEC population obtainable by the method of cell population expansion according to the invention preferably shows at least one of the following characteristics. More preferably, it shows two or more, particularly preferably all, of the following characteristics:

The cells are able to form a single layer structure. This is one of the characteristics of the corneal endothelial cell layer in the body. This may be observed by nuclear staining (e.g., with nuclear dye such as Sytox, Hoechst) followed by examination by microscopy.

The cells are able to form tight junctions. This may be checked by a standard technique known in the art, immunofluorescence staining of tight-junction marker Zonula Occludens-1 (ZO-1).

The cells are able to be regularly arranged in the cell layer. This may be checked by a standard technique known in the art, immunofluorescence staining of tight-junction marker Zonula Occludens-1 (ZO-1). In the healthy corneal endothelial cell layer in the body, the cells constituting the layer are regularly arrayed, due to which corneal endothelial cells are considered to maintain normal function and high transparency and the cornea is considered to appropriately exhibit water control function.

The cell population expanded by the method of cell population expansion according to the invention may be added to a solution and then stored, for example in a preservation or cryopreservation solution (such as those described below), or added directly to a composition suitable for ocular delivery. The preservation, cryopreservation solution or composition suitable for ocular delivery may optionally comprise a LATS inhibitor according to the invention.

In a more preferred embodiment according to the invention, the cell population preparation which is delivered to the eye comprises very low to negligible levels of a LATS inhibitor compound. Thus in a specific embodiment, the method of cell population expansion according to the invention comprises the further step of rinsing to substantially remove the compound of the present invention (such as the compound according to Formula A1 or subformulae thereof (e.g., Formula A2)). This may involve rinsing the cells after the cell population expansion phase according to the invention (directly after the cell population expansion phase and/or after the cells have been cultured to form a mature corneal endothelium in growth medium which has not been supplemented by a LATS inhibitor). To rinse the cells, the cells are centrifuged, and a cell suspension is made in PBS or growth medium according to the invention. This step may be performed multiple times, e.g. one to ten times, to rinse out the cells. Finally the cells may be resuspended in a preservation solution, cryopreservation solution, a composition suitable for ocular delivery, growth medium or combinations thereof as desired.

The expanded population of cells prepared by the method of cell population expansion and rinsed of cell proliferation medium comprising a LATS inhibitor according the invention may be transferred to a composition suitable for ocular delivery, such as for example a localising agent. Optionally the cell population is stored for a period before addition to a localising agent suitable for ocular delivery. In a preferred embodiment, the expanded cell population may first be added to a solution suitable for preservation or cryopreservation, which preferably does not comprise a LATS inhibitor, and the cell population stored (optionally with freezing) before addition to a localising agent suitable for ocular delivery, which also preferably does not comprise a LATS inhibitor.

Typical solutions for suitable for preservation of CECs are Optisol or PBS, preferably Optisol. Optisol is a corneal storage medium comprising chondroitin sulfate and dextran to enhance corneal dehydration during storage (see for example Kaufman et al., (1991) Optisol corneal storage medium; Arch Ophthalmol Jun; 109(6): 864-8). For cryopreservation, glycerol, dimethyl sulfoxide, propylene glycol or acetamide may be used in the cryopreservation solution of the present invention. The cryopreserved preparation of cells is typically kept at -20° C. or -80° C.

In one aspect the invention relates to a preserved or cryopreserved preparation of corneal endothelial cells obtainable by the method of cell population expansion according to the invention. In an alternative aspect the invention relates to a fresh cell preparation where corneal endothelial cells obtainable by the method of cell population expansion according to the invention are in suspension in PBS and/or growth medium or combined with a localising agent. The fresh cell preparation is typically kept at about 37° C. Standard cell cultures containers known in the art may be used to store the cells, such as a vial or a flask.

In a preferred embodiment according to the invention, before use in the eye, a cryopreserved preparation of cells is thawed (for example by incubating at a temperature of about 37° C. in an incubator or waterbath). Preferably 10 volumes of PBS or growth medium may be added to rinse off the cells from the cryopreservant solution. Cells may then be rinsed by centrifugation, and a cell suspension may be made in PBS and/or growth medium, before combination with a localising agent for ocular delivery, which also preferably does not comprise a LATS inhibitor.

In one aspect of the invention the expanded population of cells prepared by the method of cell population expansion, (preferably also including the step of growth in medium without supplementation with LATS inhibitor to form a mature corneal endothelium), are prepared as a suspension (for example in PBS and/or growth medium, such as for example X-VIVO medium) and combined with a localising agent suitable for ocular delivery, (such as a biomatrix like GelMA or fibrin glue). In a specific embodiment of the method of treatment according to the invention, this combination of cells, PBS and/or growth medium, and biomatrix is delivered as a suspension to the eye. In yet another specific embodiment this combination of cells, PBS and/or growth medium, and biomatrix comprises at most only trace levels of a LATS inhibitor.

Alternatively, the cells may be cultured and the cell population proliferation phase may occur in cell proliferation medium on a localising agent suitable for cell delivery to the ocular surface.

In an embodiment of the invention the cell population expanded according to the invention may be isolated as a contiguous cell sheet for delivery to the cornea, using methods known in the art (for examples, see Kim et al, JSM Biotechnol. Bioeng., 2016, p.1047). Cell sheets may be mechanically supported on a material or materials for delivery to the cornea.

In one aspect, the present invention relates to a composition comprising the modified CEC of the present invention or the modified CEC obtained by the method of the present invention or the cell population of the present invention or the population of modified CEC obtained by the method of the present invention. Suitably, the modified CEC of the composition comprises an indel formed at or near the target sequence complementary to the targeting domain of the gRNA molecule domain. Suitably, the indel comprises a deletion of 10 or greater than 10 nucleotides, optionally 11, 12, 13, 14, 15, 16, 17, 18, 19, 20, 21, 22, 23, 24, 25, 26, 27, 28, 29, 30, 31, 32, 33, 34, or 35 nucleotides. Suitably, the indel is formed in at least about 40%, e.g., at least about 50%, e.g., at least about 60%, e.g., at least about 70%, e.g., at least about 80%, e.g., at least about 90%, e.g., at least about 95%, e.g., at least about 96%, e.g., at least about 97%, e.g., at least about 98%, e.g., at least about 99%, of the cells of the population. In one embodiment, an off-target indel is detected in no more than about 5%, e.g., no more than about 1%, e.g., no more than about 0.1%, e.g., no more than about 0.01%, of the cells of the population of cells e.g., as detectible by next generation sequencing and/or a nucleotide insertional assay.

### Reduction of Immunorejection

Upon transplantation, allogeneic limbal stem cells or corneal endothelial cells are at risk of rejection by the recipient’s immune system. Immunosuppression regimens can be used to reduce the risk of immunorejection of transplanted cells, such as LSCs or CECs.

Suitable systemic immunosuppressant agents used in recipients of allogeneic LSCs or CECs include tacrolimus, mycophenolate mofetil, prednisone and prophylactic valganciclovir and trimethoprim/sulfamethoxazole. (See: Holland EJ, Mogilishetty G, Skeens HM, Hair DB, Neff KD, Biber JM, Chan CC (2012) Systemic immunosuppression in ocular surface stem cell transplantation: results of a 10-year experience. Cornea. 2012 Jun;31(6):655-61).

As the methods of cell population expansion according the present invention provide high expansion capabilities of a population of cells, optionally gene-editing technologies may be used to remove drivers of immunorejection or add genes that reduce the recipient’s immune response.

In one aspect of the invention gene editing is carried out on a cell population “ex vivo”. In another aspect of the invention gene-editing technologies may optionally be used to reduce or eliminate the expression of a gene associated with facilitating a host versus graft immune response. In a preferred embodiment the gene is selected from the group consisting of: B2M, HLA-A, HLA-B and HLA-C. In a specific embodiment the gene is B2M. B2M is beta 2 microglobulin and is a component of the class I major histocompatibility complex (MHC). It has the HUGO Gene Nomenclature Committee (HGNC) identifier 914. HLA-A is major histocompatibility complex, class I, A (HGNC ID 4931). HLA-B is major histocompatibility complex, class I, B (HGNC ID 4932). HLA-C is major histocompatibility complex, class I, C (HGNC ID 4933).

In a preferred embodiment, the gene editing method used in a method of the invention is CRISPR (CRISPR: clustered regularly interspaced short palindromic repeats, also known as CRISPR/Cas systems). In one aspect of the invention, the gene editing is carried out on a cell population “ex vivo”.

### CRISPR Gene Editing Systems

“CRISPR” as used herein refers to a set of clustered regularly interspaced short palindromic repeats, or a system comprising such a set of repeats. “Cas,” as used herein, refers to a CRISPR-associated protein. The diverse CRISPR-Cas systems can be divided into two classes according to the configuration of their effector modules: class 1 CRISPR systems utilize several Cas proteins and the crRNA to form an effector complex, whereas class 2 CRISPR systems employ a large single-component Cas protein in conjunction with crRNAs to mediate interference. One example of class 2 CRISPR-Cas system employs Cpf1 (CRISPR from Prevotella and Francisella 1). See, e.g., Zetsche et al., Cell 163:759-771 (2015), the content of which is herein incorporated by reference in its entirety. The term “Cpf1” as used herein includes all orthologs, and variants that can be used in a CRISPR system.

The terms “CRISPR system”, “Cas system” or “CRISPR/Cas system” refer to a set of molecules comprising an RNA-guided nuclease or other effector molecule and a gRNA molecule that together are necessary and sufficient to direct and effect modification of nucleic acid at a target sequence by the RNA-guided nuclease or other effector molecule. In one embodiment, a CRISPR system comprises a gRNA and a Cas protein, e.g., a Cas9 protein. Such systems comprising a Cas9 or modified Cas9 molecule are referred to herein as “Cas9 systems” or “CRISPR/Cas9 systems”. In one example, the gRNA molecule and Cas molecule may be complexed, to form a ribonuclear protein (RNP) complex.

Naturally-occurring CRISPR systems are found in approximately 40% of sequenced eubacteria genomes and 90% of sequenced archaea. Grissa et al. (2007) BMC Bioinformatics 8: 172. This system is a type of prokaryotic immune system that confers resistance to foreign genetic elements such as plasmids and phages and provides a form of acquired immunity. Barrangou et al. (2007) Science 315: 1709-1712; Marragini et al. (2008) Science 322: 1843-1845.

The CRISPR system has been modified for use in gene editing (silencing, enhancing or changing specific genes) in eukaryotes such as mice, primates and humans. Wiedenheft et al. (2012) Nature 482: 331-8. This is accomplished by, for example, introducing into the eukaryotic cell one or more vectors encoding a specifically engineered guide RNA (gRNA) (e.g., a gRNA comprising sequence complementary to sequence of a eukaryotic genome) and one or more appropriate RNA-guided nucleases, e.g., Cas proteins. The RNA guided nuclease forms a complex with the gRNA, which is then directed to the target DNA site by hybridization of the gRNA’s sequence to complementary sequence of a eukaryotic genome, where the RNA-guided nuclease then induces a double or single-strand break in the DNA. Insertion or deletion of nucleotides at or near the strand break creates the modified genome.

As these naturally occur in many different types of bacteria, the exact arrangements of the CRISPR and structure, function and number of Cas genes and their product differ somewhat from species to species. Haft et al. (2005) PLoS Comput. Biol. 1: e60; Kunin et al. (2007) Genome Biol. 8: R61; Mojica et al. (2005) J. Mol. Evol. 60: 174-182; Bolotin et al. (2005) Microbiol. 151: 2551-2561; Pourcel et al. (2005) Microbiol. 151: 653-663; and Stern et al. (2010) Trends. Genet. 28: 335-340. For example, the Cse (Cas subtype, E. coli) proteins (e.g., CasA) form a functional complex, Cascade, that processes CRISPR RNA transcripts into spacer-repeat units that Cascade retains. Brouns et al. (2008) Science 321: 960-964. In other prokaryotes, Cas6 processes the CRISPR transcript. The CRISPR-based phage inactivation in E. coli requires Cascade and Cas3, but not Cas1 or Cas2. The Cmr (Cas RAMP module) proteins in Pyrococcus furiosus and other prokaryotes form a functional complex with small CRISPR RNAs that recognizes and cleaves complementary target RNAs.

A simpler CRISPR system relies on the protein Cas9, which is a nuclease with two active cutting sites, one for each strand of the double helix. Combining Cas9 and modified CRISPR locus RNA can be used in a system for gene editing. Pennisi (2013) Science 341: 833-836.

### Cas9

In some embodiments, the RNA-guided nuclease is a Cas molecule, e.g., a Cas9 molecule.

The terms “Cas9” or “Cas9 molecule” refer to an enzyme from bacterial Type II CRISPR/Cas system responsible for DNA cleavage. Cas9 also includes wild-type protein as well as functional and nonfunctinal mutants thereof. The “Cas9 molecule,” can interact with a gRNA molecule (e.g., sequence of a domain of a tracr, also known as tracrRNA or trans activating CRISPR RNA) and, in concert with the gRNA molecule, localize (e.g., target or home) to a site which comprises a target sequence and PAM (protospacer adjacent motif) sequence. According to the present invention, Cas9 molecules used in the methods and compositions described herein can be from, derived from, or otherwise based on, the Cas9 proteins of a variety of species. For example, Cas9 molecules of, derived from, or based on, e.g., S. pyogenes, S. thermophilus, Staphylococcus aureus and/or Neisseria meningitidis Cas9 molecules, can be used in the systems, methods and compositions described herein. Additional Cas9 species include: Acidovorax avenae, Actinobacillus pleuropneumoniae, Actinobacillus succinogenes, Actinobacillus suis, Actinomyces sp., cycliphilus denitrificans, Aminomonas paucivorans, Bacillus cereus, Bacillus smithii, Bacillus thuringiensis, Bacteroides sp., Blastopirellula marina, Bradyrhiz’ obium sp., Brevibacillus latemsporus, Campylobacter coli, Campylobacter jejuni, Campylobacter lad, Candidatus Puniceispirillum, Clostridiu cellulolyticum, Clostridium perfringens, Corynebacterium accolens, Corynebacterium diphtheria, Corynebacterium matruchotii, Dinoroseobacter sliibae, Eubacterium dolichum, gamma proteobacterium, Gluconacetobacler diazotrophicus, Haemophilus parainfluenzae, Haemophilus sputorum, Helicobacter canadensis, Helicobacter cinaedi, Helicobacter mustelae, Ilyobacler polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica. Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis, Streptococcus sp., Subdoligranulum sp., Tislrella mobilis, Treponema sp., or Verminephrobacter eiseniae.

In some embodiments, the ability of an active Cas9 molecule to interact with and cleave a target nucleic acid is PAM sequence dependent. A PAM (protospacer adjacent motif) sequence is a sequence in the target nucleic acid. It is typically short, for example 2 to 7 base pairs long. In an embodiment, cleavage of the target nucleic acid occurs upstream from the PAM sequence. Active Cas9 molecules from different bacterial species can recognize different sequence motifs (e.g., PAM sequences). In an embodiment, an active Cas9 molecule of S. pyogenes recognizes the sequence motif NGG and directs cleavage of a target nucleic acid sequence 1 to 10, e.g., 3 to 5, base pairs upstream from that sequence. See, e.g., Mali el al, SCIENCE 2013; 339(6121): 823- 826. In an embodiment, an active Cas9 molecule of S. thermophilus recognizes the sequence motif NGGNG (SEQ ID NO: 4) and NNAG AAW (SEQ ID NO: 5) (W = A or T and N is any nucleobase) and directs cleavage of a core target nucleic acid sequence 1 to 10, e.g., 3 to 5, base pairs upstream from these sequences. See, e.g., Horvath et al., SCIENCE 2010; 327(5962): 167- 170, and Deveau et al, J BACTERIOL 2008; 190(4): 1390- 1400. In an embodiment, an active Cas9 molecule of S. mutans recognizes the sequence motif NGG or NAAR (R - A or G) and directs cleavage of a core target nucleic acid sequence 1 to 10, e.g., 3 to 5 base pairs, upstream from this sequence. See, e.g., Deveau et al., J BACTERIOL 2008; 190(4): 1390-1400.

In an embodiment, an active Cas9 molecule of S. aureus recognizes the sequence motif NNGRR (SEQ ID NO: 6) (R = A or G) and directs cleavage of a target nucleic acid sequence 1 to 10, e.g., 3 to 5, base pairs upstream from that sequence. See, e.g., Ran F. et al., NATURE, vol. 520, 2015, pp. 186-191. In an embodiment, an active Cas9 molecule of N. meningitidis recognizes the sequence motif NNNNGATT (SEQ ID NO: 7) and directs cleavage of a target nucleic acid sequence 1 to 10, e.g., 3 to 5, base pairs upstream from that sequence. See, e.g., Hou et al., PNAS EARLY EDITION 2013, 1 -6. The ability of a Cas9 molecule to recognize a PAM sequence can be determined, e.g., using a transformation assay described in Jinek et al, SCIENCE 2012, 337:816.

Exemplary naturally occurring Cas9 molecules are described in Chylinski et al, RNA Biology 2013; 10:5, 727-737. Such Cas9 molecules include Cas9 molecules of a cluster 1 bacterial family, cluster 2 bacterial family, cluster 3 bacterial family, cluster 4 bacterial family, cluster 5 bacterial family, cluster 6 bacterial family, a cluster 7 bacterial family, a cluster 8 bacterial family, a cluster 9 bacterial family, a cluster 10 bacterial family, a cluster 11 bacterial family, a cluster 12 bacterial family, a cluster 13 bacterial family, a cluster 14 bacterial family, a cluster 15 bacterial family, a cluster 16 bacterial family, a cluster 17 bacterial family, a cluster 18 bacterial family, a cluster 19 bacterial family, a cluster 20 bacterial family, a cluster 21 bacterial family, a cluster 22 bacterial family, a cluster 23 bacterial family, a cluster 24 bacterial family, a cluster 25 bacterial family, a cluster 26 bacterial family, a cluster 27 bacterial family, a cluster 28 bacterial family, a cluster 29 bacterial family, a cluster 30 bacterial family, a cluster 31 bacterial family, a cluster 32 bacterial family, a cluster 33 bacterial family, a cluster 34 bacterial family, a cluster 35 bacterial family, a cluster 36 bacterial family, a cluster 37 bacterial family, a cluster 38 bacterial family, a cluster 39 bacterial family, a cluster 40 bacterial family, a cluster 41 bacterial family, a cluster 42 bacterial family, a cluster 43 bacterial family, a cluster 44 bacterial family, a cluster 45 bacterial family, a cluster 46 bacterial family, a cluster 47 bacterial family, a cluster 48 bacterial family. a cluster 49 bacterial family, a cluster 50 bacterial family, a cluster 51 bacterial family, a cluster 52 bacterial family, a cluster 53 bacterial family, a cluster 54 bacterial family, a cluster 55 bacterial family, a cluster 56 bacterial family, a cluster 57 bacterial family, a cluster 58 bacterial family, a cluster 59 bacterial family, a cluster 60 bacterial family, a cluster 61 bacterial family, a cluster 62 bacterial family, a cluster 63 bacterial family, a cluster 64 bacterial family, a cluster 65 bacterial family, a cluster 66 bacterial family, a cluster 67 bacterial family, a cluster 68 bacterial family, a cluster 69 bacterial family, a cluster 70 bacterial family, a cluster 71 bacterial family, a cluster 72 bacterial family, a cluster 73 bacterial family, a cluster 74 bacterial family, a cluster 75 bacterial family, a cluster 76 bacterial family, a cluster 77 bacterial family, or a cluster 78 bacterial family.

Exemplary naturally occurring Cas9 molecules include a Cas9 molecule of a cluster 1 bacterial family. Examples include a Cas9 molecule of: S. pyogenes (e.g., strain SF370, MGAS 10270, MGAS 10750, MGAS2096, MGAS315, MGAS5005, MGAS6180, MGAS9429, NZ131 and SSI- 1), S. thermophilus (e.g., strain LMD-9), S. pseudoporcinus (e.g., strain SPIN 20026), S. mutans (e.g., strain UA 159, NN2025), S. macacae (e.g., strain NCTC1 1558), S. gallolylicus (e.g., strain UCN34, ATCC BAA-2069), S. equines (e.g., strain ATCC 9812, MGCS 124), S. dysdalactiae (e.g., strain GGS 124), S. bovis (e.g., strain ATCC 700338), S. cmginosus (e.g.; strain F021 1 ), S. agalactia* (e.g., strain NEM316, A909), Listeria monocytogenes (e.g., strain F6854), Listeria innocua (L. innocua, e.g., strain Clip 11262), EttUerococcus italicus (e.g., strain DSM 15952), or Enterococcus faecium (e.g., strain 1,23,408). Additional exemplary Cas9 molecules are a Cas9 molecule of Neisseria meningitidis (Hou et’al. PNAS Early Edition 2013, 1-6) and a S. aureus Cas9 molecule. In an embodiment, a Cas9 molecule, e.g., an active Cas9 molecule comprises an amino acid sequence: having at least 60%, 65%, 70%, 75%, 80%, 85%, 90%, 95%, 96%, 97%, 98%, or 99% homology with; differs at no more than 1%, 2%, 5%, 10%, 15%, 20%, 30%, or 40% of the amino acid residues when compared with; differs by at least 1, 2, 5, 10 or 20 amino acids but by no more than 100, 80, 70, 60, 50, 40 or 30 amino acids from; or is identical to; any Cas9 molecule sequence described herein or a naturally occurring Cas9 molecule sequence, e.g., a Cas9 molecule from a species listed herein or described in Chylinski et al., RNA Biology 2013, 10:5, ‘l2′l-T,1 Hou et al. PNAS Early Edition 2013, 1-6.

In an embodiment, a Cas9 molecule comprises an amino acid sequence having at least 60%, 65%, 70%, 75%, 80%, 85%, 90%, 95%, 96%, 97%, 98%, or 99% homology with; differs at no more than 1%, 2%, 5%, 10%, 15%, 20%, 30%, or 40% of the amino acid residues when compared with; differs by at least 1, 2, 5, 10 or 20 amino acids but by no more than 100, 80, 70, 60, 50, 40 or 30 amino acids from; or is identical to; S. pyogenes Cas9 (UniProt Q99ZW2). In one embodiment, a Cas9 molecule comprises an amino acid sequence having 60%, 65%, 70%, 75%, 80%, 85%, 90%, 95%, 96%, 97%, 98%, or 99% homology with; differs at no more than 1%, 2%, 5%, 10%, 15%, 20%, 30%, or 40% of the amino acid residues when compared with; differs by at least 1, 2, 5, 10 or 20 amino acids but by no more than 100, 80, 70, 60, 50, 40 or 30 amino acids from; or is identical to; S. pyogenes Cas9:

Met Asp Lys Lys Tyr Ser Ile Gly Leu Asp Ile Gly Thr Asn Ser Val
1 5 10 15
Gly Trp Ala Val Ile Thr Asp Glu Tyr Lys Val Pro Ser Lys Lys Phe
20 25 30
Lys Val Leu Gly Asn Thr Asp Arg His Ser Ile Lys Lys Asn Leu Ile
35 40 45
Gly Ala Leu Leu Phe Asp Ser Gly Glu Thr Ala Glu Ala Thr Arg Leu
50 55 60
Lys Arg Thr Ala Arg Arg Arg Tyr Thr Arg Arg Lys Asn Arg Ile Cys
65 70 75 80
Tyr Leu Gln Glu Ile Phe Ser Asn Glu Met Ala Lys Val Asp Asp Ser
85 90 95
Phe Phe His Arg Leu Glu Glu Ser Phe Leu Val Glu Glu Asp Lys Lys
100 105 110
His Glu Arg His Pro Ile Phe Gly Asn Ile Val Asp Glu Val Ala Tyr
115 120 125
His Glu Lys Tyr Pro Thr Ile Tyr His Leu Arg Lys Lys Leu Val Asp
130 135 140
Ser Thr Asp Lys Ala Asp Leu Arg Leu Ile Tyr Leu Ala Leu Ala His
145 150 155 160
Met Ile Lys Phe Arg Gly His Phe Leu Ile Glu Gly Asp Leu Asn Pro
165 170 175
Asp Asn Ser Asp Val Asp Lys Leu Phe Ile Gln Leu Val Gln Thr Tyr
180 185 190
Asn Gln Leu Phe Glu Glu Asn Pro Ile Asn Ala Ser Gly Val Asp Ala
195 200 205
Lys Ala Ile Leu Ser Ala Arg Leu Ser Lys Ser Arg Arg Leu Glu Asn
210 215 220
Leu Ile Ala Gln Leu Pro Gly Glu Lys Lys Asn Gly Leu Phe Gly Asn
225 230 235 240
Leu Ile Ala Leu Ser Leu Gly Leu Thr Pro Asn Phe Lys Ser Asn Phe
245 250 255
Asp Leu Ala Glu Asp Ala Lys Leu Gln Leu Ser Lys Asp Thr Tyr Asp
260 265 270
Asp Asp Leu Asp Asn Leu Leu Ala Gln Ile Gly Asp Gln Tyr Ala Asp
275 280 285
Leu Phe Leu Ala Ala Lys Asn Leu Ser Asp Ala Ile Leu Leu Ser Asp
290 295 300
Ile Leu Arg Val Asn Thr Glu Ile Thr Lys Ala Pro Leu Ser Ala Ser
305 310 315 320
Met Ile Lys Arg Tyr Asp Glu His His Gln Asp Leu Thr Leu Leu Lys
325 330 335
Ala Leu Val Arg Gln Gln Leu Pro Glu Lys Tyr Lys Glu Ile Phe Phe
340 345 350
Asp Gln Ser Lys Asn Gly Tyr Ala Gly Tyr Ile Asp Gly Gly Ala Ser
355 360 365
Gln Glu Glu Phe Tyr Lys Phe Ile Lys Pro Ile Leu Glu Lys Met Asp
370 375 380
Gly Thr Glu Glu Leu Leu Val Lys Leu Asn Arg Glu Asp Leu Leu Arg
385 390 395 400
Lys Gln Arg Thr Phe Asp Asn Gly Ser Ile Pro His Gln Ile His Leu
405 410 415
Gly Glu Leu His Ala Ile Leu Arg Arg Gln Glu Asp Phe Tyr Pro Phe
420 425 430
Leu Lys Asp Asn Arg Glu Lys Ile Glu Lys Ile Leu Thr Phe Arg Ile
435 440 445
Pro Tyr Tyr Val Gly Pro Leu Ala Arg Gly Asn Ser Arg Phe Ala Trp
450 455 460
Met Thr Arg Lys Ser Glu Glu Thr Ile Thr Pro Trp Asn Phe Glu Glu
465 470 475 480
Val Val Asp Lys Gly Ala Ser Ala Gln Ser Phe Ile Glu Arg Met Thr
485 490 495
Asn Phe Asp Lys Asn Leu Pro Asn Glu Lys Val Leu Pro Lys His Ser
500 505 510
Leu Leu Tyr Glu Tyr Phe Thr Val Tyr Asn Glu Leu Thr Lys Val Lys
515 520 525
Tyr Val Thr Glu Gly Met Arg Lys Pro Ala Phe Leu Ser Gly Glu Gln
530 535 540
Lys Lys Ala Ile Val Asp Leu Leu Phe Lys Thr Asn Arg Lys Val Thr
545 550 555 560
Val Lys Gln Leu Lys Glu Asp Tyr Phe Lys Lys Ile Glu Cys Phe Asp
565 570 575
Ser Val Glu Ile Ser Gly Val Glu Asp Arg Phe Asn Ala Ser Leu Gly
580 585 590
Thr Tyr His Asp Leu Leu Lys Ile Ile Lys Asp Lys Asp Phe Leu Asp
595 600 605
Asn Glu Glu Asn Glu Asp Ile Leu Glu Asp Ile Val Leu Thr Leu Thr
610 615 620
Leu Phe Glu Asp Arg Glu Met Ile Glu Glu Arg Leu Lys Thr Tyr Ala
625 630 635 640
His Leu Phe Asp Asp Lys Val Met Lys Gln Leu Lys Arg Arg Arg Tyr
645 650 655
Thr Gly Trp Gly Arg Leu Ser Arg Lys Leu Ile Asn Gly Ile Arg Asp
660 665 670
Lys Gln Ser Gly Lys Thr Ile Leu Asp Phe Leu Lys Ser Asp Gly Phe
675 680 685
Ala Asn Arg Asn Phe Met Gln Leu Ile His Asp Asp Ser Leu Thr Phe
690 695 700
Lys Glu Asp Ile Gln Lys Ala Gln Val Ser Gly Gln Gly Asp Ser Leu
705 710 715 720
His Glu His Ile Ala Asn Leu Ala Gly Ser Pro Ala Ile Lys Lys Gly
725 730 735
Ile Leu Gln Thr Val Lys Val Val Asp Glu Leu Val Lys Val Met Gly
740 745 750
Arg His Lys Pro Glu Asn Ile Val Ile Glu Met Ala Arg Glu Asn Gln
755 760 765
Thr Thr Gln Lys Gly Gln Lys Asn Ser Arg Glu Arg Met Lys Arg Ile
770 775 780
Glu Glu Gly Ile Lys Glu Leu Gly Ser Gln Ile Leu Lys Glu His Pro
785 790 795 800
Val Glu Asn Thr Gln Leu Gln Asn Glu Lys Leu Tyr Leu Tyr Tyr Leu
805 810 815
Gln Asn Gly Arg Asp Met Tyr Val Asp Gln Glu Leu Asp Ile Asn Arg
820 825 830
Leu Ser Asp Tyr Asp Val Asp His Ile Val Pro Gln Ser Phe Leu Lys
835 840 845
Asp Asp Ser Ile Asp Asn Lys Val Leu Thr Arg Ser Asp Lys Asn Arg
850 855 860
Gly Lys Ser Asp Asn Val Pro Ser Glu Glu Val Val Lys Lys Met Lys
865 870 875 880
Asn Tyr Trp Arg Gln Leu Leu Asn Ala Lys Leu Ile Thr Gln Arg Lys
885 890 895
Phe Asp Asn Leu Thr Lys Ala Glu Arg Gly Gly Leu Ser Glu Leu Asp
900 905 910
Lys Ala Gly Phe Ile Lys Arg Gln Leu Val Glu Thr Arg Gln Ile Thr
915 920 925
Lys His Val Ala Gln Ile Leu Asp Ser Arg Met Asn Thr Lys Tyr Asp
930 935 940
Glu Asn Asp Lys Leu Ile Arg Glu Val Lys Val Ile Thr Leu Lys Ser
945 950 955 960
Lys Leu Val Ser Asp Phe Arg Lys Asp Phe Gln Phe Tyr Lys Val Arg
965 970 975
Glu Ile Asn Asn Tyr His His Ala His Asp Ala Tyr Leu Asn Ala Val
980 985 990
Val Gly Thr Ala Leu Ile Lys Lys Tyr Pro Lys Leu Glu Ser Glu Phe
995 1000 1005
Val Tyr Gly Asp Tyr Lys Val Tyr Asp Val Arg Lys Met Ile Ala Lys
1010 1015 1020
Ser Glu Gln Glu Ile Gly Lys Ala Thr Ala Lys Tyr Phe Phe Tyr Ser
1025 1030 1035 1040
Asn Ile Met Asn Phe Phe Lys Thr Glu Ile Thr Leu Ala Asn Gly Glu
1045 1050 1055
Ile Arg Lys Arg Pro Leu Ile Glu Thr Asn Gly Glu Thr Gly Glu Ile
1060 1065 1070
Val Trp Asp Lys Gly Arg Asp Phe Ala Thr Val Arg Lys Val Leu Ser
1075 1080 1085
Met Pro Gln Val Asn Ile Val Lys Lys Thr Glu Val Gln Thr Gly Gly
1090 1095 1100
Phe Ser Lys Glu Ser Ile Leu Pro Lys Arg Asn Ser Asp Lys Leu Ile
1105 1110 1115 1120
Ala Arg Lys Lys Asp Trp Asp Pro Lys Lys Tyr Gly Gly Phe Asp Ser
1125 1130 1135
Pro Thr Val Ala Tyr Ser Val Leu Val Val Ala Lys Val Glu Lys Gly
1140 1145 1150
Lys Ser Lys Lys Leu Lys Ser Val Lys Glu Leu Leu Gly Ile Thr Ile
1155 1160 1165
Met Glu Arg Ser Ser Phe Glu Lys Asn Pro Ile Asp Phe Leu Glu Ala
1170 1175 1180
Lys Gly Tyr Lys Glu Val Lys Lys Asp Leu Ile Ile Lys Leu Pro Lys
1185 1190 1195 1200
Tyr Ser Leu Phe Glu Leu Glu Asn Gly Arg Lys Arg Met Leu Ala Ser
1205 1210 1215
Ala Gly Glu Leu Gln Lys Gly Asn Glu Leu Ala Leu Pro Ser Lys Tyr
1220 1225 1230
Val Asn Phe Leu Tyr Leu Ala Ser His Tyr Glu Lys Leu Lys Gly Ser
1235 1240 1245
Pro Glu Asp Asn Glu Gln Lys Gln Leu Phe Val Glu Gln His Lys His
1250 1255 1260
Tyr Leu Asp Glu Ile Ile Glu Gln Ile Ser Glu Phe Ser Lys Arg Val
1265 1270 1275 1280
Ile Leu Ala Asp Ala Asn Leu Asp Lys Val Leu Ser Ala Tyr Asn Lys
1285 1290 1295
His Arg Asp Lys Pro Ile Arg Glu Gln Ala Glu Asn Ile Ile His Leu
1300 1305 1310
Phe Thr Leu Thr Asn Leu Gly Ala Pro Ala Ala Phe Lys Tyr Phe Asp
1315 1320 1325
Thr Thr Ile Asp Arg Lys Arg Tyr Thr Ser Thr Lys Glu Val Leu Asp
1330 1335 1340
Ala Thr Leu Ile His Gln Ser Ile Thr Gly Leu Tyr Glu Thr Arg Ile
1345 1350 1355 1360
Asp Leu Ser Gln Leu Gly Gly Asp
1365
(SEQ ID NO: 123).

In certain embodiments, the Cas9 molecule is a S. pyogenes Cas9 variant, such as a variant described in Slaymaker et al., Science Express, available online Dec. 1, 2015 at Science DOl: 10.1126/science.aad5227; Kleinstiver et al., Nature, 529, 2016, pp. 490-495, available online Jan. 6, 2016 at doi:10.1038/nature16526; or US2016/0102324, the contents of which are incorporated herein in their entirety.

In some embodiments, the Cas9 molecule is a S. pyogenes Cas9 variant of SEQ ID NO: 123 that includes one or more mutations to positively charged amino acids (e.g., lysine, arginine or histidine) that introduce an uncharged or nonpolar amino acid, e.g., alanine, at said position. In embodiments, the mutation is to one or more positively charged amino acids in the nt-groove of Cas9. In embodiments, the Cas9 molecule is a S. pyogenes Cas9 variant of SEQ ID NO: 123 that includes a mutation at position 855 of SEQ ID NO: 123, for example a mutation to an uncharged amino acid, e.g., alanine, at position 855 of SEQ ID NO: 123. In embodiments, the Cas9 molecule has a mutation only at position 855 of SEQ ID NO: 123, relative to SEQ ID NO: 123, e.g., to an uncharged amino acid, e.g., alanine. In embodiments, the Cas9 molecule is a S. pyogenes Cas9 variant of SEQ ID NO: 123 that includes a mutationat position 810, a mutation at position 1003, and/or a mutation at position 1060 of SEQ ID NO: 123, for example a mutation to alanine at position 810, position 1003, and/or position 1060 of SEQ ID NO: 123. In embodiments, the Cas9 molecule has a mutation only at position 810, position 1003, and position 1060 of SEQ ID NO: 123, relative to SEQ ID NO: 123, e.g., where each mutation is to an uncharged amino acid, for example, alanine. In embodiments, the Cas9 molecule is a S. pyogenes Cas9 variant of SEQ ID NO: 123 that includes a mutationat position 848, a mutation at position 1003, and/or a mutation at position 1060 of SEQ ID NO: 123, for example a mutation to alanine at position 848, position 1003, and/or position 1060 of SEQ ID NO: 123. In embodiments, the Cas9 molecule has a mutation only at position 848, position 1003, and position 1060 of SEQ ID NO: 123, relative to SEQ ID NO: 123, e.g., where each mutation is to an uncharged amino acid, for example, alanine. In embodiments, the Cas9 molecule is a Cas9 molecule as described in Slaymaker et al., Science Express, available online Dec. 1, 2015 at Science DOl: 10.1126/science.aad5227.

In embodiments, the Cas9 molecule is a S. pyogenes Cas9 variant of SEQ ID NO: 123 that includes one or more mutations. In embodiments, the Cas9 variant comprises a mutation at position 80 of SEQ ID NO: 123, e.g., includes a leucine at position 80 of SEQ ID NO: 123 (i.e., comprises or consists of SEQ ID NO: 123 with a C80L mutation). In embodiments, the Cas9 variant comprises a mutation at position 574 of SEQ ID NO: 123, e.g., includes a glutamic acid at position 574 of SEQ ID NO: 123 (i.e., comprises or consists of SEQ ID NO: 123 with a C574E mutation). In embodiments, the Cas9 variant comprises a mutation at position 80 and a mutation at position 574 of SEQ ID NO: 123, e.g., includes a leucine at position 80 of SEQ ID NO: 123, and a glutamic acid at position 574 of SEQ ID NO: 123 (i.e., comprises or consists of SEQ ID NO: 123 with a C80L mutation and a C574E mutation). Without being bound by theory, it is believed that such mutations improve the solution properties of the Cas9 molecule.

In embodiments, the Cas9 molecule is a S. pyogenes Cas9 variant of SEQ ID NO: 123 that includes one or more mutations. In embodiments, the Cas9 variant comprises a mutation at position 147 of SEQ ID NO: 123, e.g., includes a tyrosine at position 147 of SEQ ID NO: 123 (i.e., comprises or consists of SEQ ID NO: 123 with a D147Y mutation). In embodiments, the Cas9 variant comprises a mutation at position 411 of SEQ ID NO: 123, e.g., includes a threonine at position 411 of SEQ ID NO: 123 (i.e., comprises or consists of SEQ ID NO: 123 with a P411T mutation). In embodiments, the Cas9 variant comprises a mutation at position 147 and a mutation at position 411 of SEQ ID NO: 123, e.g., includes a tyrosine at position 147 of SEQ ID NO: 123, and a threonine at position 411 of SEQ ID NO: 123 (i.e., comprises or consists of SEQ ID NO: 123 with a D147Y mutation and a P411T mutation). Without being bound by theory, it is believed that such mutations improve the targeting efficiency of the Cas9 molecule, e.g., in yeast.

In embodiments, the Cas9 molecule is a S. pyogenes Cas9 variant of SEQ ID NO: 123 that includes one or more mutations. In embodiments, the Cas9 variant comprises a mutation at position 1135 of SEQ ID NO: 123, e.g., includes a glutamic acid at position 1135 of SEQ ID NO: 123 (i.e., comprises or consists of SEQ ID NO: 123 with a D1135E mutation). Without being bound by theory, it is believed that such mutations improve the selectivity of the Cas9 molecule for the NGG PAM sequence versus the NAG PAM sequence.

In embodiments, the Cas9 molecule is a S. pyogenes Cas9 variant of SEQ ID NO: 123 that includes one or more mutations that introduce an uncharged or nonpolar amino acid, e.g., alanine, at certain positions. In embodiments, the Cas9 molecule is a S. pyogenes Cas9 variant of SEQ ID NO: 123 that includes a mutation at position 497, a mutation at position 661, a mutation at position 695 and/or a mutation at position 926 of SEQ ID NO: 123, for example a mutation to alanine at position 497, position 661, position 695 and/or position 926 of SEQ ID NO: 123. In embodiments, the Cas9 molecule has a mutation only at position 497, position 661, position 695, and position 926 of SEQ ID NO: 123, relative to SEQ ID NO: 123, e.g., where each mutation is to an uncharged amino acid, for example, alanine.

Without being bound by theory, it is believed that such mutations reduce the cutting by the Cas9 molecule at off-target sites

It will be understood that the mutations described herein to the Cas9 molecule may be combined, and may be combined with any of the fusions or other modifications described herein, and the Cas9 molecule may be tested in any of the assays described herein.

Various types of Cas molecules can be used herein. In some embodiments, Cas molecules of Type II Cas systems are used. In other embodiments, Cas molecules of other Cas systems are used. For example, Type I or Type III Cas molecules may be used. Exemplary Cas molecules (and Cas systems) are described, e.g., in Haft et al., PLoS COMPUTATIONAL BIOLOGY 2005, 1(6): e60 and Makarova et al., NATURE REVIEW MICROBIOLOGY 2011, 9:467-477, the contents of both references are incorporated herein by reference in their entirety.

In an embodiment, a Cas or Cas9 molecule used in the methods disclosed herein comprises one or more of the following activities: a nickase activity; a double stranded cleavage activity (e.g., an endonuclease and/or exonuclease activity); a helicase activity; or the’ ability, together with a gRNA molecule, to localize to a target nucleic acid.

In some embodiments, the Cas9 molecule, e.g., a Cas9 of S. pyogenes, may additionally comprise one or more amino acid sequences that confer additional activity. In some aspects, the Cas9 molecule may comprise one or more nuclear localization sequences (NLSs), such as at least 1, 2, 3, 4, 5, 6, 7, 8, 9, 10, or more NLSs. Typically, an NLS consists of one or more short sequences of positively charged lysines or arginines exposed on the protein surface, but other types of NLS are known. Non-limiting examples of NLSs include an NLS sequence comprising or derived from: the NLS of the SV40 virus large T-antigen, having the amino acid sequence PKKKRKV (SEQ ID NO: 8). Other suitable NLS sequences are known in the art (e.g., Sorokin, Biochemistry (Moscow) (2007) 72:13, 1439-1457; Lange J Biol Chem. (2007) 282:8, 5101-5). In any of the aforementioned embodiments, the Cas9 molecule may additionally (or alternatively) comprise a tag, e.g., a His tag, e.g., a His(6) tag (His His His His His His, SEQ ID NO: 121) or His(8) tag (His His His His His His His His, SEQ ID NO: 122) e.g., at the N terminus or the C terminus.

In specific aspects, provided herein are modified human cells, such as LSCs or CECs, with reduced or eliminated expression of B2M by a CRISPR system, e.g., S. pyogenes Cas9 CRISPR system, wherein the modified cells have been transduce to express a Cas9 suitable for gene editing. In a particular aspect, provided herein are modified human cells, such as LSCs or CECs, with reduced or eliminated expression of B2M by a CRISPR system, wherein the modified cells express a Cas9 suitable for gene editing.

In some embodiments, a Cas9 molecule comprises an amino sequence having at least about 60%, 65%, 70%, 75%, 80%, 85%, 90%, 95%, 96%, 97%, 98%, or 99% homology with; differs at no more than 1%, 2%, 5%, 10%, 15%, 20%, 30%, or 40% of the amino acid residues when compared with; differs by at least 1, 2, 5, 10 or 20 amino acids but by no more than 100, 80, 70, 60, 50, 40 or 30 amino acids from; or is identical to to a Cas9 sequence provided herein, e.g., SEQ ID NO: 123, SEQ ID NO: 106, SEQ ID NO: 107, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, or SEQ ID NO: 133. In specific embodiments, a Cas9 molecule comprises an amino sequence selected from SEQ ID NO: 123, SEQ ID NO: 106, SEQ ID NO: 107, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, and SEQ ID NO: 133.

In certain embodiments, a Cas9 protein used in a method or composition of the present invention comprises or has the sequence of iProt 20109496 (SEQ ID NO: 106):

MAPKKKRKVDKKYSIGLDIGTNSVGWAVITDEYKVPSKKFKVLGNTDRHS
lKKNLIGALLFDSGETAEATRLKRTARRRYTRRKNRILYLQElFSNEMAK
VDDSFFHRLEESFLVEEDKKHERHPIFGNIVDEVAYHEKYPTIYHLRKKL
VDSTDKADLRLIYLALAHMIKFRGHFLIEGDLNPDNSDVDKLFIQLVQTY
NQLFEENPINASGVDAKAILSARLSKSRRLENLIAQLPGEKKNGLFGNLI
ALSLGLTPNFKSNFDLAEDAKLQLSKDTYDDDLDNLLAQIGDQYADLFLA
AKNLSDAILLSDILRVNTEITKAPLSASMlKRYDEHHQDLTLLKALVRQQ
LPEKYKElFFDQSKNGYAGYIDGGASQEEFYKFlKPILEKMDGTEELLVK
LNREDLLRKQRTFDNGSIPHQIHLGELHAILRRQEDFYPFLKDNREKlEK
ILTFRIPYYVGPLARGNSRFAWMTRKSEETITPWNFEEVVDKGASAQSFI
ERMTNFDKNLPNEKVLPKHSLLYEYFTVYNELTKVKYVTEGMRKPAFLSG
EQKKAIVDLLFKTNRKVTVKQLKEDYFKKIEEFDSVEISGVEDRFNASLG
TYHDLLKIIKDKDFLDNEENEDILEDIVLTLTLFEDREMIEERLKTYAHL
FDDKVMKQLKRRRYTGWGRLSRKLINGIRDKQSGKTILDFLKSDGFANRN
FMQLIDDSLTFKEDIQKAVSGQGDSLHEHIANLAGSPAIKKGlLQTVKVV
DELVKVMGRHKPENIVIEMARENQTTQKGQKNSRERMKRIEEGIKELGSQ
ILKEHPVENTQLQNEKLYLYYLQNGRDMYVDQELDINRLSDYDVDHIVPQ
SFLKDDSIDNKVLTRSDKNRGKSDNVPSEEVVKKMKNYWRQLLNAKLITQ
RKFDNLTKAERGGLSELDKAGFIKRQLVETRQITKHVAQILDSRMNTKYD
ENDKLIREVKVITLKSKLVSDFRKDFQFYKVREINNYHHAHDAYLNAVVG
TALIKKYPKLESEFVYGDYKVYDVRKMIAKSEQEIGKATAKYFFYSNIMN
FFKTEITLANGEIRKRPLIETNGETGEIVWDKGRDFATVRKVLSMPQVNI
VKKTEVQTGGFSKESILPKRNSDKLIARKKDWDPKKYGGFDSPTVAYSVL
VVAKVEKGKSKKLKSVKELLGITIMERSSFEKNPIDFLEAKGYKEVKKDL
IIKLPKYSLFELENGRKRMLASAGELQKGNELALPSKYVNFLYLASHYEK
LKGSPEDNEQKQLFVEQHKHYLDEIIEQISEFSKRVILADANLDKVLSAY
NKHRDKPIREQAENIIHLFTLTNLGAPAAFKYFDTTIDRKRYTSTKEVLD
ATLIHQSITGLYETRIDLSQLGGDSRADHHHHHH

In certain embodiments, a Cas9 protein used in a method or composition of the present invention comprises or has the sequence of as shown in the Examples herein as SEQ ID NO: 106, omitting terminal histidine tag, e.g., a His(6) tag (His His His His His His, SEQ ID NO: 121). In certain embodiments, a Cas9 protein used in a method or composition of the present invention comprises or has the sequence of as shown in the Examples herein as SEQ ID NO: 106, omitting terminal histidine tag, e.g., a His(6) tag (His His His His His His, SEQ ID NO: 121) and an NLS sequence, e.g., the amino acid sequence PKKKRKV (SEQ ID NO: 8).

In certain embodiments, a Cas9 protein used in a method or composition of the present invention comprises or has the sequence of as shown in the Examples herein as SEQ ID NO: 107. In certain embodiments, a Cas9 protein used in a method or composition of the present invention comprises or has the sequence of as shown in the Examples herein as SEQ ID NO: 107, omitting terminal histidine tag, e.g., a His(6) tag (His His His His His His, SEQ ID NO: 121). In certain embodiments, a Cas9 protein used in a method or composition of the present invention comprises or has the sequence of as shown in the Examples herein as SEQ ID NO: 107, omitting terminal histidine tag, e.g., a His(6) tag (His His His His His His, SEQ ID NO: 121) and an NLS sequence, e.g., the amino acid sequence PKKKRKV (SEQ ID NO: 8). In certain embodiments, a Cas9 protein used in a method or composition of the present invention comprises or has the sequence of iProt105026 (also referred to as iProt106154, iProt106331, iProt106545, and PID426303, depending on the preparation of the protein) (SEQ ID NO: 107):

MAPKKKRKVD KKYSIGLDIG TNSVGWAVIT DEYKVPSKKF KVLGNTDRHS IKKNLIGALL
FDSGETAEAT RLKRTARRRY TRRKNRICYL QEIFSNEMAK VDDSFFHRLE ESFLVEEDKK
HERHPIFGNI VDEVAYHEKY PTIYHLRKKL VDSTDKADLR LIYLALAHMI KFRGHFLIEG
DLNPDNSDVD KLFIQLVQTY NQLFEENPIN ASGVDAKAIL SARLSKSRRL ENLIAQLPGE
KKNGLFGNLI ALSLGLTPNF KSNFDLAEDA KLQLSKDTYD DDLDNLLAQI GDQYADLFLA
AKNLSDAILL SDILRVNTEI TKAPLSASMI KRYDEHHQDL TLLKALVRQQ LPEKYKEIFF
DQSKNGYAGY IDGGASQEEF YKFIKPILEK MDGTEELLVK LNREDLLRKQ RTFDNGSIPH
QIHLGELHAI LRRQEDFYPF LKDNREKIEK ILTFRIPYYV GPLARGNSRF AWMTRKSEET
ITPWNFEEVV DKGASAQSFI ERMTNFDKNL PNEKVLPKHS LLYEYFTVYN ELTKVKYVTE
GMRKPAFLSG EQKKAIVDLL FKTNRKVTVK QLKEDYFKKI ECFDSVEISG VEDRFNASLG
TYHDLLKIIK DKDFLDNEEN EDILEDIVLT LTLFEDREMI EERLKTYAHL FDDKVMKQLK
RRRYTGWGRL SRKLINGIRD KQSGKTILDF LKSDGFANRN FMQLIHDDSL TFKEDIQKAQ
VSGQGDSLHE HIANLAGSPA IKKGILQTVK VVDELVKVMG RHKPENIVIE MARENQTTQK
GQKNSRERMK RIEEGIKELG SQILKEHPVE NTQLQNEKLY LYYLQNGRDM YVDQELDINR
LSDYDVDHIV PQSFLKDDSI DNKVLTRSDK NRGKSDNVPS EEVVKKMKNY WRQLLNAKLI
TQRKFDNLTK AERGGLSELD KAGFIKRQLV ETRQITKHVA QILDSRMNTK YDENDKLIRE
VKVITLKSKL VSDFRKDFQF YKVREINNYH HAHDAYLNAV VGTALIKKYP KLESEFVYGD
YKVYDVRKMI AKSEQEIGKA TAKYFFYSNI MNFFKTEITL ANGEIRKRPL IETNGETGEI
VWDKGRDFAT VRKVLSMPQV NIVKKTEVQT GGFSKESILP KRNSDKLIAR KKDWDPKKYG
GFDSPTVAYS VLVVAKVEKG KSKKLKSVKE LLGITIMERS SFEKNPIDFL EAKGYKEVKK
DLIIKLPKYS LFELENGRKR MLASAGELQK GNELALPSKY VNFLYLASHY EKLKGSPEDN
EQKQLFVEQH KHYLDEIIEQ ISEFSKRVIL ADANLDKVLS AYNKHRDKPI REQAENIIHL
FTLTNLGAPA AFKYFDTTID RKRYTSTKEV LDATLIHQSI TGLYETRIDL SQLGGDSRAD
PKKKRKVHHH HHH

In certain embodiments, a Cas9 protein used in a method or composition of the present invention comprises or has the sequence of iProt106518 (SEQ ID NO: 124):

MAPKKKRKVD KKYSIGLDIG TNSVGWAVIT DEYKVPSKKF KVLGNTDRHS IKKNLIGALL
FDSGETAEAT RLKRTARRRY TRRKNRILYL QEIFSNEMAK VDDSFFHRLE ESFLVEEDKK
HERHPIFGNI VDEVAYHEKY PTIYHLRKKL VDSTDKADLR LIYLALAHMI KFRGHFLIEG
DLNPDNSDVD KLFIQLVQTY NQLFEENPIN ASGVDAKAIL SARLSKSRRL ENLIAQLPGE
KKNGLFGNLI ALSLGLTPNF KSNFDLAEDA KLQLSKDTYD DDLDNLLAQI GDQYADLFLA
AKNLSDAILL SDILRVNTEI TKAPLSASMI KRYDEHHQDL TLLKALVRQQ LPEKYKEIFF
DQSKNGYAGY IDGGASQEEF YKFIKPILEK MDGTEELLVK LNREDLLRKQ RTFDNGSIPH
QIHLGELHAI LRRQEDFYPF LKDNREKIEK ILTFRIPYYV GPLARGNSRF AWMTRKSEET
ITPWNFEEVV DKGASAQSFI ERMTNFDKNL PNEKVLPKHS LLYEYFTVYN ELTKVKYVTE
GMRKPAFLSG EQKKAIVDLL FKTNRKVTVK QLKEDYFKKI EEFDSVEISG VEDRFNASLG
TYHDLLKIIK DKDFLDNEEN EDILEDIVLT LTLFEDREMI EERLKTYAHL FDDKVMKQLK
RRRYTGWGRL SRKLINGIRD KQSGKTILDF LKSDGFANRN FMQLIHDDSL TFKEDIQKAQ
VSGQGDSLHE HIANLAGSPA IKKGILQTVK VVDELVKVMG RHKPENIVIE MARENQTTQK
GQKNSRERMK RIEEGIKELG SQILKEHPVE NTQLQNEKLY LYYLQNGRDM YVDQELDINR
LSDYDVDHIV PQSFLKDDSI DNKVLTRSDK NRGKSDNVPS EEVVKKMKNY WRQLLNAKLI
TQRKFDNLTK AERGGLSELD KAGFIKRQLV ETRQITKHVA QILDSRMNTK YDENDKLIRE
VKVITLKSKL VSDFRKDFQF YKVREINNYH HAHDAYLNAV VGTALIKKYP KLESEFVYGD
YKVYDVRKMI AKSEQEIGKA TAKYFFYSNI MNFFKTEITL ANGEIRKRPL IETNGETGEI
VWDKGRDFAT VRKVLSMPQV NIVKKTEVQT GGFSKESILP KRNSDKLIAR KKDWDPKKYG
GFDSPTVAYS VLVVAKVEKG KSKKLKSVKE LLGITIMERS SFEKNPIDFL EAKGYKEVKK
DLIIKLPKYS LFELENGRKR MLASAGELQK GNELALPSKY VNFLYLASHY EKLKGSPEDN
EQKQLFVEQH KHYLDEIIEQ ISEFSKRVIL ADANLDKVLS AYNKHRDKPI REQAENIIHL
FTLTNLGAPA AFKYFDTTID RKRYTSTKEV LDATLIHQSI TGLYETRIDL SQLGGDSRAD
PKKKRKVHHH HHH

In certain embodiments, a Cas9 protein used in a method or composition of the present invention comprises or has the sequence of iProt106519 (SEQ ID NO: 125):

MGSSHHHHHH HHENLYFQGS MDKKYSIGLD IGTNSVGWAV ITDEYKVPSK KFKVLGNTDR
HSIKKNLIGA LLFDSGETAE ATRLKRTARR RYTRRKNRIC YLQEIFSNEM AKVDDSFFHR
LEESFLVEED KKHERHPIFG NIVDEVAYHE KYPTIYHLRK KLVDSTDKAD LRLIYLALAH
MIKFRGHFLI EGDLNPDNSD VDKLFIQLVQ TYNQLFEENP INASGVDAKA ILSARLSKSR
RLENLIAQLP GEKKNGLFGN LIALSLGLTP NFKSNFDLAE DAKLQLSKDT YDDDLDNLLA
QIGDQYADLF LAAKNLSDAI LLSDILRVNT EITKAPLSAS MIKRYDEHHQ DLTLLKALVR
QQLPEKYKEI FFDQSKNGYA GYIDGGASQE EFYKFIKPIL EKMDGTEELL VKLNREDLLR
KQRTFDNGSI PHQIHLGELH AILRRQEDFY PFLKDNREKI EKILTFRIPY YVGPLARGNS
RFAWMTRKSE ETITPWNFEE VVDKGASAQS FIERMTNFDK NLPNEKVLPK HSLLYEYFTV
YNELTKVKYV TEGMRKPAFL SGEQKKAIVD LLFKTNRKVT VKQLKEDYFK KIECFDSVEI
SGVEDRFNAS LGTYHDLLKI IKDKDFLDNE ENEDILEDIV LTLTLFEDRE MIEERLKTYA
HLFDDKVMKQ LKRRRYTGWG RLSRKLINGI RDKQSGKTIL DFLKSDGFAN RNFMQLIHDD
SLTFKEDIQK AQVSGQGDSL HEHIANLAGS PAIKKGILQT VKVVDELVKV MGRHKPENIV
IEMARENQTT QKGQKNSRER MKRIEEGIKE LGSQILKEHP VENTQLQNEK LYLYYLQNGR
DMYVDQELDI NRLSDYDVDH IVPQSFLKDD SIDNKVLTRS DKNRGKSDNV PSEEVVKKMK
NYWRQLLNAK LITQRKFDNL TKAERGGLSE LDKAGFIKRQ LVETRQITKH VAQILDSRMN
TKYDENDKLI REVKVITLKS KLVSDFRKDF QFYKVREINN YHHAHDAYLN AVVGTALIKK
YPKLESEFVY GDYKVYDVRK MIAKSEQEIG KATAKYFFYS NIMNFFKTEI TLANGEIRKR
PLIETNGETG EIVWDKGRDF ATVRKVLSMP QVNIVKKTEV QTGGFSKESI LPKRNSDKLI
ARKKDWDPKK YGGFDSPTVA YSVLVVAKVE KGKSKKLKSV KELLGITIME RSSFEKNPID
FLEAKGYKEV KKDLIIKLPK YSLFELENGR KRMLASAGEL QKGNELALPS KYVNFLYLAS
HYEKLKGSPE DNEQKQLFVE QHKHYLDEII EQISEFSKRV ILADANLDKV LSAYNKHRDK
PIREQAENII HLFTLTNLGA PAAFKYFDTT IDRKRYTSTK EVLDATLIHQ SITGLYETRI
DLSQLGGDGG GSPKKKRKV

In certain embodiments, a Cas9 protein used in a method or composition of the present invention comprises or has the sequence of iProt106520 (SEQ ID NO: 126):

MAHHHHHHGG SPKKKRKVDK KYSIGLDIGT NSVGWAVITD EYKVPSKKFK VLGNTDRHSI
KKNLIGALLF DSGETAEATR LKRTARRRYT RRKNRICYLQ EIFSNEMAKV DDSFFHRLEE
SFLVEEDKKH ERHPIFGNIV DEVAYHEKYP TIYHLRKKLV DSTDKADLRL IYLALAHMIK
FRGHFLIEGD LNPDNSDVDK LFIQLVQTYN QLFEENPINA SGVDAKAILS ARLSKSRRLE
NLIAQLPGEK KNGLFGNLIA LSLGLTPNFK SNFDLAEDAK LQLSKDTYDD DLDNLLAQIG
DQYADLFLAA KNLSDAILLS DILRVNTEIT KAPLSASMIK RYDEHHQDLT LLKALVRQQL
PEKYKEIFFD QSKNGYAGYI DGGASQEEFY KFIKPILEKM DGTEELLVKL NREDLLRKQR
TFDNGSIPHQ IHLGELHAIL RRQEDFYPFL KDNREKIEKI LTFRIPYYVG PLARGNSRFA
WMTRKSEETI TPWNFEEVVD KGASAQSFIE RMTNFDKNLP NEKVLPKHSL LYEYFTVYNE
LTKVKYVTEG MRKPAFLSGE QKKAIVDLLF KTNRKVTVKQ LKEDYFKKIE CFDSVEISGV
EDRFNASLGT YHDLLKIIKD KDFLDNEENE DILEDIVLTL TLFEDREMIE ERLKTYAHLF
DDKVMKQLKR RRYTGWGRLS RKLINGIRDK QSGKTILDFL KSDGFANRNF MQLIHDDSLT
FKEDIQKAQV SGQGDSLHEH IANLAGSPAI KKGILQTVKV VDELVKVMGR HKPENIVIEM
ARENQTTQKG QKNSRERMKR IEEGIKELGS QILKEHPVEN TQLQNEKLYL YYLQNGRDMY
VDQELDINRL SDYDVDHIVP QSFLKDDSID NKVLTRSDKN RGKSDNVPSE EVVKKMKNYW
RQLLNAKLIT QRKFDNLTKA ERGGLSELDK AGFIKRQLVE TRQITKHVAQ ILDSRMNTKY
DENDKLIREV KVITLKSKLV SDFRKDFQFY KVREINNYHH AHDAYLNAVV GTALIKKYPK
LESEFVYGDY KVYDVRKMIA KSEQEIGKAT AKYFFYSNIM NFFKTEITLA NGEIRKRPLI
ETNGETGEIV WDKGRDFATV RKVLSMPQVN IVKKTEVQTG GFSKESILPK RNSDKLIARK
KDWDPKKYGG FDSPTVAYSV LVVAKVEKGK SKKLKSVKEL LGITIMERSS FEKNPIDFLE
AKGYKEVKKD LIIKLPKYSL FELENGRKRM LASAGELQKG NELALPSKYV NFLYLASHYE
KLKGSPEDNE QKQLFVEQHK HYLDEIIEQI SEFSKRVILA DANLDKVLSA YNKHRDKPIR
EQAENIIHLF TLTNLGAPAA FKYFDTTIDR KRYTSTKEVL DATLIHQSIT GLYETRIDLS
QLGGDSRADP KKKRKV

In certain embodiments, a Cas9 protein used in a method or composition of the present invention comprises or has the sequence of iProt106521 (SEQ ID NO: 127):

MAPKKKRKVD KKYSIGLDIG TNSVGWAVIT DEYKVPSKKF KVLGNTDRHS IKKNLIGALL
FDSGETAEAT RLKRTARRRY TRRKNRICYL QEIFSNEMAK VDDSFFHRLE ESFLVEEDKK
HERHPIFGNI VDEVAYHEKY PTIYHLRKKL VDSTDKADLR LIYLALAHMI KFRGHFLIEG
DLNPDNSDVD KLFIQLVQTY NQLFEENPIN ASGVDAKAIL SARLSKSRRL ENLIAQLPGE
KKNGLFGNLI ALSLGLTPNF KSNFDLAEDA KLQLSKDTYD DDLDNLLAQI GDQYADLFLA
AKNLSDAILL SDILRVNTEI TKAPLSASMI KRYDEHHQDL TLLKALVRQQ LPEKYKEIFF
DQSKNGYAGY IDGGASQEEF YKFIKPILEK MDGTEELLVK LNREDLLRKQ RTFDNGSIPH
QIHLGELHAI LRRQEDFYPF LKDNREKIEK ILTFRIPYYV GPLARGNSRF AWMTRKSEET
ITPWNFEEVV DKGASAQSFI ERMTNFDKNL PNEKVLPKHS LLYEYFTVYN ELTKVKYVTE
GMRKPAFLSG EQKKAIVDLL FKTNRKVTVK QLKEDYFKKI ECFDSVEISG VEDRFNASLG
TYHDLLKIIK DKDFLDNEEN EDILEDIVLT LTLFEDREMI EERLKTYAHL FDDKVMKQLK
RRRYTGWGRL SRKLINGIRD KQSGKTILDF LKSDGFANRN FMQLIHDDSL TFKEDIQKAQ
VSGQGDSLHE HIANLAGSPA IKKGILQTVK VVDELVKVMG RHKPENIVIE MARENQTTQK
GQKNSRERMK RIEEGIKELG SQILKEHPVE NTQLQNEKLY LYYLQNGRDM YVDQELDINR
LSDYDVDHIV PQSFLKDDSI DNKVLTRSDK NRGKSDNVPS EEVVKKMKNY WRQLLNAKLI
TQRKFDNLTK AERGGLSELD KAGFIKRQLV ETRQITKHVA QILDSRMNTK YDENDKLIRE
VKVITLKSKL VSDFRKDFQF YKVREINNYH HAHDAYLNAV VGTALIKKYP KLESEFVYGD
YKVYDVRKMI AKSEQEIGKA TAKYFFYSNI MNFFKTEITL ANGEIRKRPL IETNGETGEI
VWDKGRDFAT VRKVLSMPQV NIVKKTEVQT GGFSKESILP KRNSDKLIAR KKDWDPKKYG
GFDSPTVAYS VLVVAKVEKG KSKKLKSVKE LLGITIMERS SFEKNPIDFL EAKGYKEVKK
DLIIKLPKYS LFELENGRKR MLASAGELQK GNELALPSKY VNFLYLASHY EKLKGSPEDN
EQKQLFVEQH KHYLDEIIEQ ISEFSKRVIL ADANLDKVLS AYNKHRDKPI REQAENIIHL
FTLTNLGAPA AFKYFDTTID RKRYTSTKEV LDATLIHQSI TGLYETRIDL SQLGGDSRAD
HHHHHH

In certain embodiments, a Cas9 protein used in a method or composition of the present invention comprises or has the sequence of iProt106522 (SEQ ID NO: 128):

MAHHHHHHGG SDKKYSIGLD IGTNSVGWAV ITDEYKVPSK KFKVLGNTDR HSIKKNLIGA
LLFDSGETAE ATRLKRTARR RYTRRKNRIC YLQEIFSNEM AKVDDSFFHR LEESFLVEED
KKHERHPIFG NIVDEVAYHE KYPTIYHLRK KLVDSTDKAD LRLIYLALAH MIKFRGHFLI
EGDLNPDNSD VDKLFIQLVQ TYNQLFEENP INASGVDAKA ILSARLSKSR RLENLIAQLP
GEKKNGLFGN LIALSLGLTP NFKSNFDLAE DAKLQLSKDT YDDDLDNLLA QIGDQYADLF
LAAKNLSDAI LLSDILRVNT EITKAPLSAS MIKRYDEHHQ DLTLLKALVR QQLPEKYKEI
FFDQSKNGYA GYIDGGASQE EFYKFIKPIL EKMDGTEELL VKLNREDLLR KQRTFDNGSI
PHQIHLGELH AILRRQEDFY PFLKDNREKI EKILTFRIPY YVGPLARGNS RFAWMTRKSE
ETITPWNFEE VVDKGASAQS FIERMTNFDK NLPNEKVLPK HSLLYEYFTV YNELTKVKYV
TEGMRKPAFL SGEQKKAIVD LLFKTNRKVT VKQLKEDYFK KIECFDSVEI SGVEDRFNAS
LGTYHDLLKI IKDKDFLDNE ENEDILEDIV LTLTLFEDRE MIEERLKTYA HLFDDKVMKQ
LKRRRYTGWG RLSRKLINGI RDKQSGKTIL DFLKSDGFAN RNFMQLIHDD SLTFKEDIQK
AQVSGQGDSL HEHIANLAGS PAIKKGILQT VKVVDELVKV MGRHKPENIV IEMARENQTT
QKGQKNSRER MKRIEEGIKE LGSQILKEHP VENTQLQNEK LYLYYLQNGR DMYVDQELDI
NRLSDYDVDH IVPQSFLKDD SIDNKVLTRS DKNRGKSDNV PSEEVVKKMK NYWRQLLNAK
LITQRKFDNL TKAERGGLSE LDKAGFIKRQ LVETRQITKH VAQILDSRMN TKYDENDKLI
REVKVITLKS KLVSDFRKDF QFYKVREINN YHHAHDAYLN AVVGTALIKK YPKLESEFVY
GDYKVYDVRK MIAKSEQEIG KATAKYFFYS NIMNFFKTEI TLANGEIRKR PLIETNGETG
EIVWDKGRDF ATVRKVLSMP QVNIVKKTEV QTGGFSKESI LPKRNSDKLI ARKKDWDPKK
YGGFDSPTVA YSVLVVAKVE KGKSKKLKSV KELLGITIME RSSFEKNPID FLEAKGYKEV
KKDLIIKLPK YSLFELENGR KRMLASAGEL QKGNELALPS KYVNFLYLAS HYEKLKGSPE
DNEQKQLFVE QHKHYLDEII EQISEFSKRV ILADANLDKV LSAYNKHRDK PIREQAENII
HLFTLTNLGA PAAFKYFDTT IDRKRYTSTK EVLDATLIHQ SITGLYETRI DLSQLGGDSR
ADPKKKRKV

In certain embodiments, a Cas9 protein used in a method or composition of the present invention comprises or has the sequence of iProt106658 (SEQ ID NO: 129):

MGSSHHHHHH HHENLYFQGS MDKKYSIGLD IGTNSVGWAV ITDEYKVPSK KFKVLGNTDR
HSIKKNLIGA LLFDSGETAE ATRLKRTARR RYTRRKNRIC YLQEIFSNEM AKVDDSFFHR
LEESFLVEED KKHERHPIFG NIVDEVAYHE KYPTIYHLRK KLVDSTDKAD LRLIYLALAH
MIKFRGHFLI EGDLNPDNSD VDKLFIQLVQ TYNQLFEENP INASGVDAKA ILSARLSKSR
RLENLIAQLP GEKKNGLFGN LIALSLGLTP NFKSNFDLAE DAKLQLSKDT YDDDLDNLLA
QIGDQYADLF LAAKNLSDAI LLSDILRVNT EITKAPLSAS MIKRYDEHHQ DLTLLKALVR
QQLPEKYKEI FFDQSKNGYA GYIDGGASQE EFYKFIKPIL EKMDGTEELL VKLNREDLLR
KQRTFDNGSI PHQIHLGELH AILRRQEDFY PFLKDNREKI EKILTFRIPY YVGPLARGNS
RFAWMTRKSE ETITPWNFEE VVDKGASAQS FIERMTNFDK NLPNEKVLPK HSLLYEYFTV
YNELTKVKYV TEGMRKPAFL SGEQKKAIVD LLFKTNRKVT VKQLKEDYFK KIECFDSVEI
SGVEDRFNAS LGTYHDLLKI IKDKDFLDNE ENEDILEDIV LTLTLFEDRE MIEERLKTYA
HLFDDKVMKQ LKRRRYTGWG RLSRKLINGI RDKQSGKTIL DFLKSDGFAN RNFMQLIHDD
SLTFKEDIQK AQVSGQGDSL HEHIANLAGS PAIKKGILQT VKVVDELVKV MGRHKPENIV
IEMARENQTT QKGQKNSRER MKRIEEGIKE LGSQILKEHP VENTQLQNEK LYLYYLQNGR
DMYVDQELDI NRLSDYDVDH IVPQSFLKDD SIDNKVLTRS DKNRGKSDNV PSEEVVKKMK
NYWRQLLNAK LITQRKFDNL TKAERGGLSE LDKAGFIKRQ LVETRQITKH VAQILDSRMN
TKYDENDKLI REVKVITLKS KLVSDFRKDF QFYKVREINN YHHAHDAYLN AVVGTALIKK
YPKLESEFVY GDYKVYDVRK MIAKSEQEIG KATAKYFFYS NIMNFFKTEI TLANGEIRKR
PLIETNGETG EIVWDKGRDF ATVRKVLSMP QVNIVKKTEV QTGGFSKESI LPKRNSDKLI
ARKKDWDPKK YGGFDSPTVA YSVLVVAKVE KGKSKKLKSV KELLGITIME RSSFEKNPID
FLEAKGYKEV KKDLIIKLPK YSLFELENGR KRMLASAGEL QKGNELALPS KYVNFLYLAS
HYEKLKGSPE DNEQKQLFVE QHKHYLDEII EQISEFSKRV ILADANLDKV LSAYNKHRDK
PIREQAENII HLFTLTNLGA PAAFKYFDTT IDRKRYTSTK EVLDATLIHQ SITGLYETRI
DLSQLGGDGG GSPKKKRKV

In certain embodiments, a Cas9 protein used in a method or composition of the present invention comprises or has the sequence of iProt106745 (SEQ ID NO: 130):

MAPKKKRKVD KKYSIGLDIG TNSVGWAVIT DEYKVPSKKF KVLGNTDRHS IKKNLIGALL
FDSGETAEAT RLKRTARRRY TRRKNRICYL QEIFSNEMAK VDDSFFHRLE ESFLVEEDKK
HERHPIFGNI VDEVAYHEKY PTIYHLRKKL VDSTDKADLR LIYLALAHMI KFRGHFLIEG
DLNPDNSDVD KLFIQLVQTY NQLFEENPIN ASGVDAKAIL SARLSKSRRL ENLIAQLPGE
KKNGLFGNLI ALSLGLTPNF KSNFDLAEDA KLQLSKDTYD DDLDNLLAQI GDQYADLFLA
AKNLSDAILL SDILRVNTEI TKAPLSASMI KRYDEHHQDL TLLKALVRQQ LPEKYKEIFF
DQSKNGYAGY IDGGASQEEF YKFIKPILEK MDGTEELLVK LNREDLLRKQ RTFDNGSIPH
QIHLGELHAI LRRQEDFYPF LKDNREKIEK ILTFRIPYYV GPLARGNSRF AWMTRKSEET
ITPWNFEEVV DKGASAQSFI ERMTNFDKNL PNEKVLPKHS LLYEYFTVYN ELTKVKYVTE
GMRKPAFLSG EQKKAIVDLL FKTNRKVTVK QLKEDYFKKI ECFDSVEISG VEDRFNASLG
TYHDLLKIIK DKDFLDNEEN EDILEDIVLT LTLFEDREMI EERLKTYAHL FDDKVMKQLK
RRRYTGWGRL SRKLINGIRD KQSGKTILDF LKSDGFANRN FMQLIHDDSL TFKEDIQKAQ
VSGQGDSLHE HIANLAGSPA IKKGILQTVK VVDELVKVMG RHKPENIVIE MARENQTTQK
GQKNSRERMK RIEEGIKELG SQILKEHPVE NTQLQNEKLY LYYLQNGRDM YVDQELDINR
LSDYDVDHIV PQSFLKDDSI DNAVLTRSDK NRGKSDNVPS EEVVKKMKNY WRQLLNAKLI
TQRKFDNLTK AERGGLSELD KAGFIKRQLV ETRQITKHVA QILDSRMNTK YDENDKLIRE
VKVITLKSKL VSDFRKDFQF YKVREINNYH HAHDAYLNAV VGTALIKKYP KLESEFVYGD
YKVYDVRKMI AKSEQEIGKA TAKYFFYSNI MNFFKTEITL ANGEIRKRPL IETNGETGEI
VWDKGRDFAT VRKVLSMPQV NIVKKTEVQT GGFSKESILP KRNSDKLIAR KKDWDPKKYG
GFDSPTVAYS VLVVAKVEKG KSKKLKSVKE LLGITIMERS SFEKNPIDFL EAKGYKEVKK
DLIIKLPKYS LFELENGRKR MLASAGELQK GNELALPSKY VNFLYLASHY EKLKGSPEDN
EQKQLFVEQH KHYLDEIIEQ ISEFSKRVIL ADANLDKVLS AYNKHRDKPI REQAENIIHL
FTLTNLGAPA AFKYFDTTID RKRYTSTKEV LDATLIHQSI TGLYETRIDL SQLGGDSRAD
PKKKRKVHHH HHH

In certain embodiments, a Cas9 protein used in a method or composition of the present invention comprises or has the sequence of iProt106746 (SEQ ID NO: 131):

MAPKKKRKVD KKYSIGLDIG TNSVGWAVIT DEYKVPSKKF KVLGNTDRHS IKKNLIGALL
FDSGETAEAT RLKRTARRRY TRRKNRICYL QEIFSNEMAK VDDSFFHRLE ESFLVEEDKK
HERHPIFGNI VDEVAYHEKY PTIYHLRKKL VDSTDKADLR LIYLALAHMI KFRGHFLIEG
DLNPDNSDVD KLFIQLVQTY NQLFEENPIN ASGVDAKAIL SARLSKSRRL ENLIAQLPGE
KKNGLFGNLI ALSLGLTPNF KSNFDLAEDA KLQLSKDTYD DDLDNLLAQI GDQYADLFLA
AKNLSDAILL SDILRVNTEI TKAPLSASMI KRYDEHHQDL TLLKALVRQQ LPEKYKEIFF
DQSKNGYAGY IDGGASQEEF YKFIKPILEK MDGTEELLVK LNREDLLRKQ RTFDNGSIPH
QIHLGELHAI LRRQEDFYPF LKDNREKIEK ILTFRIPYYV GPLARGNSRF AWMTRKSEET
ITPWNFEEVV DKGASAQSFI ERMTNFDKNL PNEKVLPKHS LLYEYFTVYN ELTKVKYVTE
GMRKPAFLSG EQKKAIVDLL FKTNRKVTVK QLKEDYFKKI ECFDSVEISG VEDRFNASLG
TYHDLLKIIK DKDFLDNEEN EDILEDIVLT LTLFEDREMI EERLKTYAHL FDDKVMKQLK
RRRYTGWGRL SRKLINGIRD KQSGKTILDF LKSDGFANRN FMQLIHDDSL TFKEDIQKAQ
VSGQGDSLHE HIANLAGSPA IKKGILQTVK VVDELVKVMG RHKPENIVIE MARENQTTQK
GQKNSRERMK RIEEGIKELG SQILKEHPVE NTQLQNEALY LYYLQNGRDM YVDQELDINR
LSDYDVDHIV PQSFLKDDSI DNKVLTRSDK NRGKSDNVPS EEVVKKMKNY WRQLLNAKLI
TQRKFDNLTK AERGGLSELD KAGFIKRQLV ETRQITKHVA QILDSRMNTK YDENDKLIRE
VKVITLKSKL VSDFRKDFQF YKVREINNYH HAHDAYLNAV VGTALIKKYP ALESEFVYGD
YKVYDVRKMI AKSEQEIGKA TAKYFFYSNI MNFFKTEITL ANGEIRKAPL IETNGETGEI
VWDKGRDFAT VRKVLSMPQV NIVKKTEVQT GGFSKESILP KRNSDKLIAR KKDWDPKKYG
GFDSPTVAYS VLVVAKVEKG KSKKLKSVKE LLGITIMERS SFEKNPIDFL EAKGYKEVKK
DLIIKLPKYS LFELENGRKR MLASAGELQK GNELALPSKY VNFLYLASHY EKLKGSPEDN
EQKQLFVEQH KHYLDEIIEQ ISEFSKRVIL ADANLDKVLS AYNKHRDKPI REQAENIIHL
FTLTNLGAPA AFKYFDTTID RKRYTSTKEV LDATLIHQSI TGLYETRIDL SQLGGDSRAD
PKKKRKVHHH HHH

In certain embodiments, a Cas9 protein used in a method or composition of the present invention comprises or has the sequence of iProt106747 (SEQ ID NO: 132):

MAPKKKRKVD KKYSIGLDIG TNSVGWAVIT DEYKVPSKKF KVLGNTDRHS IKKNLIGALL
FDSGETAEAT RLKRTARRRY TRRKNRICYL QEIFSNEMAK VDDSFFHRLE ESFLVEEDKK
HERHPIFGNI VDEVAYHEKY PTIYHLRKKL VDSTDKADLR LIYLALAHMI KFRGHFLIEG
DLNPDNSDVD KLFIQLVQTY NQLFEENPIN ASGVDAKAIL SARLSKSRRL ENLIAQLPGE
KKNGLFGNLI ALSLGLTPNF KSNFDLAEDA KLQLSKDTYD DDLDNLLAQI GDQYADLFLA
AKNLSDAILL SDILRVNTEI TKAPLSASMI KRYDEHHQDL TLLKALVRQQ LPEKYKEIFF
DQSKNGYAGY IDGGASQEEF YKFIKPILEK MDGTEELLVK LNREDLLRKQ RTFDNGSIPH
QIHLGELHAI LRRQEDFYPF LKDNREKIEK ILTFRIPYYV GPLARGNSRF AWMTRKSEET
ITPWNFEEVV DKGASAQSFI ERMTNFDKNL PNEKVLPKHS LLYEYFTVYN ELTKVKYVTE
GMRKPAFLSG EQKKAIVDLL FKTNRKVTVK QLKEDYFKKI ECFDSVEISG VEDRFNASLG
TYHDLLKIIK DKDFLDNEEN EDILEDIVLT LTLFEDREMI EERLKTYAHL FDDKVMKQLK
RRRYTGWGRL SRKLINGIRD KQSGKTILDF LKSDGFANRN FMQLIHDDSL TFKEDIQKAQ
VSGQGDSLHE HIANLAGSPA IKKGILQTVK VVDELVKVMG RHKPENIVIE MARENQTTQK
GQKNSRERMK RIEEGIKELG SQILKEHPVE NTQLQNEKLY LYYLQNGRDM YVDQELDINR
LSDYDVDHIV PQSFLADDSI DNKVLTRSDK NRGKSDNVPS EEVVKKMKNY WRQLLNAKLI
TQRKFDNLTK AERGGLSELD KAGFIKRQLV ETRQITKHVA QILDSRMNTK YDENDKLIRE
VKVITLKSKL VSDFRKDFQF YKVREINNYH HAHDAYLNAV VGTALIKKYP ALESEFVYGD
YKVYDVRKMI AKSEQEIGKA TAKYFFYSNI MNFFKTEITL ANGEIRKAPL IETNGETGEI
VWDKGRDFAT VRKVLSMPQV NIVKKTEVQT GGFSKESILP KRNSDKLIAR KKDWDPKKYG
GFDSPTVAYS VLVVAKVEKG KSKKLKSVKE LLGITIMERS SFEKNPIDFL EAKGYKEVKK
DLIIKLPKYS LFELENGRKR MLASAGELQK GNELALPSKY VNFLYLASHY EKLKGSPEDN
EQKQLFVEQH KHYLDEIIEQ ISEFSKRVIL ADANLDKVLS AYNKHRDKPI REQAENIIHL
FTLTNLGAPA AFKYFDTTID RKRYTSTKEV LDATLIHQSI TGLYETRIDL SQLGGDSRAD
PKKKRKVHHH HHH

In certain embodiments, a Cas9 protein used in a method or composition of the present invention comprises or has the sequence of iProt106884 (SEQ ID NO: 133):

MAPKKKRKVD KKYSIGLDIG TNSVGWAVIT DEYKVPSKKF KVLGNTDRHS IKKNLIGALL
FDSGETAEAT RLKRTARRRY TRRKNRICYL QEIFSNEMAK VDDSFFHRLE ESFLVEEDKK
HERHPIFGNI VDEVAYHEKY PTIYHLRKKL VDSTDKADLR LIYLALAHMI KFRGHFLIEG
DLNPDNSDVD KLFIQLVQTY NQLFEENPIN ASGVDAKAIL SARLSKSRRL ENLIAQLPGE
KKNGLFGNLI ALSLGLTPNF KSNFDLAEDA KLQLSKDTYD DDLDNLLAQI GDQYADLFLA
AKNLSDAILL SDILRVNTEI TKAPLSASMI KRYDEHHQDL TLLKALVRQQ LPEKYKEIFF
DQSKNGYAGY IDGGASQEEF YKFIKPILEK MDGTEELLVK LNREDLLRKQ RTFDNGSIPH
QIHLGELHAI LRRQEDFYPF LKDNREKIEK ILTFRIPYYV GPLARGNSRF AWMTRKSEET
ITPWNFEEVV DKGASAQSFI ERMTAFDKNL PNEKVLPKHS LLYEYFTVYN ELTKVKYVTE
GMRKPAFLSG EQKKAIVDLL FKTNRKVTVK QLKEDYFKKI ECFDSVEISG VEDRFNASLG
TYHDLLKIIK DKDFLDNEEN EDILEDIVLT LTLFEDREMI EERLKTYAHL FDDKVMKQLK
RRRYTGWGAL SRKLINGIRD KQSGKTILDF LKSDGFANRN FMALIHDDSL TFKEDIQKAQ
VSGQGDSLHE HIANLAGSPA IKKGILQTVK VVDELVKVMG RHKPENIVIE MARENQTTQK
GQKNSRERMK RIEEGIKELG SQILKEHPVE NTQLQNEKLY LYYLQNGRDM YVDQELDINR
LSDYDVDHIV PQSFLKDDSI DNKVLTRSDK NRGKSDNVPS EEVVKKMKNY WRQLLNAKLI
TQRKFDNLTK AERGGLSELD KAGFIKRQLV ETRAITKHVA QILDSRMNTK YDENDKLIRE
VKVITLKSKL VSDFRKDFQF YKVREINNYH HAHDAYLNAV VGTALIKKYP KLESEFVYGD
YKVYDVRKMI AKSEQEIGKA TAKYFFYSNI MNFFKTEITL ANGEIRKRPL IETNGETGEI
VWDKGRDFAT VRKVLSMPQV NIVKKTEVQT GGFSKESILP KRNSDKLIAR KKDWDPKKYG
GFDSPTVAYS VLVVAKVEKG KSKKLKSVKE LLGITIMERS SFEKNPIDFL EAKGYKEVKK
DLIIKLPKYS LFELENGRKR MLASAGELQK GNELALPSKY VNFLYLASHY EKLKGSPEDN
EQKQLFVEQH KHYLDEIIEQ ISEFSKRVIL ADANLDKVLS AYNKHRDKPI REQAENIIHL
FTLTNLGAPA AFKYFDTTID RKRYTSTKEV LDATLIHQSI TGLYETRIDL SQLGGDSRAD
PKKKRKVHHH HHH

In certain embodiments, a Cas9 protein used in a method or composition of the present invention comprises or has the sequence of any one of SEQ ID NOs: 124 to 133, omitting terminal histidine tag, e.g., a His(6) tag (His His His His His His, SEQ ID NO: 121). In certain embodiments, a Cas9 protein used in a method or composition of the present invention comprises or has the sequence of any one of SEQ ID NOs: 124 to 133, omitting terminal histidine tag, e.g., a His(6) tag (His His His His His His, SEQ ID NO: 121) and an NLS sequence, e.g., the amino acid sequence PKKKRKV (SEQ ID NO: 8).

In a preferred embodoiment, the CRISPR system used in the present invention comprises a Cas9 molecule comprising SEQ ID NO: 106 or 107. In certain preferred embodiments, a Cas9 protein used in a method or composition of the present invention comprises or has the sequence of SEQ ID NO: 106 or SEQ ID NO: 107, omitting terminal histidine tag, e.g., a His(6) tag (His His His His His His, SEQ ID NO: 121). In certain preferred embodiments, a Cas9 protein used in a method or composition of the present invention comprises or has the sequence of SEQ ID NO: 106 or SEQ ID NO: 107, omitting terminal histidine tag, e.g., a His(6) tag (His His His His His His, SEQ ID NO: 121) and an NLS sequence, e.g., the amino acid sequence PKKKRKV (SEQ ID NO: 8).

Thus, engineered CRISPR gene editing systems, e.g., for gene editing in eukaryotic cells, typically involve (1) a guide RNA molecule (gRNA) comprising a targeting domain (which is capable of hybridizing to the genomic DNA target sequence), and a sequence which is capable of binding to a Cas, e.g., Cas9 enzyme, and (2) a Cas, e.g., Cas9, protein. The sequence which is capable of binding to a Cas protein may comprise a domain referred to as a tracr domain or tracrRNA. The targeting domain and the sequence which is capable of binding to a Cas, e.g., Cas9 enzyme, may be disposed on the same (sometimes referred to as a single gRNA, chimeric gRNA or sgRNA) or different molecules (sometimes referred to as a dual gRNA or dgRNA). If disposed on different molecules, each includes a hybridization domain which allows the molecules to associate, e.g., through hybridization.

### gRNA

The terms “guide RNA”, “guide RNA molecule”, “gRNA molecule” or “gRNA” are used interchangeably, and refer to a set of nucleic acid molecules that promote the specific directing of a RNA-guided nuclease or other effector molecule (typically in complex with the gRNA molecule) to a target sequence. In some embodiments, said directing is accomplished through hybridization of a portion of the gRNA to DNA (e.g., through the gRNA targeting domain), and by binding of a portion of the gRNA molecule to the RNA-guided nuclease or other effector molecule (e.g., through at least the gRNA tracr). In embodiments, a gRNA molecule consists of a single contiguous polynucleotide molecule, referred to herein as a “single guide RNA” or “sgRNA” and the like. In other embodiments, a gRNA molecule consists of a plurality, usually two, polynucleotide molecules, which are themselves capable of association, usually through hybridization, referred to herein as a “dual guide RNA” or “dgRNA” and the like. gRNA molecules are described in more detail below, but generally include a targeting domain and a tracr. In embodiments the targeting domain and tracr are disposed on a single polynucleotide. In other embodiments, the targeting domain and tracr are disposed on separate polynucleotides.

The term “targeting domain” as the term is used in connection with a gRNA, is the portion of the gRNA molecule that recognizes, e.g., is complementary to, a target sequence, e.g., a target sequence within the nucleic acid of a cell, e.g., within a gene.

The term “crRNA” as the term is used in connection with a gRNA molecule, is a portion of the gRNA molecule that comprises a targeting domain and a region that interacts with a tracr to form a flagpole region.

The term “flagpole” as used herein in connection with a gRNA molecule, refers to the portion of the gRNA where the crRNA and the tracr bind to, or hybridize to, one another.

The term “tracr” as used herein in connection with a gRNA molecule, refers to the portion of the gRNA that binds to a nuclease or other effector molecule. In embodiements, the tracr comprises nucleic acid sequence that binds specifically to Cas9. In embodiments, the tracr comprises nucleic acid sequence that forms part of the flagpole.

The term “target sequence” refers to a sequence of nucleic acids complimentary, for example fully complementary, to a gRNA targeting domain. In embodiments, the target sequence is disposed on genomic DNA. In an embodiment the target sequence is adjacent to (either on the same strand or on the complementary strand of DNA) a protospacer adjacent motif (PAM) sequence recognized by a protein having nuclease or other effector activity, e.g., a PAM sequence recognized by Cas9. The target sequence refers herein to a target sequence of beta-2-microglobulin or B2M.

The term “complementary” as used in connection with nucleic acid, refers to the pairing of bases, A with T or U, and G with C. The term complementary refers to nucleic acid molecules that are completely complementary, that is, form A to T or U pairs and G to C pairs across the entire reference sequence, as well as molecules that are at least 80%, 85%, 90%, 95%, 99% complementary.

“Betamicroglobulin” or “B2M”, also known as IMD43, is a component of MHC class l molecules. B2M is a serum protein found in association with the major histocompatibility complex (MHC) class l heavy chain on the surface of nearly all nucleated cells. The protein has a predominantly beta-pleated sheet structure that can form amyloid fibrils in some pathological conditions. The encoded antimicrobial protein displays antibacterial activity in amniotic fluid. A mutation in this gene has been shown to result in hypercatabolic hypoproteinemia (NCBI: Gene ID: 567).

The term “a target sequence in the B2M gene” or “a target polynucleotide sequence in the B2M gene” refers to a contigious sequence within the B2M polynucleotide sequence (NCBI: Gene ID: 567). The B2M polynucleotide sequence encodes B2M protein, a serum protein found in association with the major histocompatibility complex (MHC) class l heavy chain on the surface of nearly all nucleated cells. The B2M gene has 4 exons which span approximately 8 kb.

In some embodiments, the target polynucleotide sequence is a variant of B2M. in some embodiments, the target polynucleotide sequence is a homolog of B2M, In some embodiments, the target polynucleotide sequence is an ortholog of B2M.

The term “genomic DNA of B2M” refers to the B2M polynucleotide sequence (NCBI: Gene ID: 567).

gRNA molecule formats are known in the art. An exemplary gRNA molecule, e.g., dgRNA molecule, as disclosed herein comprises, e.g., consists of, a first nucleic acid having the sequence:

5′nnnnnnnnnnnnnnnnnnnnGUUUUAGAGCUAUGCUGUUUUG 3′ (S
EQ ID NO: 9),

where the “n”’s refer to the residues of the targeting domain, e.g., as described herein, and may consist of 15-25 nucleotides, e.g., consists of 20 nucleotides; and a second nucleic acid sequence having the exemplary sequence:

5′AACUUACCAAGGAACAGCAUAGCAAGUUAAAAUAAGGCUAGUCCGUUA
UCAACUUGAAAAAGUGGCACCGAGUCGGUGC 3′, optionally wit
h 1, 2, 3, 4, 5, 6, or 7 (e.g., 4 or 7,e.g., 7) ad
ditional U nucleotides at the 3′ end (SEQ ID NO: 1
0).

The second nucleic acid molecule may alternatively consist of a fragment of the sequence above, wherein such fragment is capable of hybridizing to the first nucleic acid. An example of such second nucleic acid molecule is:

5′AACAGCAUAGCAAGUUAAAAUAAGGCUAGUCCGUUAUCAACUUGAAAA
AGUGGCACCGAGUCGGUGC 3′, optionally with 1, 2, 3, 4
, 5, 6, or 7 (e.g., 4 or 7, e.g., 7) additional Un
ucleotides at the 3′ end (SEQ ID NO:11).

Another exemplary gRNA molecule, e.g., a sgRNA molecule, as disclosed herein comprises, e.g., consists of a first nucleic acid having the sequence:

′nnnnnnnnnnnnnnnnnnnGUUUUAGAGCUAGAAAUAGCAAGUUAAAAU
AAGGCUAGUCCGUUAUCAACUUGAAAAAGUGGCACCGAGUCGGUGC 3′(
SEQ ID NO:12

where the “n”’s refer to the residues of the targeting domain, e.g., as described herein and may consist of 15-25 nucleotides, e.g., consist of 20 nucleotides, optionally with 1, 2, 3, 4, 5, 6, or 7 (e.g., 4 or 7, e.g., 4) additional U nucleotides at the 3′ end.

Additional components and/or elements of CRISPR gene editing systems known in the art, e.g., are described in U.S. Publication No.2014/0068797, WO2015/048577, and Cong (2013) Science 339: 819-823, the contents of which are hereby incorporated by reference in their entirety. Such systems can be generated which inhibit a target gene, by, for example, engineering a CRISPR gene editing system to include a gRNA molecule comprising a targeting domain that hybridizes to a sequence of the target gene. In embodiments, the gRNA comprises a targeting domain which is fully complementarity to 15-25 nucleotides, e.g., 20 nucleotides, of a target gene. In embodiments, the 15-25 nucleotides, e.g., 20 nucleotides, of the target gene, are disposed immediately 5′ to a protospacer adjacent motif (PAM) sequence recognized by the RNA-guided nuclease, e.g., Cas protein, of the CRISPR gene editing system (e.g., where the system comprises a S. pyogenes Cas9 protein, the PAM sequence comprises NGG, where N can be any of A, T, G or C).

In some embodiments, the gRNA molecule and RNA-guided nuclease, e.g., Cas protein, of the CRISPR gene editing system can be complexed to form a RNP (ribonucleoprotein) complex. Such RNP complexes may be used in the methods described herein. In other embodiments, nucleic acid encoding one or more components of the CRISPR gene editing system may be used in the methods described herein.

In some embodiments, foreign DNA can be introduced into the cell along with the CRISPR gene editing system, e.g., DNA encoding a desired transgene, with or without a promoter active in the target cell type. Depending on the sequences of the foreign DNA and target sequence of the genome, this process can be used to integrate the foreign DNA into the genome, at or near the site targeted by the CRISPR gene editing system. For example, 3′ and 5′ sequences flanking the transgene may be included in the foreign DNA which are homologous to the gene sequence 3′ and 5′ (respectively) of the site in the genome cut by the gene editing system. Such foreign DNA molecule can be referred to “template DNA.”

In an embodiment, the CRISPR gene editing system of the present invention comprises Cas9, e.g., S. pyogenes Cas9, and a gRNA comprising a targeting domain which hybridizes to a sequence of a gene of interest. In an embodiment, the gRNA and Cas9 are complexed to form a RNP(ribonucleoprotein). In an embodiment, the CRISPR gene editing system comprises nucleic acid encoding a gRNA and nucleic acid encoding a Cas protein, e.g., Cas9, e.g., S. pyogenes Cas9. In an embodiment, the CRISPR gene editing system comprises a gRNA and nucleic acid encoding a Cas protein, e.g., Cas9, e.g., S. pyogenes Cas9.

In some embodiments, inducible control over Cas9, sgRNA expression can be utilized to optimize efficiency while reducing the frequency of off-target effects thereby increasing safety. Examples include, but are not limited to, transcriptional and post-transcriptional switches listed as follows; doxycycline inducible transcription Loew et al. (2010) BMC Biotechnol. 10:81, Shield1 inducible protein stabilization Banaszynski et al. (2016) Cell 126: 995-1004, Tamoxifen induced protein activation Davis et al. (2015) Nat. Chem. Biol. 11: 316-318, Rapamycin or optogenetic induced activation or dimerization of split Cas9 Zetsche (2015) Nature Biotechnol. 33(2): 139-142, Nihongaki et al. (2015) Nature Biotechnol. 33(7): 755-760, Polstein and Gersbach (2015) Nat. Chem. Biol. 11: 198-200, and SMASh tag drug inducible degradation Chung et al. (2015) Nat. Chem. Biol. 11: 713-720.

In general, the CRISPR-Cas or CRISPR system refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas9, e.g., CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise any polynucleotide, such as DNA or RNA polynucleotides. In some embodiments, a target sequence is located in the nucleus or cytoplasm of a cell. In some embodiments it may be preferred in a CRISPR complex that the tracr sequence has one or more hairpins and is 30 or more nucleotides in length, 40 or more nucleotides in length, or 50 or more nucleotides in length; the guide sequence is between 10 to 30 nucleotides in length, the CRISPR/Cas enzyme is a Type II Cas9 enzyme. In embodiments of the invention the terms guide sequence and guide RNA (“gRNA”) are used interchangeably. In general, a guide sequence is any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith- Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies); ELAND (Illumina, San Diego, CA), and SOAP. In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. Preferably the guide sequence is 10 - 30 nucleotides long. The ability of a guide sequence to direct sequence- specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art. A guide sequence may be selected to target any target sequence. In some embodiments, the target sequence is a sequence within a genome of a cell. Exemplary target sequences include those that are unique in the target genome. For example, for the S. pyogenes Cas9, a unique target sequence in a genome may include a Cas9 target site of the form MM M MMMNNNNNNNNNNNNXGG (SEQ ID NO: 13), where NNN NNN NN XGG (SEQ ID NO: 179) (N is A, G, T, or C; and X can be anything) has a single occurrence in the genome. A unique target sequence in a genome may include an S. pyogenes Cas9 target site of the form MMM MMMMMNNNNNNNNNNNXGG (SEQ ID NO: 14), where N N N N XGG (N is A, G, T, or C; and X can be anything) has a single occurrence in the genome. For the S. thermophilus CRISPRI Cas9, a unique target sequence in a genome may include a Cas9 target site of the form MMMMMMMMNN N N NN XXAGAAW (SEQ ID NO: 15), where NNN NN N XXAGAAW (SEQ ID NO: 180) (N is A, G, T, or C; X can be anything; and W is A or T) has a single occurrence in the genome. A unique target sequence in a genome may include an S. thermophilus CRISPRI Cas9 target site of the form MMMMMM MN N NNN NNXXAGAAW (SEQ ID NO: 16), where NNNNNNNNNNNXXAGAAW (SEQ ID NO: 181) (N is A, G, T, or C; X can be anything; and W is A or T) has a single occurrence in the genome. For the S. pyogenes Cas9, a unique target sequence in a genome may include a Cas9 target site of the form MMMMMMMMNNNN NNNNNNXGGXG (SEQ ID NO: 17), where NNNNNNNNNNNNXGGXG (SEQ ID NO: 182) (N is A, G, T, or C; and X can be anything) has a single occurrence in the genome. A unique target sequence in a genome may include an S. pyogenes Cas9 target site of the form MMMMMMMMMNNNNNNNNNNNXGGXG (SEQ ID NO: 183) where NNNNNNNNNNNXGGXG (SEQ ID NO: 18), (N is A, G, T, or C; and X can be anything) has a single occurrence in the genome. In each of these sequences, N is any nucleobase and “M ” may be A, G, T, or C, and need not be considered in identifying a sequence as unique. In some embodiments, a guide sequence is selected to reduce the degree secondary structure within the guide sequence. In some embodiments, about or less than about 75%, 50%, 40%, 30%, 25%, 20%, 15%, 10%, 5%, 1%, or fewer of the nucleotides of the guide sequence participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g. A.R. Gruber et al., 2008, Cell 106(1): 23-24; and PA Carr and GM Church, 2009, Nature Biotechnology 27(12): 1 151-62).

### Methods for Designing gRNA Molecules

Methods for selecting, designing, and validating targeting domains for use in the gRNAs described herein are provided. Exemplary targeting domains for incorporation into gRNAs are also provided herein.

Methods for selection and validation of target sequences as well as off-target analyses have been described (see, e.g., Mali 2013; Hsu 2013; Fu 2014; Heigwer 2014; Bae 2014; and Xiao 2014). For example, target sequences can be chosen by identifying the PAM sequence for a Cas9 molecule (for example, relevant PAM e.g., NGG PAM for S. pyogenes, NNNNGATT (SEQ ID NO: 19), or NNNNGCTT PAM (SEQ ID NO: 20), for N. meningitides, and NNGRRT (SEQ ID NO: 21), or NNGRRV PAM (SEQ ID NO: 22), for S. aureus), and identifying the adjacent sequence as the target sequence for a CRISPR system (e.g., S. pyogenes Cas9 CRISPR system) using that Cas9 molecule. A software tool can be used to further refine the choice of potential targeting domains corresponding to a user’s target sequence, e.g., to minimize total off-target activity across the genome. Candidate targeting domains and gRNAs comprising those targeting domains can be functionally evaluated by using methods known in the art and/or as set forth herein.

As a non-limiting example, targeting domains for use in gRNAs for use with S. pyogenes, N. meningiitidis and S. aureus Cas9s are identified using a DNA sequence searching algorithm. 17-mer, 18-mer, 19-mer, 20-mer, 21-mer, 22-mer, 23-mer, and/or 24-mer targeting domains are designed for each Cas9. With respect to S. pyogenes Cas9, preferably, the targeting domain is a 20-mer. gRNA design is carried out using a custom gRNA design software based on the public tool cas-offinder (Bae 2014). This software scores guides after calculating their genome-wide off-target propensity.

Provided in the table below (i.e., Table 1, Table 4) are targeting domains for gRNA molecules for use in the compositions and methods of the present invention in altering expression of or altering the B2M gene.

In specific embodiments, cells described herein, such as LSCs and CECs, have reduced or eliminated expression of B2M by a CRISPR system (e.g., S. pyogenes Cas9 CRISPR system) comprising a gRNA selected from those described in Table 1 or Table 4 or Table 6. Use of CRISPR and gRNA molecules targeting the B2M gene are also described, for example, in Mandal et al., 2014, Cell Stem Cell, 15:643-652; International Patent Application Publication Nos. WO16073955, WO17093969, WO16011080, WO16183041, WO17106537, W02017143210, WO2017212072, and WO2018064594.

TABLE 1

gRNA Targeting Domains for Exemplary Allogeneic Ocular Cell Targets

| Target | Target Region (e.g., Exon: | S t r a n d | Genomic Location (hg38) | Guide RNA Sequence | SEQ ID NO: |
|---|---|---|---|---|---|
| B2M | Exon 1 | + | chr15:44711469-44711494 | UGGCUGGGCACGCGUU UAAUAUAAG | 23 |
| B2M | Exon 1 | + | chr15:44711472-44711497 | CUGGGCACGCGUUUAAU AUAAGUGG | 24 |
| B2M | Exon 1 | + | chr15:44711483-44711508 | UUUAAUAUAAGUGGAGG CGUCGCGC | 25 |
| B2M | Exon 1 | + | chr15:44711486-44711511 | AAUAUAAGUGGAGGCGU CGCGCUGG | 26 |
| B2M | Exon 1 | + | chr15:44711487-44711512 | AUAUAAGUGGAGGCGUC GCGCUGGC | 27 |
| B2M | Exon 1 | + | chr15:44711512-44711537 | GGGCAUUCCUGAAGCUG ACAGCAUU | 28 |
| B2M | Exon 1 | + | chr15:44711513-44711538 | GGCAUUCCUGAAGCUGA CAGCAUUC | 29 |
| B2M | Exon 1 | + | chr15:44711534-44711559 | AUUCGGGCCGAGAUGUC UCGCUCCG | 30 |
| B2M | Exon 1 | + | chr15:44711568-44711593 | CUGUGCUCGCGCUACUC UCUCUUUC | 31 |
| B2M | Exon 1 | + | chr15:44711573-44711598 | CUCGCGCUACUCUCUCU UUCUGGCC | 32 |
| B2M | Exon 1 | + | chr15:44711576-44711601 | GCGCUACUCUCUCUUUC UGGCCUGG | 33 |
| B2M | Exon 1 | - | chr15:44711466-44711491 | AUAUUAAACGCGUGCCC AGCCAAUC | 34 |
| B2M | Exon 1 | - | chr15:44711522-44711547 | UCUCGGCCCGAAUGCUG UCAGCUUC | 35 |
| B2M | Exon 1 | - | chr15:44711544-44711569 | GCUAAGGCCACGGAGCG AGACAUCU | 36 |
| B2M | Exon 1 | - | chr15:44711559-44711584 | AGUAGCGCGAGCACAGC UAAGGCCA | 37 |
| B2M | Exon 1 | - | chr15:44711565-44711590 | AGAGAGAGUAGCGCGAG CACAGCUA | 38 |
| B2M | Exon 1 | - | chr15:44711599-44711624 | GAGAGACUCACGCUGGA UAGCCUCC | 39 |
| B2M | Exon 1 | - | chr15:44711611-44711636 | GCGGGAGGGUAGGAGA GACUCACGC | 40 |
| B2M | Exon 2 | + | chr15:44715412-44715437 | UAUUCCUCAGGUACUCC AAAGAUUC | 41 |
| B2M | Exon 2 | + | chr15:44715440-44715465 | UUUACUCACGUCAUCCA GCAGAGAA | 42 |
| B2M | Exon 2 | + | chr15:44715473-44715498 | CAAAUUUCCUGAAUUGC UAUGUGUC | 43 |
| B2M | Exon 2 | + | chr15:44715474-44715499 | AAAUUUCCUGAAUUGCU AUGUGUCU | 44 |
| B2M | Exon 2 | + | chr15:44715515-44715540 | ACAUUGAAGUUGACUUA CUGAAGAA | 45 |
| B2M | Exon 2 | + | chr15:44715535-44715560 | AAGAAUGGAGAGAGAAU UGAAAAAG | 46 |
| B2M | Exon 2 | + | chr15:44715562-44715587 | GAGCAUUCAGACUUGUC UUUCAGCA | 47 |
| B2M | Exon 2 | + | chr15:44715567-44715592 | UUCAGACUUGUCUUUCA GCAAGGAC | 48 |
| B2M | Exon 2 | + | chr15:44715672-44715697 | UUUGUCACAGCCCAAGA UAGUUAAG | 49 |
| B2M | Exon 2 | + | chr15:44715673-44715698 | UUGUCACAGCCCAAGAU AGUUAAGU | 50 |
| B2M | Exon 2 | + | chr15:44715674-44715699 | UGUCACAGCCCAAGAUA GUUAAGUG | 51 |
| B2M | Exon 2 | - | chr15:44715410-44715435 | AUCUUUGGAGUACCUGA GGAAUAUC | 52 |
| B2M | Exon 2 | - | chr15:44715411-44715436 | AAUCUUUGGAGUACCUG AGGAAUAU | 53 |
| B2M | Exon 2 | - | chr15:44715419-44715444 | UAAACCUGAAUCUUUGG AGUACCUG | 54 |
| B2M | Exon 2 | - | chr15:44715430-44715455 | GAUGACGUGAGUAAACC UGAAUCUU | 55 |
| B2M | Exon 2 | - | chr15:44715457-44715482 | GGAAAUUUGACUUUCCA UUCUCUGC | 56 |
| B2M | Exon 2 | - | chr15:44715483-44715508 | AUGAAACCCAGACACAU AGCAAUUC | 57 |
| B2M | Exon 2 | - | chr15:44715511-44715536 | UCAGUAAGUCAACUUCA AUGUCGGA | 58 |
| B2M | Exon 2 | - | chr15:44715515-44715540 | UUCUUCAGUAAGUCAAC UUCAAUGU | 59 |
| B2M | Exon 2 | - | chr15:44715629-44715654 | CAGGCAUACUCAUCUUU UUCAGUGG | 60 |
| B2M | Exon 2 | - | chr15:44715630-44715655 | GCAGGCAUACUCAUCUU UUUCAGUG | 61 |
| B2M | Exon 2 | - | chr15:44715631-44715656 | GGCAGGCAUACUCAUCU UUUUCAGU | 62 |
| B2M | Exon 2 | - | chr15:44715632-44715657 | CGGCAGGCAUACUCAUC UUUUUCAG | 63 |
| B2M | Exon 2 | - | chr15:44715653-44715678 | GACAAAGUCACAUGGUU CACACGGC | 64 |
| B2M | Exon 2 | - | chr15:44715657-44715682 | CUGUGACAAAGUCACAU GGUUCACA | 65 |
| B2M | Exon 2 | - | chr15:44715666-44715691 | UAUCUUGGGCUGUGACA AAGUCACA | 66 |
| B2M | Exon 2 | - | chr15:44715685-44715710 | AAGACUUACCCCACUUA ACUAUCUU | 67 |
| B2M | Exon 2 | - | chr15:44715686-44715711 | UAAGACUUACCCCACUU AACUAUCU | 68 |
| B2M | Exon 3 | + | chr15:44716326-44716351 | AGAUCGAGACAUGUAAG CAGCAUCA | 69 |
| B2M | Exon 3 | + | chr15:44716329-44716354 | UCGAGACAUGUAAGCAG CAUCAUGG | 70 |
| B2M | Exon 3 | - | chr15:44716313-44716338 | AUGUCUCGAUCUAUGAA AAAGACAG | 71 |
| B2M | Exon 4 | + | chr15:44717599-44717624 | UUUUCAGGUUUGAAGAU GCCGCAUU | 72 |
| B2M | Exon 4 | + | chr15:44717604-44717629 | AGGUUUGAAGAUGCCGC AUUUGGAU | 73 |
| B2M | Exon 4 | + | chr15:44717681-44717706 | CACUUACACUUUAUGCA CAAAAUGU | 74 |
| B2M | Exon 4 | + | chr15:44717682-44717707 | ACUUACACUUUAUGCAC AAAAUGUA | 75 |
| B2M | Exon 4 | + | chr15:44717702-44717727 | AUGUAGGGUUAUAAUAA UGUUAACA | 76 |
| B2M | Exon 4 | + | chr15:44717764-44717789 | GUCUCCAUGUUUGAUGU AUCUGAGC | 77 |
| B2M | Exon 4 | + | chr15:44717776-44717801 | GAUGUAUCUGAGCAGGU UGCUCCAC | 78 |
| B2M | Exon 4 | + | chr15:44717786-44717811 | AGCAGGUUGCUCCACAG GUAGCUCU | 79 |
| B2M | Exon 4 | + | chr15:44717789-44717814 | AGGUUGCUCCACAGGUA GCUCUAGG | 80 |
| B2M | Exon 4 | + | chr15:44717790-44717815 | GGUUGCUCCACAGGUAG CUCUAGGA | 81 |
| B2M | Exon 4 | + | chr15:44717794-44717819 | GCUCCACAGGUAGCUCU AGGAGGGC | 82 |
| B2M | Exon 4 | + | chr15:44717805-44717830 | AGCUCUAGGAGGGCUG GCAACUUAG | 83 |
| B2M | Exon 4 | + | chr15:44717808-44717833 | UCUAGGAGGGCUGGCAA CUUAGAGG | 84 |
| B2M | Exon 4 | + | chr15:44717809-44717834 | CUAGGAGGGCUGGCAAC UUAGAGGU | 85 |
| B2M | Exon 4 | + | chr15:44717810-44717835 | UAGGAGGGCUGGCAACU UAGAGGUG | 86 |
| B2M | Exon 4 | + | chr15:44717846-44717871 | AUUCUCUUAUCCAACAU CAACAUCU | 87 |
| B2M | Exon 4 | + | chr15:44717945-44717970 | CAAUUUACAUACUCUGC UUAGAAUU | 88 |
| B2M | Exon 4 | + | chr15:44717946-44717971 | AAUUUACAUACUCUGCU UAGAAUUU | 89 |
| B2M | Exon 4 | + | chr15:44717947-44717972 | AUUUACAUACUCUGCUU AGAAUUUG | 90 |
| B2M | Exon 4 | + | chr15:44717948-44717973 | UUUACAUACUCUGCUUA GAAUUUGG | 91 |
| B2M | Exon 4 | + | chr15:44717973-44717998 | GGGAAAAUUUAGAAAUA UAAUUGAC | 92 |
| B2M | Exon 4 | + | chr15:44717981-44718006 | UUAGAAAUAUAAUUGAC AGGAUUAU | 93 |
| B2M | Exon 4 | + | chr15:44718056-44718081 | UACUUCUUAUACAUUUG AUAAAGUA | 94 |
| B2M | Exon 4 | + | chr15:44718061-44718086 | CUUAUACAUUUGAUAAA GUAAGGCA | 95 |
| B2M | Exon 4 | + | chr15:44718067-44718092 | CAUUUGAUAAAGUAAGG CAUGGUUG | 96 |
| B2M | Exon 4 | + | chr15:44718076-44718101 | AAGUAAGGCAUGGUUGU GGUUAAUC | 97 |
| B2M | Exon 4 | - | chr15:44717589-44717614 | CUUCAAACCUGAAAAGA AAAGAAAA | 98 |
| B2M | Exon 4 | - | chr15:44717620-44717645 | AUUUGGAAUUCAUCCAA UCCAAAUG | 99 |
| B2M | Exon 4 | - | chr15:44717642-44717667 | UAUUAAAAAGCAAGCAA GCAGAAUU | 100 |
| B2M | Exon 4 | - | chr15:44717771-44717796 | GCAACCUGCUCAGAUAC AUCAAACA | 101 |
| B2M | Exon 4 | - | chr15:44717800-44717825 | UUGCCAGCCCUCCUAGA GCUACCUG | 102 |
| B2M | Exon 4 | - | chr15:44717859-44717884 | UCAAAUCUGACCAAGAU GUUGAUGU | 103 |
| B2M | Exon 4 | - | chr15:44717947-44717972 | CAAAUUCUAAGCAGAGU AUGUAAAU | 104 |
| B2M | Exon 4 | - | chr15:44718119-44718144 | CAAGUUUUAUGAUUUAU UUAACUUG | 105 |

In specific embodiments, modified cells described herein, such as LSCs or CECs, have reduced or eliminated expression of B2M by a CRISPR system (e.g., S. pyogenes Cas9 CRISPR system) comprising a gRNA selected from those described in Table 1 or Table 4 or Table 6 in the Examples, wherein such modified cells comprise gene editing of B2M within Exon 1.

In specific embodiments, modified cells described herein, such as LSCs or CECs, have reduced or eliminated expression of B2M by a CRISPR system (e.g., S. pyogenes Cas9 CRISPR system) comprising a gRNA selected from those described in Table 1 or Table 4 or Table 6, wherein such modified cells comprise gene editing of B2M within Exon 2.

In specific embodiments, modified cells described herein, such as LSCs or CECs, have reduced or eliminated expression of B2M by a CRISPR system (e.g., S. pyogenes Cas9 CRISPR system) comprising a gRNA selected from those described in Table 1 or Table 4 or Table 6, wherein such modified cells comprise gene editing of B2M within Exon 3.

In specific embodiments, modified cells described herein, such as LSCs or CECs, have reduced or eliminated expression of B2M by a CRISPR system (e.g., S. pyogenes Cas9 CRISPR system) comprising a gRNA selected from those described in Table 1 or Table 4 or Table 6, wherein such modified cells comprise gene editing of B2M within Exon 4.

In specific embodiments, modified cells described herein, such as LSCs or CECs, have reduced or eliminated expression of B2M by a CRISPR system (e.g., S. pyogenes Cas9 CRISPR system) comprising a gRNA selected from those described in Table 1 or Table 4, wherein such modified cells comprise gene editing of B2M within a genomic location (e.g., chr15:44711469-44711494) selected from those described in Table 1 or Table 4. In some embodiments, the targeting domain of the gRNA molecule used in the present invention is complementary to a sequence within a genomic region selected from: chr15:44711469-44711494, chr15:44711472-44711497, chr15:44711483-44711508, chr15:44711486-44711511, chr15:44711487-44711512, chr15:44711512-44711537, chr15:44711513-44711538, chr15:44711534-4471 1559, chr15:44711568-44711593, chr15:44711573-44711598, chr15:44711576-44711601, chr15:44711466-44711491, chr15:44711522-44711547, chr15:44711544-44711569, chr15:44711559-44711584, chr15:44711565-44711590, chr15:44711599-44711624, chr15:44711611-44711636, chr15:44715412-44715437, chr15:44715440-44715465, chr15:44715473-44715498, chr15:44715474-44715499, chr15:44715515-44715540, chr15:44715535-44715560, chr15:44715562-44715587, chr15:44715567-44715592, chr15:44715672-44715697, chr15:44715673-44715698, chr15:44715674-44715699, chr15:44715410-44715435, chr15:44715411-44715436, chr15:44715419-44715444, chr15:44715430-44715455, chr15:44715457-44715482, chr15:44715483-44715508, chr15:44715511-44715536, chr15:44715515-44715540, chr15:44715629-44715654, chr15:44715630-44715655, chr15:44715631-44715656, chr15:44715632-44715657, chr15:44715653-44715678, chr15:44715657-44715682, chr15:44715666-44715691, chr15:44715685-44715710, chr15:44715686-44715711, chr15:44716326-44716351, chr15:44716329-44716354, chr15:44716313-44716338, chr15:44717599-44717624, chr15:44717604-44717629, chr15:44717681-44717706, chr15:44717682-44717707, chr15:44717702-44717727, chr15:44717764-44717789, chr15:44717776-44717801, chr15:44717786-44717811, chr15:44717789-44717814, chr15:44717790-44717815, chr15:44717794-44717819, chr15:44717805-44717830, chr15:44717808-44717833, chr15:44717809-44717834, chr15:44717810-44717835, chr15:44717846-44717871, chr15:44717945-44717970, chr15:44717946-44717971, chr15:44717947-44717972, chr15:44717948-44717973, chr15:44717973-44717998, chr15:44717981-44718006, chr15:44 718056-44718081, chr15:44718061-44718086, chr15:44718067-44718092, chr15:44 718076-44718101, chr15:44717589-44717614, chr15:44717620-44717645, chr15:44717642-44717667, chr15:44717771-44717796, chr15:44717800-44717825, chr15:44717859-44717884, chr15:44717947-44717972, chr15:44718119-44718144, chr15:44711563-44711585, chr15:44715428-44715450, chr15:44715509-44715531, chr15:44715513-44715535, chr15:44715417-44715439, chr15:44711540-44711562, chr15:44711574-44711596, chr15:44711597-44711619, chr15:44715446-44715468, chr15:44715651-44715673, chr15:44713812-44713834, chr15:44711579-44711601, chr15:44711542-44711564, chr15:44711557-44711579, chr15:44711609-44711631, chr15:44715678-44715700, chr15:44715683-44715705, chr15:44715684-44715706, chr15:44715480-44715502. In a specific embodiment, the targeting domain of the gRNA molecule is complementary to a sequence within a genomic region selected from: chr15:44715513-44715535, chr15:44711542-44711564, chr15:44711563-44711585, chr15:44715683-44715705, chr15:44711597-44711619, or chr15:44715446-44715468. In one embodiment, the targeting domain of the gRNA molecule is complementary to a sequence within a genomic region chr15:44711597-44711619. In another embodiment, the targeting domain of the gRNA molecule is complementary to a sequence within a genomic region chr15:44715446-44715468. In a preferred embodiment, the targeting domain of the gRNA molecule is complementary to a sequence within a genomic region chr15:44711563-44711585.

In particular embodiments, modified cells described herein, such as LSCs or CECs, have reduced or eliminated expression of B2M by a CRISPR system (e.g., S. pyogenes Cas9 CRISPR system) comprising a gRNA targeting domain sequence selected from those described in Table 1 or Table 4. In one embodiment, the targeting domain of the gRNA molecule to B2M comprises a targeting domain comprising the sequence of any one of SEQ ID NOs: 23-105 or 108-119 or 134-140. In a specific embodiment, the targeting domain of the gRNA molecule to B2M comprises a targeting domain comprising the sequence of any one of SEQ ID NOs: 108, 111, 115, 116, 134 or 138. In a preferred embodiment, the targeting domain of the gRNA molecule to B2M comprises a targeting domain comprising the sequence of SEQ ID NO: 108. In another embodiment, the targeting domain of the gRNA molecule to B2M comprises a targeting domain comprising the sequence of SEQ ID NO: 115. In another embodiment, the targeting domain of the gRNA molecule to B2M comprises a targeting domain comprising the sequence of SEQ ID NO: 116.

In some embodiments, modified cells described herein, such as LSCs or CECs, have reduced or eliminated expression of B2M by a CRISPR system (e.g., S. pyogenes Cas9 CRISPR system) comprising a gRNA targeting a sequence complementary to any of the sequences selected from those described in Table 5. In some embodiments, modified cells described herein, such as LSCs or CECs, have reduced or eliminated expression of B2M by a CRISPR system (e.g., S. pyogenes Cas9 CRISPR system) comprising a gRNA targeting a sequence complementary to any of the sequences selected from SEQ ID NOs: 141 to 159.

In particular embodiment, modified cells described herein, such as LSCs or CECs, have reduced or eliminated expression of B2M by a CRISPR system (e.g., S. pyogenes Cas9 CRISPR system) comprising a gRNA, wherein the gRNA comprises the sequence of any one of SEQ ID NO: 120, 160-177. In a specific embodiment, modified cells described herein, such as LSCs or CECs, have reduced or eliminated expression of B2M by a CRISPR system (e.g., S. pyogenes Cas9 CRISPR system) comprising a gRNA, wherein the gRNA comprises the sequence of any one of SEQ ID NO: 120, 162, 166, 167, 171, and 175. In a preferred embodiment, the gRNA comprises the sequence of SEQ ID NO: 120. In another embodiment, the gRNA comprises the sequence of SEQ ID NO: 166 or 167.

In particular embodiments, modified cells described herein, such as LSCs or CECs, have reduced or eliminated expression of B2M by a CRISPR system (e.g., S. pyogenes Cas9 CRISPR system) comprising a gRNA comprising one, two, three, four, five, six, seven or eight nucleotide modifications (e.g., addition, substitution, or deletion) relative to a gRNA sequence described in Table 1 or Table 4 or Table 6.

In one aspect, the present invention relates to a modified LSC or CEC comprising a genome in which the b2 microglobulin (B2M) gene on chromosome 15 has been edited to delete a contiguous stretch of genomic DNA comprising the sequence of any one of SEQ ID NOs: 141 to 159, thereby eliminating surface expression of MHC Class I molecules in the cell. In one embodiment, the modified LSC or CEC of the present invention comprises a genome in which the b2 microglobulin (B2M) gene on chromosome 15 has been edited to delete a contiguous stretch of genomic DNA comprising the sequence of any one of SEQ ID NOs: 141, 144, 148, 149, 153 or 157, thereby eliminating surface expression of MHC Class I molecules in the cell. In a more specific embodiment, the modified LSC or CEC of the present invention comprises a genome in which the b2 microglobulin (B2M) gene on chromosome 15 has been edited to delete a contiguous stretch of genomic DNA comprising the sequence of any one of SEQ ID NOs: 141, 148 or 149, thereby eliminating surface expression of MHC Class I molecules in the cell. In a preferred embodiment, the modified LSC or CEC comprises a genome in which the b2 microglobulin (B2M) gene on chromosome 15 has been edited to delete a contiguous stretch of genomic DNA comprising the sequence of SEQ ID NOs: 141, thereby eliminating surface expression of MHC Class I molecules in the cell.

In one aspect, the present invention relates to a modified LSC or CEC comprising a genome in which the b2 microglobulin (B2M) gene on chromosome 15 has been edited to delete a contiguous stretch of genomic DNA region selected from any one of: chr15:44711469-44711494, chr15:44711472-44711497, chr15:44711483-44711508, chr15:44711486-44711511, chr15:44711487-44711512, chr15:44711512-44711537, chr15:44711513-44711538, chr15:44711534-44711559, chr15:44711568-44711593, chr15:44711573-44711598, chr15:44711576-44711601, chr15:44711466-44711491, chr15:44711522-44711547, chr15:44711544-44711569, chr15:44711559-44711584, chr15:44711565-44711590, chr15:44711599-44711624, chr15:44711611-44711636, chr15:44715412-44715437, chr15:44715440-44715465, chr15:44715473-44715498, chr15:44715474-44715499, chr15:44715515-44715540, chr15:44715535-44715560, chr15:44715562-44715587, chr15:44715567-44715592, chr15:44715672-44715697, chr15:44715673-44715698, chr15:44715674-44715699, chr15:44715410-44715435, chr15:44715411-44715436, chr15:44715419-44715444, chr15:44715430-44715455, chr15:44715457-44715482, chr15:44715483-44715508, chr15:44715511-44715536, chr15:44715515-44715540, chr15:44715629-44715654, chr15:44715630-44715655, chr15:44715631-44715656, chr15:44715632-44715657, chr15:44715653-44715678, chr15:44715657-44715682, chr15:44715666-44715691, chr15:44715685-44715710, chr15:44715686-44715711, chr15:44716326-44716351, chr15:44716329-44716354, chr15:44716313-44716338, chr15:44717599-44717624, chr15:44717604-44717629, chr15:44717681-44717706, chr15:44717682-44717707, chr15:44717702-44717727, chr15:44717764-44717789, chr15:44717776-44717801, chr15:44717786-44717811, chr15:44717789-44717814, chr15:44717790-44717815, chr15:44717794-44717819, chr15:44717805-44717830, chr15:44717808-44717833, chr15:44717809-44717834, chr15:44717810-44717835, chr15:44717846-44717871, chr15:44717945-44717970, chr15:44717946-44717971, chr15:44717947-44717972, chr15:44717948-44717973, chr15:44717973-44717998, chr15:44717981-44718006, chr15:44 718056-44718081, chr15:44718061-44718086, chr15:44718067-44718092, chr15:44 718076-44718101, chr15:44717589-44717614, chr15:44717620-44717645, chr15:44717642-44717667, chr15:44717771-44717796, chr15:44717800-44717825, chr15:44717859-44717884, chr15:44717947-44717972, chr15:44718119-44718144, chr15:44711563-44711585, chr15:44715428-44715450, chr15:44715509-44715531, chr15:44715513-44715535, chr15:44715417-44715439, chr15:44711540-44711562, chr15:44711574-44711596, chr15:44711597-44711619, chr15:44715446-44715468, chr15:44715651-44715673, chr15:44713812-44713834, chr15:44711579-44711601, chr15:44711542-44711564, chr15:44711557-44711579, chr15:44711609-44711631, chr15:44715678-44715700, chr15:44715683-44715705, chr15:44715684-44715706, chr15:44715480-44715502, thereby eliminating surface expression of MHC Class I molecules in the cell. In one embodiment, the modified LSC or CEC of the present invention comprises a genome in which the b2 microglobulin (B2M) gene on chromosome 15 has been edited to delete a contiguous stretch of genomic DNA region selected from: chr15:44715513-44715535, chr15:44711542-44711564, chr15:44711563-44711585, chr15:44715683-44715705, chr15:44711597-44711619, or chr15:44715446-44715468. In a specific embodiment, the modified LSC or CEC of the present invention comprises a genome in which the b2 microglobulin (B2M) gene on chromosome 15 has been edited to delete a contiguous stretch of genomic DNA region selected from: chr15:44711563-44711585, chr15:44711597-44711619, or chr15:44715446-44715468, thereby eliminating surface expression of MHC Class I molecules in the cell. In a preferred embodiment, the modified LSC or CEC of the present invention comprises a genome in which the b2 microglobulin (B2M) gene on chromosome 15 has been edited to delete a contiguous stretch of genomic DNA region chr15:44711563-44711585, thereby eliminating surface expression of MHC Class I molecules in the cell.

In one aspect, the present invention relates to a modified LSC or CEC comprising a genome in which the b2 microglobulin (B2M) gene on chromosome 15 has been edited to to form an indel at or near the target sequence complementary to the targeting domain of the gRNA molecule.

An “indel,” as the term is used herein, refers to a nucleic acid comprising one or more insertions of nucleotides, one or more deletions of nucleotides, or a combination of insertions and delections of nucleotides, relative to a reference nucleic acid, that results after being exposed to a composition comprising a gRNA molecule, for example a CRISPR system. Indels can be determined by sequencing nucleic acid after being exposed to a composition comprising a gRNA molecule, for example, by NGS. With respect to the site of an indel, an indel is said to be “at or near” a reference site (e.g., a site complementary to a targeting domain of a gRNA molecule) if it comprises at least one insertion or deletion within about 10, 9, 8, 7, 6, 5, 4, 3, 2, or 1 nucleotide(s) of the reference site, or is overlapping with part or all of said refrence site (e.g., comprises at least one insertion or deletion overlapping with, or within 10, 9, 8, 7, 6, 5, 4, 3, 2, or 1 nucelotides of a site complementary to the targeting domain of a gRNA molecule, e.g., a gRNA molecule described herein).

An “indel pattern,” as the term is used herein, refers to a set of indels that results after exposure to a composition comprising a gRNA molecule. In an embodiment, the indel pattern consists of the top three indels, by frequency of appearance. In an embodiment, the indel pattern consists of the top five indels, by frequency of appearance. In an embodiment, the indel pattern consists of the indels which are present at greater than about 5% frequency relative to all sequencing reads. In an embodiment, the indel pattern consists of the indels which are present at greater than about 10% frequency relative to to total number of indel sequencing reads (i.e., those reads that do not consist of the unmodified reference nucleic acid sequence). In an embodiment, the indel pattern includes of any 3 of the top five most frequently observed indels. The indel pattern may be determined, for example, by sequencing cells of a population of cells which were exposed to the gRNA molecule.

In one aspect, the present invention provides a modified LSC or CEC comprising a genome in which the b2 microglobulin (B2M) gene on chromosome 15 has been edited to form an indel at or near the target sequence complementary to the targeting domain of the gRNA molecule comprising the sequence of any one of SEQ ID NOs: 23-105 or 108-119 or 134-140, thereby eliminating surface expression of MHC Class l molecules in the cell. In one embodiment, the modified LSC or CEC of the present invention comprises a genome in which the b2 microglobulin (B2M) gene on chromosome 15 has been edited to form an indel at or near the target sequence complementary to the targeting domain of the gRNA molecule comprising the sequence of any one of SEQ ID NOs: 108, 111, 115, 116, 134 or 138, thereby eliminating surface expression of MHC Class I molecules in the cell. In a more specific embodiment, the modified LSC or CEC of the present invention comprises a genome in which the b2 microglobulin (B2M) gene on chromosome 15 has been edited to form an indel at or near the target sequence complementary to the targeting domain of the gRNA molecule comprising the sequence of any one of SEQ ID NOs: 108, 115, or 116, thereby eliminating surface expression of MHC Class I molecules in the cell. In a preferred embodiment, the modified LSC or CEC comprises a genome in which the b2 microglobulin (B2M) gene on chromosome 15 has been edited to form an indel at or near the target sequence complementary to the targeting domain of the gRNA molecule comprising the sequence SEQ ID NOs: 108, thereby eliminating surface expression of MHC Class I molecules in the cell.

In one aspect, the present invention provides a modified LSC or CEC comprising a genome in which the b2 microglobulin (B2M) gene on chromosome 15 has been edited to form an indel at or near the genomic DNA region selected from any one of: chr15:44711469-44711494, chr15:44711472-44711497, chr15:44711483-44711508, chr15:44711486-44711511, chr15:44711487-44711512, chr15:44711512-44711537, chr15:44711513-44711538, chr15:44711534-44711559, chr15:44711568-44711593, chr15:44711573-44711598, chr15:44711576-44711601, chr15:44711466-44711491, chr15:44711522-44711547, chr15:44711544-44711569, chr15:44711559-44711584, chr15:44711565-44711590, chr15:44711599-44711624, chr15:44711611-44711636, chr15:44715412-44715437, chr15:44715440-44715465, chr15:44715473-44715498, chr15:44715474-44715499, chr15:44715515-44715540, chr15:44715535-44715560, chr15:44715562-44715587, chr15:44715567-44715592, chr15:44715672-44715697, chr15:44715673-44715698, chr15:44715674-44715699, chr15:44715410-44715435, chr15:44715411-44715436, chr15:44715419-44715444, chr15:44715430-44715455, chr15:44715457-44715482, chr15:44715483-44715508, chr15:44715511-44715536, chr15:44715515-44715540, chr15:44715629-44715654, chr15:44715630-44715655, chr15:44715631-44715656, chr15:44715632-44715657, chr15:44715653-44715678, chr15:44715657-44715682, chr15:44715666-44715691, chr15:44715685-44715710, chr15:44715686-44715711, chr15:44716326-44716351, chr15:44716329-44716354, chr15:44716313-44716338, chr15:44717599-44717624, chr15:44717604-44717629, chr15:44717681-44717706, chr15:44717682-44717707, chr15:44717702-44717727, chr15:44717764-44717789, chr15:44717776-44717801, chr15:44717786-44717811, chr15:44717789-44717814, chr15:44717790-44717815, chr15:44717794-44717819, chr15:44717805-44717830, chr15:44717808-44717833, chr15:44717809-44717834, chr15:44717810-44717835, chr15:44717846-44717871, chr15:44717945-44717970, chr15:44717946-44717971, chr15:44717947-44717972, chr15:44717948-44717973, chr15:44717973-44717998, chr15:44717981-44718006, chr15:44 718056-44718081, chr15:44718061-44718086, chr15:44718067-44718092, chr15:44 718076-44718101, chr15:44717589-44717614, chr15:44717620-44717645, chr15:44717642-44717667, chr15:44717771-44717796, chr15:44717800-44717825, chr15:44717859-44717884, chr15:44717947-44717972, chr15:44718119-44718144, chr15:44711563-44711585, chr15:44715428-44715450, chr15:44715509-44715531, chr15:44715513-44715535, chr15:44715417-44715439, chr15:44711540-44711562, chr15:44711574-44711596, chr15:44711597-44711619, chr15:44715446-44715468, chr15:44715651-44715673, chr15:44713812-44713834, chr15:44711579-44711601, chr15:44711542-44711564, chr15:44711557-44711579, chr15:44711609-44711631, chr15:44715678-44715700, chr15:44715683-44715705, chr15:44715684-44715706, chr15:44715480-44715502, thereby eliminating surface expression of MHC Class I molecules in the cell. In one embodiment, the modified LSC or CEC of the present invention comprises a genome in which the b2 microglobulin (B2M) gene on chromosome 15 has been edited to form an indel at or near the genomic DNA region selected from any one of: chr15:44715513-44715535, chr15:44711542-44711564, chr15:44711563-44711585, chr15:44715683-44715705, chr15:44711597-44711619, or chr15:44715446-44715468. In a specific embodiment, the modified LSC or CEC of the present invention comprises a genome in which the b2 microglobulin (B2M) gene on chromosome 15 has been edited to form an indel at or near the genomic DNA region selected from any one of: chr15:44711563-44711585, chr15:44711597-44711619, or chr15:44715446-44715468, thereby eliminating surface expression of MHC Class I molecules in the cell. In a preferred embodiment, the modified LSC or CEC of the present invention comprises a genome in which the b2 microglobulin (B2M) gene on chromosome 15 has been edited to form an indel at or near the genomic DNA region chr15:44711563-44711585, thereby eliminating surface expression of MHC Class I molecules in the cell.

In some embodiment, the formed indel comprises a deletion of 10 or greater than 10 nucleotides, optionally 11, 12, 13, 14, 15, 16, 17, 18, 19, 20, 21, 22, 23, 24, 25, 26, 27, 28, 29, 30, 31, 32, 33, 34, or 35 nucleotides.

In some embodiments, the indel is formed at or near the target sequence complementary to the targeting domain of the gRNA molecule in at least about 40%, e.g., at least about 50%, e.g., at least about 60%, e.g., at least about 70%, e.g., at least about 80%, e.g., at least about 90%, e.g., at least about 95%, e.g., at least about 96%, e.g., at least about 97%, e.g., at least about 98%, e.g., at least about 99%, of the cells of the population.

In some embodiments, the indel comprising a deletion of 10 or greater than 10 nucleotides is detected in at least about 5%, optionally at least about 10%, 15%, 20%, 25%, 30% or more, of the cells of the population.

In some embodiments, the indel is as measured by next generation sequencing (NGS).

In one embodiment, the present invention provides a modified LSC or CEC comprising a genome in which the b2 microglobulin (B2M) gene on chromosome 15 has been edited to form an indel at or near the target sequence, and wherein no off-target indels are formed in said modified LSC or CEC, e.g., as detectable by next generation sequencing and/or a nucleotide insertional assay. In one mebodiment, the present invention provides a population of modified LSCs or CECs comprising a genome in which the b2 microglobulin (B2M) gene on chromosome 15 has been edited to form an indel at or near the target sequence, and wherein an off-target indel is detected in no more than about 5%, e.g., no more than about 1%, e.g., no more than about 0.1%, e.g., no more than about 0.01%, of the cells of the population of the modifoed LSCs or CECs, e.g., as detectible by next generation sequencing and/or a nucleotide insertional assay.

An “off-target indel”, as the term used herein, refers to an indel at or near a site other than the target sequence of the targeting domain of the gRNA molecule. Such sites may comprise, for example, 1, 2, 3, 4, 5 or more mismatch nucleotides relative to the sequence of the targeting domain of the gRNA. In exemplary embodiments, such sites are detected using targeted sequencing of in silico predicted off-target sites, or by an insertional method known in the art.

In some embodiments, the modified LSC or CEC of the present invention is autologous with respect to a patient to be administered said cell. In other embodiments, the modified LSC or CEC of the present invention is allogeneic with respect to a patient to be administered said cell.

### Functional Analysis of Candidate Molecules

Candidate Cas9 molecules, candidate gRNA molecules, candidate Cas9 molecule/gRNA molecule complexes, can be evaluated by art-known methods or as described herein. For example, exemplary methods for evaluating the endonuclease activity of Cas9 molecule have been described previously (Jinek 2012). Each technique described herein may be used alone or in combination with one or more techniques to evaluate the candidate molecule. The techniques disclosed herein may be used for a variety of methods including, without limitation, methods of determining the stability of a Cas9 molecule/gRNA molecule complex, methods of determining a condition that promotes a stable Cas9 molecule/gRNA molecule complex, methods of screening for a stable Cas9 molecule/gRNA molecule complex, methods of identifying an optimal gRNA to form a stable Cas9 molecule/gRNA molecule complex, and methods of selecting a Cas9/gRNA complex for administration to a subject.

Binding and Cleavage Assay: Testing the endonuclease activity of Cas9 molecule The ability of a Cas9 molecule/gRNA molecule complex to bind to and cleave a target nucleic acid can be evaluated in a plasmid cleavage assay. In this assay, synthetic or in vitro-transcribed gRNA molecule is pre-annealed prior to the reaction by heating to 95° C. and slowly cooling down to room temperature. Native or restriction digest-linearized plasmid DNA (300 ng (~8 nM)) is incubated for 60 min at 37° C. with purified Cas9 protein molecule (50-500 nM) and gRNA (50-500 nM, 1: 1) in a Cas9 plasmid cleavage buffer (20 mM HEPES pH 7.5, 150 mM KC1, 0.5 mM DTT, 0.1 mM EDTA) with or without 10 mM MgCl2. The reactions are stopped with 5X DNA loading buffer (30% glycerol, 1.2% SDS, 250 mM EDTA), resolved by a 0.8 or 1% agarose gel electrophoresis and visualized by ethidium bromide staining. The resulting cleavage products indicate whether the Cas9 molecule cleaves both DNA strands, or only one of the two strands. For example, linear DNA products indicate the cleavage of both DNA strands. Nicked open circular products indicate that only one of the two strands is cleaved.

Alternatively, the ability of a Cas9 molecule/gRNA molecule complex to bind to and cleave a target nucleic acid can be evaluated in an oligonucleotide DNA cleavage assay. In this assay, DNA oligonucleotides (10 pmol) are radiolabeled by incubating with 5 units T4 polynucleotide kinase and -3-6 pmol (-20-40 mCi) [_{Y}-32P]-ATP in IX T4 polynucleotide kinase reaction buffer at 37° C. for 30 min, in a 50 microlitre reaction. After heat inactivation (65° C. for 20 min), reactions are purified through a column to remove unincorporated label. Duplex localising agents (100 nM) are generated by annealing labeled oligonucleotides with equimolar amounts of unlabeled complementary oligonucleotide at 95° C. for 3 min, followed by slow cooling to room temperature. For cleavage assays, gRNA molecules are annealed by heating to 95° C. for 30 s, followed by slow cooling to room temperature. Cas9 (500 nM final concentration) is pre-incubated with the annealed gRNA molecules (500 nM) in cleavage assay buffer (20 mM HEPES pH 7.5, 100 mM KCI, 5 mM MgC12, 1 mM DTT, 5% glycerol) in a total volume of 9 microlitre. Reactions are initiated by the addition of 1 microlitre target DNA (10 nM) and incubated for 1 h at 37° C. Reactions are quenched by the addition of 20 microlitre of loading dye (5 mM EDTA, 0.025% SDS, 5% glycerol in formamide) and heated to 95° C. for 5 min. Cleavage products are resolved on 12% denaturing polyacrylamide gels containing 7 M urea and visualized by phosphorimaging.

The resulting cleavage products indicate that whether the complementary strand, the non-complementary strand, or both, are cleaved.

One or both of these assays can be used to evaluate the suitability of a candidate gRNA molecule or candidate Cas9 molecule.

Indel Detection and Identification. Targeted genome modifications can also be detected by either Sanger or deep sequencing. For the former, genomic DNA from the modified region can be amplified with either primers flanking the target sequence of the gRNA. Amplicons can be subcloned into a plasmid such as pUC19 for transformation, and individual colonies should be sequenced to reveal the clonal genotype.

Alternatively, deep sequencing is suitable for sampling a large number of samples or target sites. NGS primers are designed for shorter amplicons, typically in the 100-200-bp size range. For the detection of indels, it is important to design primers situated at least 50 bp from the Cas9 target site to allow for the detection of longer indels. Amplicons may be assessed using commercially-available instruments, for example, the Illumina system. Detailed descriptions of NGS optimization and troubleshooting can be found in the Illumina user manual.

### Ocular Administration of the Expanded Cell Population

In one aspect of the invention the expanded cell population obtainable by the methods according to the invention as described above is delivered to the eye. The delivery is performed under aseptic conditions.

In one embodiment relating to use for limbal stem cell therapy after a 360° limbal peritomy the fibrovascular corneal pannus may be carefully removed from the surface.

In one aspect of the invention, the cell population is combined with a localising agent suitable for ocular delivery (as described further below) and delivered to the eye. In a preferred embodiment the cells and localising agent suitable for ocular delivery are combined and administered to the eye via a carrier such as for example a therapeutic contact lens or amniotic membrane. In an alternative embodiment the cells and localising agent suitable for use in the eye, such as a light curable biomatrix, like GelMA, are delivered to the eye via bioprinting.

In one embodiment, the invention provides a method of transplanting a population of cells comprising limbal stem cells or corneal endothelial cells onto the cornea of a subject, the method comprising expanding a population of cells comprising limbal stem cells or corneal endothelial cells by culturing said population with cell proliferation medium comprising a LATS inhibitor according to the invention, rinsing the expanded population of cells to substantially remove the LATS inhibitor, and administering said cells onto the cornea of said subject. Preferably said cells are combined with a biomatrix prior to said administration. In a specific embodiment said cells are combined with a biomatrix which is GelMA prior to said administration. In a more specific embodiment said corneal endothelial cells are combined with a biomatrix which is bioprinted onto the ocular surface. Particularly preferably said limbal stem cells or corneal endothelial cells are combined with a biomatrix which is GelMA and bioprinted onto the ocular surface by polymerising the GelMA by a light triggered reaction. In another embodiment said cells are combined with (1) thrombin and fibrinogen or (2) fibrin glue prior to said administration.

In another embodiment, the invention provides a method of transplanting a population of cells to the eye of a subject, comprising combining the cells with a biomatrix to form a cell/biomatrix mixture, injecting the mixture into the eye of the subject or applying the mixture onto the surface of the eye of the subject, and bioprinting the cells in or on the eye by guiding and fixing the cells, such as on the cornea, using a light source, such as an Ultraviolet A or white light source. In certain embodiments, the light source produces light of a wavelength that is at least 350 nm. In certain embodiments, the light source produces light in the 350 nm to 420 nm range. For example, an LED light source can be used to produce a light having a wavelength of 365 nm or 405 nm, or any other wavelength above 350 nm, or a mercury lamp with a bandpass filter can be used to produce a light having a wavelength of 365 nm. In another embodiment, the light source produces visible, white light having a wavelength, for example, in the 400 nm to 700 nm range. In certain embodiments, the cells are ocular cells, such as corneal cells (e.g., corneal endothelial cells), lens cells, trabecular mesh cells, or cells found in the anterior chamber. In a particular embodiment, the cells are corneal endothelial cells. Certain embodiments of such method include:

Embodiment x1. A method of transplanting a population of isolated cells to the eye of a subject, comprising combining the cells with a biomatrix to form a cell/biomatrix mixture, injecting the mixture into the eye of the subject, (e.g., into the anterior chamber) and bioprinting the cells in the eye by guiding and fixing the cells in the eye using a light source.

Embodiment x2. The method of Embodiment x1, wherein the isolated cells are combined with a biomatrix which is GelMA and bioprinted onto the cornea by polymerising the GelMA by a light triggered reaction.

Embodiment x3. The method of Embodiment x1 or Embodiment x2, wherein the light source produces a light having a wavelength in the 350 nm to 700 nm range.

Embodiment x4. The method of any one of Embodiments x1 to x3, wherein the wavelength is 350 nm to 420 nm.

Embodiment x5. The method of any one of Embodiments x1 to x4, wherein the wavelength is 365 nm.

Embodiment x6. The method of any one of Embodiments x1 to x5, wherein the isolated cells are corneal endothelial cells.

Embodiment x7. A method of transplanting a population of isolated cells to the eye of a subject, comprising combining the cells with a biomatrix to form a cell/biomatrix mixture, applying the mixture onto the eye of the subject, and bioprinting the cells on the eye by guiding and fixing the cells on the eye using a light source.

Embodiment x8. The method of Embodiment x7, wherein the isolated cells are combined with a biomatrix which is GelMA and bioprinted onto the ocular surface by polymerising the GelMA by a light triggered reaction.

Embodiment x9. The method of Embodiment x7 or Embodiment x8, wherein the light source produces a light having a wavelength in the 350 nm to 700 nm range.

Embodiment x10. The method of any one of Embodiments x7 to x9, wherein the wavelength is 350 nm to 420 nm.

Embodiment x11. The method of any one of Embodiments x7 to x10, wherein the wavelength is 365 nm.

Embodiment x12. The method of any one of Embodiments x7 to x11, wherein the isolated cells are limbal stem cells.

In an alternative embodiment the expanded cell population obtainable by the methods according to the invention as described above may be delivered directly via a therapeutic contact lens to the eye, without use of a localising agent suitable for ocular delivery (such as GelMA or fibrin glue).

### Localising Agent Suitable for Ocular Delivery

In an embodiment of the invention the cell preparation may be delivered to the eye via a localising agent suitable for ocular use. The cells may be embedded within the localising agent or adhered to the surface of the localising agent, or both.

The type of localising agent is not limited as long as it is able to carry LSCs or CECs and is suitable for use in the eye. In a preferred embodiment, the localising agent is degradable and biocompatible. Where CECs are delivered, preferably the localising agent can facilitate CEC attachment to the cornea after surgical delivery to the surface of the eye.

In a preferred embodiment the cells are only combined with the localising agent after cell population expansion. In a particularly preferred embodiment the expanded cell population is combined with the localising agent suitable for ocular delivery after rinsing the cell population to substantially remove the presence of the LATs inhibitor according to the invention. In one embodiment, the LSCs or CECs and localising agent are combined and stored in a form suitable for ocular use. In another embodiment, the LSCs or CECs and localising agent are stored separately and combined immediately prior to ocular use.

The localising agent is preferably selected from the list consisting of fibrin, collagen, gelatin, cellulose, amniotic membrane, fibrin glue, a combination of thrombin and fibrinogen, polyethylene (glycol) diacrylate (PEGDA), GelMA, (which is methacrylamide modified gelatin, and is also known as gelatin methacrylate), localising agents comprising a polymer, cross-linked polymer, or hydrogel comprising one or more of hyaluronic acid, polyethylene glycol, polypropylene glycol, polyethylene oxide, polypropylene oxide, poloxamer, polyvinyl alcohol, polyacrylic acid, polymethacrylic acid, polyvinyl pyrrolidone, poly(lactide-co-glycolide), alginate, gelatin, collagen, fibrinogen, cellulose, methylcellulose, hydroxyethylcellulose, hydroxypropyl cellulose, hydroxypropylmethylcellulose, hydroxypropyl-guar, gellan gum, guar gum, xanthan gum and carboxymethylcellulose, as well as derivatives thereof, co-polymers thereof, and combinations thereof.

In a more preferred embodiment the localising agent is selected from the list consisting of fibrin, collagen, gelatin, amniotic membrane, fibrin glue, a combination of thrombin and fibrinogen, polyethylene (glycol) diacrylate (PEGDA), GelMA, localising agents comprising a polymer, cross-linked polymer, or hydrogel comprising one or more of hyaluronic acid, polyethylene glycol, polypropylene glycol, polyethylene oxide, polypropylene oxide, poloxamer, polyacrylic acid, poly(lactide-co-glycolide), alginate, gelatin, collagen, fibrinogen, hydroxypropylmethylcellulose and hydroxypropyl-guar, as well as derivatives thereof, co-polymers thereof, and combinations thereof.

In a preferred embodiment the expanded cell population according to the invention may be delivered to a recipient via a localising agent which is a biomatrix. In a more preferred embodiment the localising agent is a light curable, degradable biomatrix. Preferably this is able to be injected into the eye. A specific example of a biomatrix is GelMA, which is methacrylamide modified gelatin, and is also known as gelatin methacrylate.

GelMA may be prepared according to standard protocols known in the art (Van Den Bulcke et al., Biomacromolecules, 2000, p.31-38; Yue et al., Biomaterials, 2015, p.254-271). For example, gelatin from porcine skin (gel strength 300 g Bloom, Type A) is dissolved in PBS without calcium and magnesium (Dulbeccos PBS), and methacrylic anhydride may be added with strong agitation into the gelatin solution to reach the desired concentration (e.g., 8% (vol/vol). The mixture may be stirred before and after adding further DPBS. The diluted mixture may be purified via dialysis against Milli-Q water using dialysis tubing to remove methacrylic acid. The purified samples may optionally be lyophilized and the solid stored at -80° C., -20° C., or 4° C. until further use.

A GelMA stock solution is prepared by dissolving lyophilized GelMA in a formulation suitable for ocular use comprising pharmaceutically acceptable excipients. To prepare a GelMA stock solution, lyophilized GelMA may be dissolved in DPBS. After the GelMA is fully dissolved, a photoinitiator (for example such as lithium phenyl-2,4,6-trimethylbenzoylphosphinate) may be introduced into the GelMA solution. To adjust the pH to neutral, NaOH may be added to the solution before filtering using 0.22 micrometre sterile membranes. The final filtrate may be separated into aliquots and stored at 4° C. until further use.

In one aspect according to the invention, the cells are encapsulated within the biomatrix using a photoinitiator to polymerise the biomatrix, which is preferably GelMa. Suitable photoinitiator agents are Irgacure 2959, lithium phenyl-2,4,6-trimethylbenzoylphosphinate, sodium phenyl-2,4,6-trimethylbenzoylphosphinate, lithium bis(2,4,6-trimethylbenzoyl)phosphinate, sodium bis(2,4,6-trimethylbenzoyl)phosphinate, Diphenyl(2,4,6-trimethylbenzoyl)phosphine oxide, eosin Y, riboflavin phosphate, camphorquinone, Quantacure BPQ, Irgacure 819, Irgacure 1850, and Darocure 1173. In a preferred embodiment, the photoiniator is lithium phenyl-2,4,6-trimethylbenzoylphosphinate, sodium phenyl-2,4,6-trimethylbenzoylphosphinate, riboflavin phosphate. In another embodiment the photoinitiator is lithium phenyl-2,4,6-trimethylbenzoylphosphinate.

Prior to polymerization, the light curable biomatrix is combined with a suitable photoinitiator in a formulation suitable for ocular use comprising pharmaceutically acceptable excipients in suitable containers known in the art such as vials. The photoinitiator may be combined with the biomatrix prior to mixing with cells; alternatively the photoinitiator may be combined with the biomatrix after mixing with cells; alternatively the photoiniator may be added to the cells first, then combined with the biomatrix. The concentration of biomatrix and photoinitiator is dependent on the specific biomatrix and specific photoinitiator used, but is chosen to provide polymerization within a convenient light exposure duration, typically less than about 5 minutes; preferably less than about 2 minutes; more preferably less than about one minute. In one embodiment the photoinitiator is lithium phenyl-2,4,6-trimethylbenzoylphosphinate and its concentration in the formulation for cell delivery to the eye is about 0.01% w/v to about 0.15% w/v. In another aspect the lithium phenyl-2,4,6-trimethylbenzoylphosphinate concentration in the formulation for cell delivery to the eye is about 0.05% w/v or about 0.075% w/v. LAP may be synthesized using published procedure (Biomaterials 2009, 30, 6702-6707) and is also available from TCI (Prod. # L0290) and Biobots (BioKey).

The cells may be added to the GelMA in suitable containers known in the art such as vials or tubes. The cells may for example be added by pipetting into the GelMA and mixing by gentle pipetting up and down. In one embodiment the GelMA concentration in the composition suitable for ocular delivery is about 10 to about 200 mg/mL, or about 25 to about 150 mg/mL, or about 25 to about 75 mg/mL. In a preferred embodiment the GelMA concentration in the composition suitable for ocular delivery is about 25 mg/mL, about 50 mg/mL or about 75 mg/mL.

To polymerise the light curable biomatrix, the biomatrix, photoinitiator, and cells are exposed to a light source for a preferred duration, as described above. The wavelength of light used for polymerization will depend on the photochemical properties of the specific photoinitiator used. For example, photoinitation of polymerization for Irgacure 2959 will occur with light of wavelength between 300-370 nm; photoinitation of polymerization for lithium phenyl-2,4,6-trimethylbenzoylphosphinate will occur with light of wavelength between 300-420 nm; photoinitation of polymerization for riboflavin-5′-phosphate will occur with light of wavelength between 300-500 nm. The light source used may emit a range of wavelengths, like that achieved with incandescent lamps, gas discharge lamps, or metal vapor lamps; alternatively, the light source used may emit a narrow range of wavelengths, like that achieved with optical filters or with an light emitting diode (LED). Preferably, the light source used does not emit light with wavelength less than 315 nm to avoid the damaging effects of UV irradiation on cells. In one embodiment, the light source is a white light source with a spectral range of 415-700 nm. In another embodiment the light source is a LED light source with spectral range of about 365±5 nm, about 375±5 nm, about 385±5 nm, about 395±5 nm, about 405±5 nm, about 415±5 nm, about 425±5 nm, about 435±5 nm, about 445±5 nm, about 455±5 nm, or about 465±5 nm. The intensity of light is chosen to minimize phototoxicity and provide polymerization within a convenient light exposure duration, typically less than about 5 minutes; preferably less than about 2 minutes; more preferably less than about one minute. One indication of polymerization is an increase in solution viscosity. Another indication of polymerization is the onset of gelation.

The polymerization of the biomatrix may occur on the ocular surface via bioprinting techniques, or alternatively on a carrier that is then transplanted to the ocular surface. Optionally the polymerization of the biomatrix may occur on the cornea surface in the anterior chamber, or alternatively on a carrier that is then transplanted to the cornea surface in the anterior chamber.

### Carrier

The cells (e.g., modified LSCs) and localising agent suitable for ocular delivery are preferably delivered via a carrier such as a contact lens or amniotic membrane.

Contact lenses suitable for use according to the invention (e.g., for use with modified LSCs) are preferably those which conform to the patient’s corneal curvature and are able to be well tolerated by the patient in clinical practice for continuous use as bandage contact lenses for several days.

Examples of suitable types of contact lens according to the invention are consistent with what has been extensively validated in clinical use for long-term bandage contact lens use with Boston keratoprosthesis type 1 (which can be also used in patients with limbal stem cell deficiency) and described in: Thomas, Merina M.D.; Shorter, Ellen O.D.; Joslin, Charlotte E. O.D., Ph.D.; McMahon, Timothy J. O.D.; Cortina, M. Soledad M.D.Contact Lens Use in Patients With Boston Keratoprosthesis Type 1: Fitting, Management, and Complications. Eye Contact Lens. 2015 Nov;41 (6):334-40.

A contact lens can be of any appropriate material known in the art or later developed, and can be a soft lens, a hard lens, or a hybrid lens, preferably a soft lens, more preferably a conventional hydrogel contact lens or a silicone hydrogel (SiHy) contact lens.

A “conventional hydrogel contact lens” refers to a contact lens comprising a hydrogel bulk (core) material which is a water-insoluble, crosslinked polymeric material, is theoretically free of silicone, and can contain at least 10% by weight of water within its polymer matrix when fully hydrated. A conventional hydrogel contact lens typically is obtained by copolymerization of a conventional hydrogel lens formulation (i.e., polymerizable composition) comprising silicone-free, hydrophilic polymerizable components known to a person skilled in the art.

Examples of conventional hydrogel lens formulation for making commercial hydrogel contact lenses include, without limitation, alfafilcon A, acofilcon A, deltafilcon A, etafilcon A, focofilcon A, helfilcon A, helfilcon B, hilafilcon B, hioxifilcon A, hioxifilcon B, hioxifilcon D, methafilcon A, methafilcon B, nelfilcon A, nesofilcon A, ocufilcon A, ocufilcon B, ocufilcon C, ocufilcon D, omafilcon A, phemfilcon A, polymacon, samfilcon A, telfilcon A, tetrafilcon A, and vifilcon A.

A “SiHy contact lens” refers to a contact lens comprising a silicone hydrogel bulk (core) material which is a water-insoluble, crosslinked polymeric material containing silicone and can contains at least 10% by weight of water within its polymer matrix when fully hydrated. A silicone hydrogel contact lens typically is obtained by copolymerization of a silicone hydrogel lens formulation comprising at least silicone-containing polymerizable component and hydrophilic polymerizable components known to a person skilled in the art.

Examples of SiHy lens formulation for making commercial SiHy contact lenses include, without limitation, asmofilcon A, balafilcon A, comfilcon A, delefilcon A, efrofilcon A, enfilcon A, fanfilcon A, galyfilcon A, lotrafilcon A, lotrafilcon B, narafilcon A, narafilcon B, senofilcon A, senofilcon B, senofilcon C, smafilcon A, somofilcon A, and stenfilcon A.

In a preferred embodiment the carrier is a contact lens selected from the group consisting of Balafilcon A, Lotrafilcon A, Lotrafilcon B, Senofilcon A and methafilcon A.

In a particularly preferred embodiment the carrier is a contact lens, which is Lotrafilcon B.

The carrier may be held in place on the ocular surface using fibrin glue or sutures to prevent eye movements from dislodging the construct.

The carrier combined with biomatrix and cells may be left on the eye for a range of times in order to deliver the cells, for example a few days to one week, preferably one week.

### Other Delivery Methods:

In an alternative embodiment, the LSCs may be delivered as a cell suspension to the ocular surface (without a localising agent such as a biomatrix and with/or without a carrier such as a contact lens). Compounds and excipients known in the art to improve tissue adhesion such as mucoadhesive agents, viscosity enhancers, or reverse thermal gelators may be included in the formulation.

### Bioprinting Step

The population of ocular cells, e.g., corneal endothelial cells, obtainable according to the method of cell population expansion according to the invention may be grafted to the eye of a subject, e.g., to the cornea of a subject.

The cell population according to the invention may be delivered via a localising agent suitable for ocular use which is a light curable, degradable biomatrix such as GelMA. The following methods describe procedures for controlling the delivery to the inner wall of the cornea.

### Method 1. Bubble Depression Method

The dysfunctional endothelial cells may first be detached from the inner wall of the cornea by peeling/scraping or in a controlled manner using photodisruption with a femtosecond laser. A small bolus of the cell-laden biomatrix is then injected near the interior surface of the cornea. This may be done manually using a standard syringe or custom applicator. It can also be controlled through a surgical system (e.g., constellation) or syringe pumps. A gas bubble is then injected beneath the bolus. The gas bubble squeezes the bolus against the posterior cornea, creating a thin coating. The entire gel is then cured using a using a UV or near UV light source, or any other spectral band needed to cure the biomatrix. Alternatively, the dysfunctional tissue may be left, and the biomatrix cured over top of it. The light source can be focused into different sizes using other optical focusing methods to control the curing area. The remaining uncured area can be flushed out using irrigating/aspirating canula.

### Method 2. Subtractive Method Using Femtosecond Laser

The dysfunctional endothelial cells may first be detached from the inner wall of the cornea by peeling/scraping or in a controlled manner using photodisruption with a femtosecond laser. Alternatively, they may be left in place. The cell-laden biomatrix is then injected onto the interior surface of the cornea covering the void where tissue was removed or over the dysfunctional tissue. This may be done manually using a standard syringe or custom applicator. It can also be controlled through a surgical system (e.g. constellation) or syringe pumps. The biomatrix is then cured using a using a UV or near UV light source, or any other spectral band needed to cure the biomatrix. The femtosecond laser is then used to detach excess material, controlling the thickness and area to a desired distribution. The excess material is then removed with forceps through a corneal incision.

### Method 3. Stain Mask and Absorption Based Thickness Control

A biocompatible stain (Trypan Blue, Brilliant Blue, etc.) is firstly used to dye the inner surface of the cornea. The dysfunctional endothelial cells are then detached from the inner wall of the cornea by peeling/scraping. The cell-laden biomatrix containing the biocompatible stain is then injected onto the interior surface of the cornea covering the void where tissue was removed. The biomatrix is then cured using a using a UV or near UV light source, or any other spectral band needed to cure the biomatrix. The stain in the corneal tissue increases the light absorption acting as a mask to control the area of the cured biomatrix. Similarly, the stain in the biomatrix increases the absorption of light thereby controlling the depth/thickness of the cured material. Uncured gel material is then flushed from the anterior chamber using an irrigating/aspirating cannula.

### Method 4. Dry Anterior Chamber Application

The dysfunctional endothelial cells may first be detached from the inner wall of the cornea by peeling/scraping or in a controlled manner using photodisruption with a femtosecond laser. Alternatively it may be left in place. The anterior chamber of the anterior segment is then drained of aqueous and replaced with gas (e.g. air). The cell-laden biomatrix is then applied to interior surface of the cornea in small controlled droplets (allowing surface tension to disperse the drops), or painted using a brush or soft tip cannula. Hyaluronic acid may be applied to the biomatrix to alter its viscous properties and enable better control over dispensing/application. The entire biomatrix is then cured using a using a UV or near UV light source, or any other spectral band needed to cure the biomatrix. Finally, the anterior chamber is then filled again with balanced salt solution.

### Method 5. Naturally Buoyant Formulation

The dysfunctional endothelial cells may first be detached from the inner wall of the cornea by peeling/scraping or in a controlled manner using photodisruption with a femtosecond laser. A small bolus of the cell-laden biomatrix is then injected near the interior surface of the cornea. The biomatrix is formulated to be naturally buoyant relative to aqueous humor or aerated to achieve the same effect. This causes the biomatrix to naturally rise to posterior cornea, creating a thin coating. The entire biomatrix is then cured using a using a UV or near UV light source, or any other spectral band needed to cure the biomatrix. Alternatively, the dysfunctional tissue may be left, and the biomatrix cured over top of it. The UV light source can be focused into different sizes using optical focusing methods to control the curing area. The remaining uncured area can be flushed out using aspiration canula.

### Other Delivery Methods

In an alternative embodiment an expanded cell population, such as CECs as described herein, may be delivered as a cell suspension (without a localising agent such as a light curable, degradable biomatrix) and left to attach by gravity by having the patient look down for 3 hours. Compounds and excipients known in the art to improve tissue adhesion such as adhesive agents, viscosity enhancers, or reverse thermal gelators may be included in the formulation.

In yet another alternative embodiment an expanded cell population, such as CECs as described herein can also be delivered by using magnetic beads. A suspension of CECs/beads in a medium suitable for ocular delivery is prepared and this is then injected into the eye. Cell attachment is promoted by a magnet applied to the eye. (Magnetic field-guided cell delivery with nanoparticle-loaded human corneal endothelial cells. Moysidis SN, Alvarez-Delfin K, Peschansky VJ, Salero E, Weisman AD, Bartakova A, Raffa GA, Merkhofer RM Jr, Kador KE, Kunzevitzky NJ, Goldberg JL.Nanomedicine. 2015 Apr;11(3):499-509. doi: 10.1016/j.nano.2014.12.002.)

### Therapeutic Uses

The modified ocular cell or the ocular cell population according to the present disclosure (e.g., LSC, CEC, LSC population or CEC populiation) may be used in a method of treatment or prophylaxis of an ocular disease or disorder comprising administering to a subject in need thereof of a therapeutically effective amount of a cell population comprising ocular cells (e.g., LSCs or CECs).

The limbal stem cell population according to the invention (e.g., LSCs with reduced or eliminated expression of B2M by a CRISPR system, e.g., S. pyogenes Cas9 CRISPR system) may be used in a method of treatment or prophylaxis of an ocular disease or disorder comprising administering to a subject in need thereof of a therapeutically effective amount of a cell population comprising limbal stem cells. Preferably the ocular disease or disorder is associated with limbal stem cell deficiency.

Limbal stem cell deficiency may arise as a result of several diverse conditions including but not limited to:
direct stem cell damage from chemical or thermal burns or radiation injury;
congenital conditions such as aniridia, sclerocornea, multiple endocrine neoplasia;
autoimmune disorders such as Stevens Johnson syndrome or ocular cicatricial pemphigoid or collagen vascular diseases;
chronic non-auto-immune inflammatory disorders such as contact lens use, dry eye disease, rosacea, staph marginal, keratitis (bacterial, fungal & viral), pterygia or neoplasm;
iatrogenic, such as after multiple eye surgeries, excision of pterygia or neoplasm, cryotherapy;
as a result of medication toxicity such as preservatives (thimerosal, benzalkonium), topical anesthetics, pilocarpine, beta blockers, mitomycin, 5-fluorouracil, silver nitrate, and oral medications causing Stevens Johnson syndrome.
(See: Dry Eye: a practical guide to ocular surface disorders and stem cell surgery. SLACK 2006 - Rzany B, Mockenhaupt M, Baur S et al. J. Clin. Epidemiol. 49, 769-773 (1996)).

The most commonly encountered causes of limbal stem cell deficiency in clinical practice are chemical burns, aniridia, Stevens Johnson Syndrome and contact lens use.

More preferably the ocular disease or disorder is limbal stem cell deficiency which arises due an injury or disease or disorder selected from the group consisting of chemical burns, thermal burns, radiation injury, aniridia, sclerocornea, multiple endocrine neoplasia, Stevens Johnson syndrome, ocular cicatricial pemphigoid, collagen vascular diseases, chronic non-auto-immune inflammatory disorders arising from contact lens use, dry eye disease, rosacea, staph marginal, keratitis (including bacterial, fungal & viral keratitis), pterygia or neoplasm, limbal stem cell deficiency arising after multiple eye surgeries or excision of pterygia or neoplasm or cryotherapy; and limbal stem cell deficiency arising as a result of medication toxicity from a medication such as a medication selected from the group consisting of preservatives (e.g., thimerosal, benzalkonium), topical anaesthetics, pilocarpine, beta blockers, mitomycin, 5-fluorouracil, silver nitrate, and oral medications causing Stevens Johnson syndrome.

In a specific embodiment, the present invention provides a method of treating limbal stem cell deficiency by administering to a subject in need thereof an effective amount of a limbal stem cell population (e.g., limbal stem cell population with reduced or eliminated expression of B2M by a CRISPR system, e.g., S. pyogenes Cas9 CRISPR system) obtainable by the method of cell population expansion according to the invention.

In a more specific embodiment, the present invention provides a method of treating limbal stem cell deficiency which arises due an injury or disorder selected from the group consisting of chemical burns, thermal burns, radiation injury, aniridia, sclerocornea, multiple endocrine neoplasia, Stevens Johnson syndrome, ocular cicatricial pemphigoid, collagen vascular diseases, chronic non-auto-immune inflammatory disorders arising from contact lens use, dry eye disease, rosacea, staph marginal, keratitis (including bacterial, fungal & viral keratitis), pterygia or neoplasm, limbal stem cell deficiency arising after multiple eye surgeries, or excision of pterygia or neoplasm or cryotherapy; and limbal stem cell deficiency arising as a result of medication toxicity from a medication selected from the group consisting of preservatives (thimerosal, benzalkonium), topical anesthetics, pilocarpine, beta blockers, mitomycin, 5-fluorouracil, silver nitrate, and oral medications causing Stevens Johnson syndrome by administering to a subject in need thereof a therapeutically effective amount of a limbal stem cell population (e.g., limbal stem cell population with reduced or eliminated expression of B2M by a CRISPR system, e.g., S. pyogenes Cas9 CRISPR system) obtainable by the method of cell population expansion according to the invention.

In yet a more specific embodiment, the present invention provides a method of treating limbal stem cell deficiency which arises due an injury or disease or disorder selected from the group consisting of chemical burns, aniridia, Stevens Johnson Syndrome and contact lens use by administering to a subject in need thereof a thereapeutically effective amount of a limbal stem cell population (e.g., limbal stem cell population with reduced or eliminated expression of B2M by a CRISPR system, e.g., S. pyogenes Cas9 CRISPR system) obtainable by the method of cell population expansion according to the invention.

When an adult is a recipient (transplant recipient), in a particular embodiment, greater than 1,000 p63alpha expressing cells may be administered to a patient in the methods of treamtent according to the invention. In a particular embodiment, 1,000 to 100,000 p63alpha expressing cells may be administered to a patient in the methods of treatment according to the invention.

The corneal endothelial cell population (e.g., corneal endothelial cell population with reduced or eliminated expression of B2M by a CRISPR system, e.g., S. pyogenes Cas9 CRISPR system) according to the invention may be used in a method of treatment or prophylaxis of an ocular disease or disorder comprising administering to a subject in need thereof of a therapeutically effective amount of a cell population comprising corneal endothelial cells. Preferably the ocular disease or disorder is associated with decreased corneal endothelial cell density. In a preferred embodiment the ocular disease or disorder is corneal endothelial dysfunction.

More preferably the ocular disease or disorder is corneal endothelial dysfunction which is selected from the group consisting of Fuchs endothelial corneal dystrophy, bullous keratopathy (including pseudophakic bullous keratopathy and aphakic bullous keratopathy), corneal transplant failure, posterior polymorphous corneal dystrophy, congenital hereditary endothelial dystrophy, X-linked endothelial corneal dystrophy, aniridia, and corneal endothelitis. In a specific embodiment the ocular disease or disorder is selected from the group consisting of Fuchs endothelial corneal dystrophy, bullous keratopathy (including pseudophakic bullous keratopathy and aphakic bullous keratopathy) and corneal transplant failure.

In a specific embodiment, the present invention provides a method of treating corneal endothelial dysfunction by administering to a subject in need thereof an effective amount of a corneal endothelial cell population (e.g., corneal endothelial cell population with reduced or eliminated expression of B2M by a CRISPR system, e.g., S. pyogenes Cas9 CRISPR system) obtainable by the method of cell population expansion according to the invention.

In a more specific embodiment, the present invention provides a method of treating corneal endothelial dysfunction which is selected from the group consisting of Fuchs endothelial corneal dystrophy, bullous keratopathy (including pseudophakic bullous keratopathy and aphakic bullous keratopathy), corneal transplant failure, posterior polymorphous corneal dystrophy, congenital hereditary endothelial dystrophy, X-linked endothelial corneal dystrophy, aniridia, and corneal endothelitis by administering to a subject in need thereof an effective amount of a corneal endothelial cell population (e.g., corneal endothelial cell population with reduced or eliminated expression of B2M by a CRISPR system, e.g., S. pyogenes Cas9 CRISPR system) obtainable by the method of cell population expansion according to the invention.

In yet a more specific embodiment, the present invention provides a method of treating corneal endothelial dysfunction selected from the group consisting of Fuchs endothelial corneal dystrophy, bullous keratopathy (including pseudophakic bullous keratopathy and aphakic bullous keratopathy) and corneal transplant failure by administering to a subject in need thereof an effective amount of a corneal endothelial cell population (e.g., corneal endothelial cell population with reduced or eliminated expression of B2M by a CRISPR system, e.g., S. pyogenes Cas9 CRISPR system) obtainable by the method of cell population expansion according to the invention.

When an adult is a recipient (transplant recipient), in particular aspects, the corneal endothelial cell population (e.g., corneal endothelial cell population with reduced or eliminated expression of B2M by a CRISPR system, e.g., S. pyogenes Cas9 CRISPR system) for use in the method of treatment according to the invention preferably has a final cell density in the eye of about at least 500 cells/mm² (area), preferably 1,000 to 3,500 cells/mm² (area), more preferably 2,000 to about 4,000 cells/mm² (area).

In certain embodiments, a patient’s vision is improved by a method of treatment provided herein. Visual acuity tests are well known in the art, including, for example the Snellen and Sloan acuity tests, and Early Treatment Diabetic Retinopathy Study (ETDRS) acuity test. An improvement in vision can be measured, for example, using a best corrected visual acuity (BCVA) measurement. In certain embodiments, the BCVA of a patient treated as provided herein improves by at least 1, 2, 3, 4, 5 or more lines as measured by ETDRS letters following treatment with a modified cell or cell population or composition of the invention as provided herein.

### EXAMPLES

The following examples are provided to further illustrate the invention but not to limit its scope. Other variants of the invention will be readily apparent to one of ordinary skill in the art and are encompassed by the appended claims.

Unless defined otherwise, the technical and scientific terms used herein have the same meaning as that usually understood by a specialist familiar with the field to which the disclosure belongs.

### Example 1: Human Limbal Epithelial Cell Isolation

Research-consented cadaveric human corneas were obtained from eye banks. Limbal rims were dissected and partially dissociated in a 1.2 mg/ml dispase solution for 2 hours at 37° C. followed by 10 minutes in TrypLE (Life Technologies). Pieces of limbal crypts were then carefully cut out of the partially dissociated limbal rims and rinsed by centrifugation). Cells obtained in this manner were used in the Examples below.

### Example 2: Exposure of Cells to LATS Inhibitors and Measurement of Intracellular YAP distribution

Cells obtained as described in Example 1 were plated in glass-bottom black wall 24-well dishes in limbal epithelium cell culture medium (DMEM F12 supplemented with 10% human serum and 1.3 mM calcium chloride) supplemented with LATS inhibitor compound example no. 4 or 3 at a concentration of 10 micromolar or supplemented in DMSO as a negative control. Cells were cultured under these conditions for 24 hours at 37° C. in 5% CO2.

To measure the effect of the LATS inhibitors on the downstream target YAP, intracellular YAP distribution was analyzed by immunohistochemistry. Cell cultures were fixed with 4% PFA for 20 minutes, permeabilized and blocked in a blocking solution of 0.3% Triton X-100 (Sigma-Aldrich) and 3% donkey serum in PBS for 30 minutes. Cells were then labeled with primary antibody in the blocking solution for 12 hours at 4° C. Primary antibody used was anti-YAP from Santa Cruz Biotechnology. Samples were washed in PBS three times and donkey-raised secondary antibody Alexa Fluor 488 (Molecular Probes) at 1:500 dilution were applied for 30 minutes at room temperature. Negative control was omitted primary antibody (data not shown). Fluorescence was observed using a Zeiss LSM 880 confocal microscope.

Only weak YAP immunostaining was observed in the nucleus of LSCs cultured without the LATS inhibitors (DMSO control). YAP immunostaining was stronger in the nucleus of LSCs exposed to the LATS inhibitor compound 2-(3-methyl-1H-pyrazol-4-yl)-N-(1-methylcyclopropyl)pyrido[3,4-d]pyrimidin-4-amine or 2,4-dimethyl-4-{[2-(pyridin-4-yl)pyrido[3,4-d]pyrimidin-4-yl]amino}pentan-2-ol prepared as described in U.S. Pat. Application No. 15/963,816 and International Application No. PCT/IB2018/052919 (WO 2018/198077), filed Apr. 26, 2018 (data not shown).

### Example 3: Exposure of Cells to LATS Inhibitors and Measurement of YAP Phosphorylation

Cells obtained as described in Example 1 were detached from the culture dish with Accutase for 10 minutes at 37° C., cell suspensions were rinsed by centrifugation and plated in DMEM F12 supplemented with 10% human serum and 1.3 mM calcium chloride in 6-well plates (Corning) and cultured without LATS inhibitor compounds for 2-4 days.

The medium was then replaced by fresh limbal epithelium cell culture medium (DMEM F12 supplemented with 10% human serum and 1.3 mM calcium chloride) supplemented with LATS inhibitor compound example no. 4 or 3 at a concentration of 10 micromolar or supplemented in DMSO as a negative control. Cells were cultured under these conditions for 1 hour at 37° C. in 5% CO2.

To measure the effect of the LATS inhibitors on the downstream target YAP, the YAP phosphorylation levels were measured by western blot as follows. The cell pellets were obtained by trypsin dissociation and centrifugation and washed with PBS. The pellets were lysed with 30 microlitres of RIPA lysis buffer containing protease inhibitor cocktail (Life Technologies) for 30 minutes, with vortexing every 10 minutes. The cell debris were then pelleted at 4° C. for 15 minutes at 14 k rpm and the protein lysate was collected. Protein concentration was quantified using a micro BCA kit (Pierce). Fifteen micrograms of total protein was loaded in each well of 4-20% TGX gels (BioRad) and Western blotting was performed according to the manufacturer’s instructions. Membranes were probed with phospho-YAP (ser127) (CST, 1:500) or total Yap (Abnova, 1:500) antibody and actin (Abcam) labelling was used as loading control. Membranes were stained with HRP-conjugated secondary antibodies, rinsed and imaged using a ChemiDoc system (Biorad) according to the manufacturer’s instructions.

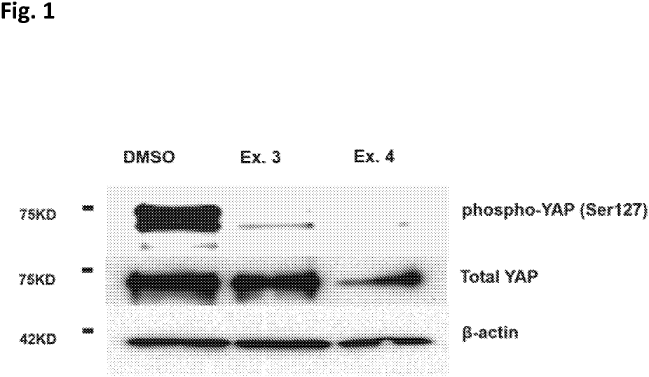

Western blot analysis (see FIG. 1) showed that both compound example no. 4 and 3 caused a reduction in YAP phosphorylation levels in human LSCs.These results suggest that the LATS inhibitor compound example no. 4 and 3 can activate YAP signaling in human LSCs.

### Example 4: Human Limbal Stem Cell Population Expansion and Immunohistochemical Observation of Cellular Phenotype

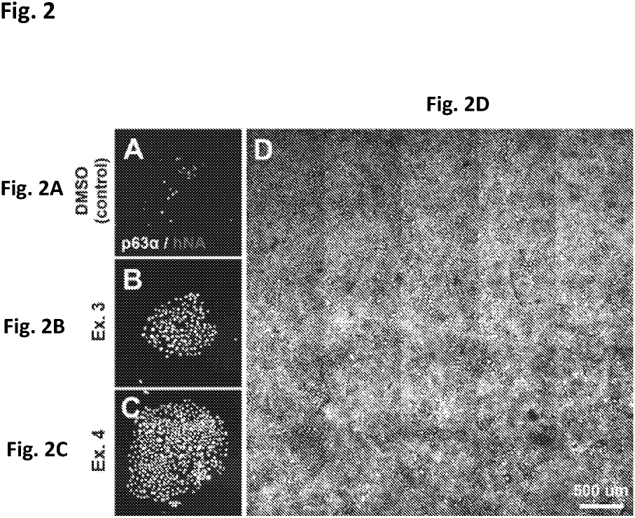

Cells obtained as described in Example 1 were plated in 24-well plates (Corning) in limbal epithelium cell culture medium (DMEM F12 supplemented with 10% human serum and 1.3 mM calcium chloride) supplemented with LATS inhibitor compound example no. 4 or 3 at a concentration of 10 micromolar or supplemented in DMSO as a negative control. Cells were first cultured at 37° C. in 5% CO2 for 6 days after isolation without passaging (FIGS. 2A, 2B and 2C).

To evaluate the ability of the compounds to enable LSC expansion after two passages, LSCs were passaged and cultured for two weeks in the presence of compound example 3 to enable expansion (FIG. 2D). Limbal stem cells (LSCs) were passaged by treating cultures with Accutase for 10 minutes at 37° C., rinsing the cell suspension by centrifugation and plating cells in fresh LSC culture medium supplemented with LATS inhibitor compound example 3.

In order to observe that the expanded cell population expressed p63alpha, this was measured by immunohistochemistry as follows. Cell cultures were fixed with 4% PFA for 20 minutes, permeabilized and blocked in a blocking solution of 0.3% Triton X-100 (Sigma-Aldrich) and 3% donkey serum in PBS for 30 minutes. Cells were then labeled with primary antibody in the blocking solution for 12 hours at 4° C. Primary antibody used was p63alpha from Cell Signalling. Samples were washed in PBS three times and donkey-raised secondary antibody Alexa Fluor 488 (Molecular Probes) at 1:500 dilution were applied for 30 minutes at room temperature. Cells were counter-stained with a human nuclear antigen antibody (Millipore) at a 1:500 dilution in order to label all cells in the culture and confirm their human identity. Negative control was omitted primary antibody (data not shown). Fluorescence was observed using a Zeiss LSM 880 confocal microscope.

FIG. 2A shows that in the presence of growth medium and DMSO, only a few isolated cells attach to the culture dish and survive up to 6 days. Most cells expressed the human nuclear marker, but few expressed p63alpha. In contrast, in the presence of LATS inhibitors compound example no. 4 (FIG. 2B) and compound example no. 3 (FIG. 2C), the cells formed colonies and expressed p63alpha. This result indicated that the LATS inhibitors promote the expansion of the population of cells with the p63alpha-positive phenotype. FIG. 2D: Passaging cells and culturing them in the presence of LATS inhibitor compound example no. 3 for two weeks enabled cell population expansion and the formation of confluent cultures expressing p63alpha.

### Example 5: Human Limbal Stem Cell Population Expansion and Measurement Thereof

Cells obtained as described in Example 1 were plated in 48-well plates (Corning) in XVIVO15 medium (Lonza) supplemented with LATS inhibitors (as listed in Table 2 and 3 below) at a concentration of 10 micromolar or supplemented in DMSO as a negative control. Cells were cultured at 37° C. in 5% CO2.

For each compound, two sets of cultures were generated. A first set of cultures was fixed in 4% PFA for 20 minutes at room temperature after cells isolated from the cornea had attached to the cell culture dish (typically 24 h after cell plating). A second set of cultures was fixed in 4% PFA for 20 minutes at room temperature after being cultured for two passages. Cells were passaged when they reached 90-100% confluence.

In order to observe that the expanded cell population expressed p63alpha, this was measured by immunohistochemistry as follows. The fixed cell cultures were permeabilized and blocked in a blocking solution of 0.3% Triton X-100 (Sigma-Aldrich) and 3% donkey serum in PBS for 30 minutes. Cells were then labeled with primary antibody in the blocking solution for 12 hours at 4° C. Primary antibody used was p63alpha from Cell Signalling. Samples were washed in PBS three times and donkey-raised secondary antibody Alexa Fluor 488 (Molecular Probes) at 1:500 dilution were applied for 30 minutes at room temperature. Cell nuclei were then labeled in a solution of 0.5 micromolar of Sytox Orange (ThermoFisher) in PBS for 5 minutes at room temperature.

To evaluate the percentage of p63alpha-positive cells, the number of cells labeled by the anti-p63alpha antibody was counted and the total number of cells was determined by counting the number of nuclei stained by Sytox Orange. The proportion of p63alpha-positive cells was then determined by calculating the percentage of Sytox-orange-positive nuclei that also expressed p63alpha.

To evaluate cell expansion ratios, nuclei were counted using a Zeiss LSM 880 confocal microscope. The expansion factor was then determined by calculating the ratio of the expanded population of cells to population of seeded cells.

Results in the Tables below indicate that the LATS inhibitors enabled cell population expansion. In the presence of the LATS inhibitors, 57 to 97 percent of the cells express the p63alpha-positive phenotype.

In addition to the compounds listed in Table 2, the LATS inhibitor dimethyl(3-methyl-3-{[2-(pyridin-4-yl)pyrido[3,4-d]pyrimidin-4-yl]amino}butyl)amine was demonstarted to enable limbal stem cell population expansion.

TABLE 2

| Compound | Expansion factor | Compound Example No. | Expansio n factor |
|---|---|---|---|
| N-methyl-2-(pyridin-4-yl)-N-(1,1,1-trifluoropropan-2-yl)pyrido[3,4-d]pyrimidin-4-amine | 2137 | 2-(pyridin-4-yl)-4-(3-(trifluoromethyl)piperazi n-1-yl)pyrido[3,4-d]pyrimidine | 1051 |
| 2-methyl-1-(2-methyl-2-{[2-(pyridin-4-yl)pyrido[3,4-d]pyrimidin-4-yllamino}propoxy)propan-2-ol | 2087 | N-cyclopentyl-2-(pyridin-4-yl)pyrido[3,4-d]pyrimidin-4-amine | 1048 |
| 2,4-dimethyl-4-{[2-(pyridin-4-yl)pyrido[3,4-d]pyrimidin-4-yl]amino}pentan-2-ol(Ex. 3) | 2029 | N-propyl-2-(3-(trifluoromethyl)-1 H-pyrazol-4-yl)pyrido[3,4-d]pyrimidin-4-amine | 991 |
| N-(tert-butyl)-2-(pyridin-4-yl)-1,7-naphthyridin-4-amine | 1717 | N-(2-methylcyclopentyl)-2-(pyridin-4-yl)pyrido[3,4-d]pyrimidin-4-amine | 976 |
| 2-(pyridin-4-yl)-N-[1-(trifluoromethyl)cyclobutyl]pyrido[3, 4-d]pyrimidin-4-amine | 1712 | 2-(3-chloropyridin-4-yl)-N-(1,1,1-trifluoro-2-methylpropan-2-yl)pyrido[3,4-d]pyrimidin-4-amine | 961 |
| N-propyl-2-(pyridin-4-yl)pyrido[3,4-d]pyrimidin-4-amine | 1423 | 2-(2-methyl-2-{[2-(pyridin-4-yl)pyrido[3,4-d]pyrimidin-4-yl]amino}propoxy)ethan-1-ol | 705 |
| N-(propan-2-yl)-2-(pyridin-4-yl)pyrido[3,4-d]pyrimidin-4-amine | 1275 | N-(1-methylcyclopropyl)-7-(pyridin-4-yl)isoquinolin-5-amine | 681 |
| 3-(pyridin-4-yl)-N-(1-(trifluoromethyl)cyclopropyl)-2,6-naphthyridin-1-amine | 1241 | (1S,2S)-2-{[2-(pyridin-4-yl)pyrido[3,4-d]pyrimidin-4-yl]amino}cyclopentan-1-ol | 39 |
| 2-(3-methyl-1 H-pyrazol-4-yl)-N-(1-methylcyclopropyl)pyrido[3,4-d]pyrimidin-4-amine (Ex. 4) | 1205 | DMSO | 35 |
| 2-methyl-2-{[2-(pyridin-4-yl)pyrido[3,4-d]pyrimidin-4-yl]amino}propan-1-ol | 1160 | | |

TABLE 3

| Compound | Percentage of p63a-positive cells | Compound Example No. | Percentag e of p63a-positive cells |
|---|---|---|---|
| N-methyl-2-(pyridin-4-yl)-N-(1,1,1-trifluoropropan-2-yl)pyrido[3,4-d]pyrimidin-4-amine | 97 | 2-(pyridin-4-yl)-4-(3-(trifluoromethyl)piperazi n-1-yl)pyrido[3,4-d]pyrimidine | 90 |
| 2-methyl-1-(2-methyl-2-{[2-(pyridin-4-yl)pyrido[3,4-d]pyrimidin-4-yllamino}propoxy)propan-2-ol | 95 | N-cyclopentyl-2-(pyridin-4-yl)pyrido[3,4-d]pyrimidin-4-amine | 87 |
| 2,4-dimethyl-4-{[2-(pyridin-4-yl)pyrido[3,4-d]pyrimidin-4-yllamino}pentan-2-ol (Ex. 3) | 92 | N-propyl-2-(3-(trifluoromethyl)-1 H-pyrazol-4-yl)pyrido[3,4-d]pyrimidin-4-amine | 86 |
| N-(tert-butyl)-2-(pyridin-4-yl)-1,7-naphthyridin-4-amine | 93 | N-(2-methylcyclopentyl)-2- | 86 |
| | | (pyridin-4-yl)pyrido[3,4-d]pyrimidin-4-amine | |
| 2-(pyridin-4-yl)-N-[1-(trifluoromethyl)cyclobutyl]pyrido[3,4-d]pyrimidin-4-amine | 95 | 2-(3-chloropyridin-4-yl)-N-(1,1,1-trifluoro-2-methylpropan-2-yl)pyrido[3,4-d]pyrimidin-4-amine | 87 |
| N-propyl-2-(pyridin-4-yl)pyrido[3,4-d]pyrimidin-4-amine | 93 | 2-(2-methyl-2-{[2-(pyridin-4-yl)pyrido[3,4-d]pyrimidin-4-yl]amino}propoxy)ethan -1-ol | 86 |
| N-(propan-2-yl)-2-(pyridin-4-yl)pyrido[3,4-d]pyrimidin-4-amine | 93 | N-(1-methylcyclopropyl)-7-(pyridin-4-yl)isoquinolin-5-amine | 80 |
| 3-(pyridin-4-yl)-N-(1-(trifluoromethyl)cyclopropyl)-2,6-naphthyridin-1-amine | 95 | (1S,2S)-2-{[2-(pyridin-4-yl)pyrido[3,4-d]pyrimidin-4-yl]amino}cyclopentan-1-ol | 6 |
| 2-(3-methyl-1 H-pyrazol-4-yl)-N-(1-methylcyclopropyl)pyrido[3,4-d]pyrimidin-4-amine (Ex. 4) | 89 | DMSO | 3 |
| 2-methyl-2-{[2-(pyridin-4-yl)pyrido[3,4-d]pyrimidin-4-yllamino}propan-1-ol | 89 | | |

### Example 6: Reducing Immune Rejection by CRISPR/Cas9-Mediated Deletion of the Beta-2-Microglobulin Gene in HEK293

In the example below, HLA class I expression was eliminated from the HEK293 surface by CRISPR-mediated deletion of the beta-2-microglobulin gene.

Guide RNA (gRNA) targeting B2M were obtained from Dharmacon (Layfette, CO) (sequences 1-5 in Table 4). Seven additional gRNA were also designed (6-12 in Table 4). Table 5 shows the PAM sequence for each gRNA ID, the target sequence location, the sequence of B2M gene that corresponds to the gRNA targeting domain and is complementary to the target sequence in the B2M gene. Table 6 represents sequences of sgRNAs. These gRNAs (SEQ ID NO 108-119) were tested for the ability to reduce or eliminate expression of B2M in HEK293 cells using a lipofection approach as follows

TABLE 4

| gRNA ID | SEQ ID NO: | Sequence of the targeting domain of the gRNA |
|---|---|---|
| 1-CR004366 | 108 | GAGUAGCGCGAGCACAGCUA |
| 2-CR004366 | 109 | CGUGAGUAAACCUGAAUCUU |
| 3-CR004366 | 110 | AAGUCAACUUCAAUGUCGGA |
| 4-CR004366 | 111 | CAGUAAGUCAACUUCAAUGU |
| 5-CR004366 | 112 | CUGAAUCUUUGGAGUACCUG |
| 6-HEYJA000001 | 113 | GGCCGAGAUGUCUCGCUCCG |
| 7-HEYJA000003 | 114 | CUCGCGCUACUCUCUCUUUC |
| 8-HEYJA000004 | 115 | ACUCACGCUGGAUAGCCUCC |
| 9-HEYJA000005 | 116 | UCACGUCAUCCAGCAGAGAA |
| 10-HEYJA000007 | 117 | AGUCACAUGGUUCACACGGC |
| 11-H EYJA000008 | 118 | CCACCUCUUGAUGGGGCUAG |
| 12-HEYJA000009 | 119 | GCUACUCUCUCUUUCUGGCC |
| CR000442 | 134 | GGCCACGGAGCGAGACAUCU |
| CR000443 | 135 | CGCGAGCACAGCUAAGGCCA |
| CR000446 | 136 | AGGGUAGGAGAGACUCACGC |
| CR000453 | 137 | CACAGCCCAAGAUAGUUAAG |
| CR000455 | 138 | UUACCCCACUUAACUAUCUU |
| CR000456 | 139 | CUUACCCCACUUAACUAUCU |
| CR006979 | 140 | UCCUGAAUUGCUAUGUGUCU |

TABLE 5

| gRNA ID | PAM | Target Sequence Location | Sequence of B2M gene that corresponds to gRNA targeting domain | SEQ ID NO of sequence of B2M gene that correspond s to gRNA targeting domain |
|---|---|---|---|---|
| 1-CR004366 | AGG | chr15:44711563 -44711585 | GAGTAGCGCGAGCACAGCT A | 141 |
| 2-CR004366 | TGG | chr15:44715428 -44715450 | CGTGAGTAAACCTGAATCTT | 142 |
| 3-CR004366 | TGG | chr15:44715509 -44715531 | AAGTCAACTTCAATGTCGGA | 143 |
| 4-CR004366 | CGG | chr15:44715513 -44715535 | CAGTAAGTCAACTTCAATGT | 144 |
| 5-CR004366 | AGG | chr15:44715417 -44715439 | CTGAATCTTTGGAGTACCTG | 145 |
| 6-HEYJA000001 | TGG | chr15:44711540 -44711562 | GGCCGAGATGTCTCGCTCC G | 146 |
| 7-HEYJA000003 | TGG | chr15:44711574 -44711596 | CTCGCGCTACTCTCTCTTTC | 147 |
| 8-HEYJA000004 | AGG | chr15:44711597 -44711619 | ACTCACGCTGGATAGCCTC C | 148 |
| 9-HEYJA000005 | TGG | chr15:44715446 -44715468 | TCACGTCATCCAGCAGAGA A | 149 |
| 10-HEYJA000007 | AAG | chr15:44715651 -44715673 | AGTCACATGGTTCACACGG C | 150 |
| 11-HEYJA000008 | TAG | chr15:44713812 -44713834 | CCACCTCTTGATGGGGCTA G | 151 |
| 12-HEYJA000009 | TGG | chr15:44711579 -44711601 | GCTACTCTCTCTTTCTGGCC | 152 |
| CR000442 | CGG | chr15:44711542 -44711564 | GGCCACGGAGCGAGACATC T | 153 |
| CR000443 | CGG | chr15:44711557 -44711579 | CGCGAGCACAGCTAAGGCC A | 154 |
| CR000446 | TGG | chr15:44711609 -44711631 | AGGGTAGGAGAGACTCACG C | 155 |
| CR000453 | TGG | chr15:44715678 -44715700 | CACAGCCCAAGATAGTTAA G | 156 |
| CR000455 | GGG | chr15:44715683 -44715705 | TTACCCCACTTAACTATCTT | 157 |
| CR000456 | TGG | chr15:44715684 -44715706 | CTTACCCCACTTAACTATCT | 158 |
| CR006979 | GGG | chr15:44715480 -44715502 | TCCTGAATTGCTATGTGTCT | 159 |

PAM=Protospacer adjacent Motif; gRNAs 1-5 from Dharmacon

TABLE 6

| gRNA ID | SEQ ID NO: | Sequence of the sgRNA |
|---|---|---|
| 1-CR004366 | 120 | GAGUAGCGCGAGCACAGCUAGUUUUAGAG CUAGAAAUAGCAAGUUAAAAUAAGGCUAGU CCGUUAUCAACUUGAAAAAGUGGCACCGA GUCGGUGCUUUU |
| 2-CR004366 | 160 | CGUGAGUAAACCUGAAUCUUGUUUUAGAG CUAGAAAUAGCAAGUUAAAAUAAGGCUAGU CCGUUAUCAACUUGAAAAAGUGGCACCGA GUCGGUGCUUUU |
| 3-CR004366 | 161 | AAGUCAACUUCAAUGUCGGAGUUUUAGAG CUAGAAAUAGCAAGUUAAAAUAAGGCUAGU CCGUUAUCAACUUGAAAAAGUGGCACCGA GUCGGUGCUUUU |
| 4-CR004366 | 162 | CAGUAAGUCAACUUCAAUGUGUUUUAGAG CUAGAAAUAGCAAGUUAAAAUAAGGCUAGU CCGUUAUCAACUUGAAAAAGUGGCACCGA GUCGGUGCUUUU |
| 5-CR004366 | 163 | CUGAAUCUUUGGAGUACCUGGUUUUAGAG CUAGAAAUAGCAAGUUAAAAUAAGGCUAGU CCGUUAUCAACUUGAAAAAGUGGCACCGA GUCGGUGCUUUU |
| 6-HEYJA000001 | 164 | GGCCGAGAUGUCUCGCUCCGGUUUUAGA GCUAGAAAUAGCAAGUUAAAAUAAGGCUA GUCCGUUAUCAACUUGAAAAAGUGGCACC GAGUCGGUGCUUUU |
| 7-HEYJA000003 | 165 | CUCGCGCUACUCUCUCUUUCGUUUUAGAG CUAGAAAUAGCAAGUUAAAAUAAGGCUAGU CCGUUAUCAACUUGAAAAAGUGGCACCGA GUCGGUGCUUUU |
| 8-HEYJA000004 | 166 | ACUCACGCUGGAUAGCCUCCGUUUUAGAG CUAGAAAUAGCAAGUUAAAAUAAGGCUAGU CCGUUAUCAACUUGAAAAAGUGGCACCGA GUCGGUGCUUUU |
| 9-HEYJA000005 | 167 | UCACGUCAUCCAGCAGAGAAGUUUUAGAG CUAGAAAUAGCAAGUUAAAAUAAGGCUAGU CCGUUAUCAACUUGAAAAAGUGGCACCGA GUCGGUGCUUUU |
| 10-HEYJA000007 | 168 | AGUCACAUGGUUCACACGGCGUUUUAGAG CUAGAAAUAGCAAGUUAAAAUAAGGCUAGU CCGUUAUCAACUUGAAAAAGUGGCACCGA GUCGGUGCUUUU |
| 11-HEYJA000008 | 169 | CCACCUCUUGAUGGGGCUAGGUUUUAGAG CUAGAAAUAGCAAGUUAAAAUAAGGCUAGU CCGUUAUCAACUUGAAAAAGUGGCACCGA GUCGGUGCUUUU |
| 12-HEYJA000009 | 170 | GCUACUCUCUCUUUCUGGCCGUUUUAGAG CUAGAAAUAGCAAGUUAAAAUAAGGCUAGU CCGUUAUCAACUUGAAAAAGUGGCACCGA GUCGGUGCUUUU |
| CR000442 | 171 | GGCCACGGAGCGAGACAUCUGUUUUAGAG CUAGAAAUAGCAAGUUAAAAUAAGGCUAGU CCGUUAUCAACUUGAAAAAGUGGCACCGA GUCGGUGCUUUU |
| CR000443 | 172 | CGCGAGCACAGCUAAGGCCAGUUUUAGAG CUAGAAAUAGCAAGUUAAAAUAAGGCUAGU CCGUUAUCAACUUGAAAAAGUGGCACCGA GUCGGUGCUUUU |
| CR000446 | 173 | AGGGUAGGAGAGACUCACGCGUUUUAGAG CUAGAAAUAGCAAGUUAAAAUAAGGCUAGU |
| | | CCGUUAUCAACUUGAAAAAGUGGCACCGA GUCGGUGCUUUU |
| CR000453 | 174 | CACAGCCCAAGAUAGUUAAGGUUUUAGAG CUAGAAAUAGCAAGUUAAAAUAAGGCUAGU CCGUUAUCAACUUGAAAAAGUGGCACCGA GUCGGUGCUUUU |
| CR000455 | 175 | UUACCCCACUUAACUAUCUUGUUUUAGAG CUAGAAAUAGCAAGUUAAAAUAAGGCUAGU CCGUUAUCAACUUGAAAAAGUGGCACCGA GUCGGUGCUUUU |
| CR000456 | 176 | CUUACCCCACUUAACUAUCUGUUUUAGAG CUAGAAAUAGCAAGUUAAAAUAAGGCUAGU CCGUUAUCAACUUGAAAAAGUGGCACCGA GUCGGUGCUUUU |
| CR006979 | 177 | UCCUGAAUUGCUAUGUGUCUGUUUUAGAG CUAGAAAUAGCAAGUUAAAAUAAGGCUAGU CCGUUAUCAACUUGAAAAAGUGGCACCGA GUCGGUGCUUUU |

### Lipofection:

One day before transfection, 500′000 HEK293cells (ATCC, Manassas, VA) were plated in a 35 mm dish and grown in DMEM/10%FBS. The following day, cells were transfected with a mixture of tracrRNA-gRNA-Cas9 mRNA. A stock of 10 micromolar was prepared resuspending 20 nanomoles of gRNA and 20 nanomoles tracrRNA in 2000 microlitre 10 millimolar Tris buffer pH7.4 each. Additionally, Cas9 mRNA was pre-diluted 1:10 adding 10 microlitre of 1 microgram/microlitre Cas9 mRNA to 90 microlitre 10 millimolar Tris buffer pH7.4.

To obtain the mixture for the size of a 35 mm petri dish, 12.5 microlitre of 10 micromolar tracrRNA (Dharmacon, Cat # U-002000-20), 12.5 microlitre of 10 micromolar gRNA targeting human B2M (Table 4; SEQ ID NOs: 108-119), 50 microlitre of 0.1 microgram/microlitre Cas9 mRNA (Dharmacon, Cat #CAS11195), and 15 microlitre of DharmaFECT Duo Transfection Reagent (Dharmacon, Cat #T-2010-02) were combined and incubated for 20 minutes at room temperature. The mixture was added drop wise to the culture dish in 2.5 ml DMEM/10%FBS medium. The transfection reagent alone represented the transfection negative control.

After 6 h incubation in 5% CO₂ at 37° C. medium was replaced with fresh DMEM/10%FBS medium. After 72 h in a 5% CO2 incubator cells were prepared for FACS analysis. FACS analysis: HEK293 cells were treated with Accutase (ThermoFisher, Cat # A1110501) for 20 minutes in 5% CO2 at 37° C. The reaction was stopped by using cell culture medium containing 10% Serum and transferred to a falcon tube for a centrifugation step (1000 rpm, 5 minutes). After aspirating the medium cells were resuspended in 200 microlitre FACS buffer (PBS/10%FBS).

To analyze the expression of B2M and HLA-ABC, 5 microlitre APC mouse anti-human β2-microglobulin antibody (Biolegend, Cat #316312) and 20 microlitre PE mouse anti-human HLA-ABC antibody (BD Bioscience, Cat # 560168) were added to the cell suspension and incubated for 30 minutes on ice. Cells were washed 3 times after antibody labelling with FACS buffer and resuspended in 500 microlitre in FACS buffer.

Each sample was transferred to one well of a round bottom 96 well plate and analyzed on a BD LSRFortessa X-20 device. FACS data were analyzed using BD FACSDiva software.

The results are shown in Table 7 below.

TABLE 7

| gRNA ID | B2M knockout efficiency in HEK293 [%) |
|---|---|
| 1-CR004366 | 70 |
| 2-CR004366 | 33 |
| 3-CR004366 | 12 |
| 4-CR004366 | 48 |
| 5-CR004366 | 33 |
| 6-HEYJA000001 | 47 |
| 7-HEYJA000003 | 37 |
| 8-HEYJA000004 | 65 |
| 9-HEYJA000005 | 39 |
| 10-HEYJA000007 | 13 |
| 11-HEYJA000008 | 3 |
| 12-HEYJA000009 | 17 |

### Example 7: Reducing Immune Rejection by CRISPR/Cas9-mediated Deletion of the Beta-2-Microglobulin Gene in LSCs

In the example below, HLA class I expression was eliminated from the LSC surface by CRISPR-mediated deletion of the beta-2-microglobulin gene.

The sgRNA ID SEQ NO 120 was tested for the ability to reduce or eliminate expression of B2M in LSCs using a nucleofection approach as follows.

### Nucleofection:

LSCs at passage 0 were trypsinized with TryLE®Express Enzym (ThermoFisher, Cat #12605010) for 15 min in 5%CO2 at 37° C. After scraping the cells, the reaction was stopped by using cell culture medium containing 10% Serum and transferred to a falcon tube. After counting cells using Vi-cell 200′000 cells were prepared per reaction by transferring 200′000 cells in single tubes and centrifuged at 1000 rpm for 5 min.

The supernatant was aspirated using manually pipetting to avoid cell loss and the cells were resuspended in the Stem cell nucleofector solution II (Lonza, Cat #VPH-5022). Resuspend cells in nucleofector solution immediately before adding the Cas9 protein:sgRNA mixture. A stock of 100 µM (3.23 µg/µl) was prepared resuspending 5.1 nanomoles of single-guideRNA (sgRNA) in 51 µl 10 mM Tris buffer pH7.4. To obtain the nucleofection mixture, 8 µg high concentrated (≥5 µg/µl) Cas9 protein (shown below) (volume = 1.6 µl) was mixed with a 16.2 µg sgRNA combined with a sequence targeting 1-CR004366 sequence from Table 4 (shown below, SEQ ID NO: 120) (volume = 5 µl) and incubated for 20 min at room temperature to form a Cas9 protein-sgRNA complex. A molar ratio of 1:10 (50 pmol Cas9 protein: 500 pmol sgRNA) was used. Cas9 Protein (SEQ ID NO: 107)

MAPKKKRKVD KKYSIGLDIG TNSVGWAVIT DEYKVPSKKF KVLGNTDRHS
IKKNLIGALL FDSGETAEAT RLKRTARRRY TRRKNRICYL QEIFSNEMAK
VDDSFFHRLE ESFLVEEDKK HERHPIFGNI VDEVAYHEKY PTIYHLRKKL
VDSTDKADLR LIYLALAHMI KFRGHFLIEG DLNPDNSDVD KLFIQLVQTY
NQLFEENPIN ASGVDAKAIL SARLSKSRRL ENLIAQLPGE KKNGLFGNLI
ALSLGLTPNF KSNFDLAEDA KLQLSKDTYD DDLDNLLAQI GDQYADLFLA
AKNLSDAILL SDILRVNTEI TKAPLSASMI KRYDEHHQDL TLLKALVRQQ
LPEKYKEIFF DQSKNGYAGY IDGGASQEEF YKFIKPILEK MDGTEELLVK
LNREDLLRKQ RTFDNGSIPH QIHLGELHAI LRRQEDFYPF LKDNREKIEK
ILTFRIPYYV GPLARGNSRF AWMTRKSEET ITPWNFEEVV DKGASAQSFI
ERMTNFDKNL PNEKVLPKHS LLYEYFTVYN ELTKVKYVTE GMRKPAFLSG
EQKKAIVDLL FKTNRKVTVK QLKEDYFKKI ECFDSVEISG VEDRFNASLG
TYHDLLKIIK DKDFLDNEEN EDILEDIVLT LTLFEDREMI EERLKTYAHL
FDDKVMKQLK RRRYTGWGRL SRKLINGIRD KQSGKTILDF LKSDGFANRN
FMQLIHDDSL TFKEDIQKAQ VSGQGDSLHE HIANLAGSPA IKKGILQTVK
VVDELVKVMG RHKPENIVIE MARENQTTQK GQKNSRERMK RIEEGIKELG
SQILKEHPVE NTQLQNEKLY LYYLQNGRDM YVDQELDINR LSDYDVDHIV
PQSFLKDDSI DNKVLTRSDK NRGKSDNVPS EEVVKKMKNY WRQLLNAKLI
TQRKFDNLTK AERGGLSELD KAGFIKRQLV ETRQITKHVA QILDSRMNTK
YDENDKLIRE VKVITLKSKL VSDFRKDFQF YKVREINNYH HAHDAYLNAV
VGTALIKKYP KLESEFVYGD YKVYDVRKMI AKSEQEIGKA TAKYFFYSNI
MNFFKTEITL ANGEIRKRPL IETNGETGEI VWDKGRDFAT VRKVLSMPQV
NIVKKTEVQT GGFSKESILP KRNSDKLIAR KKDWDPKKYG GFDSPTVAYS
VLVVAKVEKG KSKKLKSVKE LLGITIMERS SFEKNPIDFL EAKGYKEVKK
DLIIKLPKYS LFELENGRKR MLASAGELQK GNELALPSKY VNFLYLASHY
EKLKGSPEDN EQKQLFVEQH KHYLDEIIEQ ISEFSKRVIL ADANLDKVLS
AYNKHRDKPI REQAENIIHL FTLTNLGAPA AFKYFDTTID RKRYTSTKEV
LDATLIHQSI TGLYETRIDL SQLGGDSRAD PKKKRKVHHH HHH

sgRNA (SEQ ID NO:120)GAGUAGCGCGAGCACAGCUAGUUUUAGAG
CUAGAAAUAGCAAGUUAAAAUAAGGCUAGUCCGUUAUCAACUUGAAAAAG
UGGCACCGAGUCGGUGCUUUU

The Cas9 protein-sgRNA complex was added to the cell suspension and transferred to the electroporation cuvette immediately. Cells were transfected using the nucelofector device (Lonza, Amaxa Nucleofector II) and program A023. After nucleofection cells were transferred from cuvette to one well of a 48 well synthemax coated plate containing pre-warmed LSC medium including 3 µM LATS inhibitor and 10 µM Rockinhibitor Y-27632 (Nature 1997, vol. 389, pp. 990-994). Incubate LSCs in a 5% CO2 incubator for around 5 days until cells are 90% confluent.

### FACS Analysis:

LSCs were treated with TryLE®Express Enzym (ThermoFisher, Cat #12605010) for 15 minutes in 5% CO2 at 37° C. After scraping the cells, the reaction was stopped by using cell culture medium containing 10% Serum and transferred to a falcon tube for a centrifugation step (1000 rpm, 5 minutes). After aspirating the medium cells were resuspended in 200 µl FACS buffer (PBS/10%FBS).

To analyze the expression of B2M and HLA-ABC, 5 µl APC mouse anti-human β2-microglobulin antibody (Biolegend, Cat#316312) and 20 µl PE mouse anti-human HLA-ABC antibody (BD Bioscience, Cat # 560168) were added to the cell suspension and incubated for 30 minutes on ice.

The same amount and incubation time of isotype control was used for each color (5 µl of Biolegend APC Mouse IgG1, ĸ Isotype Ctrl (FC) Antibody #316311 and 20 µl of BD Biosciences PE mouse IgG1, ĸ Isotype Ctrl #555749) to set up the negative control gate later in FACS. Cells were washed 3 times after antibody labelling with FACS buffer and resuspended in 500 µl in FACS buffer (depending on the cell number). Before FACS sorting, cells were filtered through a 70 µm filter and stored on ice until sorting.

In order to prevent cells sticking to the wall, collection tubes were filled with FACS buffer for 30 minutes before the sort and aspirated before adding the collection medium. Cells were sorted on a BD FACSAria II instrument into prepared collection tubes, using human serum enriched LSC medium including compound. FACS data were analyzed using BD FACSDiva software and FlowJo software.

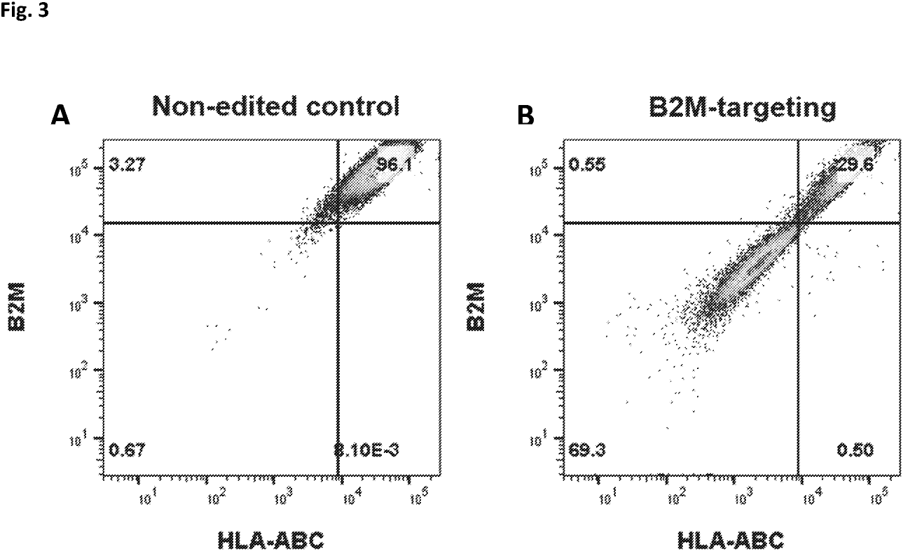

The results confirmed about 70% of the cells CRISPR-edited with sgRNA SEQ ID NO: 120 did not express B2M and eliminate HLA I expression on cell surface of limbal stem cells (FIG. 3).

### LSC/T-cell Reaction Assay:

An LSC/T-cell assay was performed in flat bottom 96well synthemax coated plates in duplicates and incubated in 5% CO2 at 37° C. for 10 days. RPMI-1640 supplemented with HEPES (100 µM), non-essential aas (10x), sodium pyruvate (10 mM), 2-Mercapthoethanol (10x), 10% FBS and 1% Penicillin-Streptomycin (Gibco by Life Technologies) was used as medium for co-culture. Alternatively, RPMI-1640 supplemented with HEPES (10 mM), non-essential aas (1x), sodium pyruvate (1 mM), 2-Mercapthoethanol (1x), 10% FBS and 1% Penicillin-Streptomycin (Gibco by Life Technologies) was used as medium for co-culture.

One day before co-culture, LSCs (stimulator cells) were passed and cultured to a confluency of around 70% (30′000 - 50′000 cells) and cultured with LSC medium including compound. On day two, peripheral blood mononuclear cells (PBMC) were separated using EDTA blood with the Ficoll-Paque method (GE Healthcare Life Sciences, cat #17-1440-03). After PBMC isolation, the CD8+ T-cell isolation Kit (Miltenyi Biotec, Cat #130-096-495) was used to separate CD8+ cells from all other cell populations. The cell suspension with 1-10×10^6 CD8+ cells were stained with 1 µM CellTrace Violet (Invitrogen, Cat #C34557) and were incubated for 20 minutes at 37° C. in the dark. After incubation 2ml ice cold heat inactivated FBS was added to each 5 ml cell suspension and cells were incubated for additional 5 minutes at 37° C. After 3 washing steps with culture medium, stained CD8+ cells were diluted to a final concentration of 100′000 cells per well and 100 µl of CD8+ cell dilution was added to each well containing LSCs after washing off LSC medium. For positive control, stained CD8+ cells were incubated in a pre-coated 10 µg/ml anti-human CD3+ (eBioscience, Cat #16-0037-85) well including diluted 3 µg/ml anti-human CD28 (eBioscience, Cat # 16-0289-85). One separate duplicate with stained CD8+ cells with medium only was used as negative control.

After 10 days, CD8+ cells were transferred to a U-bottomed 96-well plate and washed 3 times using autoMAC rinsing solution (Miltenyi Biotec, Cat #130-091-222) including MACS BSA stock solution (Miltenyi Biotec, Cat #130-091-376). Cells were measured on a BD LSRFortessa X-20. FACS data were analyzed using BD FACSDiva software and FlowJo software.

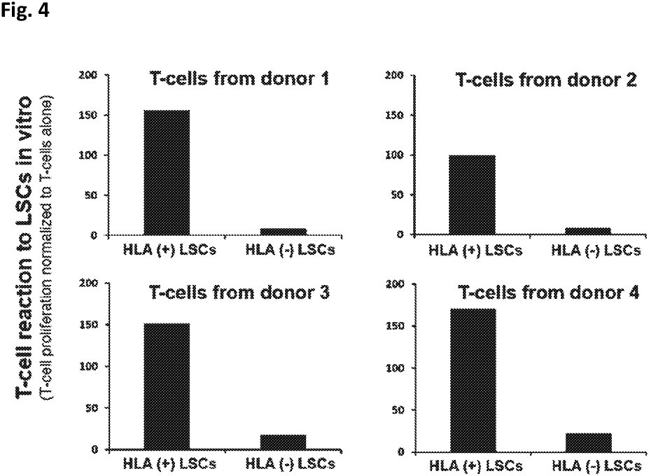

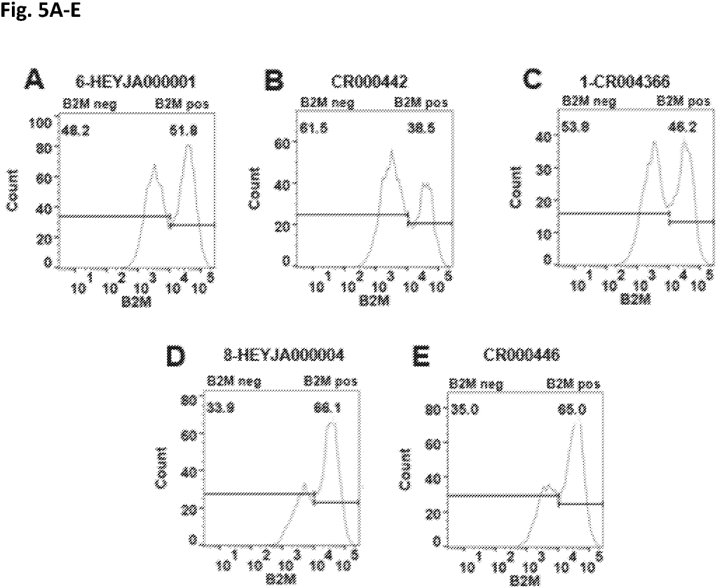

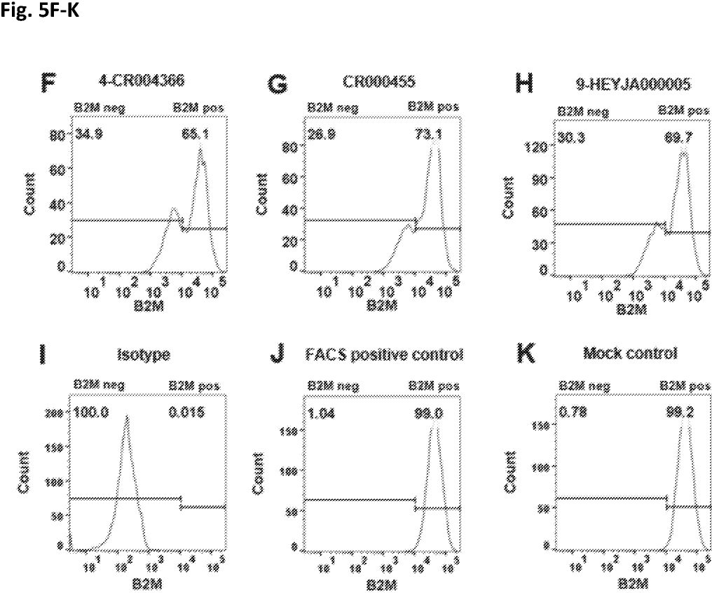

FIG. 4 shows gene edited limbal stem cells (LSCs) co-cultured with CD8+ T-cells from 4 different donors. In all 4 donors T-cell immunresponse was almost completely eliminated co-cultured with B2M/HLA-Class I negative LSCs, which were CRISPR-edited with sgRNA SEQ ID NO: 120.

### Example 8: Screening for Efficiency of sgRNAs in Reducing or Eliminating Expression of B2M in LSCs and Elimination of HLA I Expression on Cell Surface of Limbal Stem Cells

### Limbal Stem Cell Isolation and Culture:

Cells obtained as described in Example 1 were plated in a 10 cm synthemax coated petri dish in limbal epithelium cell culture medium (DMEM F12 supplemented with 10% human serum and 1.3 mM calcium chloride) supplemented with 3 µM LATS inhibitor compound and 10 µM Rock inhibitor Y-27632 (Nature 1997, vol. 389, pp. 990-994). Cells were cultured under these conditions for 24-48 hours at 37° C. in 5% CO2.

LSCs were nuclofected with selected gRNAs (Table 6) followed by FACS analysis / MACS separation.

Nucleofection approach for sgRNA screening (SEQ ID NO 120 and 160-177)-in LSCs was performed as follows: LSCs at passage 3 were trypsinized with TryLETMExpress Enzym (ThermoFisher, Cat #12605010) for 15 min in 5%CO2 at 37° C. After scraping the cells, the reaction was stopped by using cell culture medium containing 10% Serum and transferred to a falcon tube. After counting cells using Vi-cell 300′000 cells were prepared per reaction by transferring 300′000 cells in single tubes and centrifuged at 1000 rpm for 5 min. The supernatant was aspirated using manually pipetting to avoid cell loss and the cells were resuspended in the Stem cell nucleofector solution II (Lonza, Cat #VPH-5022). Resuspend cells in nucleofector solution immediately before adding the Cas9 RNP:sgRNA mixture. To obtain the nucleofection mixture, 5 µg high concentrated (≥5 µg/µl) Cas9 protein of SEQ ID NO: 106 (volume = 0.78 µl) was mixed with 19.5 µg sgRNA of Table 6 (volume = 12.2 µl) and incubated for 20 min at room temperature. A molar ratio of ~1:20 (31.5 pmol Cas9 RNP: 605pmol sgRNA) was used. The Cas9 protein-guideRNA complex was added to the cell suspension and transferred to the electroporation cuvette immediately. Cells were transfected using the nucelofector device (Lonza, Amaxa Nucleofector II) and program A023. After nucleofection cells were transferred from cuvette to one well of a 24 well synthemax coated plate containing pre-warmed LSC medium including 3 µM LATS compound and 10 µM Rock inhibitor Y-27632 (Nature 1997, vol. 389, pp. 990-994). Incubate LSCs in a 5% CO2 incubator for around 3 days until cells are 90% confluent.

### FACS Analysis:

LSCs were treated with TryLE®Express Enzym (ThermoFisher, Cat #12605010) for 15 minutes in 5% CO2 at 37° C. After scraping the cells, the reaction was stopped by using cell culture medium containing 10% Serum and transferred to a falcon tube for a centrifugation step (1000 rpm, 5 minutes). After aspirating the medium cells were resuspended in 200 µl FACS buffer (PBS/10%FBS).

To analyze the expression of B2M and HLA-ABC, 5 µl APC mouse anti-human β2-microglobulin antibody (Biolegend, Cat#316312) and 20 µl PE mouse anti-human HLA-ABC antibody (BD Bioscience, Cat # 560168) were added to the cell suspension and incubated for 30 minutes on ice.

The same amount and incubation time of isotype control was used for each color (5 µl of Biolegend APC Mouse IgG1, ĸ Isotype Ctrl (FC) Antibody #316311 and 20 µl of BD Biosciences PE mouse IgG1, ĸ Isotype Ctrl #555749) to set up the negative control gate later in FACS. Cells were washed 3 times after antibody labelling with FACS buffer and resuspended in 200 µl in FACS buffer (depending on the cell number).

FACS data were analyzed using BD FACSDiva software and FlowJo software.

The results of B2M knockout efficiency in LSCs after nucleofection are shown in Table 8 below.

TABLE 8

| gRNA ID | B2M knockout efficiency in LSCs after nucleofection [%) |
|---|---|
| 1-CR004366 | 55 |
| 2-CR004366 | 2 |
| 3-CR004366 | 2 |
| 4-CR004366 | 22 |
| 5-CR004366 | 2 |
| 6-HEYJA000001 | 33 |
| 7-HEYJA000003 | 8 |
| 8-HEYJA000004 | 22 |
| 9-HEYJA000005 | 12 |
| 10-HEYJA000007 | 1 |
| 11-HEYJA000008 | 1 |
| 12-HEYJA000009 | 1 |
| CR000442 | 62 |
| CR000443 | 25 |
| CR000446 | 27 |
| CR000453 | 5 |
| CR000455 | 24 |
| CR000456 | 3 |
| CR006979 | 2 |

### Example 9: Efficiency of sgRNAs in Reducing or Eliminating Expression of B2M in LSCs and Elimination of HLA I Expression on Cell Surface Of Limbal Stem Cells FACS and MACS of B2M-Negative LSCs

Limbal Stem Cell Isolation and Culture as performed in Example 8.

### Nucleofection for sgRNA Selected for On/off Target Analysis:

LSCs at passage 3 were trypsinized with TryLE®Express Enzym (ThermoFisher, Cat #12605010) for 15 min in 5%CO2 at 37° C. After scraping the cells, the reaction was stopped by using cell culture medium containing 10% Serum and transferred to a falcon tube. After counting cells using Vi-cell 1′000′000 cells were prepared per reaction by transferring 1′000′000 cells in single tubes and centrifuged at 1000 rpm for 5 min. The supernatant was aspirated using manually pipetting to avoid cell loss and the cells were resuspended in Stem cell nucleofector solution II (Lonza, Cat #VPH-5022). Resuspend cells in nucleofector solution immediately before adding the Cas9 RNP:sgRNA mixture. To obtain the nucleofection mixture, 10 µg high concentrated (≥5 µg/µl) Cas9 protein (volume = 1.56 µl; SEQ ID NO: 106) was mixed with 40.2 µg sgRNA (volume = 25 µl; sequences of sgRNAs are presented in Table 6: SEQ ID NO 120, 162, 164, 166, 167, 171, 173, 175) and incubated for 20 min at room temperature. A molar ratio of 1:20 (62.5 pmol Cas9 RNP: 1250 pmol sgRNA) was used.

The Cas9 protein-guideRNA complex was added to the cell suspension and transferred to the electroporation cuvette immediately. Cells were transfected using the nucelofector device (Lonza, Amaxa Nucleofector II) and program A023. After nucleofection cells were transferred from cuvette to one well of a 12 well synthemax coated plate containing pre-warmed LSC medium including 3 µM LATS compound and 10 µM Rockinhibitor Y-27632 (Nature 1997, vol. 389, pp. 990-994). Incubate LSCs in a 5% CO2 incubator for around 3 days until cells are 90% confluent.

### Facs:

LSCs were treated with TryLETMExpress Enzym (ThermoFisher, Cat #12605010) for 15 minutes in 5% CO2 at 37° C. After scraping the cells, the reaction was stopped by using cell culture medium containing 10% Serum and transferred to a falcon tube for a centrifugation step (1000 rpm, 5 minutes). After aspirating the medium cells were resuspended in 200 µl FACS buffer (PBS/10%FBS).

To analyze the expression of B2M and HLA-ABC, 2.5 µl APC mouse anti-human β2-microglobulin antibody (Biolegend, Cat#316312) and 10 µl PE mouse anti-human HLA-ABC antibody (BD Bioscience, Cat # 560168) were added to the cell suspension and incubated for 30 minutes on ice.

The same amount and incubation time of isotype control was used for each color (2.5 µl of Biolegend APC Mouse IgG1, ĸ Isotype Ctrl (FC) Antibody #316311 and 10 µl of BD Biosciences PE mouse IgG1, ĸ Isotype Ctrl #555749) to set up the negative control gate later in FACS. Cells were washed 3 times after antibody labelling with FACS buffer and resuspended in 300 µl in FACS buffer. A small aliquot of labelled LSCs (~15′000 LSCs) were analyzed by FACS to confirm B2M knockout after nucleofection. FACS data were analyzed using BD FACSDiva software and FlowJo software. To obtain a purified B2M negative LSC culture, the second and bigger portion of antibody labelled LSCs were sorted using MACS to separete B2M negative from B2M positives.

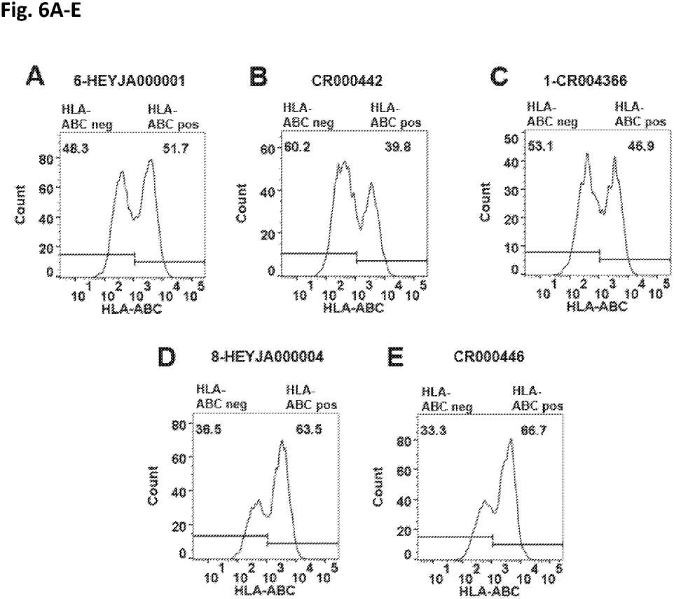

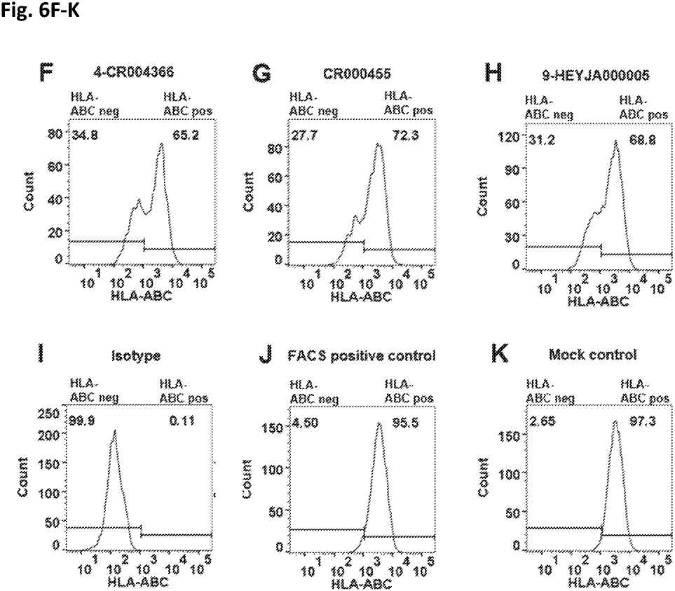

The results of B2M knockout efficiency in LSCs after nucleofection are shown on FIG. 5. Efficiency of elimination of HLA I expression on cell surface of limbal stem cells after nucleofection are shown on FIG. 6.

### Macs:

To obtain a purified B2M negative LSC culture, the second and bigger portion of antibody labelled LSCs were sorted using MACS to separate B2M negative from B2M positives.

After labelling LSCs with B2M and HLA-ABC antibodies as decribed above, the reaction was stopped by adding 2 ml MACS buffer (Miltenyi Biotec, #130-091-222) and centrifuged at 1000 rpm for 5 minutes. For each step MACS buffer was always supplemented with 3 µM LATS inhibitor compound, 10 µM Rock inhibitor Y-27632 (Nature 1997, vol. 389, pp. 990-994) and BSA (Miltenyi Biotec, #130-091-376).

After aspirating the supernatant, cells were resuspended in 80 µl MACS buffer and 10 µl of anti-APC micorbeads (Miltenyi Biotec, #130-090-855) and 10 µl anti-PE microbeads (Miltenyi Biotec, #130-048-801) were added to the cell suspension. Antibody labelled LSCs including magnetic beads were incubated for 15 minutes in the refrigerator in the dark. After incubation cells were washed by adding 2 ml of MACS buffer and centrifudged for 5 minutes at 1000 rpm. 500 µl of MACS buffer was added after aspiration of supernatant.

To prepare the LS columns (Miltenyi Biotec, #130-042-401) for separation of B2M negative from B2M positive LSCs, LS columns were placed on the magnet device (Miltenyi Biotec, Quadro magnet) and washed with 3 ml MACS buffer. The flow through was discarded.

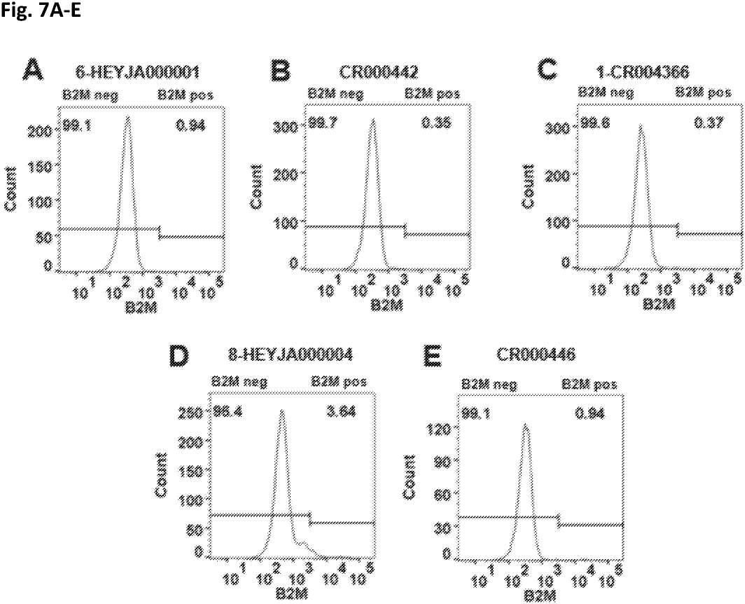

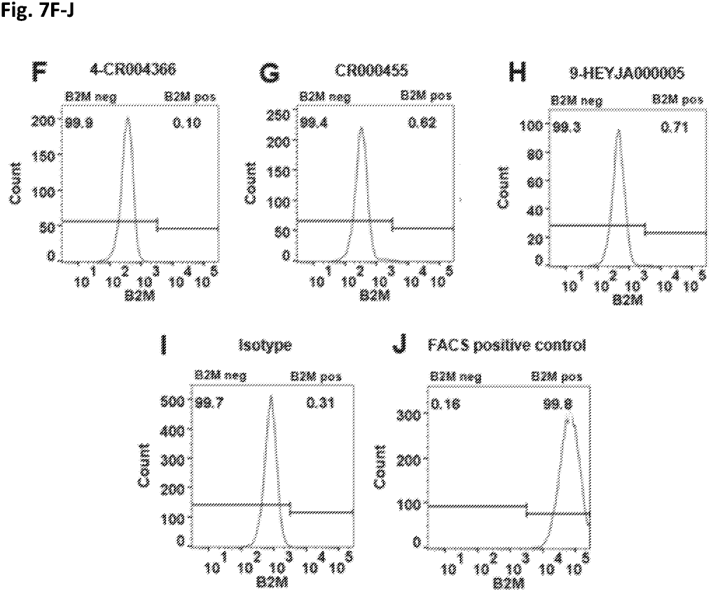

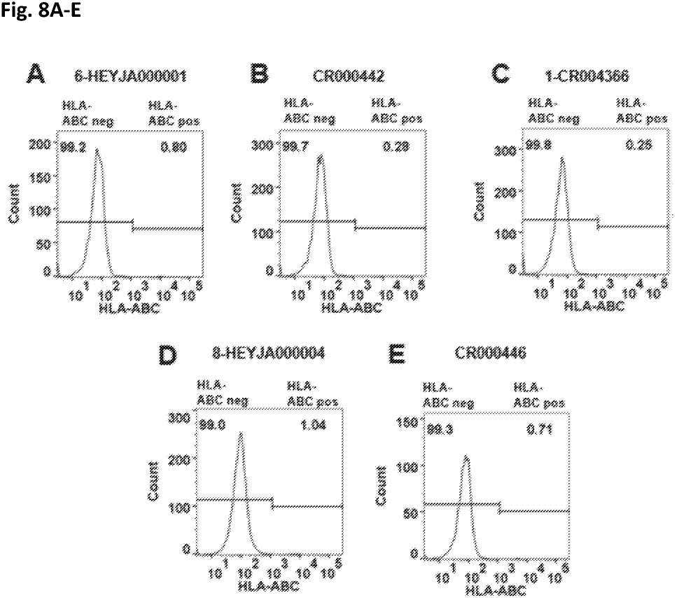

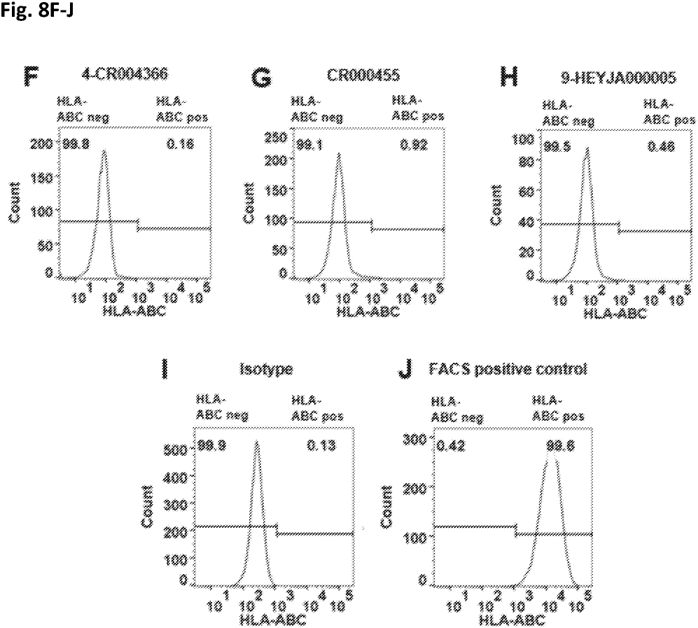

Cell supsension was applied on top of column and flow through was collected in a separate 15 ml falcon tube to collect B2M negative LSCs. Once all of the cell suspension was in the flow through fraction, 3 ml MACS buffer was applied on the column. This step was repeated 3 times by adding new MACS buffer when the column reservoir was empty. B2M negative LSC fraction was centrifuged for 5 minutes at 1000 rpm. After aspirating the supernatant, B2M negative LSCs were resuspended in LSC media including 3 µM LATS inhibitor compound and 3 µM Rock inhibitor Y-27632 (Nature 1997, vol. 389, pp. 990-994) and plated on 1 well of a 48 synthemax coated plate. After 8-21 days (depending on cell expansion) LSCs were treated with TryLE®Express Enzym (ThermoFisher, Cat #12605010) for 15 minutes in 5% CO2 at 37° C. and a small aliquot of B2M negative was prepared for FACS to confirm purity of B2M negative LSC culture (FIGS. 7 and 8) and the second and bigger fraction was prepared for On/off-target analysis.

FIGS. 7 and 8 show FACS data detecting B2M and HLA-ABC surface protein on gene edited limbal stem cells, which were MACS treated after nucleofection to obtain a B2M/HLA-ABC negative LSC culture. All sgRNAs tested showed a pure (~99-100%) B2M/HLA-ABC negative LSC culture.

### Example 10: Characterization of gRNA Specificity and Analysis of CRISPR/Cas9-Mediated Off-Target Editing Events

A biochemical method (See, e.g., Cameron et al., Nature Methods. 6, 600-606; 2017) was used to determine potential off-target genomic sites cleaved by Cas9 and selected B2M guides. Guides showing B2M indel activity were tested for potential off-target genomic cleavage sites with this assay. In this experiment, 11 sgRNAs targeting human B2M were screened using genomic DNA purified from male human peripheral blood mononuclear cells (PBMCs) alongside a control guide with a known off-target profile. The number of potential off-target sites detected using a guide concentration of 64 nM in the biochemical assay are shown in Table 10. As a result of this analysis, several gRNAs were selected for analysis of potential off-target activity in limbal stem cells.

### Off-Target Activity Detection in Limbal Stem Cells:

Potential CRISPR/Cas9-mediated cutting sites identified above were evaluated using targeted PCR and NGS in genome-edited expanded LSCs.

Selected sgRNAs (SEQ ID NO: 120, 162, 166, 167, 171, and 175) were further analyzed by amplicon sequencing in edited and non-edited cells. Primers flanking the potential off-target sites for each guide were used to detect indels by NGS analysis in edited LSCs and non-edited peripheral blood mononuclear cells. Sites that had either (1) a difference in mean indel percentage between edited and unedited cells greater than 0.5%; or (2) a p value less than 0.05 between edited and unedited indel were further analyzed. NGS sequence reads for such sites were assessed for characteristic indel patterns near the putative Cas9 cut site. Based on the results, we could assess the specificity of gRNAs and their suitability for therapeutic applications.

### Results:

The gRNA on-target and off-target results are shown below. All sgRNAs of Table 10 were analyzed by biochemical assay, with select results further analyzed by amplicon sequencing. NGS results showed B2M sgRNAs (SEQ ID NO: 120, 162, 166, 167, 171, and 175) can achieve ~99% indels in purified LSC populations. In the NGS results, no predicted sites tested positive for off-target activity with any of the sgRNAs (SEQ ID NO: 120, 162, 166, 167, 171, and 175). For SEQ ID NO: 120, 64 out of 69 off-target loci were sequenced, and zero validated indels for off-target activity in LSCs were identified. For SEQ ID NO: 162, 88 out of 92 off-target loci were sequenced, and zero validated indels for off-target activity in LSCs were identified. For SEQ ID NO: 166, 60 out of 62 off-target loci were sequenced, and zero validated indels for off-target activity in LSCs were identified. For SEQ ID NO: 167, 35 out of 35 off-target loci were sequenced, and zero validated indels for off-target activity in LSCs were identified. For SEQ ID NO: 171, 28 out of 29 off-target loci were sequenced, and zero validated indels for off-target activity in LSCs were identified. For SEQ ID NO: 175, 46 out of 48 off-target loci were sequenced, and zero validated indels for off-target activity in LSCs were identified.

TABLE 10

Off-Target Analysis

| gRNA ID (sgRNA SEQ ID NO) | Target | Sequence of the targeting domain of the gRNA (SEQ ID NO) | off-target Site_ Count | B2M purified rhAmpSeq indel | Number of sites positive for off-target activity in LSCs |
|---|---|---|---|---|---|
| 6-HEYJA000001 (SEQ ID NO: 164) | B2M | GGCCGAGAUGUCU CGCUCCG (SEQ ID NO: 113) | 8 | ND | ND |
| CR000442 (SEQ ID NO: 171) | B2M | GGCCACGGAGCGA GACAUCU (SEQ ID NO:134) | 29 | 99.10±0.26% | 0 of 28 |
| 1-CR004366 (SEQ ID NO: 120) | B2M | GAGUAGCGCGAGCA CAGCUA (SEQ ID NO: 108) | 69 | 99.60±0.44% | 0 of 64 |
| CR000443 (SEQ ID NO: 172) | B2M | CGCGAGCACAGCUA AGGCCA (SEQ ID NO: 135) | 117 | ND | ND |
| 8-HEYJA000004 (SEQ ID NO: 166) | B2M | ACUCACGCUGGAUA GCCUCC (SEQ ID NO: 115) | 62 | 99.95±0.07% | 0 of 60 |
| CR000446 (SEQ ID NO: 173) | B2M | AGGGUAGGAGAGAC UCACGC (SEQ ID NO: 136) | 98 | ND | ND |
| 4-CR004366 (SEQ ID NO: 162) | B2M | CAGUAAGUCAACUU CAAUGU (SEQ ID NO: 111) | 92 | 99.85±0.21% | 0 of 88 |
| CR000453 (SEQ ID NO: 174) | B2M | CACAGCCCAAGAUA GUUAAG (SEQ ID NO: 137) | 82 | ND | ND |
| CR000455 (SEQ ID NO: 175) | B2M | UUACCCCACUUAAC UAUCUU (SEQ ID NO: 138) | 48 | 99.33±0.32% | 0 of 46 |
| CR000456 (SEQ ID NO: 176) | B2M | CUUACCCCACUUAA CUAUCU (SEQ ID NO: 139) | 66 | ND | ND |
| 9-HEYJA000005 (SEQ ID NO: 167) | B2M | UCACGUCAUCCAGC AGAGAA (SEQ ID NO: 116) | 35 | 99.80±0.26% | 0 of 35 |
| Control | VEGF A | GACCCCCUCCACCC CGCCUC (SEQ ID NO: 178) | 756 | ND | ND |

ND: no data

Unless indicated otherwise, all methods, steps, techniques and manipulations that are not specifically described in detail can be performed and have been performed in a manner known per se, as will be clear to the skilled person. Reference is for example again made to the standard handbooks and the general background art mentioned herein and to the further references cited therein. Unless indicated otherwise, each of the references cited herein is incorporated in its entirety by reference.

Claims to the invention are non-limiting and are provided below. Although particular embodiments and claims have been disclosed herein in detail, this has been done by way of example for purposes of illustration only, and is not intended to be limiting with respect to the scope of the appended claims, or the scope of subject matter of claims of any corresponding future application. In particular, it is contemplated by the inventors that various substitutions, alterations, and modifications may be made to the disclosure without departing from the spirit and scope of the disclosure as defined by the claims. The choice of nucleic acid starting material, clone of interest, or library type is believed to be a matter of routine for a person of ordinary skill in the art with knowledge of the embodiments described herein. Other embodiments, advantages, and modifications are considered to be within the scope of the following claims. Those skilled in the art will recognize or be able to ascertain, using no more than routine experimentation, many equivalents of the specific embodiments of the invention described herein.

### Example 11: In Vitro TK6 Micronucleus Assay

An in vitro TK6 Micronucleus Assay was performed to evaluate the clastogenic and aneugenic potential of a LATS inhibitor by its effects on the frequency of micronuclei in cultured human lymphoblastoid TK6 cells treated for 30 hours with no recovery in the absence of an Aroclor-induced rat liver metabolizing system (S-9) (30+0 hour -S-9) and for 3 hours in the presence of S-9 with 27 hours of recovery (3+27 hours +S-9).

TK6 cells were cultured in HEPES-buffered RPMI 1640 medium with GlutaMAXTM-1 including 10% (v/v) heat inactivated foetal calf serum, 100 Units/mL/100 µg/mL penicillin / streptomycin. Liver S-9 mix from male rats induced with Aroclor 1254 was added as a 10% S-9 mix to achieve a final concentration of 1% in the test system. Vehicle and positive control treatments comprised additions at the same volume per well (1% (v/v)) as the tested LATS inhibitors solutions. Standard vehicle used was DMSO.

### Evaluation Criteria:

For valid data, the test item was considered to induce clastogenic and/or aneugenic events if:
A statistically significant increase in the frequency of micronucleated mononucleate (MNMON) cells at one or more concentrations was observed
An incidence of MNMON cells at such a concentration exceeded the normal range in both replicate cultures was observed
A concentration-related increase in the proportion of MNMON cells was observed (positive trend test).

The test item was considered positive in this assay if all of the above criteria were met. The test item was considered negative in this assay if none of the above criteria were met.

It was concluded that dimethyl(3-methyl-3-{[2-(pyridin-4-yl)pyrido[3,4-d]pyrimidin-4-yl]amino}butyl)amine and N¹,N¹,3-trimethyl-N³-(2-(3-methyl-1H-pyrazol-4-yl)pyrido[3,4-d]pyrimidin-4-yl)butane-1,3-diamine did not induce micronuclei in cultured human lymphoblastoid TK6 cells when tested up to its limit of cytotoxicity, following 30 hours treatment with no recovery in the absence of an Aroclor-induced rat liver metabolising system (S-9) and up to 1 mM, following 3 hours treatment in the presence of S-9 with 27 hours of recovery (Table 11).

TABLE 11

TK6 MNT screen

| | | |
|---|---|---|
| Compound | Maximum concentration limited by | Conclusion TK6 MNT screen result |
| dimethyl(3-methyl-3-{[2-(pyridin-4-yl)pyrido[3,4-d]pyrimid in-4-yl]amino}butyl)amine | Cytotoxicity, for -S-9; 1 mM, for +S-9 | “negative” |
| N¹,N¹,3-trimethyl-N³-(2-(3-methyl-1H-pyrazol-4-yl)pyrido[3,4-d]pyrimidin-4-yl)butane-1,3-diamine | Cytotoxicity, for -S-9; 1 mM, for +S-9 | “negative” |

- S-9: No statistically significant (p≤0.05) increases in micronucleated mononucleate (MNMON) cells were observed for any concentration analysed up to 40.00 µg/mL, inducing 58% and 57%cytotoxicities for dimethyl(3-methyl-3-{[2-(pyridin-4-yl)pyrido[3,4-d]pyrimidin-4-yl]amino}butyl)amine and N¹,N¹,3-trimethyl-N³-(2-(3-methyl-1H-pyrazol-4-yl)pyrido[3,4-d]pyrimidin-4-yl)butane-1,3-diamine, respectively. The MNMON cell frequency of all test article treated cultures fell within the 95^{th} percentile of the current observed historical vehicle control (normal) range.

+S-9: No statistically significant (p≤0.05) increases in MNMON cells were observed for any concentration analysed up to 337.0 µg/mL (equivalent to approximately 1 mM), inducing 39% and 33% cytotoxicities for dimethyl(3-methyl-3-{[2-(pyridin-4-yl)pyrido[3,4-d]pyrimidin-4-yl]amino}butyl)amine and N¹,N¹,3-trimethyl-N³-(2-(3-methyl-1H-pyrazol-4-yl)pyrido[3,4-d]pyrimidin-4-yl)butane-1,3-diamine, respectively. The MNMON cell frequency of all test article treated cultures fell within the normal range.

### Example 12. Bacterial Reverse Mutation Assay

A Bacterial Reverse Mutation Assay (“Ames Test”) was performed to evaluate the mutagenic potential of a LATS inhibitor by its effects on one or more histidine-requiring strains of Salmonella typhimurium (TA98 and TA100.) in the absence and presence of a liver metabolizing system.

The 6-well plate Ames test is a miniaturized screening version of the original test using smaller volumes and test item amounts, see e.g. P.A. Escobar et al. (2013), Mut. Res. 752, 99-118. The highest concentration of 500 µg/well used in this 6-well Ames screen corresponds to about the top concentration of 2500 µg/plate in the standard Ames test. Liver S-9 mix from male rats induced with Aroclor 1254 was added as a 5% S-9 mix to the test system. Vehicle and positive control treatments comprised additions at the same volume per well (20 µL) as the tested LATS inhibitors solutions. Standard vehicle used was DMSO.

### Evaluation Criteria

For valid data, the test item was considered mutagenic in this assay if:
A concentration related increase in revertant numbers of ≥2-fold the concurrent vehicle control values was observed.

The test article was regarded positive in this assay if this criterion is met, otherwise it was regarded as negative.

It was concluded that dimethyl(3-methyl-3-{[2-(pyridin-4-yl)pyrido[3,4-d]pyrimidin-4-yl]amino}butyl)amine and N¹,N¹,3-trimethyl-N³-(2-(3-methyl-1H-pyrazol-4-yl)pyrido[3,4-d]pyrimidin-4-yl)butane-1,3-diamine did not show evidence of a mutagenic potential in Salmonella typhimurium strains TA98 and TA100 in the absence or presence of metabolic activation (rat liver S-9 mix) (Table 12).

TABLE 12

Bacterial Reverse Mutation Assay (AMES screen)

| Compound | Maximum Concentration (µg/well) | AMES screen result |
|---|---|---|
| dimethyl(3-methyl-3-{[2-(pyridin-4-yl)pyrido[3,4-d]pyrimidin-4-yl]amino}butyl)amine | 500 | “negative” |
| N¹,N¹,3-trimethyl-N³-(2-(3-methyl-1H-pyrazol-4-yl)pyrido[3,4-d]pyrimidin-4-yl)butane-1,3-diamine | 500 | “negative” |

1. A modified limbal stem cell, which has reduced or eliminated expression of beta-2-microglobulin (B2M) relative to an unmodified limbal stem cell, wherein the B2M expression is reduced or eliminated by a CRISPR system comprising a gRNA molecule comprising a targeting domain complementary to a target sequence in the B2M gene.

2. A modified limbal stem cell, which has reduced or eliminated expression of beta-2-microglobulin (B2M) relative to an unmodified limbal stem cell, wherein the B2M expression is reduced or eliminated by a CRISPR system comprising a nucleic acid molecule encoding a gRNA molecule comprising a targeting domain complementary to a target sequence in the B2M gene.

3. The modified limbal stem cell of claim 1 or 2, wherein the modified limbal stem cell was cultured in media comprising a large tumor suppressor kinase (“LATS”) inhibitor, optionally wherein the LATS inhibitor is a compound of Formula A1 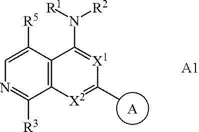
or a salt thereof, wherein
X¹ and X² are each independently CH or N;
Ring A is
(a) a 5- or 6-membered monocyclic heteroaryl that is linked to the remainder of the molecule through a carbon ring member and comprises, as ring member, 1 to 4 heteroatoms that are independently selected from N, O and S, provided that at least one of the heteroatom ring member is an unsubstituted nitrogen (—N═) positioned at the 3- or the 4-position relative to the linking carbon ring member of the 5-membered heteroaryl or at the para ring position of the 6-membered heteroaryl; or
(b) a 9-membered fused bicyclic heteroaryl that is selected from 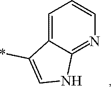 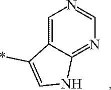 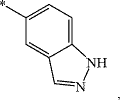 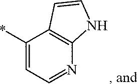 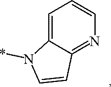
wherein “*” represents the point of attachment of ring A to the remainder of the molecule;
wherein ring A is unsubstituted or substituted by 1 to 2 substituents independently selected from halogen, cyano, C₁₋₆alkyl, C₁₋₆haloalkyl, —NH₂, C₁₋₆alkylamino, di-(C₁₋₆alkyl)amino, C₃₋₆cycloalkyl, and phenylsulfonyl;
R⁰ is hydroxyl or C₁₋₆alkoxy;
R¹ is hydrogen or C₁₋₆alkyl;
R² is selected from
(a) C₁₋₈alkyl that is unsubstituted or substituted by 1 to 3 substituents independently selected from
(i) halogen;
(ii) cyano;
(iii) oxo;
(iv) C₂alkenyl;
(v) C₂alkynyl;
(vi) C₁₋₆haloalkyl;
(vii) —OR⁶, wherein R⁶ is selected from hydrogen, C₁₋₆alkyl that is unsubstituted or substituted by R⁰ or —C(O)R⁰;
(viii) —NR^{7a}R^{7b}, wherein R^{7a} is hydrogen or C₁₋₆alkyl, and R^{7b} is selected from hydrogen, —C(O)R⁰, C₁₋₆alkyl that is unsubstituted or substituted by —C(O)R⁰;
(ix) —C(O)R⁸, wherein R⁸ is R⁰ or —NH—C₁₋₆alkyl-C(O)R⁰;
(x) -S(O)₂C₁₋₆alkyl;
(xi) monocyclic C₃₋₆cycloalkyl or polycyclic C₇₋₁₀cycloalkyl that are each unsubstituted or substituted by 1 to 2 substituents independently selected from halogen, C₁₋ ₆alkyl, hydroxyC₁₋₆alkyl, C₁₋₆haloalkyl, R⁰, —NH₂, C₁₋₆alkylamino, and di-(C₁₋ ₆alkyl)amino;
(xii) 6-membered heterocycloalkyl comprising, as ring members, 1 to 2 heteroatoms independently selected from N, O and S and that is unsubstituted or substituted by 1 to 2 substituents independently selected from hydroxyl, halogen, C₁₋₆alkyl, C₁₋₆alkylamino, and di-(C₁₋₆alkyl)amino;
(xiii) phenyl that is unsubstituted or substituted by halogen;
(xiv) 5- or 6-membered monocyclic heteroaryl comprising, as ring members, 1 to 4 heteroatoms independently selected from N and O; and
(xv) 9- or 10-membered fused bicyclic heteroaryl comprising, as ring member, 1 to 2 heteroatoms independently selected from N and O;
(b) -S(O)₂C₁₋₆alkyl;
(c) phenyl that is unsubstituted or substituted by 1 to 2 substituents independently selected from halogen, C₁₋₆alkyl and R⁰;
(d) C₃₋₆cycloalkyl that is unsubstituted or substituted by 1 to 2 substituents independently selected from C₁₋₆haloalkyl, R⁰, C₁₋₆alkylamino, di-(C₁₋₆alkyl)amino, —C(O)R⁰, and C₁₋ ₆alkyl that is unsubstituted or substituted by R⁰ or —C(O)R⁰; and
(e) 4-membered heterocycloalkyl comprising, as ring members, 1 to 2 heteroatoms selected from N, O and S and that is unsubstituted or substituted by 1 to 2 substituents independently selected from C₁₋₆haloalkyl, R⁰, C₁₋₆alkylamino, di-(C₁₋ ₆alkyl)amino, —C(O)R⁰, and C₁₋₆alkyl that is unsubstituted or substituted by R⁰ or —C(O)R⁰;
or R¹ and R² can be taken together with the nitrogen atom to which both are bound to form a 4- to 6-membered heterocycloalkyl that can include, as ring members, 1 to 2 additional heteroatoms independently selected from N, O, and S, wherein the 4- to 6-membered heterocycloalkyl formed by R¹ and R² taken together with the nitrogen atom to which both are bound is unsubstituted or substituted by 1 to 3 substituents independently selected from halogen, C₁₋₆alkyl, C₁₋₆haloalkyl, and R⁰;
R³ is selected from hydrogen, halogen and C₁₋₆alkyl; and
R⁵ is selected from hydrogen, halogen and —NH—(3- to 8-membered heteroalkyl), wherein the 3- to 8-membered heteroC₃₋₈alkyl of the —NH—(3- to 8-membered heteroalkyl) comprises 1 to 2 oxygen atoms as chain members and is unsubstituted or substituted by R⁰.

4. The modified limbal stem cell according to claim 3, wherein the compound is selected from: dimethyl(3-methyl-3-{[2-(pyridin-4-yl)pyrido[3,4-d]pyrimidin-4-yl]amino}butyl)amine and N¹,N¹,3-trimethyl-N³-(2-(3-methyl-1H-pyrazol-4-yl)pyrido[3,4-d]pyrimidin-4-yl)butane-1,3-diamine.

5. The modified limbal stem cell according to claim 3, wherein the compound is dimethyl(3-methyl-3-{[2-(pyridin-4-yl)pyrido[3,4-d]pyrimidin-4-yl]amino}butyl)amine.

6. The modified limbal stem cell according to any one of claims 3 to 5, wherein the compound is present in a concentration of 3 to 10 micromolar.

7. The modified limbal stem cell of claim any one of claims 1-6, wherein the targeting domain of the gRNA molecule is complementary to a sequence within a genomic region selected from: chr15:44711469-44711494, chr15:44711472-44711497, chr15:44711483-44711508, chr15:44711486-44711511, chr15:44711487-44711512, chr15:44711512-44711537, chr15:44711513-44711538, chr15:44711534-44711559, chr15:44711568-44711593, chr15:44711573-44711598, chr15:44711576-44711601, chr15:44711466-44711491, chr15:44711522-44711547, chr15:44711544-44711569, chr15:44711559-44711584, chr15:44711565-44711590, chr15:44711599-44711624, chr15:44711611-44711636, chr15:44715412-44715437, chr15:44715440-44715465, chr15:44715473-44715498, chr15:44715474-44715499, chr15:44715515-44715540, chr15:44715535-44715560, chr15:44715562-44715587, chr15:44715567-44715592, chr15:44715672-44715697, chr15:44715673-44715698, chr15:44715674-44715699, chr15:44715410-44715435, chr15:44715411-44715436, chr15:44715419-44715444, chr15:44715430-44715455, chr15:44715457-44715482, chr15:44715483-44715508, chr15:44715511-44715536, chr15:44715515-44715540, chr15:44715629-44715654, chr15:44715630-44715655, chr15:44715631-44715656, chr15:44715632-44715657, chr15:44715653-44715678, chr15:44715657-44715682, chr15:44715666-44715691, chr15:44715685-44715710, chr15:44715686-44715711, chr15:44716326-44716351, chr15:44716329-44716354, chr15:44716313-44716338, chr15:44717599-44717624, chr15:44717604-44717629, chr15:44717681-44717706, chr15:44717682-44717707, chr15:44717702-44717727, chr15:44717764-44717789, chr15:44717776-44717801, chr15:44717786-44717811, chr15:44717789-44717814, chr15:44717790-44717815, chr15:44717794-44717819, chr15:44717805-44717830, chr15:44717808-44717833, chr15:44717809-44717834, chr15:44717810-44717835, chr15:44717846-44717871, chr15:44717945-44717970, chr15:44717946-44717971, chr15:44717947-44717972, chr15:44717948-44717973, chr15:44717973-44717998, chr15:44717981-44718006, chr15:44718056-44718081, chr15:44718061-44718086, chr15:44718067-44718092, chr15:44718076-44718101, chr15:44717589-44717614, chr15:44717620-44717645, chr15:44717642-44717667, chr15:44717771-44717796, chr15:44717800-44717825, chr15:44717859-44717884, chr15:44717947-44717972, chr15:44718119-44718144, chr15:44711563-44711585, chr15:44715428-44715450, chr15:44715509-44715531, chr15:44715513-44715535, chr15:44715417-44715439, chr15:44711540-44711562, chr15:44711574-44711596, chr15:44711597-44711619, chr15:44715446-44715468, chr15:44715651-44715673, chr15:44713812-44713834, chr15:44711579-44711601, chr15:44711542-44711564, chr15:44711557-44711579, chr15:44711609-44711631, chr15:44715678-44715700, chr15:44715683-44715705, chr15:44715684-44715706, chr15:44715480-44715502.

8. The modified limbal stem cell of claim 7, wherein the targeting domain of the gRNA molecule is complementary to a sequence within a genomic region selected from:
chr15:44715513-44715535, chr15:44711542-44711564, chr15:44711563-44711585, chr15:44715683-44715705, chr15:44711597-44711619, or chr15:44715446-44715468.

9. The modified limbal stem cell of claim 7, wherein the targeting domain of the gRNA molecule is complementary to a sequence within a genomic region chr15:44711563-44711585.

10. The modified limbal stem cell of any one of claims 1-6, wherein the targeting domain of the gRNA molecule to B2M comprises a targeting domain comprising the sequence of any one of SEQ ID NOs: 23-105 or 108-119 or 134-140.

11. The modified limbal stem cell of claim 10, wherein the targeting domain of the gRNA molecule to B2M comprises a targeting domain comprising the sequence of any one of SEQ ID NOs: 108, 111, 115, 116, 134 or 138.

12. The modified limbal stem cell of claim 10, wherein the targeting domain of the gRNA molecule to B2M comprises a targeting domain comprising the sequence of SEQ ID NO: 108.

13. The modified limbal stem cell of claim 10, wherein the targeting domain of the gRNA molecule to B2M comprises a targeting domain comprising the sequence of SEQ ID NO: 115.

14. The modified limbal stem cell of claim 10, wherein the targeting domain of the gRNA molecule to B2M comprises a targeting domain comprising the sequence of SEQ ID NO: 116.

15. The modified limbal stem cell of any one of claims 1-6, wherein the gRNA comprises the sequence of any one of SEQ ID NOs: 120, 160-177.

16. The modified limbal stem cell of claim 15, wherein the gRNA comprises the sequence of any one of SEQ ID NOs: 120, 162, 166, 167, 171, and 175.

17. The modified limbal stem cell of claim 15, wherein the gRNA comprises the sequence of SEQ ID NO: 120.

18. The modified limbal stem cell of claim 15, wherein the gRNA comprises the sequence of SEQ ID NO: 166.

19. The modified limbal stem cell of claim 15, wherein the gRNA comprises the sequence of SEQ ID NO: 167.

20. The modified limbal stem cell of claims 1-19, wherein the CRISPR system is an S. pyogenes Cas9 CRISPR system.

21. The modified limbal stem cell of claim 20, wherein the CRISPR system comprises a Cas9 molecule comprising SEQ ID NO: 106 or 107 or any of SEQ ID NOs: 124 to 134.

22. The modified limbal stem cell of claim 20, wherein the CRISPR system comprises a Cas9 molecule comprising SEQ ID NO: 106 or 107.

23. A modified limbal stem cell comprising a genome in which the b2 microglobulin (B2M) gene on chromosome 15 has been edited
(a) to delete a contiguous stretch of genomic DNA comprising the sequence of any one of SEQ ID NOs: 141 to 159, thereby eliminating surface expression of MHC Class I molecules in the cell, or
(b) to form an indel at or near the target sequence complementary to the targeting domain of the gRNA molecule comprising the sequence of any one of SEQ ID NOs: 23-105 or 108-119 or 134-140, thereby eliminating surface expression of MHC Class I molecules in the cell.

24. The modified limbal stem cell of claim 23 comprising a genome in which the b2 microglobulin (B2M) gene on chromosome 15 has been edited:
(a) to delete a contiguous stretch of genomic DNA comprising the sequence of any one of SEQ ID NOs: 141, 148 or 149, thereby eliminating surface expression, of MHC Class I molecules in the cell, or
(b) to form an indel at or near the target sequence complementary to the targeting domain of the gRNA molecule domain comprising the sequence of any one of SEQ ID NOs: 108, 111, 115, 116, 134 or 138, thereby eliminating surface expression of MHC Class I molecules in the cell.

25. The modified limbal stem cell of claim 23 comprising a genome in which the b2 microglobulin (B2M) gene on chromosome 15 has been:
(a) edited to delete a contiguous stretch of genomic DNA comprising the sequence of SEQ ID NO: 141, thereby eliminating surface expression, of MHC Class I molecules in the cell, or
(b) to form an indel at or near the target sequence complementary to the targeting domain of the gRNA molecule domain comprising the sequence of SEQ ID NO: 108, thereby eliminating surface expression of MHC Class I molecules in the cell.

26. A modified limbal stem cell comprising a genome in which the b2 microglobulin (B2M) gene on chromosome 15 has been edited:
(a) to delete a contiguous stretch of genomic DNA region selected from any one of:
chr15:44711469-44711494, chr15:44711472-44711497, chr15:44711483-44711508, chr15:44711486-44711511, chr15:44711487-44711512, chr15:44711512-44711537, chr15:44711513-44711538, chr15:44711534-44711559, chr15:44711568-44711593, chr15:44711573-44711598, chr15:44711576-44711601, chr15:44711466-44711491, chr15:44711522-44711547, chr15:44711544-44711569, chr15:44711559-44711584, chr15:44711565-44711590, chr15:44711599-44711624, chr15:44711611-44711636, chr15:44715412-44715437, chr15:44715440-44715465, chr15:44715473-44715498, chr15:44715474-44715499, chr15:44715515-44715540, chr15:44715535-44715560, chr15:44715562-44715587, chr15:44715567-44715592, chr15:44715672-44715697, chr15:44715673-44715698, chr15:44715674-44715699, chr15:44715410-44715435, chr15:44715411-44715436, chr15:44715419-44715444, chr15:44715430-44715455, chr15:44715457-44715482, chr15:44715483-44715508, chr15:44715511-44715536, chr15:44715515-44715540, chr15:44715629-44715654, chr15:44715630-44715655, chr15:44715631-44715656, chr15:44715632-44715657, chr15:44715653-44715678, chr15:44715657-44715682, chr15:44715666-44715691, chr15:44715685-44715710, chr15:44715686-44715711, chr15:44716326-44716351, chr15:44716329-44716354, chr15:44716313-44716338, chr15:44717599-44717624, chr15:44717604-44717629, chr15:44717681-44717706, chr15:44717682-44717707, chr15:44717702-44717727, chr15:44717764-44717789, chr15:44717776-44717801, chr15:44717786-44717811, chr15:44717789-44717814, chr15:44717790-44717815, chr15:44717794-44717819, chr15:44717805-44717830, chr15:44717808-44717833, chr15:44717809-44717834, chr15:44717810-44717835, chr15:44717846-44717871, chr15:44717945-44717970, chr15:44717946-44717971, chr15:44717947-44717972, chr15:44717948-44717973, chr15:44717973-44717998, chr15:44717981-44718006, chr15:44718056-44718081, chr15:44718061-44718086, chr15:44718067-44718092, chr15:44718076-44718101, chr15:44717589-44717614, chr15:44717620-44717645, chr15:44717642-44717667, chr15:44717771-44717796, chr15:44717800-44717825, chr15:44717859-44717884, chr15:44717947-44717972, chr15:44718119-44718144, chr15:44711563-44711585, chr15:44715428-44715450, chr15:44715509-44715531, chr15:44715513-44715535, chr15:44715417-44715439, chr15:44711540-44711562, chr15:44711574-44711596, chr15:44711597-44711619, chr15:44715446-44715468, chr15:44715651-44715673, chr15:44713812-44713834, chr15:44711579-44711601, chr15:44711542-44711564, chr15:44711557-44711579, chr15:44711609-44711631, chr15:44715678-44715700, chr15:44715683-44715705, chr15:44715684-44715706, chr15:44715480-44715502, thereby eliminating surface expression of MHC Class I molecules in the cell, or
(b) to form an indel at or near the genomic DNA region selected from any one of:
chr15:44711469-44711494, chr15:44711472-44711497, chr15:44711483-44711508, chr15:44711486-44711511, chr15:44711487-44711512, chr15:44711512-44711537, chr15:44711513-44711538, chr15:44711534-44711559, chr15:44711568-44711593, chr15:44711573-44711598, chr15:44711576-44711601, chr15:44711466-44711491, chr15:44711522-44711547, chr15:44711544-44711569, chr15:44711559-44711584, chr15:44711565-44711590, chr15:44711599-44711624, chr15:44711611-44711636, chr15:44715412-44715437, chr15:44715440-44715465, chr15:44715473-44715498, chr15:44715474-44715499, chr15:44715515-44715540, chr15:44715535-44715560, chr15:44715562-44715587, chr15:44715567-44715592, chr15:44715672-44715697, chr15:44715673-44715698, chr15:44715674-44715699, chr15:44715410-44715435, chr15:44715411-44715436, chr15:44715419-44715444, chr15:44715430-44715455, chr15:44715457-44715482, chr15:44715483-44715508, chr15:44715511-44715536, chr15:44715515-44715540, chr15:44715629-44715654, chr15:44715630-44715655, chr15:44715631-44715656, chr15:44715632-44715657, chr15:44715653-44715678, chr15:44715657-44715682, chr15:44715666-44715691, chr15:44715685-44715710, chr15:44715686-44715711, chr15:44716326-44716351, chr15:44716329-44716354, chr15:44716313-44716338, chr15:44717599-44717624, chr15:44717604-44717629, chr15:44717681-44717706, chr15:44717682-44717707, chr15:44717702-44717727, chr15:44717764-44717789, chr15:44717776-44717801, chr15:44717786-44717811, chr15:44717789-44717814, chr15:44717790-44717815, chr15:44717794-44717819, chr15:44717805-44717830, chr15:44717808-44717833, chr15:44717809-44717834, chr15:44717810-44717835, chr15:44717846-44717871, chr15:44717945-44717970, chr15:44717946-44717971, chr15:44717947-44717972, chr15:44717948-44717973, chr15:44717973-44717998, chr15:44717981-44718006, chr15:44718056-44718081, chr15:44718061-44718086, chr15:44718067-44718092, chr15:44718076-44718101, chr15:44717589-44717614, chr15:44717620-44717645, chr15:44717642-44717667, chr15:44717771-44717796, chr15:44717800-44717825, chr15:44717859-44717884, chr15:44717947-44717972, chr15:44718119-44718144, chr15:44711563-44711585, chr15:44715428-44715450, chr15:44715509-44715531, chr15:44715513-44715535, chr15:44715417-44715439, chr15:44711540-44711562, chr15:44711574-44711596, chr15:44711597-44711619, chr15:44715446-44715468, chr15:44715651-44715673, chr15:44713812-44713834, chr15:44711579-44711601, chr15:44711542-44711564, chr15:44711557-44711579, chr15:44711609-44711631, chr15:44715678-44715700, chr15:44715683-44715705, chr15:44715684-44715706, chr15:44715480-44715502, thereby eliminating surface expression of MHC Class I molecules in the cell.

27. The modified limbal stem cell of claim 26 comprising a genome in which the b2 microglobulin (B2M) gene on chromosome 15 has been edited:
(a) to delete a contiguous stretch of genomic DNA region selected from:
chr15:44715513-44715535, chr15:44711542-44711564, chr15:44711563-44711585, chr15:44715683-44715705, chr15:44711597-44711619, or chr15:44715446-44715468, or
(b) to form an indel at or near the genomic DNA region selected from any one of:
chr15:44715513-44715535, chr15:44711542-44711564, chr15:44711563-44711585, chr15:44715683-44715705, chr15:44711597-44711619, or chr15:44715446-44715468.

28. The modified limbal stem cell of claim 26 comprising a genome in which the b2 microglobulin (B2M) gene on chromosome 15 has been edited
(a) to delete a contiguous stretch of genomic DNA region chr15:44711563-44711585, thereby eliminating surface expression of MHC Class I molecules in the cell, or
(b) to form an indel at or near the genomic DNA region chr15:44711563-44711585, thereby eliminating surface expression of MHC Class I molecules in the cell.

29. The modified limbal stem cell of any one of the previous claims,wherein the modified limbal stem cell comprises an indel formed at or near the target sequence complementary to the targeting domain of the gRNA molecule.

30. The modified limbal stem cell of any one of claims 23(b), 24(b), 25(b), 26(b), 27(b) or 28(b) or 29, wherein wherein the indel comprises a deletion of 10 or greater than 10 nucleotides, optionally 11, 12, 13, 14, 15, 16, 17, 18, 19, 20, 21, 22, 23, 24, 25, 26, 27, 28, 29, 30, 31, 32, 33, 34, or 35 nucleotides.

31. The modified limbal stem cell any one of claims 23 to 30, wherein the modified limbal stem cell was cultured in media comprising a large tumor suppressor kinase (“LATS”) inhibitor, optionally wherein the LATS inhibitor is a compound of Formula A1 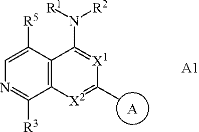
or a salt thereof, wherein
X¹ and X² are each independently CH or N;
Ring A is
(a) a 5- or 6-membered monocyclic heteroaryl that is linked to the remainder of the molecule through a carbon ring member and comprises, as ring member, 1 to 4 heteroatoms that are independently selected from N, O and S, provided that at least one of the heteroatom ring member is an unsubstituted nitrogen (—N═) positioned at the 3- or the 4-position relative to the linking carbon ring member of the 5-membered heteroaryl or at the para ring position of the 6-membered heteroaryl; or
(b) a 9-membered fused bicyclic heteroaryl that is selected from 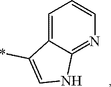 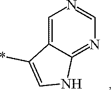 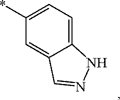 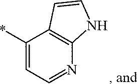 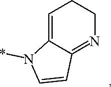
wherein “*” represents the point of attachment of ring A to the remainder of the molecule;
wherein ring A is unsubstituted or substituted by 1 to 2 substituents independently selected from halogen, cyano, C₁₋₆alkyl, C₁₋₆haloalkyl, —NH₂, C₁₋₆alkylamino, di-(C₁₋₆alkyl)amino, C₃₋₆cycloalkyl, and phenylsulfonyl;
R⁰ is hydroxyl or C₁₋₆alkoxy;
R¹ is hydrogen or C₁₋₆alkyl;
R² is selected from
(a) C₁₋₈alkyl that is unsubstituted or substituted by 1 to 3 substituents independently selected from
(i) halogen;
(ii) cyano;
(iii) oxo;
(iv) C₂alkenyl;
(v) C₂alkynyl;
(vi) C₁₋₆haloalkyl;
(vii) —OR⁶, wherein R⁶ is selected from hydrogen, C₁₋₆alkyl that is unsubstituted or substituted by R⁰ or —C(O)R⁰;
(viii) —NR^{7a}R^{7b}, wherein R^{7a} is hydrogen or C₁₋₆alkyl, and R^{7b} is selected from hydrogen, —C(O)R⁰, C₁₋₆alkyl that is unsubstituted or substituted by —C(O)R⁰;
(ix) —C(O)R⁸, wherein R⁸ is R⁰ or —NH—C₁₋₆alkyl-C(O)R⁰;
(x) -S(O)₂C₁₋₆alkyl;
(xi) monocyclic C₃₋₆cycloalkyl or polycyclic C₇₋₁₀cycloalkyl that are each unsubstituted or substituted by 1 to 2 substituents independently selected from halogen, C₁₋ ₆alkyl, hydroxyC₁₋₆alkyl, C₁₋₆haloalkyl, R⁰, —NH₂, C₁₋₆alkylamino, and di-(C₁₋ ₆alkyl)amino;
(xii) 6-membered heterocycloalkyl comprising, as ring members, 1 to 2 heteroatoms independently selected from N, O and S and that is unsubstituted or substituted by 1 to 2 substituents independently selected from hydroxyl, halogen, C₁₋₆alkyl, C₁₋₆alkylamino, and di-(C₁₋₆alkyl)amino;
(xiii) phenyl that is unsubstituted or substituted by halogen;
(xiv) 5- or 6-membered monocyclic heteroaryl comprising, as ring members, 1 to 4 heteroatoms independently selected from N and O; and
(xv) 9- or 10-membered fused bicyclic heteroaryl comprising, as ring member, 1 to 2 heteroatoms independently selected from N and O;
(b) -S(O)₂C₁₋₆alkyl;
(c) phenyl that is unsubstituted or substituted by 1 to 2 substituents independently selected from halogen, C₁₋₆alkyl and R⁰;
(d) C₃₋₆cycloalkyl that is unsubstituted or substituted by 1 to 2 substituents independently selected from C₁₋₆haloalkyl, R⁰, C₁₋₆alkylamino, di-(C₁₋₆alkyl)amino, —C(O)R⁰, and C₁₋ ₆alkyl that is unsubstituted or substituted by R⁰ or —C(O)R⁰; and
(e) 4-membered heterocycloalkyl comprising, as ring members, 1 to 2 heteroatoms selected from N, O and S and that is unsubstituted or substituted by 1 to 2 substituents independently selected from C₁₋₆haloalkyl, R⁰, C₁₋₆alkylamino, di-(C₁₋ ₆alkyl)amino, —C(O)R⁰, and C₁₋₆alkyl that is unsubstituted or substituted by R⁰ or —C(O)R⁰;
or R¹ and R² can be taken together with the nitrogen atom to which both are bound to form a 4- to 6-membered heterocycloalkyl that can include, as ring members, 1 to 2 additional heteroatoms independently selected from N, O, and S, wherein the 4- to 6-membered heterocycloalkyl formed by R¹ and R² taken together with the nitrogen atom to which both are bound is unsubstituted or substituted by 1 to 3 substituents independently selected from halogen, C₁₋₆alkyl, C₁₋₆haloalkyl, and R⁰;
R³ is selected from hydrogen, halogen and C₁₋₆alkyl; and
R⁵ is selected from hydrogen, halogen and —NH—(3- to 8-membered heteroalkyl), wherein the 3- to 8-membered heteroC₃₋₈alkyl of the —NH—(3- to 8-membered heteroalkyl) comprises 1 to 2 oxygen atoms as chain members and is unsubstituted or substituted by R⁰.

32. The modified limbal stem cell according to claim 31, wherein the compound is selected from: dimethyl(3-methyl-3-{[2-(pyridin-4-yl)pyrido[3,4-d]pyrimidin-4-yl]amino}butyl)amine and N¹,N¹,3-trimethyl-N³-(2-(3-methyl-1H-pyrazol-4-yl)pyrido[3,4-d]pyrimidin-4-yl)butane-1,3-diamine.

33. The modified limbal stem cell according to claim 31, wherein the compound is dimethyl(3-methyl-3-{[2-(pyridin-4-yl)pyrido[3,4-d]pyrimidin-4-yl]amino}butyl)amine.

34. The modified limbal stem cell according to any one of claims 31 to 33, wherein the compound is present in a concentration of 3 to 10 micromolar.

35. The modified limbal stem cell of any of claims 1-34, wherein the cell is autologous with respect to a patient to be administered said cell.

36. The modified limbal stem cell of any of claims 1-34, wherein the cell is allogeneic with respect to a patient to be administered said cell.

37. A method of preparing a modified limbal stem cell or a population of modified limbal stem cells for ocular cell therapy comprising,
a) modifying a limbal stem cell or a population of limbal stem cells by reducing or eliminating expression of B2M comprising introducing into the limbal stem cell or the population of limbal stem cells a CRISPR system comprising a gRNA molecule with a targeting domain
(i) comprising the sequence of any one of SEQ ID NOs: 23-105 or 108-119, or 134 to 140, or
(ii) complementary to a sequence within a genomic region selected from:
chr15:44711469-44711494, chr15:44711472-44711497, chr15:44711483-44711508, chr15:44711486-44711511, chr15:44711487-44711512, chr15:44711512-44711537, chr15:44711513-44711538, chr15:44711534-44711559, chr15:44711568-44711593, chr15:44711573-44711598, chr15:44711576-44711601, chr15:44711466-44711491, chr15:44711522-44711547, chr15:44711544-44711569, chr15:44711559-44711584, chr15:44711565-44711590, chr15:44711599-44711624, chr15:44711611-44711636, chr15:44715412-44715437, chr15:44715440-44715465, chr15:44715473-44715498, chr15:44715474-44715499, chr15:44715515-44715540, chr15:44715535-44715560, chr15:44715562-44715587, chr15:44715567-44715592, chr15:44715672-44715697, chr15:44715673-44715698, chr15:44715674-44715699, chr15:44715410-44715435, chr15:44715411-44715436, chr15:44715419-44715444, chr15:44715430-44715455, chr15:44715457-44715482, chr15:44715483-44715508, chr15:44715511-44715536, chr15:44715515-44715540, chr15:44715629-44715654, chr15:44715630-44715655, chr15:44715631-44715656, chr15:44715632-44715657, chr15:44715653-44715678, chr15:44715657-44715682, chr15:44715666-44715691, chr15:44715685-44715710, chr15:44715686-44715711, chr15:44716326-44716351, chr15:44716329-44716354, chr15:44716313-44716338, chr15:44717599-44717624, chr15:44717604-44717629, chr15:44717681-44717706, chr15:44717682-44717707, chr15:44717702-44717727, chr15:44717764-44717789, chr15:44717776-44717801, chr15:44717786-44717811, chr15:44717789-44717814, chr15:44717790-44717815, chr15:44717794-44717819, chr15:44717805-44717830, chr15:44717808-44717833, chr15:44717809-44717834, chr15:44717810-44717835, chr15:44717846-44717871, chr15:44717945-44717970, chr15:44717946-44717971, chr15:44717947-44717972, chr15:44717948-44717973, chr15:44717973-44717998, chr15:44717981-44718006, chr15:44718056-44718081, chr15:44718061-44718086, chr15:44718067-44718092, chr15:44718076-44718101, chr15:44717589-44717614, chr15:44717620-44717645, chr15:44717642-44717667, chr15:44717771-44717796, chr15:44717800-44717825, chr15:44717859-44717884, chr15:44717947-44717972, chr15:44718119-44718144, chr15:44711563-44711585, chr15:44715428-44715450, chr15:44715509-44715531, chr15:44715513-44715535, chr15:44715417-44715439, chr15:44711540-44711562, chr15:44711574-44711596, chr15:44711597-44711619, chr15:44715446-44715468, chr15:44715651-44715673, chr15:44713812-44713834, chr15:44711579-44711601, chr15:44711542-44711564, chr15:44711557-44711579, chr15:44711609-44711631, chr15:44715678-44715700, chr15:44715683-44715705, chr15:44715684-44715706, chr15:44715480-44715502,
wherein the limbal stem cell or the population of limbal stem cells have optionally been cultured in the presence of a LATS inhibitor; and
b) further expanding the modified limbal stem cell or thepopulation of modified limbal stem cells in cell culture media comprising a LATS inhibitor; and
c) optionally, enriching the population of limbal stem cells with the limbal stem cells having reduced or eliminated expression of B2M by fluorescene activated cell sorting (FACS) or magnetic activated cell sorting (MACS).

38. The method of claim 37, wherein the LATS inhibitor is a compound of Formula A1 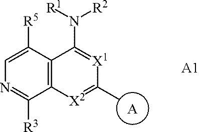
or a salt thereof, wherein
X¹ and X² are each independently CH or N;
Ring A is
(a) a 5- or 6-membered monocyclic heteroaryl that is linked to the remainder of the molecule through a carbon ring member and comprises, as ring member, 1 to 4 heteroatoms that are independently selected from N, O and S, provided that at least one of the heteroatom ring member is an unsubstituted nitrogen (—N═) positioned at the 3- or the 4-position relative to the linking carbon ring member of the 5-membered heteroaryl or at the para ring position of the 6-membered heteroaryl; or
(b) a 9-membered fused bicyclic heteroaryl that is selected from 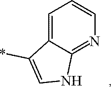 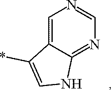 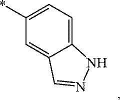 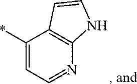 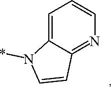
wherein “*” represents the point of attachment of ring A to the remainder of the molecule;
wherein ring A is unsubstituted or substituted by 1 to 2 substituents independently selected from halogen, cyano, C₁₋₆alkyl, C₁₋₆haloalkyl, —NH₂, C₁₋₆alkylamino, di-(C₁₋₆alkyl)amino, C₃₋₆cycloalkyl, and phenylsulfonyl;
R⁰ is hydroxyl or C₁₋₆alkoxy;
R¹ is hydrogen or C₁₋₆alkyl;
R² is selected from
(a) C₁₋₈alkyl that is unsubstituted or substituted by 1 to 3 substituents independently selected from
(i) halogen;
(ii) cyano;
(iii) oxo;
(iv) C₂alkenyl;
(v) C₂alkynyl;
(vi) C₁₋₆haloalkyl;
(vii) —OR⁶, wherein R⁶ is selected from hydrogen, C₁₋₆alkyl that is unsubstituted or substituted by R⁰ or —C(O)R⁰;
(viii) —NR^{7a}R^{7b}, wherein R^{7a} is hydrogen or C₁₋₆alkyl, and R^{7b} is selected from hydrogen, —C(O)R⁰, C₁₋₆alkyl that is unsubstituted or substituted by —C(O)R⁰;
(ix) —C(O)R⁸, wherein R⁸ is R⁰ or —NH—C₁₋₆alkyl-C(O)R⁰;
(x) -S(O)₂C₁₋₆alkyl;
(xi) monocyclic C₃₋₆cycloalkyl or polycyclic C₇₋₁₀cycloalkyl that are each unsubstituted or substituted by 1 to 2 substituents independently selected from halogen, C₁₋ ₆alkyl, hydroxyC₁₋₆alkyl, C₁₋₆haloalkyl, R⁰, —NH₂, C₁₋₆alkylamino, and di-(C₁₋ ₆alkyl)amino;
(xii) 6-membered heterocycloalkyl comprising, as ring members, 1 to 2 heteroatoms independently selected from N, O and S and that is unsubstituted or substituted by 1 to 2 substituents independently selected from hydroxyl, halogen, C₁₋₆alkyl, C₁₋₆alkylamino, and di-(C₁₋₆alkyl)amino;
(xiii) phenyl that is unsubstituted or substituted by halogen;
(xiv) 5- or 6-membered monocyclic heteroaryl comprising, as ring members, 1 to 4 heteroatoms independently selected from N and O; and
(xv) 9- or 10-membered fused bicyclic heteroaryl comprising, as ring member, 1 to 2 heteroatoms independently selected from N and O;
(b) -S(O)₂C₁₋₆alkyl;
(c) phenyl that is unsubstituted or substituted by 1 to 2 substituents independently selected from halogen, C₁₋₆alkyl and R⁰;
(d) C₃₋₆cycloalkyl that is unsubstituted or substituted by 1 to 2 substituents independently selected from C₁₋₆haloalkyl, R⁰, C₁₋₆alkylamino, di-(C₁₋₆alkyl)amino, —C(O)R⁰, and C₁₋ ₆alkyl that is unsubstituted or substituted by R⁰ or —C(O)R⁰; and
(e) 4-membered heterocycloalkyl comprising, as ring members, 1 to 2 heteroatoms selected from N, O and S and that is unsubstituted or substituted by 1 to 2 substituents independently selected from C₁₋₆haloalkyl, R⁰, C₁₋₆alkylamino, di-(C₁₋ ₆alkyl)amino, —C(O)R⁰, and C₁₋₆alkyl that is unsubstituted or substituted by R⁰ or —C(O)R⁰;
or R¹ and R² can be taken together with the nitrogen atom to which both are bound to form a 4- to 6-membered heterocycloalkyl that can include, as ring members, 1 to 2 additional heteroatoms independently selected from N, O, and S, wherein the 4- to 6-membered heterocycloalkyl formed by R¹ and R² taken together with the nitrogen atom to which both are bound is unsubstituted or substituted by 1 to 3 substituents independently selected from halogen, C₁₋₆alkyl, C₁₋₆haloalkyl, and R⁰;
R³ is selected from hydrogen, halogen and C₁₋₆alkyl; and
R⁵ is selected from hydrogen, halogen and —NH—(3- to 8-membered heteroalkyl), wherein the 3- to 8-membered heteroC₃₋₈alkyl of the —NH—(3- to 8-membered heteroalkyl) comprises 1 to 2 oxygen atoms as chain members and is unsubstituted or substituted by R⁰.

39. The method according to claim 38, wherein the compound is selected from: dimethyl(3-methyl-3-{[2-(pyridin-4-yl)pyrido[3,4-d]pyrimidin-4-yl]amino}butyl)amine and N¹,N¹,3-trimethyl-N³-(2-(3-methyl-1H-pyrazol-4-yl)pyrido[3,4-d]pyrimidin-4-yl)butane-1,3-diamine.

40. The method according to claim 38, wherein the compound is dimethyl(3-methyl-3-{[2-(pyridin-4-yl)pyrido[3,4-d]pyrimidin-4-yl]amino}butyl)amine.

41. The method according to any one of claims 38 to 40, wherein the compound is present in a concentration of 3 to 10 micromolar.

42. The method of any one of claims 37-41, wherein the CRISPR system is an S. pyogenes Cas9 CRISPR system.

43. The method of claim 42, wherein the CRISPR system comprises a Cas 9 molecule comprising SEQ ID NO: 106 or 107 or any of SEQ ID NOs: 124 to 134.

44. The method of claim 42, wherein the CRISPR system comprises a Cas 9 molecule comprising SEQ ID NO: 106 or 107.

45. A cell population comprising the modified limbal stem cell of any one of claims 1 to 36 or the modified limbal stem cell obtained by the method of any one of claims 37-44.

46. The cell population of claim 45, wherein the modified limbal stem cell comprises an indel formed at or near the target sequence complementary to the targeting domain of the gRNA molecule domain.

47. The cell population of claim 46, wherein the indel comprises a deletion of 10 or greater than 10 nucleotides, optionally 11, 12, 13, 14, 15, 16, 17, 18, 19, 20, 21, 22, 23, 24, 25, 26, 27, 28, 29, 30, 31,32, 33, 34, or 35 nucleotides.

48. The cell population of claim 45 or 46, wherein the indel is formed in at least about 40%, e.g., at least about 50%, e.g., at least about 60%, e.g., at least about 70%, e.g., at least about 80%, e.g., at least about 90%, e.g., at least about 95%, e.g., at least about 96%, e.g., at least about 97%, e.g., at least about 98%, e.g., at least about 99%, of the cells of the cell population, e.g., as detectible by next generation sequencing and/or a nucleotide insertional assay.

49. The cell population of any one of claims 45 to 48, wherein an off-target indel is detected in no more than about 5%, e.g., no more than about 1%, e.g., no more than about 0.1%, e.g., no more than about 0.01%, of the cells of the cell population, e.g., as detectible by next generation sequencing and/or a nucleotide insertional assay.

50. A composition comprising the modified limbal stem cell of any one of claims 1 to 36 or the modified limbal stem cell obtained by the method of any one of claims 37-44 or the cell population of any one of claims 45-49 or the population of modified limbal stem cells obtained by the method of any one of claims 37-44.

51. The composition of claim 50, wherein the modified limbal stem cell comprises an indel formed at or near the target sequence complementary to the targeting domain of the gRNA molecule domain.

52. The composition of claim 51, wherein the indel comprises a deletion of 10 or greater than 10 nucleotides, optionally 11, 12, 13, 14, 15, 16, 17, 18, 19, 20, 21, 22, 23, 24, 25, 26, 27, 28, 29, 30, 31,32, 33, 34, or 35 nucleotides.

53. The composition of claim 51 or 52, wherein the indel is formed in at least about 40%, e.g., at least about 50%, e.g., at least about 60%, e.g., at least about 70%, e.g., at least about 80%, e.g., at least about 90%, e.g., at least about 95%, e.g., at least about 96%, e.g., at least about 97%, e.g., at least about 98%, e.g., at least about 99%, of the cells of the population.

54. The composition of any one of claims 51 to 53, wherein an off-target indel is detected in no more than about 5%, e.g., no more than about 1%, e.g., no more than about 0.1%, e.g., no more than about 0.01%, of the cells of the population of cells e.g., as detectible by next generation sequencing and/or a nucleotide insertional assay.

55. The modified limbal stem cell of any one of claims 1 to 36 or the cell population of any one of claims 45 to 49 or the composition of any one of claims 50 to 54 for use in treatment of an an ocular disease.

56. The modified limbal stem cell or the cell population or the composition for use according to claim 55, wherein the ocular disease is limbal stem cell deficiency.

57. The modified limbal stem cell or the cell population or the composition for use according to claim 56, wherein the ocular disease is unilateral limbal stem cell deficiency.

58. The modified limbal stem cell or the cell population or the composition for use according to claim 56, wherein the ocular disease is bilateral limbal stem cell deficiency.

59. The modified limbal stem cell or the cell population or the composition for use according to any one of claims 50 to 53, wherein the cell is autologous with respect to a patient to be administered said cell.

60. The modified limbal stem cell or the cell population or the composition for use according to any one of claims 50 to 53, wherein the cell is allogeneic with respect to a patient to be administered said cell.

61. A method of treating a patient suffering from an ocular disease comprising the step of administering to the patient in need thereof the modified limbal stem cell of any one of claims 1-36 or the cell population of any one of claims 45 to 49 or the composition of any one of claims 50 to 54.

62. The method of claim 61, wherein the ocular disease is limbal stem cell deficiency.

63. The method of claim 62, wherein the ocular disease is unilateral limbal stem cell deficiency.

64. The method of claim 62, wherein the ocular disease is bilateral limbal stem cell deficiency.

65. The method of any one of claims 62 to 64, wherein the cell is autologous with respect to a patient to be administered said cell.

66. The method of any one of claims 62 to 64, wherein the cell is allogeneic with respect to a patient to be administered said cell.

67. Use of the modified limbal stem cell of any one of claims 1 to 36 or the cell population of any one of claims 45 to 49 or the composition of any one of claims 50 to 54 for the treatment of an ocular disease.

68. Use of claim 67, wherein the ocular disease is limbal stem cell deficiency.


SEQUENCE LISTING (183 sequences)

SEQ ID NO: 1 - protein, 1130 aa, Homo sapiens
MKRSEKPEGYRQMRPKTFPASNYTVSSRQMLQEIRESLRNLSKPSDAAKAEHNMSKMSTEDPRQVRNPPKFGTHHKALQEIRNSLLPFANETNSSRSTSEVNPQMLQDLQAAGFDEDMVIQALQKTNNRSIEAAIEFISKMSYQDPRREQMAAAAARPINASMKPGNVQQSVNRKQSWKGSKESLVPQRHGPPLGESVAYHSESPNSQTDVGRPLSGSGISAFVQAHPSNGQRVNPPPPPQVRSVTPPPPPRGQTPPPRGTTPPPPSWEPNSQTKRYSGNMEYVISRISPVPPGAWQEGYPPPPLNTSPMNPPNQGQRGISSVPVGRQPIIMQSSSKFNFPSGRPGMQNGTGQTDFMIHQNVVPAGTVNRQPPPPYPLTAANGQSPSALQTGGSAAPSSYTNGSIPQSMMVPNRNSHNMELYNISVPGLQTNWPQSSSAPAQSSPSSGHEIPTWQPNIPVRSNSFNNPLGNRASHSANSQPSATTVTAITPAPIQQPVKSMRVLKPELQTALAPTHPSWIPQPIQTVQPSPFPEGTASNVTVMPPVAEAPNYQGPPPPYPKHLLHQNPSVPPYESISKPSKEDQPSLPKEDESEKSYENVDSGDKEKKQITTSPITVRKNKKDEERRESRIQSYSPQAFKFFMEQHVENVLKSHQQRLHRKKQLENEMMRVGLSQDAQDQMRKMLCQKESNYIRLKRAKMDKSMFVKIKTLGIGAFGEVCLARKVDTKALYATKTLRKKDVLLRNQVAHVKAERDILAEADNEWVVRLYYSFQDKDNLYFVMDYIPGGDMMSLLIRMGIFPESLARFYIAELTCAVESVHKMGFIHRDIKPDNILIDRDGHIKLTDFGLCTGFRWTHDSKYYQSGDHPRQDSMDFSNEWGDPSSCRCGDRLKPLERRAARQHQRCLAHSLVGTPNYIAPEVLLRTGYTQLCDWWSVGVILFEMLVGQPPFLAQTPLETQMKVINWQTSLHIPPQAKLSPEASDLIIKLCRGPEDRLGKNGADEIKAHPFFKTIDFSSDLRQQSASYIPKITHPTDTSNFDPVDPDKLWSDDNEEENVNDTLNGWYKNGKHPEHAFYEFTFRRFFDDNGYPYNYPKPIEYEYINSQGSEQQSDEDDQNTGSEIKNRDLVYV

SEQ ID NO: 2 - protein, 690 aa, Homo sapiens
MKRSEKPEGYRQMRPKTFPASNYTVSSRQMLQEIRESLRNLSKPSDAAKAEHNMSKMSTEDPRQVRNPPKFGTHHKALQEIRNSLLPFANETNSSRSTSEVNPQMLQDLQAAGFDEDMVIQALQKTNNRSIEAAIEFISKMSYQDPRREQMAAAAARPINASMKPGNVQQSVNRKQSWKGSKESLVPQRHGPPLGESVAYHSESPNSQTDVGRPLSGSGISAFVQAHPSNGQRVNPPPPPQVRSVTPPPPPRGQTPPPRGTTPPPPSWEPNSQTKRYSGNMEYVISRISPVPPGAWQEGYPPPPLNTSPMNPPNQGQRGISSVPVGRQPIIMQSSSKFNFPSGRPGMQNGTGQTDFMIHQNVVPAGTVNRQPPPPYPLTAANGQSPSALQTGGSAAPSSYTNGSIPQSMMVPNRNSHNMELYNISVPGLQTNWPQSSSAPAQSSPSSGHEIPTWQPNIPVRSNSFNNPLGNRASHSANSQPSATTVTAITPAPIQQPVKSMRVLKPELQTALAPTHPSWIPQPIQTVQPSPFPEGTASNVTVMPPVAEAPNYQGPPPPYPKHLLHQNPSVPPYESISKPSKEDQPSLPKEDESEKSYENVDSGDKEKKQITTSPITVRKNKKDEERRESRIQSYSPQAFKFFMEQHVENVLKSHQQRLHRKKQLENEMMRVKPFKMSIFILNHLFAWCLF

SEQ ID NO: 3 - protein, 1088 aa, Homo sapiens
MRPKTFPATTYSGNSRQRLQEIREGLKQPSKSSVQGLPAGPNSDTSLDAKVLGSKDATRQQQQMRATPKFGPYQKALREIRYSLLPFANESGTSAAAEVNRQMLQELVNAGCDQEMAGRALKQTGSRSIEAALEYISKMGYLDPRNEQIVRVIKQTSPGKGLMPTPVTRRPSFEGTGDSFASYHQLSGTPYEGPSFGADGPTALEEMPRPYVDYLFPGVGPHGPGHQHQHPPKGYGASVEAAGAHFPLQGAHYGRPHLLVPGEPLGYGVQRSPSFQSKTPPETGGYASLPTKGQGGPPGAGLAFPPPAAGLYVPHPHHKQAGPAAHQLHVLGSRSQVFASDSPPQSLLTPSRNSLNVDLYELGSTSVQQWPAATLARRDSLQKPGLEAPPRAHVAFRPDCPVPSRTNSFNSHQPRPGPPGKAEPSLPAPNTVTAVTAAHILHPVKSVRVLRPEPQTAVGPSHPAWVPAPAPAPAPAPAPAAEGLDAKEEHALALGGAGAFPLDVEYGGPDRRCPPPPYPKHLLLRSKSEQYDLDSLCAGMEQSLRAGPNEPEGGDKSRKSAKGDKGGKDKKQIQTSPVPVRKNSRDEEKRESRIKSYSPYAFKFFMEQHVENVIKTYQQKVNRRLQLEQEMAKAGLCEAEQEQMRKILYQKESNYNRLKRAKMDKSMFVKIKTLGIGAFGEVCLACKVDTHALYAMKTLRKKDVLNRNQVAHVKAERDILAEADNEWVVKLYYSFQDKDSLYFVMDYIPGGDMMSLLIRMEVFPEHLARFYIAELTLAIESVHKMGFIHRDIKPDNILIDLDGHIKLTDFGLCTGFRWTHNSKYYQKGSHVRQDSMEPSDLWDDVSNCRCGDRLKTLEQRARKQHQRCLAHSLVGTPNYIAPEVLLRKGYTQLCDWWSVGVILFEMLVGQPPFLAPTPTETQLKVINWENTLHIPAQVKLSPEARDLITKLCCSADHRLGRNGADDLKAHPFFSAIDFSSDIRKQPAPYVPTISHPMDTSNFDPVDEESPWNDASEGSTKAWDTLTSPNNKHPEHAFYEFTFRRFFDDNGYPFRCPKPSGAEASQAESSDLESSDLVDQTEGCQPVYV

SEQ ID NO: 4 - DNA, 5 bp, Unknown
  Description of Unknown: Target sequence
  modified_base (1)..(1): a, c, t, g, unknown or other
  misc_feature (1)..(1): n is a, c, g, or t
  modified_base (4)..(4): a, c, t, g, unknown or other
  misc_feature (4)..(4): n is a, c, g, or t
nggng

SEQ ID NO: 5 - DNA, 7 bp, Unknown
  Description of Unknown: Target sequence
  modified_base (1)..(2): a, c, t, g, unknown or other
  misc_feature (1)..(2): n is a, c, g, or t
nnagaaw

SEQ ID NO: 6 - DNA, 5 bp, Unknown
  Description of Unknown: Target sequence
  modified_base (1)..(2): a, c, t, g, unknown or other
  misc_feature (1)..(2): n is a, c, g, or t
nngrr

SEQ ID NO: 7 - DNA, 8 bp, Unknown
  Description of Unknown: Target sequence
  modified_base (1)..(4): a, c, t, g, unknown or other
  misc_feature (1)..(4): n is a, c, g, or t
nnnngatt

SEQ ID NO: 8 - protein, 7 aa, Simian virus 40
PKKKRKV

SEQ ID NO: 9 - RNA, 42 nt, Artificial Sequence
  Description of Artificial Sequence: Synthetic oligonucleotide
  modified_base (1)..(20): a, c, u, g, unknown or other
  misc_feature (1)..(20): n is a, c, g, or u
nnnnnnnnnnnnnnnnnnnnguuuuagagcuaugcuguuuug

SEQ ID NO: 10 - RNA, 86 nt, Artificial Sequence
  Description of Artificial Sequence: Synthetic oligonucleotide
  misc_feature (80)..(86): This region may encompass 1-7 nucleotides
  misc_feature (80)..(86): This region may or may not be present
aacuuaccaaggaacagcauagcaaguuaaaauaaggcuaguccguuaucaacuugaaaaaguggcaccgagucggugcuuuuuuu

SEQ ID NO: 11 - RNA, 74 nt, Artificial Sequence
  Description of Artificial Sequence: Synthetic oligonucleotide
  misc_feature (68)..(74): This region may or may not be present
  misc_feature (68)..(74): This region may encompass 1-7 nucleotides
aacagcauagcaaguuaaaauaaggcuaguccguuaucaacuugaaaaaguggcaccgagucggugcuuuuuuu

SEQ ID NO: 12 - RNA, 102 nt, Artificial Sequence
  Description of Artificial Sequence: Synthetic polynucleotide
  modified_base (1)..(19): a, c, u, g, unknown or other
  misc_feature (1)..(19): n is a, c, g, or u
  modified_base (96)..(102)
  modified_base (96)..(102)
nnnnnnnnnnnnnnnnnnnguuuuagagcuagaaauagcaaguuaaaauaaggcuaguccguuaucaacuugaaaaaguggcaccgagucggugcuuuuuuu

SEQ ID NO: 13 - DNA, 21 bp, Unknown
  Description of Unknown: Target sequence
  modified_base (1)..(18)
  misc_feature (1)..(19): n is a, c, g, or t
  modified_base (19)..(19)
nnnnnnnnnnnnnnnnnnngg

SEQ ID NO: 14 - DNA, 19 bp, Unknown
  Description of Unknown: Target sequence
  modified_base (1)..(16)
  misc_feature (1)..(17): n is a, c, g, or t
  modified_base (17)..(17)
nnnnnnnnnnnnnnnnngg

SEQ ID NO: 15 - DNA, 21 bp, Unknown
  Description of Unknown: Target sequence
  modified_base (1)..(14): a, c, t, or g
  misc_feature (1)..(16): n is a, c, g, or t
  modified_base (15)..(16): a, c, t, g, unknown or other
nnnnnnnnnnnnnnnnagaaw

SEQ ID NO: 16 - DNA, 21 bp, Unknown
  Description of Unknown: Target sequence
  modified_base (1)..(14): a, c, t, or g
  misc_feature (1)..(16): n is a, c, g, or t
  modified_base (15)..(16): a, c, t, g, unknown or other
nnnnnnnnnnnnnnnnagaaw

SEQ ID NO: 17 - DNA, 23 bp, Unknown
  Description of Unknown: Target sequence
  modified_base (1)..(18): a, c, t, or g
  misc_feature (1)..(19): n is a, c, g, or t
  modified_base (19)..(19): a, c, t, g, unknown or other
  modified_base (22)..(22): a, c, t, g, unknown or other
  misc_feature (22)..(22): n is a, c, g, or t
nnnnnnnnnnnnnnnnnnnggng

SEQ ID NO: 18 - DNA, 16 bp, Unknown
  Description of Unknown: Target sequence
  modified_base (1)..(11): a, c, t, or g
  misc_feature (1)..(12): n is a, c, g, or t
  modified_base (12)..(12): a, c, t, g, unknown or other
  modified_base (15)..(15): a, c, t, g, unknown or other
  misc_feature (15)..(15): n is a, c, g, or t
nnnnnnnnnnnnggng

SEQ ID NO: 19 - DNA, 8 bp, Unknown
  Description of Unknown: Target sequence
  modified_base (1)..(4): a, c, t, g, unknown or other
  misc_feature (1)..(4): n is a, c, g, or t
nnnngatt

SEQ ID NO: 20 - DNA, 8 bp, Unknown
  Description of Unknown: Target sequence
  modified_base (1)..(4): a, c, t, g, unknown or other
  misc_feature (1)..(4): n is a, c, g, or t
nnnngctt

SEQ ID NO: 21 - DNA, 6 bp, Unknown
  Description of Unknown: Target sequence
  modified_base (1)..(2): a, c, t, g, unknown or other
  misc_feature (1)..(2): n is a, c, g, or t
nngrrt

SEQ ID NO: 22 - DNA, 6 bp, Unknown
  Description of Unknown: Target sequence
  modified_base (1)..(2): a, c, t, g, unknown or other
  misc_feature (1)..(2): n is a, c, g, or t
nngrrv

SEQ ID NO: 23 - RNA, 25 nt, Homo sapiens
uggcugggcacgcguuuaauauaag

SEQ ID NO: 24 - RNA, 25 nt, Homo sapiens
cugggcacgcguuuaauauaagugg

SEQ ID NO: 25 - RNA, 25 nt, Homo sapiens
uuuaauauaaguggaggcgucgcgc

SEQ ID NO: 26 - RNA, 25 nt, Homo sapiens
aauauaaguggaggcgucgcgcugg

SEQ ID NO: 27 - RNA, 25 nt, Homo sapiens
auauaaguggaggcgucgcgcuggc

SEQ ID NO: 28 - RNA, 25 nt, Homo sapiens
gggcauuccugaagcugacagcauu

SEQ ID NO: 29 - RNA, 25 nt, Homo sapiens
ggcauuccugaagcugacagcauuc

SEQ ID NO: 30 - RNA, 25 nt, Homo sapiens
auucgggccgagaugucucgcuccg

SEQ ID NO: 31 - RNA, 25 nt, Homo sapiens
cugugcucgcgcuacucucucuuuc

SEQ ID NO: 32 - RNA, 25 nt, Homo sapiens
cucgcgcuacucucucuuucuggcc

SEQ ID NO: 33 - RNA, 25 nt, Homo sapiens
gcgcuacucucucuuucuggccugg

SEQ ID NO: 34 - RNA, 25 nt, Homo sapiens
gcgcuacucucucuuucuggccugg

SEQ ID NO: 35 - RNA, 25 nt, Homo sapiens
ucucggcccgaaugcugucagcuuc

SEQ ID NO: 36 - RNA, 25 nt, Homo sapiens
gcuaaggccacggagcgagacaucu

SEQ ID NO: 37 - RNA, 25 nt, Homo sapiens
aguagcgcgagcacagcuaaggcca

SEQ ID NO: 38 - RNA, 25 nt, Homo sapiens
agagagaguagcgcgagcacagcua

SEQ ID NO: 39 - RNA, 25 nt, Homo sapiens
gagagacucacgcuggauagccucc

SEQ ID NO: 40 - RNA, 25 nt, Homo sapiens
gcgggaggguaggagagacucacgc

SEQ ID NO: 41 - RNA, 25 nt, Homo sapiens
uauuccucagguacuccaaagauuc

SEQ ID NO: 42 - RNA, 25 nt, Homo sapiens
uuuacucacgucauccagcagagaa

SEQ ID NO: 43 - RNA, 25 nt, Homo sapiens
caaauuuccugaauugcuauguguc

SEQ ID NO: 44 - RNA, 25 nt, Homo sapiens
aaauuuccugaauugcuaugugucu

SEQ ID NO: 45 - RNA, 25 nt, Homo sapiens
acauugaaguugacuuacugaagaa

SEQ ID NO: 46 - RNA, 25 nt, Homo sapiens
aagaauggagagagaauugaaaaag

SEQ ID NO: 47 - RNA, 25 nt, Homo sapiens
gagcauucagacuugucuuucagca

SEQ ID NO: 48 - RNA, 25 nt, Homo sapiens
uucagacuugucuuucagcaaggac

SEQ ID NO: 49 - RNA, 25 nt, Homo sapiens
uuugucacagcccaagauaguuaag

SEQ ID NO: 50 - RNA, 25 nt, Homo sapiens
uugucacagcccaagauaguuaagu

SEQ ID NO: 51 - RNA, 25 nt, Homo sapiens
ugucacagcccaagauaguuaagug

SEQ ID NO: 52 - RNA, 25 nt, Homo sapiens
aucuuuggaguaccugaggaauauc

SEQ ID NO: 53 - RNA, 25 nt, Homo sapiens
aaucuuuggaguaccugaggaauau

SEQ ID NO: 54 - RNA, 25 nt, Homo sapiens
uaaaccugaaucuuuggaguaccug

SEQ ID NO: 55 - RNA, 25 nt, Homo sapiens
gaugacgugaguaaaccugaaucuu

SEQ ID NO: 56 - RNA, 25 nt, Homo sapiens
ggaaauuugacuuuccauucucugc

SEQ ID NO: 57 - RNA, 25 nt, Homo sapiens
augaaacccagacacauagcaauuc

SEQ ID NO: 58 - RNA, 25 nt, Homo sapiens
ucaguaagucaacuucaaugucgga

SEQ ID NO: 59 - RNA, 25 nt, Homo sapiens
uucuucaguaagucaacuucaaugu

SEQ ID NO: 60 - RNA, 25 nt, Homo sapiens
caggcauacucaucuuuuucagugg

SEQ ID NO: 61 - RNA, 25 nt, Homo sapiens
gcaggcauacucaucuuuuucagug

SEQ ID NO: 62 - RNA, 25 nt, Homo sapiens
ggcaggcauacucaucuuuuucagu

SEQ ID NO: 63 - RNA, 25 nt, Homo sapiens
cggcaggcauacucaucuuuuucag

SEQ ID NO: 64 - RNA, 25 nt, Homo sapiens
gacaaagucacaugguucacacggc

SEQ ID NO: 65 - RNA, 25 nt, Homo sapiens
cugugacaaagucacaugguucaca

SEQ ID NO: 66 - RNA, 25 nt, Homo sapiens
uaucuugggcugugacaaagucaca

SEQ ID NO: 67 - RNA, 25 nt, Homo sapiens
aagacuuaccccacuuaacuaucuu

SEQ ID NO: 68 - RNA, 25 nt, Homo sapiens
uaagacuuaccccacuuaacuaucu

SEQ ID NO: 69 - RNA, 25 nt, Homo sapiens
agaucgagacauguaagcagcauca

SEQ ID NO: 70 - RNA, 25 nt, Homo sapiens
ucgagacauguaagcagcaucaugg

SEQ ID NO: 71 - RNA, 25 nt, Homo sapiens
augucucgaucuaugaaaaagacag

SEQ ID NO: 72 - RNA, 25 nt, Homo sapiens
uuuucagguuugaagaugccgcauu

SEQ ID NO: 73 - RNA, 25 nt, Homo sapiens
agguuugaagaugccgcauuuggau

SEQ ID NO: 74 - RNA, 25 nt, Homo sapiens
cacuuacacuuuaugcacaaaaugu

SEQ ID NO: 75 - RNA, 25 nt, Homo sapiens
acuuacacuuuaugcacaaaaugua

SEQ ID NO: 76 - RNA, 25 nt, Homo sapiens
auguaggguuauaauaauguuaaca

SEQ ID NO: 77 - RNA, 25 nt, Homo sapiens
gucuccauguuugauguaucugagc

SEQ ID NO: 78 - RNA, 25 nt, Homo sapiens
gauguaucugagcagguugcuccac

SEQ ID NO: 79 - RNA, 25 nt, Homo sapiens
agcagguugcuccacagguagcucu

SEQ ID NO: 80 - RNA, 25 nt, Homo sapiens
agguugcuccacagguagcucuagg

SEQ ID NO: 81 - RNA, 25 nt, Homo sapiens
gguugcuccacagguagcucuagga

SEQ ID NO: 82 - RNA, 25 nt, Homo sapiens
gcuccacagguagcucuaggagggc

SEQ ID NO: 83 - RNA, 25 nt, Homo sapiens
agcucuaggagggcuggcaacuuag

SEQ ID NO: 84 - RNA, 25 nt, Homo sapiens
ucuaggagggcuggcaacuuagagg

SEQ ID NO: 85 - RNA, 25 nt, Homo sapiens
cuaggagggcuggcaacuuagaggu

SEQ ID NO: 86 - RNA, 25 nt, Homo sapiens
uaggagggcuggcaacuuagaggug

SEQ ID NO: 87 - RNA, 25 nt, Homo sapiens
auucucuuauccaacaucaacaucu

SEQ ID NO: 88 - RNA, 25 nt, Homo sapiens
caauuuacauacucugcuuagaauu

SEQ ID NO: 89 - RNA, 25 nt, Homo sapiens
aauuuacauacucugcuuagaauuu

SEQ ID NO: 90 - RNA, 25 nt, Homo sapiens
auuuacauacucugcuuagaauuug

SEQ ID NO: 91 - RNA, 25 nt, Homo sapiens
uuuacauacucugcuuagaauuugg

SEQ ID NO: 92 - RNA, 25 nt, Homo sapiens
gggaaaauuuagaaauauaauugac

SEQ ID NO: 93 - RNA, 25 nt, Homo sapiens
uuagaaauauaauugacaggauuau

SEQ ID NO: 94 - RNA, 25 nt, Homo sapiens
uacuucuuauacauuugauaaagua

SEQ ID NO: 95 - RNA, 25 nt, Homo sapiens
cuuauacauuugauaaaguaaggca

SEQ ID NO: 96 - RNA, 25 nt, Homo sapiens
cauuugauaaaguaaggcaugguug

SEQ ID NO: 97 - RNA, 25 nt, Homo sapiens
aaguaaggcaugguugugguuaauc

SEQ ID NO: 98 - RNA, 25 nt, Homo sapiens
cuucaaaccugaaaagaaaagaaaa

SEQ ID NO: 99 - RNA, 25 nt, Homo sapiens
auuuggaauucauccaauccaaaug

SEQ ID NO: 100 - RNA, 25 nt, Homo sapiens
uauuaaaaagcaagcaagcagaauu

SEQ ID NO: 101 - RNA, 25 nt, Homo sapiens
gcaaccugcucagauacaucaaaca

SEQ ID NO: 102 - RNA, 25 nt, Homo sapiens
uugccagcccuccuagagcuaccug

SEQ ID NO: 103 - RNA, 25 nt, Homo sapiens
ucaaaucugaccaagauguugaugu

SEQ ID NO: 104 - RNA, 25 nt, Homo sapiens
caaauucuaagcagaguauguaaau

SEQ ID NO: 105 - RNA, 25 nt, Homo sapiens
caaguuuuaugauuuauuuaacuug

SEQ ID NO: 106 - protein, 1386 aa, Artificial Sequence
  Synthetic peptide
MAPKKKRKVDKKYSIGLDIGTNSVGWAVITDEYKVPSKKFKVLGNTDRHSIKKNLIGALLFDSGETAEATRLKRTARRRYTRRKNRILYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIEEFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADHHHHHH

SEQ ID NO: 107 - protein, 1393 aa, Artificial Sequence
  synthetic peptide
MAPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVHHHHHH

SEQ ID NO: 108 - RNA, 20 nt, Homo sapiens
gaguagcgcgagcacagcua

SEQ ID NO: 109 - RNA, 20 nt, Homo sapiens
cgugaguaaaccugaaucuu

SEQ ID NO: 110 - RNA, 20 nt, Homo sapiens
aagucaacuucaaugucgga

SEQ ID NO: 111 - RNA, 20 nt, Homo sapiens
caguaagucaacuucaaugu

SEQ ID NO: 112 - RNA, 20 nt, Homo sapiens
cugaaucuuuggaguaccug

SEQ ID NO: 113 - RNA, 20 nt, Homo sapiens
ggccgagaugucucgcuccg

SEQ ID NO: 114 - RNA, 20 nt, Homo sapiens
cucgcgcuacucucucuuuc

SEQ ID NO: 115 - RNA, 20 nt, Homo sapiens
acucacgcuggauagccucc

SEQ ID NO: 116 - RNA, 20 nt, Homo sapiens
ucacgucauccagcagagaa

SEQ ID NO: 117 - RNA, 20 nt, Homo sapiens
agucacaugguucacacggc

SEQ ID NO: 118 - RNA, 20 nt, Homo sapiens
ccaccucuugauggggcuag

SEQ ID NO: 119 - RNA, 20 nt, Homo sapiens
gcuacucucucuuucuggcc

SEQ ID NO: 120 - RNA, 100 nt, Artificial Sequence
  synthetic nucleotide
gaguagcgcgagcacagcuaguuuuagagcuagaaauagcaaguuaaaauaaggcuaguccguuaucaacuugaaaaaguggcaccgagucggugcuuuu

SEQ ID NO: 121 - protein, 6 aa, Artificial Sequence
  Description of Artificial Sequence: Synthetic 6xHis tag
HHHHHH

SEQ ID NO: 122 - protein, 8 aa, Artificial Sequence
  Description of Artificial Sequence: Synthetic 8xHis tag
HHHHHHHH

SEQ ID NO: 123 - protein, 1368 aa, Artificial Sequence [sequence omitted]

SEQ ID NO: 124 - protein, 1393 aa, Artificial Sequence
  synthetic polypeptide
MAPKKKRKVDKKYSIGLDIGTNSVGWAVITDEYKVPSKKFKVLGNTDRHSIKKNLIGALLFDSGETAEATRLKRTARRRYTRRKNRILYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIEEFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVHHHHHH

SEQ ID NO: 125 - protein, 1399 aa, Artificial Sequence
  synthetic polypeptide
MGSSHHHHHHHHENLYFQGSMDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKV

SEQ ID NO: 126 - protein, 2792 aa, Artificial Sequence
  synthetic polypeptide
MAHHHHHHGGSPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVMAHHHHHHGGSPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKV

SEQ ID NO: 127 - protein, 1386 aa, Artificial Sequence
  synthetic polypeptide
MAPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADHHHHHH

SEQ ID NO: 128 - protein, 1389 aa, Artificial Sequence
  synthetic polypeptide
MAHHHHHHGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKV

SEQ ID NO: 129 - protein, 1399 aa, Artificial Sequence; identical to SEQ ID NO: 125
  synthetic polypeptide

SEQ ID NO: 130 - protein, 1393 aa, Artificial Sequence
  synthetic polypeptide
MAPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNAVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVHHHHHH

SEQ ID NO: 131 - protein, 1393 aa, Artificial Sequence
  synthetic polypeptide
MAPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEALYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPALESEFVYGDYKVYDVRKMIAKSEQEIGKATAKYFFYSNIMNFFKTEITLANGEIRKAPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVHHHHHH

SEQ ID NO: 132 - protein, 1393 aa, Artificial Sequence
  synthetic polypeptide
MAPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLADDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPALESEFVYGDYKVYDVRKMIAKSEQEIGKATAKYFFYSNIMNFFKTEITLANGEIRKAPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVHHHHHH

SEQ ID NO: 133 - protein, 1393 aa, Artificial Sequence
  synthetic polypeptide
MAPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTAFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGALSRKLINGIRDKQSGKTILDFLKSDGFANRNFMALIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRAITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVHHHHHH

SEQ ID NO: 134 - RNA, 20 nt, Homo sapiens
ggccacggagcgagacaucu

SEQ ID NO: 135 - RNA, 20 nt, Homo sapiens
cgcgagcacagcuaaggcca

SEQ ID NO: 136 - RNA, 20 nt, Homo sapiens
aggguaggagagacucacgc

SEQ ID NO: 137 - RNA, 20 nt, Homo sapiens
cacagcccaagauaguuaag

SEQ ID NO: 138 - RNA, 20 nt, Homo sapiens
uuaccccacuuaacuaucuu

SEQ ID NO: 139 - RNA, 20 nt, Homo sapiens
cuuaccccacuuaacuaucu

SEQ ID NO: 140 - RNA, 20 nt, Homo sapiens
uccugaauugcuaugugucu

SEQ ID NO: 141 - DNA, 20 bp, Homo sapiens
gagtagcgcgagcacagcta

SEQ ID NO: 142 - DNA, 20 bp, Homo sapiens
cgtgagtaaacctgaatctt

SEQ ID NO: 143 - DNA, 20 bp, Homo sapiens
aagtcaacttcaatgtcgga

SEQ ID NO: 144 - DNA, 20 bp, Homosapiens
cagtaagtcaacttcaatgt

SEQ ID NO: 145 - DNA, 20 bp, Homosapiens
ctgaatctttggagtacctg

SEQ ID NO: 146 - DNA, 20 bp, Homosapiens
ggccgagatgtctcgctccg

SEQ ID NO: 147 - DNA, 20 bp, Homo sapiens
ctcgcgctactctctctttc

SEQ ID NO: 148 - DNA, 20 bp, Homo sapiens
actcacgctggatagcctcc

SEQ ID NO: 149 - DNA, 20 bp, Homo sapiens
tcacgtcatccagcagagaa

SEQ ID NO: 150 - DNA, 20 bp, Homo sapiens
agtcacatggttcacacggc

SEQ ID NO: 151 - DNA, 20 bp, Homo sapiens
ccacctcttgatggggctag

SEQ ID NO: 152 - DNA, 20 bp, Homo sapiens
gctactctctctttctggcc

SEQ ID NO: 153 - DNA, 20 bp, Homo sapiens
ggccacggagcgagacatct

SEQ ID NO: 154 - DNA, 20 bp, Homo sapiens
cgcgagcacagctaaggcca

SEQ ID NO: 155 - DNA, 20 bp, Homo sapiens
agggtaggagagactcacgc

SEQ ID NO: 156 - DNA, 20 bp, Homo sapiens
cacagcccaagatagttaag

SEQ ID NO: 157 - DNA, 20 bp, Homo sapiens
ttaccccacttaactatctt

SEQ ID NO: 158 - DNA, 20 bp, Homos apiens
cttaccccacttaactatct

SEQ ID NO: 159 - DNA, 20 bp, Homosapiens
tcctgaattgctatgtgtct

SEQ ID NO: 160 - RNA, 100 nt, Artificial Sequence
  synthetic polynucleotide
cgugaguaaaccugaaucuuguuuuagagcuagaaauagcaaguuaaaauaaggcuaguccguuaucaacuugaaaaaguggcaccgagucggugcuuuu

SEQ ID NO: 161 - RNA, 100 nt, Artificial Sequence
  synthetic polynuceotide
aagucaacuucaaugucggaguuuuagagcuagaaauagcaaguuaaaauaaggcuaguccguuaucaacuugaaaaaguggcaccgagucggugcuuuu

SEQ ID NO: 162 - RNA, 100 nt, Artificial Sequence
  synthetic polynucleotide
caguaagucaacuucaauguguuuuagagcuagaaauagcaaguuaaaauaaggcuaguccguuaucaacuugaaaaaguggcaccgagucggugcuuuu

SEQ ID NO: 163 - RNA, 100 nt, Artificial Sequence
  synthetic polynucleotide
cugaaucuuuggaguaccugguuuuagagcuagaaauagcaaguuaaaauaaggcuaguccguuaucaacuugaaaaaguggcaccgagucggugcuuuu

SEQ ID NO: 164 - RNA, 100 nt, Artificial Sequence
  synthetic polynucleotide
ggccgagaugucucgcuccgguuuuagagcuagaaauagcaaguuaaaauaaggcuaguccguuaucaacuugaaaaaguggcaccgagucggugcuuuu

SEQ ID NO: 165 - RNA, 100 nt, Artificial Sequence
  synthetic polynuceotide
cucgcgcuacucucucuuucguuuuagagcuagaaauagcaaguuaaaauaaggcuaguccguuaucaacuugaaaaaguggcaccgagucggugcuuuu

SEQ ID NO: 166 - RNA, 100 nt, Artificial Sequence
  synthetic polynuceotide
acucacgcuggauagccuccguuuuagagcuagaaauagcaaguuaaaauaaggcuaguccguuaucaacuugaaaaaguggcaccgagucggugcuuuu

SEQ ID NO: 167 - RNA, 100 nt, Artificial Sequence
  synthetic polynuceotide
ucacgucauccagcagagaaguuuuagagcuagaaauagcaaguuaaaauaaggcuaguccguuaucaacuugaaaaaguggcaccgagucggugcuuuu

SEQ ID NO: 168 - RNA, 100 nt, Artificial Sequence
  synthetic polynuceotide
agucacaugguucacacggcguuuuagagcuagaaauagcaaguuaaaauaaggcuaguccguuaucaacuugaaaaaguggcaccgagucggugcuuuu

SEQ ID NO: 169 - RNA, 100 nt, Artificial Sequence
  synthetic polynuceotide
ccaccucuugauggggcuagguuuuagagcuagaaauagcaaguuaaaauaaggcuaguccguuaucaacuugaaaaaguggcaccgagucggugcuuuu

SEQ ID NO: 170 - RNA, 100 nt, Artificial Sequence
  synthetic polynuceotide
gcuacucucucuuucuggccguuuuagagcuagaaauagcaaguuaaaauaaggcuaguccguuaucaacuugaaaaaguggcaccgagucggugcuuuu

SEQ ID NO: 171 - RNA, 100 nt, Artificial Sequence
  synthetic polynuceotide
ggccacggagcgagacaucuguuuuagagcuagaaauagcaaguuaaaauaaggcuaguccguuaucaacuugaaaaaguggcaccgagucggugcuuuu

SEQ ID NO: 172 - RNA, 100 nt, Artificial Sequence
  synthetic polynuceotide
cgcgagcacagcuaaggccaguuuuagagcuagaaauagcaaguuaaaauaaggcuaguccguuaucaacuugaaaaaguggcaccgagucggugcuuuu

SEQ ID NO: 173 - RNA, 100 nt, Artificial Sequence
  synthetic polynuceotide
aggguaggagagacucacgcguuuuagagcuagaaauagcaaguuaaaauaaggcuaguccguuaucaacuugaaaaaguggcaccgagucggugcuuuu

SEQ ID NO: 174 - RNA, 100 nt, Artificial Sequence
  synthetic polynucleotide
cacagcccaagauaguuaagguuuuagagcuagaaauagcaaguuaaaauaaggcuaguccguuaucaacuugaaaaaguggcaccgagucggugcuuuu

SEQ ID NO: 175 - RNA, 100 nt, Artificial Sequence
  synthetic polynuceotide
uuaccccacuuaacuaucuuguuuuagagcuagaaauagcaaguuaaaauaaggcuaguccguuaucaacuugaaaaaguggcaccgagucggugcuuuu

SEQ ID NO: 176 - RNA, 100 nt, Artificial Sequence
  synthetic polynuceotide
cuuaccccacuuaacuaucuguuuuagagcuagaaauagcaaguuaaaauaaggcuaguccguuaucaacuugaaaaaguggcaccgagucggugcuuuu

SEQ ID NO: 177 - RNA, 100 nt, Artificial Sequence
  synthetic polynuceotide
uccugaauugcuaugugucuguuuuagagcuagaaauagcaaguuaaaauaaggcuaguccguuaucaacuugaaaaaguggcaccgagucggugcuuuu

SEQ ID NO: 178 - RNA, 20 nt, Artificial Sequence
  synthetic polynuceotide
gacccccuccaccccgccuc

SEQ ID NO: 179 - DNA, 11 bp, Unknown
  Target sequence
  modified_base (1)..(9): a, c, t, g, unknown or other
  misc_feature (1)..(9): n is a, c, g, or t
nnnnnnnnngg

SEQ ID NO: 180 - DNA, 13 bp, Unknown
  target sequence
  modified_base (1)..(8): a, c, t, g, unknown or other
  misc_feature (1)..(8): n is a, c, g, or t
nnnnnnnnagaaw

SEQ ID NO: 181 - DNA, 18 bp, Unknown
  target sequence
  modified_base (1)..(13): a, c, t, g, unknown or other
  misc_feature (1)..(13): n is a, c, g, or t
nnnnnnnnnnnnnagaaw

SEQ ID NO: 182 - DNA, 17 bp, Unknown
  target sequence
  modified_base (1)..(13): a, c, t, g, unknown or other
  misc_feature (1)..(13): n is a, c, g, or t
  modified_base (16)..(16): a, c, t, g, unknown or other
  misc_feature (16)..(16): n is a, c, g, or t
nnnnnnnnnnnnnggng

SEQ ID NO: 183 - DNA, 25 bp, Unknown
  target sequence
  modified_base (10)..(21): a, c, t, g, unknown or other
  misc_feature (10)..(21): n is a, c, g, or t
  modified_base (24)..(24): a, c, t, g, unknown or other
  misc_feature (24)..(24): n is a, c, g, or t
mmmmmmmmmnnnnnnnnnnnnggng